(19) United States
(12) Patent Application Publication
(10) Pub. No.: US 2026/0132149 A1
(43) Pub. Date: May 14, 2026

(54) Bridged Cyclic 5-Amino-6,8-Dihydro-1H-Furo[3,4-D]Pyrrolo[3,2-B]Pyridine-2-Carboxamide Compounds, Compositions Thereof, and Methods of Treatment Therewith

(71) Applicant: BeOne Medicines I GmbH, Basel (CH)

(72) Inventors: Sanjia Xu, Beijing (CN); Yian Guo, Beijing (CN); Jing Li, Beijing (CN); Zhiwei Wang, Beijing (CN)

(73) Assignee: BeOne Medicines I GmbH, Basel (CH)

(21) Appl. No.: 19/386,841

(22) Filed: Nov. 12, 2025

(30) Foreign Application Priority Data
Nov. 13, 2024 (WO) PCT/CN2024/131771
Sep. 15, 2025 (WO) PCT/CN2025/121345

(51) Int. Cl. C07D 519/00; A61K 31/439; A61K 31/4995; A61P 35/00

(52) U.S. Cl. CPC C07D 519/00; A61K 31/439; A61K 31/4995; A61P 35/00

(57) ABSTRACT

Provided herein are compounds having the following structure:

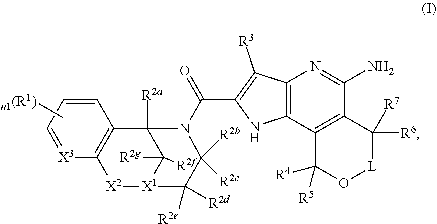

or an N-oxide thereof, or a pharmaceutically acceptable salt, stereoisomer, tautomer, or a deuterated analog thereof, wherein the substituents are as defined herein, their pharmaceutical compositions and uses for modulating the activity of PRMT5 in cooperation with methylthioadenosine (MTA) in tumors bearing MTAP^{DEL} mutations, and their pharmaceutical compositions and methods of use.

### CROSS-REFERENCE TO RELATED APPLICATIONS

This application claims priority to International Application No. PCT/CN2024/131771, filed Nov. 13, 2024, and International Application No. PCT/CN2025/121345, filed Sep. 15, 2025, the contents of each of which are hereby incorporated by reference in their entireties.

### FIELD

Provided herein are bridged cyclic 5-amino-6,8-dihydro-1H-furo[3,4-d]pyrrolo[3,2-b]pyridine-2-carboxamide compounds that inhibit the activity of PRMT5 in cooperation with methylthioadenosine (MTA) in tumors bearing MTAP^{DEL} mutations, and their pharmaceutical compositions and methods of use.

### BACKGROUND

Epigenetic modification is a process that can modify genetic output changing the primary DNA sequence. Epigenetic modification plays an important role in gene expression and regulation, protein production and cell differentiation in multiple dimensions. Typically, this process is reversible and selective, on DNA, its regulatory proteins such as histones and other proteins such as transcription factors [Bradbury, E. M, BioEssays, 1992, 14 (1): pp. 9-16]. PMTs (protein methyltransferases) are central players on epigenetic modifications, consisting of two sub-families named PKMTs (protein lysine methyltransferases) and PRMTs (protein arginine methyltransferases) [Copeland, R. A., et al., Oncogene, 2012. 32 (8): pp. 939-46]. PMTs are associated with various human diseases and considered as potential therapeutic targets [Copeland, R. A., et al., Oncogene, 2012, 32 (8): pp. 939-46].

As the name implies, PRMTs catalyze the methylation of the arginine residues of proteins. Besides their primary functions of methylating the histone tails, PRMTs also target other cellular proteins such as NAB2p, FOXO1, PABP1, Sm D1, etc. [Bedford, M T., et al., Molecular Cell, 2005, 18 (3): pp. 263-72]. Divided by the products, the 9 mammalian PRMTs can be classified into 3 subtypes: type I (PRMT1, PRMT2, PRMT3, PRMT4, PRMT6 and PRMT8) catalyzes aDMA (asymmetrical dimethylated arginine) formation; type II (PRMT5, PRMT9) catalyzes sDMA (symmetrical dimethylated arginine); and type III (PRMT7) catalyzes MMA (monomethylated arginine) formation [Yang, Y., et al., Nature Reviews Cancer, 2012, 13 (1): pp. 37-50]. In addition, type I/II PRMTs can also catalyze MMA formation as an intermediate to aDMA and sDMA. The PRMTs comprise a pocket to interact with its cofactor SAM (S-adenosyl methionine), and an adjacent pocket to interact with the arginine residue on a protein, namely SAM-pocket and substrate-pocket. The methylation process involves an S_{N}2-like mechanism of transferring an activated methyl group from cofactor SAM to the guanidino group on the arginine residue. [Bedford, M T., et al., Molecular Cell, 2005, 18 (3): pp. 263-72]. The side product of the process is SAH (S-adenosyl-L-homocysteine).

The overall arginine level in cells is roughly 1500:3:2:1 for Arg:aDMA:MMA:sDMA, and PRMT5 accounts for the vast majority of sDMA formation [Dhar, S., et al., Scientific Reports, 2013, 3: 1311]. In contrast with PRMT1, the major type I PRMT which functions on its own in cells, PRMT5 binds to MEP50 (methylosome protein 50) to form a heterocomplex that is often elevated in cancer cells and correlates to poor patient survival [Gao, G., et al., Nucleic Acids Research, 2019, 47 (10): pp. 5038-48]. PRMT5 promotes tumorigenesis by varied mechanisms. PRMT5 is a strong repressor of numerous genes; when PRMT5 methylates histones H2a and H4 on Arg3 and histone H3 on Arg8, it represses gene transcripts involved in differentiation, transformation, cell-cycle progression and tumor suppression [Karkhanis, V., et al., Trends in Biochemical Sciences, 2011, 36 (12): pp. 633-41]. Besides its epigenetic roles, PRMT5 may also regulate RNA-binding proteins such as splicing factors. For instance, a reproducible event was observed in PRMT5 knockout mice, in which exon 6 skipping of MDM4 (murine double minute 4) occurred and p53 was released to upregulate p53 pathway [Gerhart, S. V., et al., Scientific Reports, 2018, 8: 9711]. In addition, PRMT5 could directly influence key proliferation pathways by direct methylation of p53 [Jansson, M, et al., Nature Cell Biology, 2008, 10 (12): pp. 1431-9], EGFR [Hsu, J.-M, et al., Nature Cell Biology, 2011, 13 (2): pp. 174-81], PI3K [Wei, T.-Y. W., et al., Cellular Signaling, 2014, 26 (12): pp. 2940-50], etc. Thus, PRMT5 has a good potential to become a clinically relevant target.

On the other hand, PRMT5 is an essential gene in normal tissues, and the systemic inhibition of PRMT5 may result in significant liabilities, especially hematologic toxicity [Ahnert, J. R., et al., Journal of Clinical Oncology, 2021, 39 (15-suppl): p. 3019]. Therefore, strategies to selectively block the PRMT5 activities in tumor cells are required for a safer therapy.

Homozygous deletion of tumor depressor CDKN2A (cyclin dependent kinase inhibitor 2A) occurs in about 15% of all tumor types. Interestingly, the mutation frequently involves the co-deletion of proximate genes existing in 9p21, including the gene that encodes MTAP (methylthioadenosine phosphorylase) [Firestone, R. S., et al., Journal of American Chemical Society, 2017, 139 (39): p. 13754-60]. As a result of MTAP deletion, MTA (methylthioadenosine), the substrate of MTAP, accumulates. MTA is structurally related to SAM, and is a weak ligand/inhibitor of PRMT5 that occupies the same pocket with SAM. The formation of MTA-PRMT5 complex provides chances for further PRMT5 inhibition by formation of a tertiary complex. In such way, a correlation of MTAP null status and dependency of PRMT5 is established through MTA concentration level, to provide a precise oncological therapy.

Currently, there thus remain unmet and continuous medical needs for potent and selective MTA-cooperative PRMT5 inhibitors.

Citation or identification of any reference in this section of this application is not to be construed as an admission that the reference is prior art to the present application.

### SUMMARY

Provided herein is a compound of Formula (I):

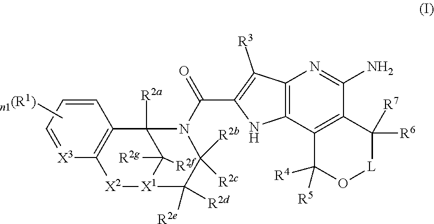

or an N-oxide thereof, or a pharmaceutically acceptable salt thereof, or a stereoisomer thereof, or a tautomer thereof, or a deuterated analog thereof, wherein:
L is a single bond or —C(R⁸R⁹)—;
n1 is 0, 1, 2, 3 or 4;
X¹ is CR^{2h} or N;
X² is C(R²ⁱR^{2j}), O, or NR^{2k}, provided that X² is C(R²ⁱR^{2j}) when X¹ is N;
X³ is N or CH;
R¹ is each independently selected from hydrogen, halogen, substituted or unsubstituted —C₁₋₈alkyl, substituted or unsubstituted —C₂₋₈alkenyl, substituted or unsubstituted —C₂₋₈alkynyl, substituted or unsubstituted —C₃-C₈cycloalkyl, substituted or unsubstituted 3- to 12-membered heterocyclyl, substituted or unsubstituted —C₆-C₁₂aryl, substituted or unsubstituted 5- to 12-membered heteroaryl, —OR^{1a}, —SO₂R^{1a}, —SO₂NR^{1a}R^{1b}, —COR^{1a}, —CO₂R^{1a}, —CONR^{1a}R^{1b}, —NR^{1a}R^{1b}, —NR^{1a}COR^{1b}, —NR^{1a}CO₂R^{1b}, —NR^{1a}SO₂R^{1b}, or —CN;
R^{1a} and R^{1b} are each independently selected from hydrogen, substituted or unsubstituted —C₁₋₈alkyl, substituted or unsubstituted —C₂₋₈alkenyl, substituted or unsubstituted —C₂₋₈alkynyl, substituted or unsubstituted —C₃-C₈cycloalkyl, substituted or unsubstituted 3- to 12-membered heterocyclyl, substituted or unsubstituted —C₆-C₁₂aryl and substituted or unsubstituted 5- to 12-membered heteroaryl;
R^{2a}, R^{2b}, R^{2c}, R^{2d}, R^{2e}, R^{2f}, R^{2g}, R^{2h}, R²ⁱ and R^{2j} are each independently selected from hydrogen, halogen, substituted or unsubstituted —C₁₋₈alkyl, substituted or unsubstituted —C₂₋₈alkenyl, substituted or unsubstituted —C₂₋₈alkynyl, substituted or unsubstituted —C₃-C₈cycloalkyl and substituted or unsubstituted 3- to 8-membered heterocyclyl; or
(R^{2b} and R^{2c}), (R^{2d} and R^{2e}), (R^{2f} and R^{2g}) or (R²ⁱ and R^{2j}) taking together with the atom to which they are attached, form a 3- to 8-membered unsaturated or saturated substituted or unsubstituted ring, said ring comprising 0-3 heteroatoms independently selected from nitrogen, oxygen or sulfur;
R^{2k} are each independently selected from hydrogen, substituted or unsubstituted —C₁₋₈alkyl, substituted or unsubstituted —C₂₋₈alkenyl, substituted or unsubstituted —C₂₋₈alkynyl, substituted or unsubstituted —C₃-C₈cycloalkyl and substituted or unsubstituted 3- to 8-membered heterocyclyl;
R³ is selected from hydrogen, halogen, substituted or unsubstituted —C₁₋₈alkyl, substituted or unsubstituted —C₂₋₈alkenyl, substituted or unsubstituted —C₂₋₈alkynyl, substituted or unsubstituted —C₃-C₈cycloalkyl, —CN, —OH, or —NH₂;
R⁴, R⁵, R⁶, R⁷, R⁸ and R⁹ are each independently selected from hydrogen, halogen, substituted or unsubstituted —C₁₋₈alkyl or substituted or unsubstituted —C₃-C₈cycloalkyl.

Also provided herein is a pharmaceutical composition comprising a compound of Formula (I) or a pharmaceutically acceptable salt, hydrate, or solvate thereof, and a pharmaceutically acceptable excipient.

Also provided herein is a method for inhibiting the activity of PRMT5 in cooperation with methylthioadenosine (MTA) in a cell, comprising contacting said cell with a therapeutically effective amount of a compound of Formula (I), or a pharmaceutically acceptable salt, hydrate, or solvate thereof.

Also provided herein is a method for treating cancer comprising administering to a subject in need thereof a therapeutically effective amount of a compound of formula (I), or a pharmaceutically acceptable salt, hydrate, or solvate thereof.

### DETAILED DESCRIPTION

### Definitions

Unless specifically defined elsewhere in this document, all other technical and scientific terms used herein have the meaning commonly understood by one of ordinary skill in the art.

It is meant to be understood that proper valences are maintained for all moieties and combinations thereof, that monovalent moieties having more than one atom are drawn from left to right and are attached through their left ends, and that divalent moieties are also drawn from left to right.

It is also meant to be understood that a specific embodiment of a variable moiety may be the same or different as another specific embodiment having the same identifier.

The indefinite articles “a” and “an” and the definite article “the” include plural as well as single referents, unless the context clearly indicates otherwise.

“Alkenyl” means a straight or branched hydrocarbon chain having from 2 to 12 carbon atoms and including at least one carbon-carbon double bond. Typically the alkenyl group has from 2 to 8 carbon atoms, preferably from 2 to 6 carbon atoms, and more preferably from 2 to 4 carbon atoms. Representative alkenyl groups include, but are not limited to, -vinyl, -allyl, -1-butenyl, -2-butenyl, -isobutylenyl, -1-pentenyl, 2pentenyl, -3-methyl-1-butenyl, -2-methyl-2-butenyl, -2,3-dimethyl-2-butenyl, -1-hexenyl, 2-hexenyl, -3-hexenyl, -1-heptenyl, -2-heptenyl, -3-heptenyl, -1-octenyl, -2-octenyl, -3-octenyl and the like.

“Alkenylene” means a bivalent straight or branched hydrocarbon radical derived from an alkenyl, as defined above, by removing one hydrogen from a carbon of the alkenyl. The bi-valency can be on the same carbon. Representative alkenylene groups include, but are not limited to, —CH═CH—, —CH₂—CH═CH—, —CH₂—CH═CH—CH₂— and the like.

“Alkyl” means a saturated straight or branched hydrocarbon chain having from 1 to 12 carbon atoms, typically from 1 to 8 carbon atoms, preferably from 1 to 6 carbon atoms, and more preferable from 1 to 4 carbon atoms. Representative alkyl groups include, but are not limited to, -methyl, -ethyl, -n-propyl, -n-butyl, -n-pentyl, -n-hexyl, -isopropyl, -sec-butyl, -isobutyl, tert-butyl, -isopentyl, -neopentyl, tert-pentyl, -2methylpentyl, -3methylpentyl, 4methylpentyl, 2,3dimethylbutyl and the like.

“Alkylene” means a bivalent straight or branched hydrocarbon radical derived from an alkyl, as defined above, by removing one hydrogen from a carbon of the alkyl. The bi-valency can be on the same carbon. Representative alkylene groups include, but are not limited to, —CH₂—, —CH₂CH₂—, —CH(CH₃)—, —(CH₂)₅—, and the like.

“Alkynyl” means a straight or branched hydrocarbon chain having from 2 to 12 carbon atoms and including at least one carbon-carbon triple bonds. Typically the alkynyl group has from 2 to 8 carbon atoms, preferably from 2 to 6 carbon atoms, and more preferable from 2 to 4 carbon atoms. Representative alkynyl groups include, but are not limited to, ethynyl, propynyl, and butynyl.

“Alkynylene” means a bivalent straight or branched hydrocarbon radical derived from an alkynyl, as defined above, by removing one hydrogen from a carbon of the alkynyl. The bi-valency can be on the same carbon. Representative alkynylene groups include, but are not limited to, —C≡C—, —CH₂—C≡C—, —CH₂—C≡C—CH₂— and the like.

“Alkoxy” or “alkoxyl” means —O-alkyl, wherein alkyl is defined herein.

“Amino” means a radical of the formula: —NH₂.

“Aryl” means an aromatic carbocyclic group having from 6 to 14 ring carbon atoms and having a single ring (e.g., phenyl) or multiple fused rings (e.g., naphthyl or anthryl). Preferably, the aryl group is phenyl or naphthyl, and more preferably phenyl.

“Atropisomer” means a stereoisomer resulting from hindered rotation about a single bond axis where the rotational barrier is high enough to allow for the isolation of the individual rotational isomer.

“Bridged ring” means a ring system formed by connecting two non-adjacent atoms (usually carbon atoms) of a ring (mono- or fused multi cyclic) with an atom or chain of atoms. These two non-adjacent atoms are called “bridgeheads”. A bridged ring system may include only carbon atoms as ring atoms or may include carbon atoms and one to four heteroatoms (i.e., N, O, or S) as ring atoms. Examples of bridged ring systems include 1-bicyclo[1.1.1]pentyl, bicyclo[2.1.1]hexyl, bicyclo[2.2.1]heptyl, bicyclo[2.2.2]octyl, adamantanyl, norbornanyl, bicyclo[3.2.1]octyl, bicyclo[2.2.2]octyl, bicyclo[3.3.1]nonyl, bicyclo[3.2.3]nonyl, 2-oxa-bicyclo[2.2.2]octyl, 1-aza-bicyclo[2.2.2]octyl, 3-aza-bicyclo[3.2.1]octyl, and 2,6-dioxa-tricyclo[3.3.1.03,7]nonyl.

“Carboxy” means a radical of the formula: C(═O)OH.

“Cycloalkyl” means a saturated, monocyclic or multiple fused ring system having from 3 to 14 ring carbon atoms and no heteroatoms. Typically, the cycloalkyl group is a monocyclic or bicyclic group having 3 to 10 ring carbon atoms, preferably a monocyclic group having 3 to 8 ring carbon atoms, more preferably a monocyclic group having 3 to 6 ring carbon atoms, and even more preferably a monocyclic group having 5 to 6 ring carbon atoms. Representative cycloalkyl groups include, but are not limited to cyclopropyl, cyclobutyl, cyclopentyl, cyclohexyl, cycloheptyl, and cyclooctyl.

“Cycloalkenyl” means a non-aromatic, unsaturated, monocyclic or multiple fused ring systems group having from 3 to 14 ring carbon atoms and no ring heteroatoms, and having at least one carbon-carbon double bond. Typically, the cycloalkenyl group is a monocyclic or bicyclic group having 3 to 10 ring carbon atoms, preferably a monocyclic group having 3 to 8 ring carbon atoms, more preferably a monocyclic group having 3 to 6 ring carbon atoms, and even more preferably a monocyclic group having 5 to 6 ring carbon atoms. Representative cycloalkenyl groups include, but are not limited to, cyclohexenyl, cyclopentenyl, cyclohexadienyl, butadienyl, pentadienyl, hexadienyl, 1,2,3,4-tetrahydronaphthyl.

“Cycloalkynyl” means a non-aromatic, unsaturated, monocyclic or multiple fused ring system having from 8 to 14 ring carbon atoms and no ring heteroatoms and including at least one carbon-carbon triple bond. Typically, the cycloalkynyl group is a monocyclic group having 8 to 10 ring carbon atoms, preferably 9 to 10 ring carbon atoms. Representative cycloalkynyl groups include, but are not limited to, cyclooctynyl, cyclononynyl, and cyclodecynyl.

The term “deuterated analog” means a compound described herein, whereby an H-isotope, i.e., hydrogen/protium (¹H), is substituted by an H-isotope, i.e., deuterium (²H). Deuterium substitution can be partial or complete. Partial deuterium substitution means that at least one hydrogen is substituted by at least one deuterium. In certain embodiments, the isotope is 90, 95 or 99% or more enriched in an isotope at any location of interest. In some embodiments it is deuterium that is 90, 95 or 99% enriched at a desired location.

“Halogen” means fluorine, chlorine, bromine or iodine.

“Heteroaryl” means an aromatic monocyclic or multiple fused ring system in which at least one, typically one to four, preferably one to three, more preferably one to two, or even more preferably one of the ring carbon atoms are independently replaced with a heteroatom selected from the group consisting of O, S and N. Typically, the heteroaryl group is a monocyclic or bicyclic ring system having 5 to 14 ring atoms, preferably a monocyclic or bicyclic ring system having 5 to 10 ring atoms, and more preferably a monocyclic ring having 5 to 6 ring atoms. Heteroaryl groups can be bonded to other groups at any ring atom (i.e., at any carbon atom or heteroatom of the heteroaryl ring system), provided that proper valences are maintained. Representative heteroaryl groups include, but are not limited to, pyrrolyl, pyrazolyl, imidazolyl, triazolyl, tetrazolyl, oxazolyl, isoxazolyl, benzisoxazolyl (e.g., benzo[d]isoxazolyl), thiazolyl, pyrolyl, pyridazinyl, pyrimidyl, pyrazinyl, thiophenyl, benzothiophenyl, furanyl, benzofuranyl, indolyl (e.g., indolyl-2-onyl or isoindolin-1-onyl), azaindolyl (pyrrolopyridyl or 1H-pyrrolo[2,3-b]pyridyl), indazolyl, benzimidazolyl (e.g., 1Hbenzo[d]imidazolyl), imidazopyridyl (e.g., azabenzimidazolyl or 1H-imidazo[4,5-b]pyridyl), pyrazolopyridyl, triazolopyridyl, benzotriazolyl (e.g., 1H-benzo[d][1,2,3]triazolyl), benzoxazolyl (e.g., benzo[d]oxazolyl), benzothiazolyl, benzothiadiazolyl, isoxazolopyridyl, thianaphthalenyl, purinyl, quinolinyl, isoquinolinyl, quinoxalinyl, and quinazolinyl groups.

“Heterocyclyl” means a non-aromatic monocyclic or multiple fused ring system in which at least one, preferably one to four, more preferably one to three, or even more preferably one to two of the ring carbon atoms are independently replaced with a heteroatom selected from the group consisting of O, S and N, wherein the nitrogen and sulfur heteroatoms may optionally be oxidized and/or the nitrogen heteroatom may be optionally be quarternized. The heterocyclyl group can be saturated or unsaturated. Typically, the heterocyclyl group is a monocyclic or bicyclic ring system having 3 to 14 ring atoms, preferably a monocyclic or bicyclic ring system having 3 to 10 ring atoms, more preferably a monocyclic ring having 3 to 6 ring atoms and even more preferably a monocyclic ring having 5 to 6 atoms. ring atoms The heterocyclyl group can be bonded to other groups at any ring atom (i.e., at any carbon atom or heteroatom of the heterocyclic ring system), provided that proper valences are maintained. Representative heterocyclyl groups include, but are not limited to, aziridinyl, azetidinyl, azepanyl, oxetanyl, pyrrolidyl, imidazolidinyl (e.g., imidazolidin-4-onyl or imidazolidin-2,4-dionyl), pyrazolidinyl, thiazolidinyl, tetrahydrothiophenyl, tetrahydrofuranyl, dioxolyl, pyrrolinyl, imidazolinyl, pyrazolinyl, thiazolinyl, piperidyl, piperazinyl (e.g., piperazin-2-onyl), morpholinyl, thiomorpholinyl, tetrahydropyranyl (e.g., tetrahydro-2H-pyranyl), tetrahydrothiopyranyl, oxathianyl, dioxyl, dithianyl, pyranyl, dihydropyridyl, dihydrodithiinyl, dihydrodithionyl, 1,4-dioxaspiro[4.5]decanyl, 2-oxo-1-oxa-3,8-diazaspiro[4.5]decane, 1-oxo-2,8-diazaspiro[4.5]decane, 3-oxo-2,8-diazaspiro[4.5]decane, 3-oxo-1-oxa-4,9-diazaspiro[5.5]undecane, 2-oxo-1-oxa-3,9-diazaspiro[5.5]undecane, homopiperazinyl, quinuclidyl, indolinyl, isoindolinyl, quinolizinyl, dihydrobenzothiazinyl, dihydrobenzofuranyl, dihydroindolyl, dihydrobenzodioxinyl, tetrahydroquinolinyl groups, and the like.

“Hydrate” means a solvate wherein the solvent molecule is H₂O.

“Oxo” means ═O wherein the double bond is attached to a carbon, sulfur, or nitrogen.

“Pharmaceutically acceptable” means suitable for use in contact with the tissues of human beings without excessive toxicity, irritation, allergic response, or other problem or complication, and commensurate with a reasonable benefit/risk ratio.

“Pharmaceutically acceptable excipient” means a pharmaceutically acceptable inert ingredient or vehicle, such as a liquid or solid filler, diluent, excipient, solvent or encapsulating material. Each excipient must be “acceptable” in the sense of being compatible with the other ingredients of the formulation and not deleterious to the recipients thereof.

“Pharmaceutically acceptable salt(s)” means a salt prepared from a pharmaceutically acceptable non-toxic acid or base including an inorganic acid and base and an organic acid and base.

“Solvate” means a physical association of a compound of formula (I) or a pharmaceutically acceptable salt thereof with one or more pharmaceutically acceptable solvent molecules. This physical association involves varying degrees of ionic and covalent bonding, including hydrogen bonding.

“Spiro ring” means a ring system that contains a bicyclic ring sharing a single ring atom (usually a quaternary carbon atom) between two ring members. Typically the spiro ring is a bicyclic ring system. The individual rings within a spiro ring system may be identical or different, may contain carbon atoms only as ring atoms, or may contain one to four heteroatoms as ring atom(s). Examples of spiro ring systems include spiropentane, spirohexane, spiro[5.4]decane, spiro[4.3]octane, spiro[5.2]octane, and spiropyran.

“Stereoisomer” or “stereomerically pure” means one stereoisomer of a compound that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound.

Stereogenicity at nitrogen in tertiary amines is generally overlooked due to the low energy barriers for pyramidal inversion between the nitrogen-based conformers. embedding nitrogen in bridgehead positions in rigid and noninterconvertible architectures can fix the configuration of a nitrogen center. In a particular scenario, the configuration of Formula (VIc′):

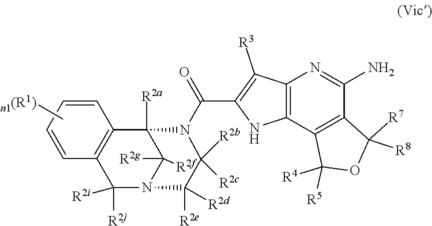

### and Formula (VIc″):

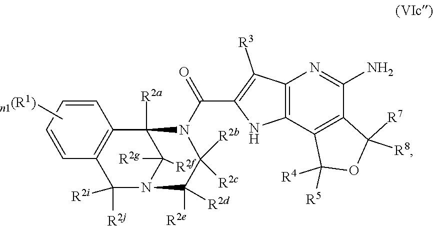

and the stereo equivalents are fixed. No pyramidal inversion on the nitrogen in bridgehead positions can happen.

“Subject” means a mammal. In some preferred embodiments, “Subject” means a human.

“Substituted” means that any one or more hydrogens on the designated atom or group is replaced with a selection (i.e., substituent) from the indicated groups, provided that the designated atom's normal valence is not exceeded. It will be understood by those skilled in the art, with respect to any group containing one or more substituents, that such groups are not intended to introduce any substitution or substitution patterns that are sterically impractical, synthetically non-feasible and/or inherently unstable.

“Tautomer” means an isomeric form of a compound that is in equilibrium with one or more other isomers due to a tautomeric shift, resulting in different arrangements of atoms and chemical bonds. The concentration of the isomeric form will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

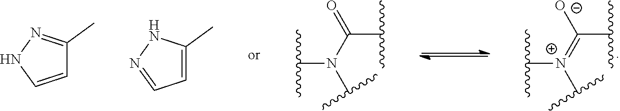

“Therapeutically effective amount” means an amount capable of treating a disorder, disease or condition, or symptoms thereof, disclosed herein.

The terms “treating”, “treatment” and the like, shall include the management and care of a subject or patient, preferably a mammal, more preferably a human, for the purpose of combating a disease, condition, or disorder and includes the administration of a compound of the present invention to prevent the onset of the symptoms or complications, alleviate the symptoms or complications, slow the progression of the disease or disorder, or eliminate the disease, condition, or disorder. The terms “treating” or “treatment” further include: (a) inhibiting the disease-state, i.e., arresting its development, and/or (b) relieving the disease-state, i.e., causing regression of the disease state.

### Compounds

The following aspects and embodiments are not intended to be an explicit or implicit admission that these aspects or embodiments are independent or distinct nor should it be interpreted as such. Rather, it is intended to convey information so that the full breadth of the present disclosure can be understood. Furthermore, the following aspects and embodiments are not meant to be limiting on the full breadth of the disclosure as recited herein.

Aspect 1. A compound of Formula (I):

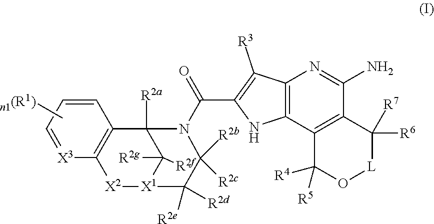

or a hydrate thereof, or a solvate thereof, or an N-oxide thereof, or a pharmaceutically acceptable salt thereof, or a stereoisomer thereof, or a tautomer thereof, or a deuterated analog thereof, wherein:
L is a single bond or —C(R⁸R⁹)—;
n1 is 0, 1, 2, 3 or 4;
X¹ is CR^{2h} or N;
X² is C(R²ⁱR^{2j}), O, NR^{2k} or; provided that X² is C(R²ⁱR^{2j}) when X¹ is N;
X³ is N or CH;
R¹ is each independently selected from hydrogen, halogen, substituted or unsubstituted —C₁₋₈alkyl, substituted or unsubstituted —C₂₋₈alkenyl, substituted or unsubstituted —C₂₋₈alkynyl, substituted or unsubstituted —C₃-C₈cycloalkyl, substituted or unsubstituted 3- to 12-membered heterocyclyl, substituted or unsubstituted —C₆-C₁₂aryl, substituted or unsubstituted 5- to 12-membered heteroaryl, —OR^{1a}, —SO₂R^{1a}, —SO₂NR^{1a}R^{1b}, —COR^{1a}, —CO₂R^{1a}, —CONR^{1a}R^{1b}, —NR^{1a}R^{1b}, —NR^{1a}COR^{1b}, —NR^{1a}CO₂R^{1b}, —NR^{1a}SO₂R^{1b}, and —CN;
R^{1a} and R^{1b} are each independently selected from hydrogen, substituted or unsubstituted —C₁₋₈alkyl, substituted or unsubstituted —C₂₋₈alkenyl, substituted or unsubstituted —C₂₋₈alkynyl, substituted or unsubstituted —C₃-C₈cycloalkyl, substituted or unsubstituted 3- to 12-membered heterocyclyl, substituted or unsubstituted —C₆-C₁₂aryl, and substituted or unsubstituted 5- to 12-membered heteroaryl;
R^{2a}, R^{2b}, R^{2c}, R^{2d}, R^{2e}, R^{2f}, R^{2g}, R^{2h}, R²ⁱ and R^{2j} are each independently selected from hydrogen, halogen, substituted or unsubstituted —C₁₋₈alkyl, substituted or unsubstituted —C₂₋₈alkenyl, substituted or unsubstituted —C₂₋₈alkynyl, substituted or unsubstituted —C₃-C₈cycloalkyl, and substituted or unsubstituted 3- to 8-membered heterocyclyl; or
(R^{2b} and R^{2c}), (R^{2d} and R^{2e}), (R^{2f} and R^{2g}) or (R²ⁱ and R^{2j}) taking together with the atom to which they are attached, form a 3- to 8-membered unsaturated or saturated substituted or unsubstituted ring, said ring comprising 0-3 heteroatoms independently selected from nitrogen, oxygen, and sulfur;
R^{2k} are each independently selected from hydrogen, substituted or unsubstituted —C₁₋₈alkyl, substituted or unsubstituted —C₂₋₈alkenyl, substituted or unsubstituted —C₂₋₈alkynyl, substituted or unsubstituted —C₃-C₈cycloalkyl, and substituted or unsubstituted 3- to 8-membered heterocyclyl;
R³ is selected from hydrogen, halogen, substituted or unsubstituted —C₁₋₈alkyl, substituted or unsubstituted —C₂₋₈alkenyl, substituted or unsubstituted —C₂₋₈alkynyl, substituted or unsubstituted —C₃-C₈cycloalkyl, —CN, —OH, and —NH₂;
R⁴, R⁵, R⁶, R⁷, R⁸ and R⁹ are each independently selected from hydrogen, halogen, substituted or unsubstituted —C₁₋₈alkyl, and substituted or unsubstituted —C₃-C₈cycloalkyl.
   Aspect 2. The compound, or the hydrate, or the solvate, or the N-oxide, or the pharmaceutically acceptable salt, or the stereoisomer, or the tautomer, or the deuterated analog of Aspect 1, wherein:
L is a single bond or —C(R⁸R⁹)—;
n1 is 0, 1, 2, 3 or 4;
X¹ is CR^{2h} or N;
X² is C(R²ⁱR^{2j}), O, NR^{2k} or; provided that X² is C(R²ⁱR^{2j})₂ when X¹ is N;
X³ is N or CH;
R¹ is each independently selected from hydrogen, halogen, —C₁₋₈alkyl, —C₂₋₈alkenyl, —C₂₋₈alkynyl, —C₃-C₈cycloalkyl, 3- to 12-membered heterocyclyl, —C₆-C₁₂aryl, 5- to 12-membered heteroaryl, —OR^{1a}, —SO₂R^{1a}, —SO₂NR^{1a}R^{1b}, —COR^{1a}, —CO₂R^{1a}, —CONR^{1a}R^{1b}, —NR^{1a}R^{1b}, —NR^{1a}COR^{1b}, —NR^{1a}CO₂R^{1b}, —NR^{1a}SO₂R^{1b}, and —CN, wherein each of said —C₁₋₈alkyl, —C₂₋₈alkenyl, —C₂₋₈alkynyl, —C₃-C₈cycloalkyl, 3- to 12-membered heterocyclyl, —C₆-C₁₂aryl and 5- to 12-membered heteroaryl is optionally substituted with at least one substituent R^{1c};
R^{1a} and R^{1b} are each independently selected from hydrogen, —C₁₋₈alkyl, —C₃-C₈cycloalkyl, 3- to 12-membered heterocyclyl, —C₆-C₁₂aryl, and 5- to 12-membered heteroaryl, wherein each of said —C₁₋₈alkyl, —C₃-C₈cycloalkyl, 3- to 12-membered heterocyclyl, —C₆-C₁₂aryl and 5- to 12-membered heteroaryl is optionally substituted with at least one substituent Rid;
R^{1c} and R^{1d} are each independently hydrogen, halogen, —C₁₋₈alkyl, —C₃-C₈cycloalkyl, —C₃-C₁₂cycloalkenyl, 3- to 12-membered heterocyclyl, —C₆-C₁₂aryl, 5- to 12-membered heteroaryl, —OR^{1e}, —SO₂R^{1e}, —SO₂NR^{1e}R^{1f}, —COR^{1e}, —CO₂R^{1e}, —CONR^{1e}R^{1f}, —NR^{1e}R^{1f}, —NR^{1e}COR^{1f}, —NR^{1e}COR₂R^{1f}, —NR^{1e}SO₂R^{1f}, oxo, or —CN, wherein each of said —C₁₋₈alkyl, —C₃-C₈cycloalkyl, —C₃-C₁₂cycloalkenyl, 3- to 12-membered heterocyclyl, —C₆-C₁₂aryl and 5- to 12-membered heteroaryl is optionally substituted with at least one substituent R^{1g};
R^{1e} and R^{1f} are each independently selected from hydrogen, —C₁₋₈alkyl, —C₃-C₈cycloalkyl, 3- to 12-membered heterocyclyl, —C₆-C₁₂aryl, and 5- to 12-membered heteroaryl, wherein each of said —C₁₋₈alkyl, —C₃-C₈cycloalkyl, 3- to 12-membered heterocyclyl, —C₆-C₁₂aryl and 5- to 12-membered heteroaryl is optionally substituted with at least one substituent R^{1h};
R^{1g} and R^{1h} are each independently selected from hydrogen, halogen, —C₁₋₈alkyl, —C₃-C₈cycloalkyl, 3- to 12-membered heterocyclyl, —C₆-C₁₂aryl, and 5- to 12-membered heteroaryl, wherein each of said —C₁₋₈alkyl, —C₃-C₈cycloalkyl, 3- to 12-membered heterocyclyl, —C₆-C₁₂aryl and 5- to 12-membered heteroaryl is optionally substituted with at least one substituent selected from the group consisting of halogen, —C₁₋₈alkyl, —C₁₋₈alkoxy, —C₂₋₈alkenyl, —C₂₋₈alkynyl, —C₃-C₈cycloalkyl, 3- to 8-membered heterocyclyl, —C₆-C₁₂aryl, 5- to 12-membered heteroaryl, —CN, —OH, —NH₂, and oxo;
R^{2a}, R^{2b}, R^{2c}, R^{2d}, R^{2e}, R^{2f}, R^{2g}, R^{2h}, R²ⁱ and R^{2j} are each independently selected from hydrogen, halogen, —C₁₋₈alkyl, —C₂₋₈alkenyl, —C₂₋₈alkynyl, —C₃-C₈cycloalkyl, and 3- to 8-membered heterocyclyl, wherein each of said —C₁₋₈alkyl, —C₂₋₈alkenyl, —C₂₋₈alkynyl, —C₃-C₈cycloalkyl and 3- to 8-membered heterocyclyl is optionally substituted with at least one substituent R^{2aa}; or
(R^{2b} and R^{2c}), (R^{2d} and R^{2e}), (R^{2f} and R^{2g}) or (R²ⁱ and R^{2j}) taking together with the atom to which they are attached, form a 3- to 8-membered unsaturated or saturated ring, said ring comprising 0-3 heteroatoms independently selected from nitrogen, oxygen, and sulfur, and said ring is optionally substituted with at least one substituent R^{2aa};
R^{2aa} is independently hydrogen, halogen, —C₁₋₈alkyl, —C₃-C₈cycloalkyl, —C₃-C₁₂cycloalkenyl, 3- to 12-membered heterocyclyl, —C₆-C₁₂aryl, 5- to 12-membered heteroaryl, —OR^{2ab}, —SO₂R^{2ab}, —SO₂NR^{2ab}R^{2ac}, —COR^{2ab}, —CO₂R^{2ab}, —CONR^{2ab}R^{2ac}, —NR^{2ab}R^{2ac}, —NR^{2ab}COR^{2ac}, —NR^{2ab}CO₂R^{2ac}, —NR^{2ab}SO₂R^{2ac}, oxo, or —CN, wherein each of said —C₁₋₈alkyl, —C₃-C₈cycloalkyl, —C₃-C₁₂cycloalkenyl, 3- to 12-membered heterocyclyl, —C₆-C₁₂aryl and 5- to 12-membered heteroaryl is optionally substituted with at least one substituent R^{2ad};
R^{2ab} and R^{2ac} are each independently selected from hydrogen, —C₁₋₈alkyl, —C₃-C₈cycloalkyl, 3- to 12-membered heterocyclyl, —C₆-C₁₂aryl, and 5- to 12-membered heteroaryl, wherein each of said —C₁₋₈alkyl, —C₃-C₈cycloalkyl, 3- to 12-membered heterocyclyl, —C₆-C₁₂aryl and 5- to 12-membered heteroaryl is optionally substituted with at least one substituent R^{2ae};
R^{2ad} and R^{2ae} are each independently hydrogen, halogen, —C₁₋₈alkyl, —C₃-C₈cycloalkyl, —C₃-C₁₂cycloalkenyl, 3- to 12-membered heterocyclyl, —C₆-C₁₂aryl, 5- to 12-membered heteroaryl, —OR^{2af}, —SO₂R^{2af}, —SO₂NR^{2af}R^{2ag}, —COR^{2af}, —CO₂R^{2af}, —CONR^{2af}R^{2ag}, —NR^{2af}R^{2ag}, —NR^{2af}COR^{2ag}, —NR^{2af}CO₂R^{2ag}, —NR^{2af}SO₂R^{2ag}, oxo, or —CN, wherein each of said —C₁₋₈alkyl, —C₃-C₈cycloalkyl, —C₃-C₁₂cycloalkenyl, 3- to 12-membered heterocyclyl, —C₆-C₁₂aryl and 5- to 12-membered heteroaryl is optionally substituted with at least one substituent R^{2ah};
R^{2af} and R^{2ag} are each independently selected from hydrogen, —C₁₋₈alkyl, —C₃-C₈cycloalkyl, 3- to 12-membered heterocyclyl, —C₆-C₁₂aryl, and 5- to 12-membered heteroaryl, wherein each of said —C₁₋₈alkyl, —C₃-C₈cycloalkyl, 3- to 12-membered heterocyclyl, —C₆-C₁₂aryl and 5- to 12-membered heteroaryl is optionally substituted with at least one substituent R^{2ai};
R^{2ah} and R^{2ai} are each independently hydrogen, halogen, —C₁₋₈alkyl, —C₂₋₈alkynyl, —C₃-C₈cycloalkyl, 3- to 12-membered heterocyclyl, —C₆-C₁₂aryl and 5- to 12-membered heteroaryl, wherein each of said —C₁₋₈alkyl, —C₂₋₈alkynyl, —C₃-C₈cycloalkyl, 3- to 12-membered heterocyclyl, —C₆-C₁₂aryl and 5- to 12-membered heteroaryl is optionally substituted with at least one substituent halogen, —C₁₋₈alkyl, —C₁₋₈alkoxy, —C₂₋₈alkenyl, —C₂₋₈alkynyl, —C₃-C₈cycloalkyl, 3- to 8-membered heterocyclyl, —C₆-C₁₂aryl, 5- to 12-membered heteroaryl, —CN, —OH, —NH₂ or oxo;
R^{2k} are each independently selected from hydrogen, halogen, —C₁₋₈alkyl, —C₂₋₈alkenyl, —C₂₋₈alkynyl, —C₃-C₈cycloalkyl, and 3- to 8-membered heterocyclyl, wherein each of said —C₁₋₈alkyl, —C₂₋₈alkenyl, —C₂₋₈alkynyl, —C₃-C₈cycloalkyl and 3- to 8-membered heterocyclyl is optionally substituted with at least one substituent halogen, —C₁₋₈alkyl, —C₁₋₈alkoxy, —C₂₋₈alkenyl, —C₂₋₈alkynyl, —C₃-C₈cycloalkyl, 3- to 8-membered heterocyclyl, —C₆-C₁₂aryl, 5- to 12-membered heteroaryl, —CN, —OH, —NH₂ or oxo;
R³ is selected from hydrogen, halogen, —C₁₋₈alkyl, —C₃-C₈cycloalkyl, —CN, —OH, and —NH₂, wherein each of said —C₁₋₈alkyl and —C₃-C₈cycloalkyl is optionally substituted with at least one substituent selected from the group consisting of halogen, —C₁₋₈alkoxy, —C₁₋₈alkyl, —C₃-C₈cycloalkyl, and 3- to 8-membered heterocyclyl;
R⁴, R⁵, R⁶, R⁷, R⁸ and R⁹ are each independently selected from hydrogen, halogen, —C₁₋₈alkyl, and —C₃-C₈cycloalkyl, wherein each of said —C₁₋₈alkyl and —C₃-C₈cycloalkyl is optionally substituted with at least one substituent selected from the group consisting of halogen, —C₁₋₈alkoxy, —C₁₋₈alkyl, —C₃-C₈cycloalkyl, 3- to 8-membered heterocyclyl, C₆-C₁₂aryl, 5- to 12-membered heteroaryl, oxo, —CN, —OR^{4a}, —SO₂R^{4a}, —SO₂NR^{4a}R^{4b}, —COR^{4a}, —CO₂R^{4a}, —CONR^{4a}R^{4b}, —NR^{4a}R^{4b}, —NR^{4a}COR^{4b}, —NR^{4a}CO₂R^{4b}, and —NR^{4a}SO₂R^{4b};
R^{4a} and R^{4b} are each independently hydrogen, —C₁₋₈alkyl, C₃-C₈cycloalkyl, 3- to 8-membered heterocyclyl, —C₆-C₁₂aryl, or 5- to 12-membered heteroaryl; each of said —C₁₋₈alkyl, C₃-C₈cycloalkyl, 3- to 8-membered heterocyclyl, —C₆-C₁₂aryl, or 5- to 12-membered heteroaryl is optionally substituted with at least one halogen, —OH, —C₁₋₈alkyl, —C₁₋₈alkoxy, C₁₋₈alkoxy-C₁₋₈alkyl-, —C₂₋₈alkenyl, —C₂₋₈alkynyl, —C₃-C₈cycloalkyl, 3- to 8-membered heterocyclyl, —C₆-C₁₂aryl, or 5- to 12-membered heteroaryl.
   In one embodiment, the compound is Formula (IIa):

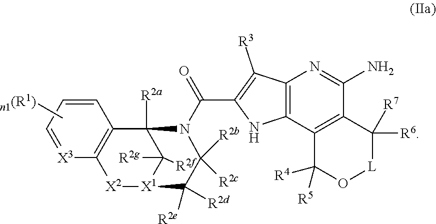

In one embodiment, the compound is Formula (IIb):

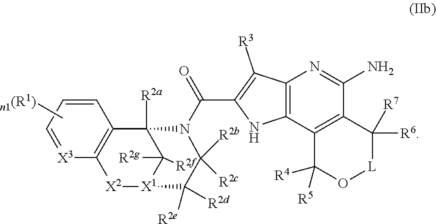

In one embodiment, the compound is Formula (IIIa):

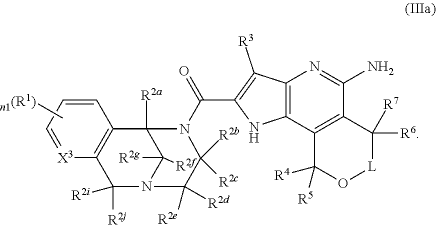

In one embodiment, the compound is Formula (IIIb):

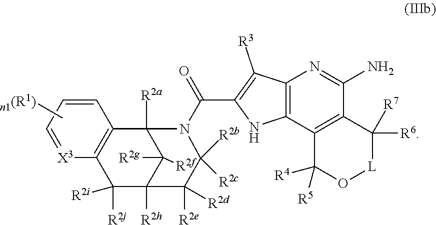

In one embodiment, the compound is Formula (IIIc):

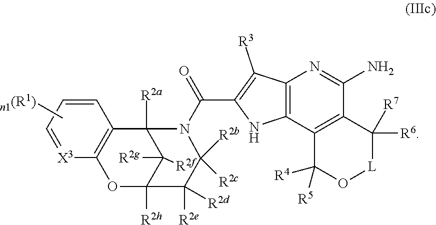

In one embodiment, the compound is Formula (IIId):

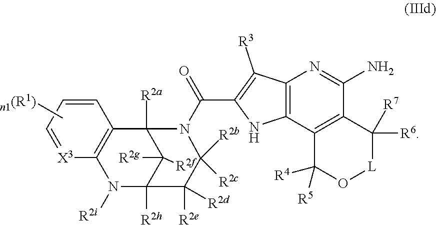

In one embodiment, the compound is Formula (IVa):

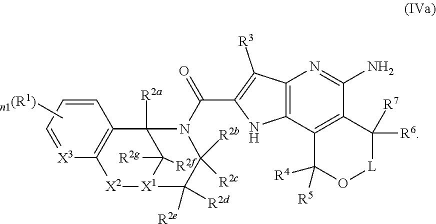

In one embodiment, the compound is Formula (IVb):

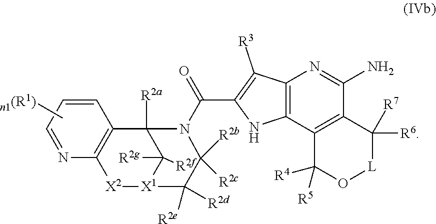

In one embodiment, the compound is Formula (V):

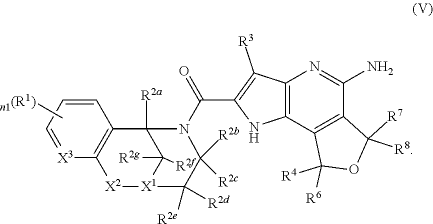

In one embodiment, the compound is Formula (VIa):

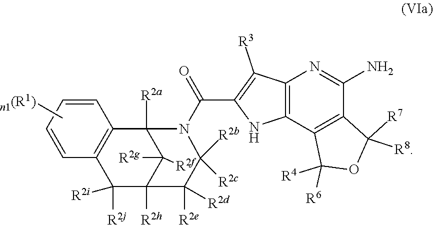

In one embodiment, the compound is Formula (VIa′):

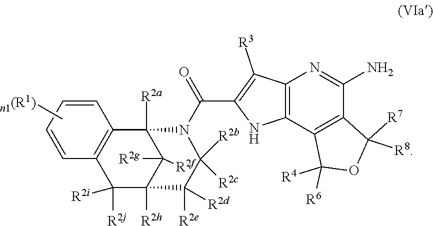

In one embodiment, the compound is Formula (VIa″):

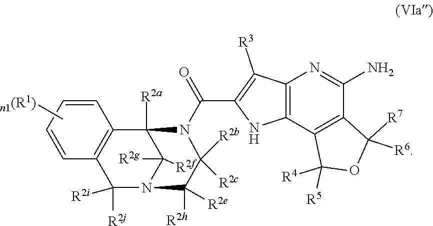

In one embodiment, the compound is Formula (VIb):

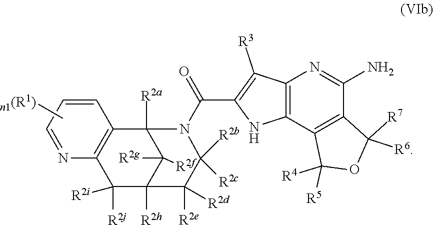

In one embodiment, the compound is Formula (VIb′):

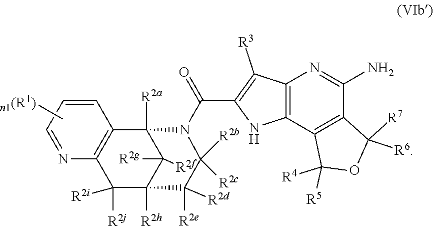

In one embodiment, the compound is Formula (VIb″)

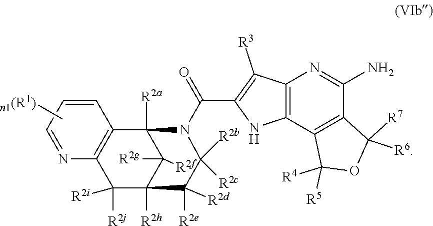

In one embodiment, the compound is Formula (VIc):

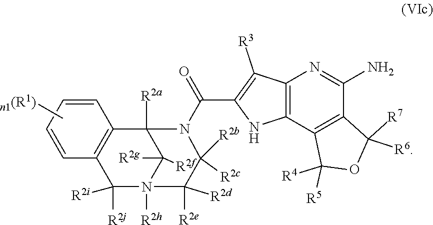

In one embodiment, the compound is Formula (VIc′):

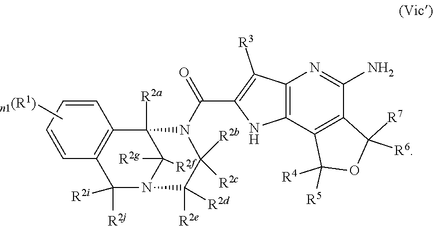

In one embodiment, the compound is Formula (VIc″):

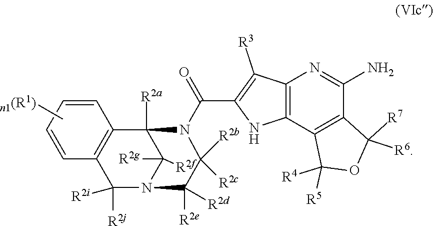

In one embodiment, the compound is Formula (VId):

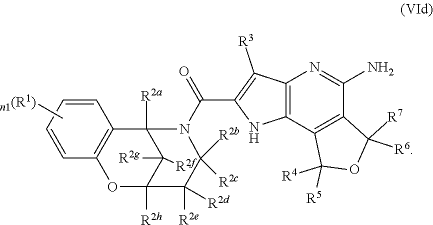

In one embodiment, the compound is Formula (VId′):

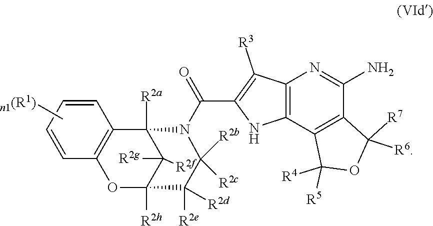

In one embodiment, the compound is Formula (VId″):

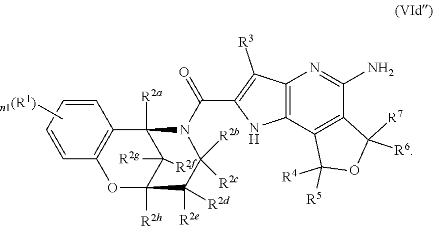

In one embodiment, the compound is Formula (VIe):

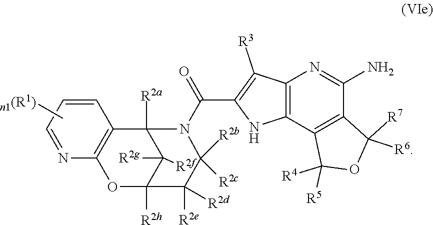

In one embodiment, the compound is Formula (VIe′):

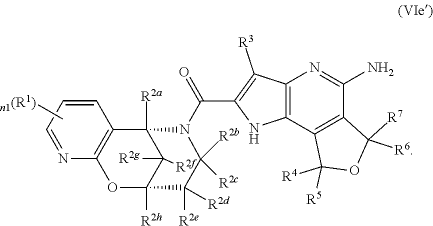

In one embodiment, the compound is Formula (VIe″):

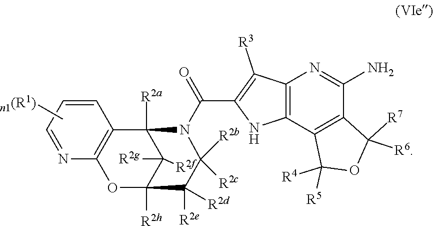

In one embodiment, the compound is Formula (VIf):

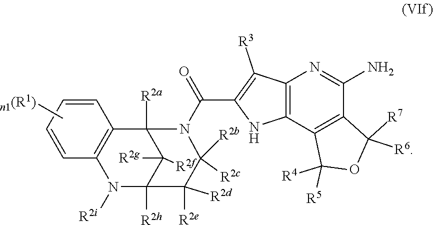

In one embodiment, the compound is Formula (VIf′):

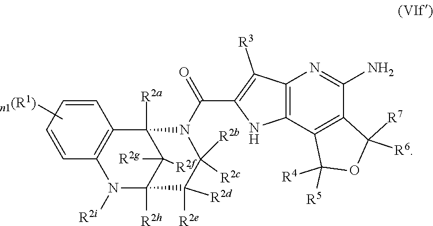

In one embodiment, the compound is Formula (VIf″):

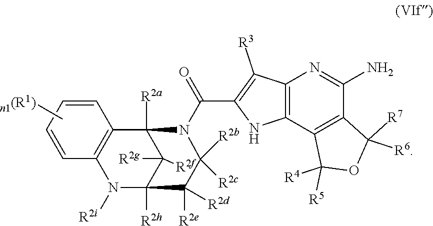

In one embodiment, the compound is Formula (VIIa):

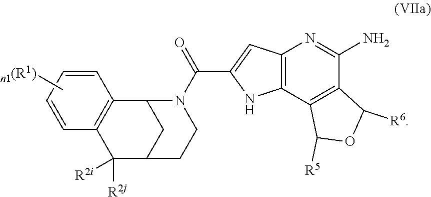

In one embodiment, the compound is Formula (VIIb):

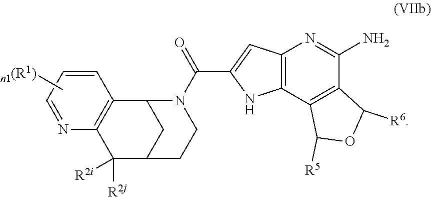

In one embodiment, the compound is Formula (VIIc):

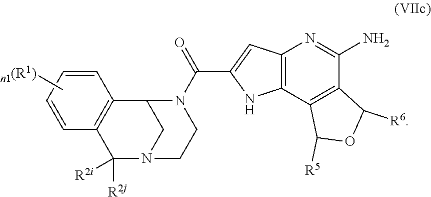

In one embodiment, the compound is Formula (VIId):

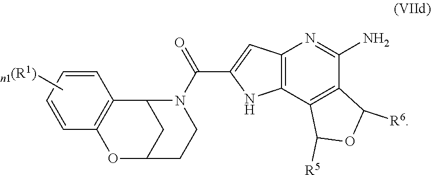

In one embodiment, the compound is Formula (VIIe):

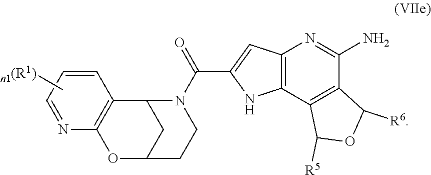

In one embodiment, the compound is Formula (VIIf):

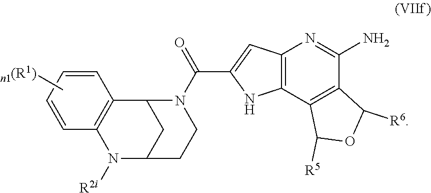

In one embodiment, the compound is Formula (VIIIa):

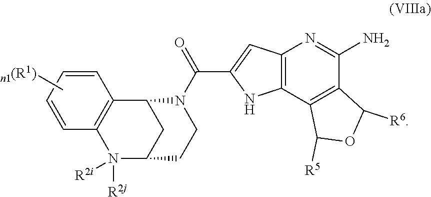

In one embodiment, the compound is Formula (VIIIb):

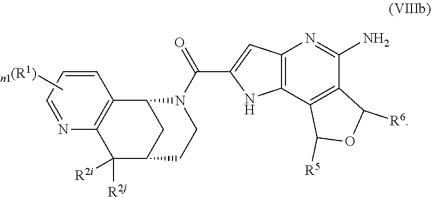

In one embodiment, the compound is Formula (VIIIc):

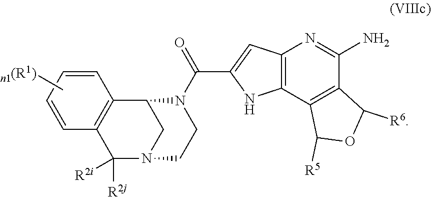

In one embodiment, the compound is Formula (VIIId):

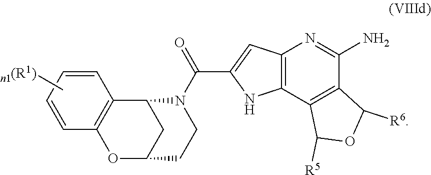

In one embodiment, the compound is Formula (VIIIe):

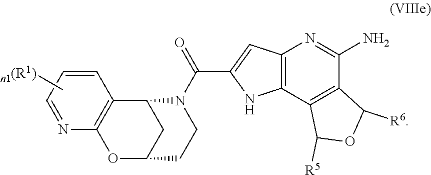

In one embodiment, the compound is Formula (VIIIf):

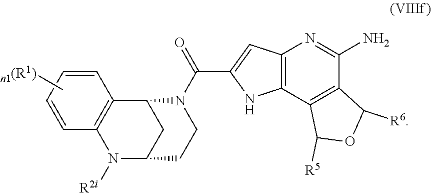

Aspect 3. The compound, or the hydrate, or the solvate, or the N-oxide, or the pharmaceutically acceptable salt, or the stereoisomer, or the tautomer, or the deuterated analog of any one of Aspects 1-2, wherein L is a single bond.
Aspect 4. The compound, or the hydrate, or the solvate, or the N-oxide, or the pharmaceutically acceptable salt, or the stereoisomer, or the tautomer, or the deuterated analog of any one of Aspects 1-3, wherein R¹ is each independently selected from hydrogen, halogen, —C₁₋₆alkyl, —C₂₋₆alkenyl, —C₂₋₆alkynyl, —C₃-C₈cycloalkyl, 3- to 8-membered heterocyclyl, —OR^{1a}, —COR^{1a}, —CO₂R^{1a}, —CONR^{1a}R^{1b}, —NR^{1a}R^{1b}, —NR^{1a}COR^{1b}, and —CN, wherein each of said —C₁₋₈alkyl, —C₂₋₈alkenyl, —C₂₋₈alkynyl, —C₃-C₈cycloalkyl and 3- to 8-membered heterocyclyl is optionally substituted with at least one substituent R^{1c};
R^{1a} and R^{1b} are each independently selected from hydrogen, —C₁₋₆alkyl, —C₃-C₈cycloalkyl and 3- to 8-membered heterocyclyl, wherein each of said —C₁₋₆alkyl, —C₃-C₈cycloalkyl, and 3- to 8-membered heterocyclyl is optionally substituted with at least one substituent R^{1d};
R^{1c} and R^{1d} are each independently hydrogen, halogen, —C₁₋₆alkyl, —C₃-C₈cycloalkyl, 3- to 8-membered heterocyclyl, —OR^{1e}, —COR^{1e}, —CO₂R^{1e}, —CONR^{1e}R^{1f}, —NR^{1e}R^{1f}, —NR^{1e}COR^{1f}, oxo, or —CN, wherein each of said —C₁₋₆alkyl, —C₃-C₈cycloalkyl and 3- to 8-membered heterocyclyl is optionally substituted with at least one substituent R^{1g};
R^{1e} and R^{1f} are each independently selected from hydrogen, —C₁₋₆alkyl, —C₃-C₈cycloalkyl and 3- to 8-membered heterocycly, wherein each of said —C₁₋₆alkyl, —C₃-C₈cycloalkyl, and 3- to 8-membered heterocycly is optionally substituted with at least one substituent R^{1h};
R^{1g} and R^{1h} are each independently selected from hydrogen, halogen, —C₁₋₆alkyl, —C₃-C₈cycloalkyl, and 3- to 8-membered heterocyclyl, wherein each of said —C₁₋₆alkyl, —C₃-C₈cycloalkyl and 3- to 8-membered heterocyclyl is optionally substituted with at least one substituent selected from the group consisting of halogen, —C₁₋₈alkyl, —C₁₋₈alkoxy, —C₂₋₈alkenyl, —C₂₋₈alkynyl, —C₃-C₈cycloalkyl, 3- to 8-membered heterocyclyl, —C₆-C₁₂aryl, 5- to 12-membered heteroaryl, —CN, —OH, —NH₂, and oxo.
   Aspect 5. The compound, or the hydrate, or the solvate, or the N-oxide, or the pharmaceutically acceptable salt, or the stereoisomer, or the tautomer, or the deuterated analog of any one of Aspects 1-4, wherein R¹ is each independently selected from hydrogen, halogen, —C₁₋₆alkyl, —C₂₋₆alkenyl, —C₂₋₆alkynyl, —C₃-C₈cycloalkyl, and 3- to 8-membered heterocyclyl, wherein each of said —C₁₋₈alkyl, —C₂₋₈alkenyl, —C₂₋₈alkynyl, —C₃-C₈cycloalkyl and 3- to 8-membered heterocyclyl is optionally substituted with at least one substituent R^{1e};
R^{1e} is each independently hydrogen, halogen, —C₁₋₆alkyl, —C₃-C₈cycloalkyl, 3- to 8-membered heterocyclyl, —OR^{1e}, —COR^{1e}, —CO₂R^{1e}, —CONR^{1e}R^{1f}, —NR^{1e}R^{1f}, —NR^{1e}COR^{1f}, oxo, or —CN, wherein each of said —C₁₋₆alkyl, —C₃-C₈cycloalkyl and 3- to 8-membered heterocyclyl is optionally substituted with at least one substituent R^{1g};
R^{1e} and R^{1f} are each independently selected from hydrogen, —C₁₋₆alkyl, —C₃-C₈cycloalkyl and 3- to 8-membered heterocycly, wherein each of said —C₁₋₆alkyl, —C₃-C₈cycloalkyl, and 3- to 8-membered heterocycly is optionally substituted with at least one substituent R^{1h};
R^{1g} and R^{1h} are each independently selected from hydrogen, halogen, —C₁₋₆alkyl, —C₃-C₈cycloalkyl and 3- to 8-membered heterocyclyl, wherein each of said —C₁₋₆alkyl, —C₃-C₈cycloalkyl, and 3- to 8-membered heterocyclyl is optionally substituted with at least one substituent selected from the group consisting of halogen, —C₁₋₈alkyl, —C₁₋₈alkoxy, —C₂₋₈alkenyl, —C₂₋₈alkynyl, —C₃-C₈cycloalkyl, 3- to 8-membered heterocyclyl, —C₆-C₁₂aryl, 5- to 12-membered heteroaryl, —CN, —OH, —NH₂, and oxo.
   Aspect 6. The compound, or the hydrate, or the solvate, or the N-oxide, or the pharmaceutically acceptable salt, or the stereoisomer, or the tautomer, or the deuterated analog of any one of Aspects 1-5, wherein R¹ is each independently selected from hydrogen, halogen, —C₁₋₆alkyl, —C₂₋₆alkenyl, —C₂₋₆alkynyl, and —C₃-C₈cycloalkyl, wherein each of said —C₁₋₈alkyl, —C₂₋₈alkenyl, —C₂₋₈alkynyl and —C₃-C₈cycloalkyl is optionally substituted with at least one substituent halogen, —C₁₋₈alkyl, —C₁₋₈alkoxy, —C₂₋₈alkenyl, —C₂₋₈alkynyl, —C₃-C₈cycloalkyl, 3- to 8-membered heterocyclyl, —C₆-C₁₂aryl, 5- to 12-membered heteroaryl, —CN, —OH, —NH₂ or oxo.
   Aspect 7. The compound, or the hydrate, or the solvate, or the N-oxide, or the pharmaceutically acceptable salt, or the stereoisomer, or the tautomer, or the deuterated analog of any one of Aspects 1-6, wherein R¹ is each independently selected from hydrogen, —F, —Br, —Cl, —I, —CH₃, —C₂H₅, —C₃H₇, —C₄H₉, ethynyl, —CF₃, cyclopropyl, cyclobutyl, and cyclopentyl.
   In one embodiment, R¹ is hydrogen. In one embodiment, R¹ is —F. In one embodiment, R¹ is —Cl. In one embodiment, R¹ is —Br. In one embodiment, R¹ is —I. In one embodiment, R¹ is —CH₃. In one embodiment, R¹ is —C₂H₅. In one embodiment, R¹ is —C₃H₇. In one embodiment, R¹ is —C₄H₉. In one embodiment, R¹ is ethynyl. In one embodiment, R¹ is —CF₃. In one embodiment, R¹ is cyclopropyl. In one embodiment, R¹ is cyclobutyl. In one embodiment, R¹ is cyclobutyl.
   Aspect 8. The compound, or the hydrate, or the solvate, or the N-oxide, or the pharmaceutically acceptable salt, or the stereoisomer, or the tautomer, or the deuterated analog of any one of Aspects 1-7, wherein R^{2a}, R^{2b}, R^{2c}, R^{2d}, R^{2e}, R^{2f}, R^{2g}, R^{2h}, R²ⁱ and R^{2j} are each independently selected from hydrogen, halogen, —C₁₋₆alkyl, —C₃-C₈cycloalkyl, and 3- to 8-membered heterocyclyl, wherein each of said —C₁₋₆alkyl, —C₃-C₈cycloalkyl and 3- to 8-membered heterocyclyl is optionally substituted with at least one substituent R^{2aa};
R^{2aa} is independently hydrogen, halogen, —C₁₋₈alkyl, —C₃-C₈cycloalkyl, —C₃-C₁₂cycloalkenyl, 3- to 12-membered heterocyclyl, —C₆-C₁₂aryl, 5- to 12-membered heteroaryl, —OR^{2ab}, —COR^{2ab}, —CO₂R^{2ab}, —CONR^{2ab}R^{2ac}, —NR^{2ab}R^{2ac}, —NR^{2ab}COR^{2ac}, oxo, or —CN, wherein each of said —C₁₋₈alkyl, —C₃-C₈cycloalkyl, —C₃-C₁₂cycloalkenyl, 3- to 12-membered heterocyclyl, —C₆-C₁₂aryl and 5- to 12-membered heteroaryl is optionally substituted with at least one substituent halogen, —C₁₋₈alkyl, —C₁₋₈alkoxy, —C₃-C₈cycloalkyl, 3- to 8-membered heterocyclyl, —C₆-C₁₂aryl, 5- to 12-membered heteroaryl, —CN, —OH, —NH₂ or oxo;
R^{2ab} and R^{2ac} are each independently selected from hydrogen, —C₁₋₈alkyl, —C₃-C₈cycloalkyl, 3- to 12-membered heterocyclyl, —C₆-C₁₂aryl, and 5- to 12-membered heteroaryl, wherein each of said —C₁₋₈alkyl, —C₃-C₈cycloalkyl, 3- to 12-membered heterocyclyl, —C₆-C₁₂aryl and 5- to 12-membered heteroaryl is optionally substituted with at least one substituent halogen, —C₁₋₈alkyl, —C₁₋₈alkoxy, —C₃-C₈cycloalkyl, 3- to 8-membered heterocyclyl, —C₆-C₁₂aryl, 5- to 12-membered heteroaryl, —CN, —OH, —NH₂ or oxo.
   Aspect 9. The compound, or the hydrate, or the solvate, or the N-oxide, or the pharmaceutically acceptable salt, or the stereoisomer, or the tautomer, or the deuterated analog of any one of Aspects 1-8, wherein R^{2a}, R^{2b}, R^{2c}, R^{2d}, R^{2e}, R^{2f}, R^{2g}, R^{2h}, R²ⁱ and R^{2j} are each independently selected from hydrogen, halogen, —C₁₋₄alkyl, —C₃-C₆cycloalkyl, and 3- to 6-membered heterocyclyl, wherein each of said —C₁₋₄alkyl, —C₃-C₆cycloalkyl and 3- to 6-membered heterocyclyl is optionally substituted with at least one substituent halogen, —C₁₋₈alkyl, —C₁₋₈alkoxy, —C₃-C₈cycloalkyl, 3- to 8-membered heterocyclyl, —C₆-C₁₂aryl, 5- to 12-membered heteroaryl, —CN, —OH, —NH₂ or oxo.
   Aspect 10. The compound, or the hydrate, or the solvate, or the N-oxide, or the pharmaceutically acceptable salt, or the stereoisomer, or the tautomer, or the deuterated analog of any one of Aspects 1-9, wherein R^{2a}, R^{2b}, R^{2c}, R^{2d}, R^{2e}, R^{2f}, R^{2g}, R^{2h}, R²ⁱ and R^{2j} are each independently selected from hydrogen, —F, —Cl, —Br, —I, methyl, ethyl, propyl, butyl, cyclopropyl, cyclobutyl, and cyclopentyl.
   Aspect 11. The compound, or the hydrate, or the solvate, or the N-oxide, or the pharmaceutically acceptable salt, or the stereoisomer, or the tautomer, or the deuterated analog of any one of Aspects 1-10, wherein R^{2a}, R^{2b}, R^{2c}, R^{2d}, R^{2e}, R^{2f}, R^{2g} and R^{2h} are each independently selected from hydrogen and, R²ⁱ and R^{2j} are each independently selected from hydrogen, —F, —Cl, —Br, —I, methyl, ethyl, propyl, butyl, cyclopropyl, cyclobutyl, and cyclopentyl; or
R^{2a}, R^{2b}, R^{2c}, R^{2d}, R^{2e}, R^{2f}, R^{2g}, R²ⁱ and R^{2j} are each independently selected from hydrogen, and R^{2h} is selected from hydrogen, —F, —Cl, —Br, —I, methyl, ethyl, propyl, butyl, cyclopropyl, cyclobutyl, and cyclopentyl; or
R^{2a}, R^{2b}, R^{2c}, R^{2f}, R^{2g}, R^{2h}, R²ⁱ and R^{2j} are each independently selected from hydrogen, and R^{2d} and R^{2e} are each independently selected from hydrogen, —F, —Cl, —Br, —I, methyl, ethyl, propyl, butyl, cyclopropyl, cyclobutyl, and cyclopentyl.
   Aspect 12. The compound, or the hydrate, or the solvate, or the N-oxide, or the pharmaceutically acceptable salt, or the stereoisomer, or the tautomer, or the deuterated analog of any one of Aspects 1-7, wherein
(R^{2b} and R^{2c}), (R^{2d} and R^{2e}), (R^{2f} and R^{2g}) or (R²ⁱ and R^{2j}) taking together with the atom to which they are attached, form a 3-, 4-, 5- or 6-membered unsaturated or saturated ring, said ring comprising 0-3 heteroatoms independently selected from nitrogen, oxygen, and sulfur and said ring is optionally substituted with at least one substituent R^{2aa};
R^{2aa} is independently hydrogen, halogen, —C₁₋₈alkyl, —C₃-C₈cycloalkyl, —C₃-C₁₂cycloalkenyl, 3- to 12-membered heterocyclyl, —C₆-C₁₂aryl, 5- to 12-membered heteroaryl, —OR^{2ab}, —COR^{2ab}, —CO₂R^{2ab}, —CONR^{2ab}R^{2ac}, —NR^{2ab}R^{2ac}, —NR^{2ab}COR^{2ac}, oxo, or —CN, wherein each of said —C₁₋₈alkyl, —C₃-C₈cycloalkyl, —C₃-C₁₂cycloalkenyl, 3- to 12-membered heterocyclyl, —C₆-C₁₂aryl and 5- to 12-membered heteroaryl is optionally substituted with at least one substituent halogen, —C₁₋₈alkyl, —C₁₋₈alkoxy, —C₃-C₈cycloalkyl, 3- to 8-membered heterocyclyl, —C₆-C₁₂aryl, 5- to 12-membered heteroaryl, —CN, —OH, —NH₂ or oxo;
R^{2ab} and R^{2ac} are each independently selected from hydrogen, —C₁₋₈alkyl, —C₃-C₈cycloalkyl, 3- to 12-membered heterocyclyl, —C₆-C₁₂aryl, and 5- to 12-membered heteroaryl, wherein each of said —C₁₋₈alkyl, —C₃-C₈cycloalkyl, 3- to 12-membered heterocyclyl, —C₆-C₁₂aryl and 5- to 12-membered heteroaryl is optionally substituted with at least one substituent halogen, —C₁₋₈alkyl, —C₁₋₈alkoxy, —C₃-C₈cycloalkyl, 3- to 8-membered heterocyclyl, —C₆-C₁₂aryl, 5- to 12-membered heteroaryl, —CN, —OH, —NH₂ or oxo.
   Aspect 13. The compound, or the hydrate, or the solvate, or the N-oxide, or the pharmaceutically acceptable salt, or the stereoisomer, or the tautomer, or the deuterated analog of any one of Aspects 1-7, wherein
(R^{2d} and R^{2e}) taking together with the atom to which they are attached, form a 3-, 4-, 5- or 6-membered unsaturated or saturated ring, said ring comprising 0-3 heteroatoms independently selected from nitrogen, oxygen, and sulfur and said ring is optionally substituted with at least one substituent R^{2aa};
R^{2aa} is independently hydrogen, halogen, —C₁₋₈alkyl, —C₃-C₈cycloalkyl, —C₃-C₁₂cycloalkenyl, 3- to 12-membered heterocyclyl, —C₆-C₁₂aryl, 5- to 12-membered heteroaryl, —OR^{2ab}, —COR^{2ab}, —CO₂R^{2ab}, —CONR^{2ab}R^{2ac}, —NR^{2ab}R^{2ac}, —NR^{2ab}COR^{2ac}, oxo, or —CN, wherein each of said —C₁₋₈alkyl, —C₃-C₈cycloalkyl, —C₃-C₁₂cycloalkenyl, 3- to 12-membered heterocyclyl, —C₆-C₁₂aryl and 5- to 12-membered heteroaryl is optionally substituted with at least one substituent halogen, —C₁₋₈alkyl, —C₁₋₈alkoxy, —C₃-C₈cycloalkyl, 3- to 8-membered heterocyclyl, —C₆-C₁₂aryl, 5- to 12-membered heteroaryl, —CN, —OH, —NH₂ or oxo;
R^{2ab} and R^{2ac} are each independently selected from hydrogen, —C₁₋₈alkyl, —C₃-C₈cycloalkyl, 3- to 12-membered heterocyclyl, —C₆-C₁₂aryl, and 5- to 12-membered heteroaryl, wherein each of said —C₁₋₈alkyl, —C₃-C₈cycloalkyl, 3- to 12-membered heterocyclyl, —C₆-C₁₂aryl and 5- to 12-membered heteroaryl is optionally substituted with at least one substituent halogen, —C₁₋₈alkyl, —C₁₋₈alkoxy, —C₃-C₈cycloalkyl, 3- to 8-membered heterocyclyl, —C₆-C₁₂aryl, 5- to 12-membered heteroaryl, —CN, —OH, —NH₂ or oxo.
   Aspect 14. The compound, or the hydrate, or the solvate, or the N-oxide, or the pharmaceutically acceptable salt, or the stereoisomer, or the tautomer, or the deuterated analog of Aspect 13, wherein R^{2aa} is independently hydrogen, halogen, —C₁₋₈alkyl, —C₁₋₈alkoxy, —C₃-C₈cycloalkyl, 3- to 8-membered heterocyclyl, —C₆-C₁₂aryl, 5- to 12-membered heteroaryl, —CN, —OH, —NH₂ or oxo.
   Aspect 15. The compound, or the hydrate, or the solvate, or the N-oxide, or the pharmaceutically acceptable salt, or the stereoisomer, or the tautomer, or the deuterated analog of any one of Aspects 1-7, wherein
(R^{2d} and R^{2e}) taking together with the atom to which they are attached, form a 3-, 4- or 5-membered saturated ring, said ring is optionally substituted with at least one substituent R^{2aa};
R^{2aa} is independently hydrogen, halogen, —C₁₋₈alkyl, —C₁₋₈alkoxy, —C₃-C₈cycloalkyl, 3- to 8-membered heterocyclyl, —C₆-C₁₂aryl, 5- to 12-membered heteroaryl, —CN, —OH, —NH₂ or oxo.

In one embodiment, (R^{2d} and R^{2e}) taking together with the atom to which they are attached, form a 3-membered saturated ring, said ring is optionally substituted with at least one substituent R^{2aa};
R^{2aa} is independently hydrogen, halogen, —C₁₋₈alkyl, —C₁₋₈alkoxy, —C₃-C₈cycloalkyl, 3- to 8-membered heterocyclyl, —C₆-C₁₂aryl, 5- to 12-membered heteroaryl, —CN, —OH, —NH₂ or oxo.

In one embodiment, (R^{2d} and R^{2e}) taking together with the atom to which they are attached, form a 4-membered saturated ring, said ring is optionally substituted with at least one substituent R^{2aa};
R^{2aa} is independently hydrogen, halogen, —C₁₋₈alkyl, —C₁₋₈alkoxy, —C₃-C₈cycloalkyl, 3- to 8-membered heterocyclyl, —C₆-C₁₂aryl, 5- to 12-membered heteroaryl, —CN, —OH, —NH₂ or oxo.

In one embodiment, (R^{2d} and R^{2e}) taking together with the atom to which they are attached, form a 5-membered saturated ring, said ring is optionally substituted with at least one substituent R^{2aa};
R^{2aa} is independently hydrogen, halogen, —C₁₋₈alkyl, —C₁₋₈alkoxy, —C₃-C₈cycloalkyl, 3- to 8-membered heterocyclyl, —C₆-C₁₂aryl, 5- to 12-membered heteroaryl, —CN, —OH, —NH₂ or oxo.
   Aspect 16. The compound, or the hydrate, or the solvate, or the N-oxide, or the pharmaceutically acceptable salt, or the stereoisomer, or the tautomer, or the deuterated analog of any one of Aspects 1-7, wherein
(R²ⁱ and R^{2j}) taking together with the atom to which they are attached, form a 3-, 4-, 5- or 6-membered unsaturated or saturated ring, said ring comprising 0-3 heteroatoms independently selected from nitrogen, oxygen, and sulfur, and said ring is optionally substituted with at least one substituent R^{2aa};
R^{2aa} is independently hydrogen, halogen, —C₁₋₈alkyl, —C₃-C₈cycloalkyl, —C₃-C₁₂cycloalkenyl, 3- to 12-membered heterocyclyl, —C₆-C₁₂aryl, 5- to 12-membered heteroaryl, —OR^{2ab}, —COR^{2ab}, —CO₂R^{2ab}, —CONR^{2ab}R^{2ac}, —NR^{2ab}R^{2ac}, —NR^{2ab}COR^{2ac}, oxo, or —CN, wherein each of said —C₁₋₈alkyl, —C₃-C₈cycloalkyl, —C₃-C₁₂cycloalkenyl, 3- to 12-membered heterocyclyl, —C₆-C₁₂aryl and 5- to 12-membered heteroaryl is optionally substituted with at least one substituent halogen, —C₁₋₈alkyl, —C₁₋₈alkoxy, —C₃-C₈cycloalkyl, 3- to 8-membered heterocyclyl, —C₆-C₁₂aryl, 5- to 12-membered heteroaryl, —CN, —OH, —NH₂ or oxo;
R^{2ab} and R^{2ac} are each independently selected from hydrogen, —C₁₋₈alkyl, —C₃-C₈cycloalkyl, 3- to 12-membered heterocyclyl, —C₆-C₁₂aryl, and 5- to 12-membered heteroaryl, wherein each of said —C₁₋₈alkyl, —C₃-C₈cycloalkyl, 3- to 12-membered heterocyclyl, —C₆-C₁₂aryl and 5- to 12-membered heteroaryl is optionally substituted with at least one substituent halogen, —C₁₋₈alkyl, —C₁₋₈alkoxy, —C₃-C₈cycloalkyl, 3- to 8-membered heterocyclyl, —C₆-C₁₂aryl, 5- to 12-membered heteroaryl, —CN, —OH, —NH₂ or oxo.
   Aspect 17. The compound, or the hydrate, or the solvate, or the N-oxide, or the pharmaceutically acceptable salt, or the stereoisomer, or the tautomer, or the deuterated analog of Aspect 16, wherein R^{2aa} is independently hydrogen, halogen, —C₁₋₈alkyl, —C₁₋₈alkoxy, —C₃-C₈cycloalkyl, 3- to 8-membered heterocyclyl, —C₆-C₁₂aryl, 5- to 12-membered heteroaryl, —CN, —OH, —NH₂ or oxo.
   Aspect 18. The compound, or the hydrate, or the solvate, or the N-oxide, or the pharmaceutically acceptable salt, or the stereoisomer, or the tautomer, or the deuterated analog of any one of Aspects 1-7, wherein
(R²ⁱ and R^{2j}) taking together with the atom to which they are attached, form a 3-, 4- or 5-membered saturated ring, said ring is optionally substituted with at least one substituent R^{2aa};
R^{2aa} is independently hydrogen, halogen, —C₁₋₈alkyl, —C₁₋₈alkoxy, —C₃-C₈cycloalkyl, 3- to 8-membered heterocyclyl, —C₆-C₁₂aryl, 5- to 12-membered heteroaryl, —CN, —OH, —NH₂ or oxo.

In one embodiment, (R²ⁱ and R^{2j}) taking together with the atom to which they are attached, form a 3-membered saturated ring, said ring is optionally substituted with at least one substituent R^{2aa};
R^{2aa} is independently hydrogen, halogen, —C₁₋₈alkyl, —C₁₋₈alkoxy, —C₃-C₈cycloalkyl, 3- to 8-membered heterocyclyl, —C₆-C₁₂aryl, 5- to 12-membered heteroaryl, —CN, —OH, —NH₂ or oxo.

In one embodiment, (R²ⁱ and R^{2j}) taking together with the atom to which they are attached, form a 4-membered saturated ring, said ring is optionally substituted with at least one substituent R^{2aa};
R^{2aa} is independently hydrogen, halogen, —C₁₋₈alkyl, —C₁₋₈alkoxy, —C₃-C₈cycloalkyl, 3- to 8-membered heterocyclyl, —C₆-C₁₂aryl, 5- to 12-membered heteroaryl, —CN, —OH, —NH₂ or oxo.

In one embodiment, (R²ⁱ and R^{2j}) taking together with the atom to which they are attached, form a 5-membered saturated ring, said ring is optionally substituted with at least one substituent R^{2aa};
R^{2aa} is independently hydrogen, halogen, —C₁₋₈alkyl, —C₁₋₈alkoxy, —C₃-C₈cycloalkyl, 3- to 8-membered heterocyclyl, —C₆-C₁₂aryl, 5- to 12-membered heteroaryl, —CN, —OH, —NH₂ or oxo.
   Aspect 19. The compound, or the hydrate, or the solvate, or the N-oxide, or the pharmaceutically acceptable salt, or the stereoisomer, or the tautomer, or the deuterated analog of any one of Aspects 1-18, wherein R³ is selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, —CN, —OH, and —NH₂.
   Aspect 20. The compound, or the hydrate, or the solvate, or the N-oxide, or the pharmaceutically acceptable salt, or the stereoisomer, or the tautomer, or the deuterated analog of any one of Aspects 1-19, wherein R³ is selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, —CN, and —OH.
   Aspect 21. The compound, or the hydrate, or the solvate, or the N-oxide, or the pharmaceutically acceptable salt, or the stereoisomer, or the tautomer, or the deuterated analog of any one of Aspects 1-20, wherein R³ is selected from hydrogen, —F, —Cl, methyl, ethyl, propyl, and butyl.
   Aspect 22. The compound, or the hydrate, or the solvate, or the N-oxide, or the pharmaceutically acceptable salt, or the stereoisomer, or the tautomer, or the deuterated analog of any one of Aspects 1-21, wherein R³ is hydrogen.
   Aspect 23. The compound, or the hydrate, or the solvate, or the N-oxide, or the pharmaceutically acceptable salt, or the stereoisomer, or the tautomer, or the deuterated analog of any one of Aspects 1-22, wherein R⁴, R⁵, R⁶, R⁷, R⁸ and R⁹ are each independently selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, and cyclooctyl, wherein each of said methyl, ethyl, propyl, butyl, pentyl, hexyl, heptyl, octyl, cyclopropyl, cyclobutyl, cyclopentyl, cyclohexyl, cycloheptyl or cyclooctyl is optionally substituted with at least one substituent selected from —F, —Cl, —Br, —I, methoxy, ethoxy, propoxy, butoxy, pentoxy, hexoxy, hepthoxy, octoxy, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo, —CN, —OR^{4a}, —SO₂R^{4a}, —SO₂NR^{4a}R^{4b}, —COR^{4a}, —CO₂R^{4a}, —CONR^{4a}R^{4b}, —NR^{4a}R^{4b}, —NR^{4a}COR^{4b}, —NR^{4a}CO₂R^{4b}, and —NR^{4a}SO₂R^{4b}; and
R^{4a} and R^{4b} are each independently hydrogen, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl, or 5- to 12-membered heteroaryl; each of said methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl, or 5- to 12-membered heteroaryl is optionally substituted with at least one —F, —Cl, —Br, —I, —OH, methyl, ethyl, propyl, butyl, pentyl, hexyl, heptyl, octyl, methoxy, ethoxy, propoxy, butoxy, pentoxy, hexoxy, hepthoxy, octoxy, C₁₋₈alkoxy-C₁₋₈alkyl-, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl.
   Aspect 24. The compound, or the hydrate, or the solvate, or the N-oxide, or the pharmaceutically acceptable salt, or the stereoisomer, or the tautomer, or the deuterated analog of any one of Aspects 1-23, wherein R⁴, R⁵, R⁶, R⁷, R⁸ and R⁹ are each independently selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, and cyclooctyl.
   Aspect 25. The compound, or the hydrate, or the solvate, or the N-oxide, or the pharmaceutically acceptable salt, or the stereoisomer, or the tautomer, or the deuterated analog of any one of Aspects 1-24, wherein R⁴, R⁵, R⁶, R⁷, R⁸ and R⁹ are each independently selected from hydrogen, —F, —Cl, —Br, —I, methyl, ethyl, propyl, cyclopropyl, and cyclobutyl.
   Aspect 26. The compound, or the hydrate, or the solvate, or the N-oxide, or the pharmaceutically acceptable salt, or the stereoisomer, or the tautomer, or the deuterated analog of any one of Aspects 1-25, wherein R⁴, R⁵, R⁶, R⁷, R⁸ and R⁹ are each independently selected from hydrogen, —F, —Cl, —Br, —I, and methyl.
   Aspect 27. The compound, or the hydrate, or the solvate, or the N-oxide, or the pharmaceutically acceptable salt, or the stereoisomer, or the tautomer, or the deuterated analog of any one of Aspects 1-26, wherein R⁴, R⁵, R⁶, R⁷, R⁸ and R⁹ are each independently hydrogen or methyl.
   In one embodiment, R⁴ is hydrogen. In one embodiment, R⁴ is methyl. In one embodiment, R⁵ is hydrogen.

In one embodiment, R⁵ is methyl. In one embodiment, R⁴ is hydrogen, and R⁵ is methyl. In one embodiment, R⁴ is hydrogen, and R⁵ is hydrogen.

In one embodiment, R⁶ is hydrogen. In one embodiment, R⁶ is methyl. In one embodiment, R⁷ is hydrogen.

In one embodiment, R⁷ is methyl. In one embodiment, R⁶ is hydrogen, and R⁷ is methyl. In one embodiment, R⁶ is hydrogen, and R⁷ is hydrogen.

In one embodiment, R⁸ is hydrogen, and R⁹ is hydrogen.
Aspect 28. The compound, or the hydrate, or the solvate, or the N-oxide, or the pharmaceutically acceptable salt, or the stereoisomer, or the tautomer, or the deuterated analog of Aspect 1, wherein the

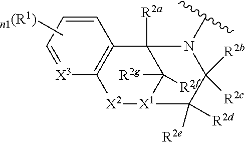

moiety is selected from

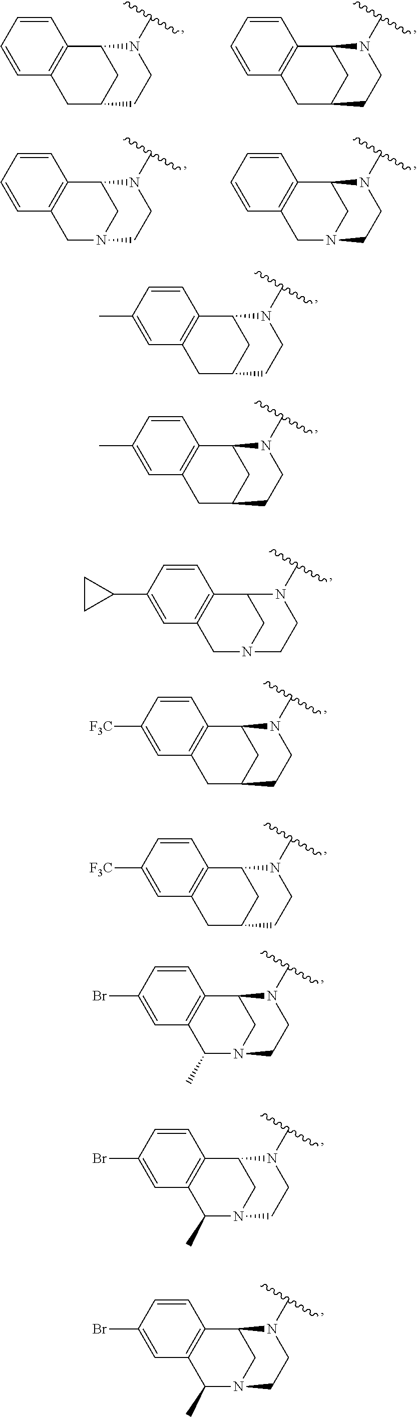

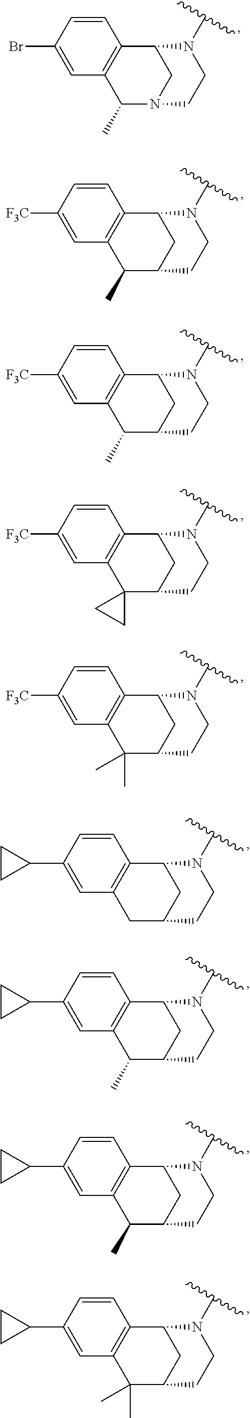

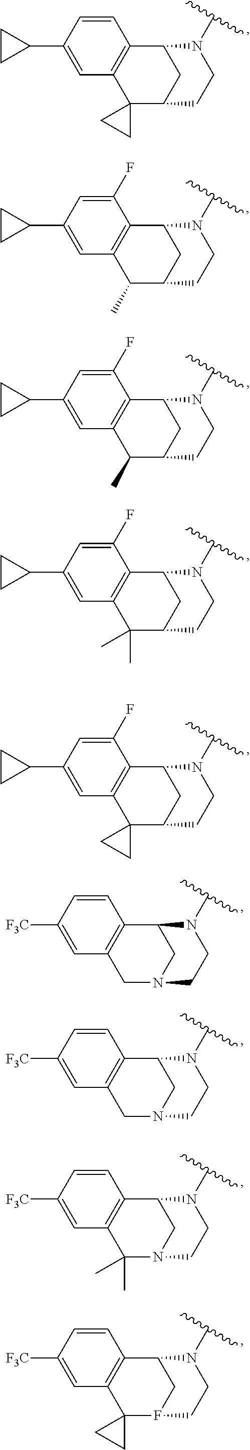

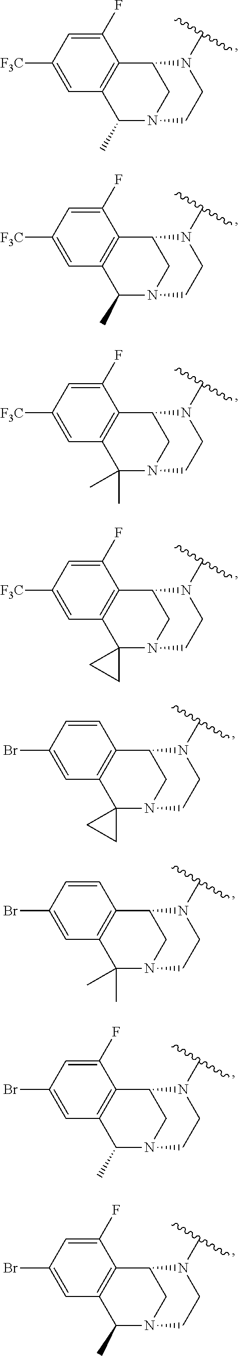

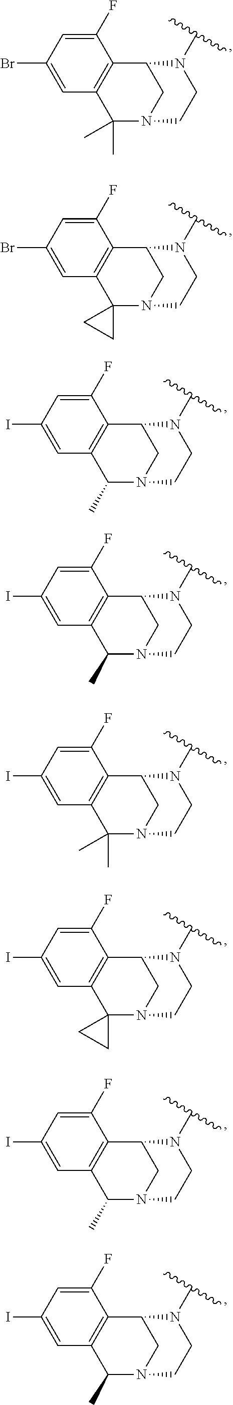

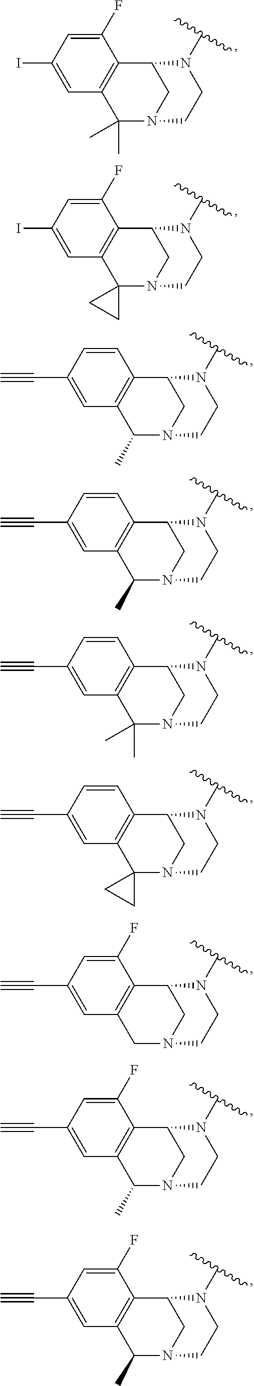

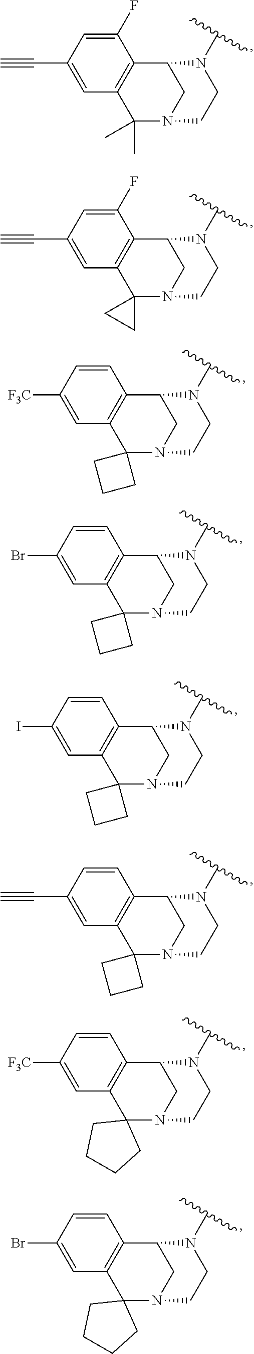

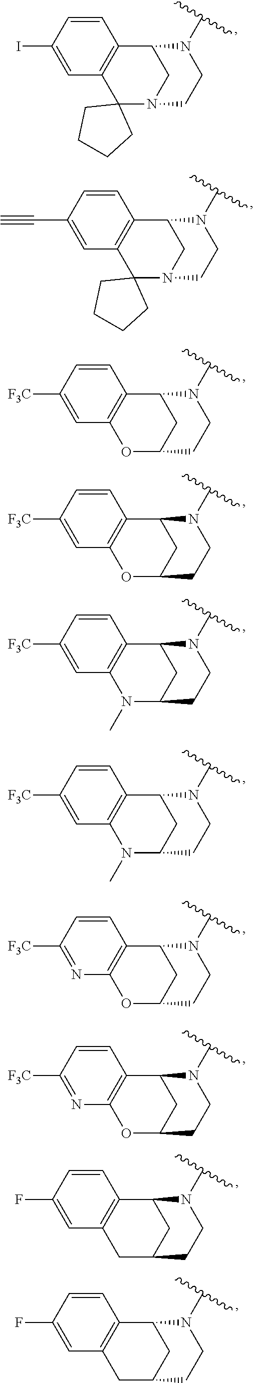

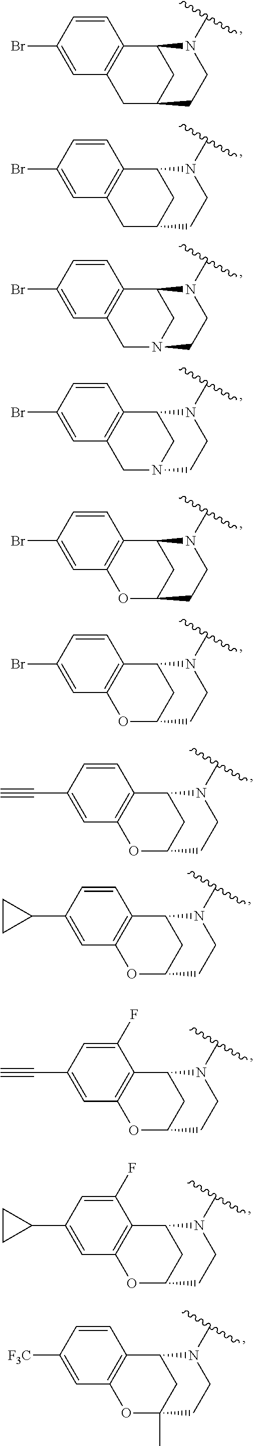

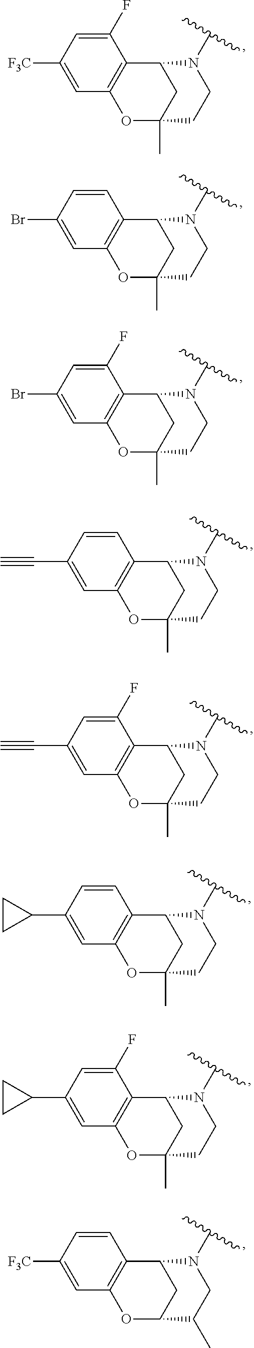

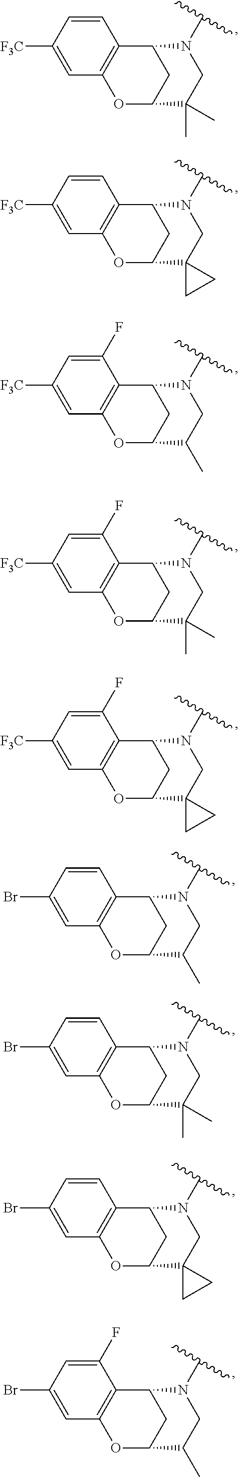

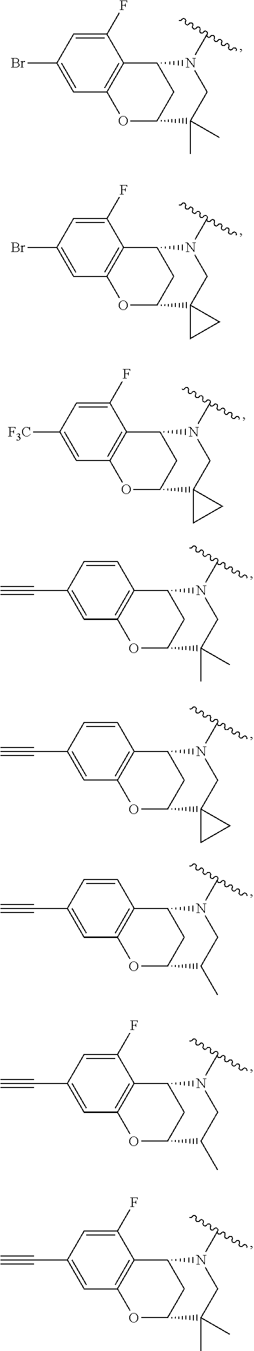

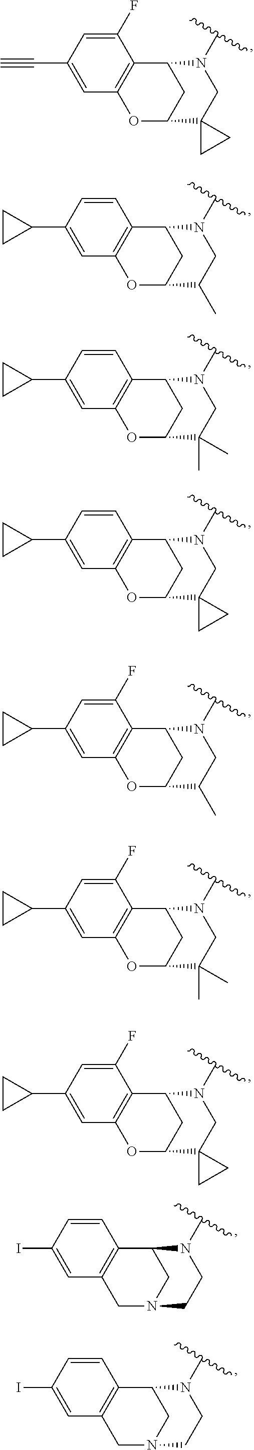

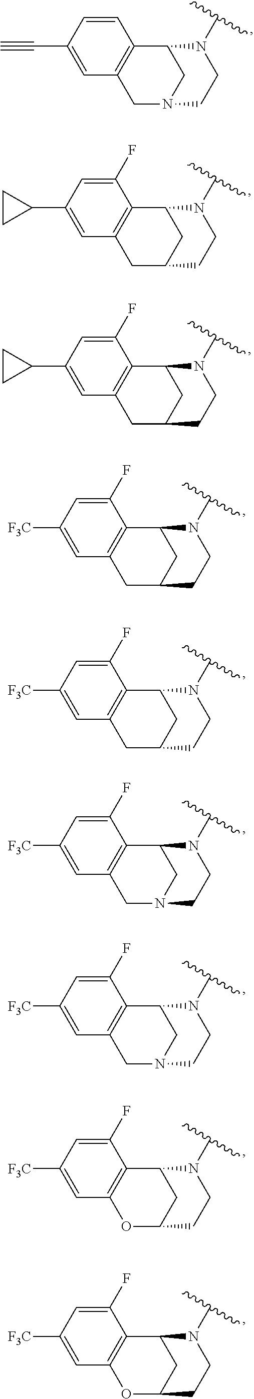

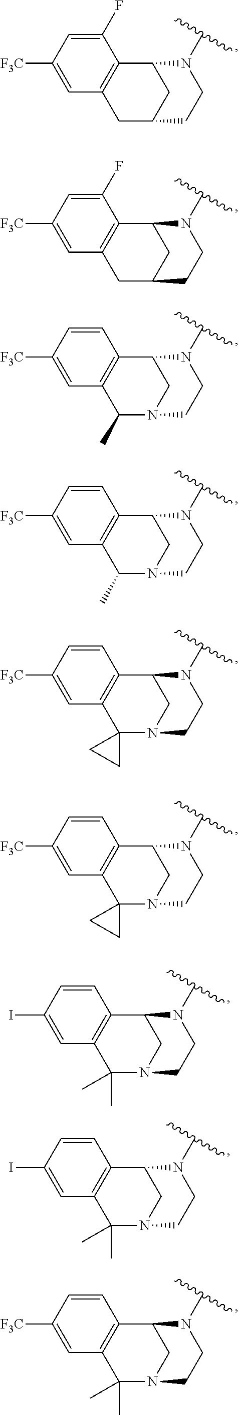

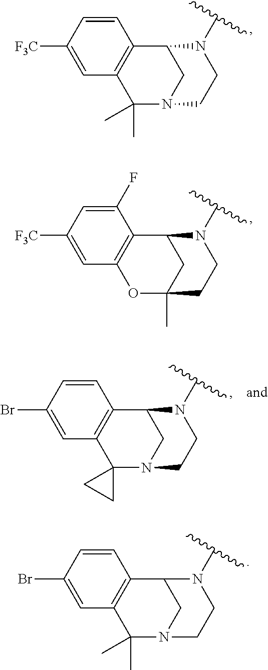

Aspect 29. A compound, or a hydrate thereof, or a solvate thereof, or an N-oxide thereof, or a pharmaceutically acceptable salt thereof, or a stereoisomer thereof, or a tautomer thereof, or a deuterated analog thereof, wherein the compound is selected from:

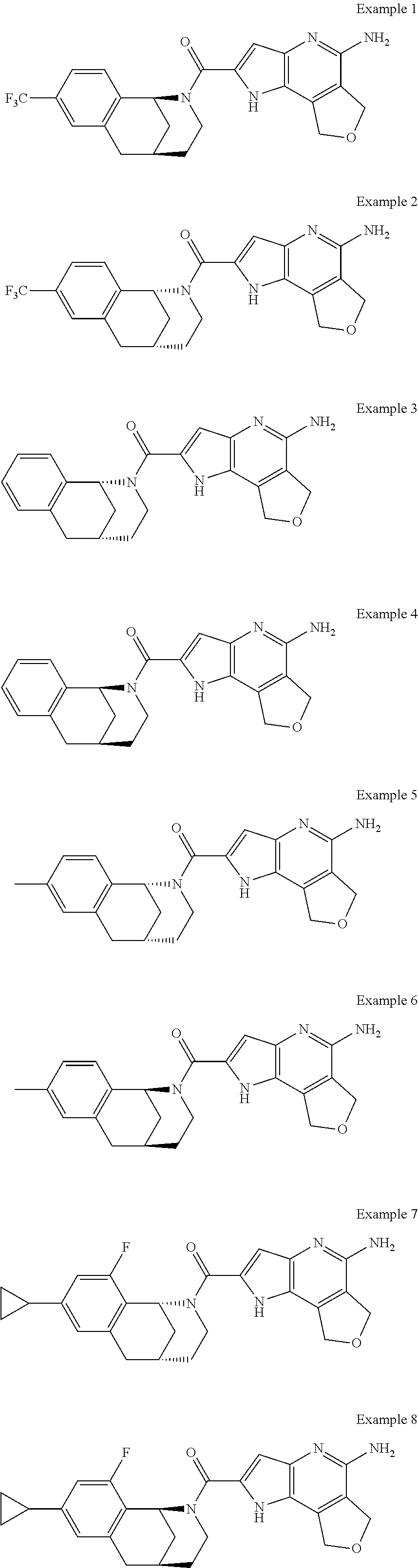

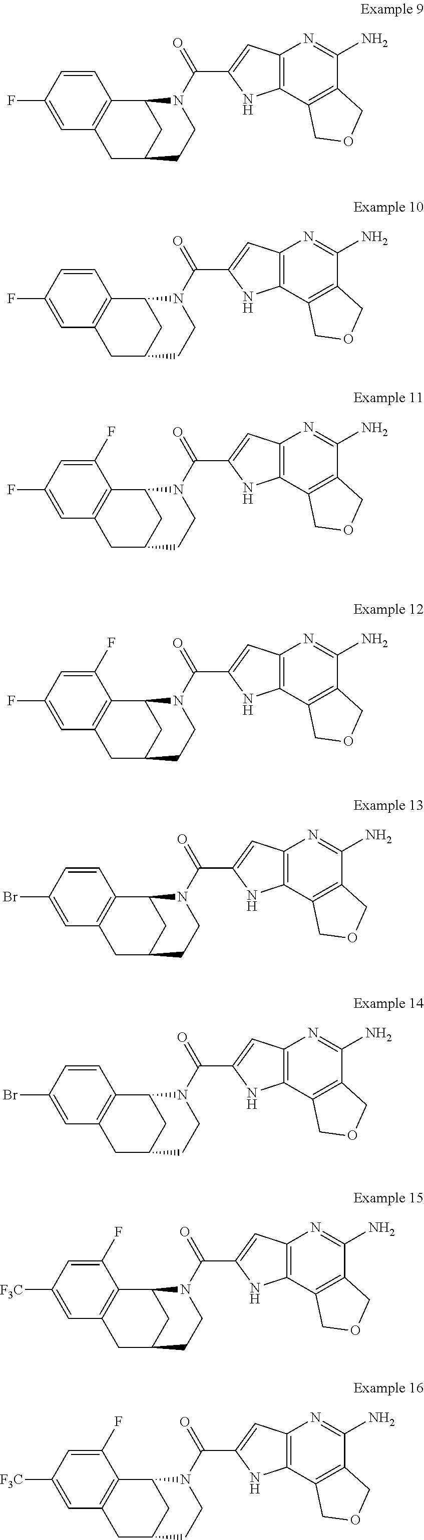

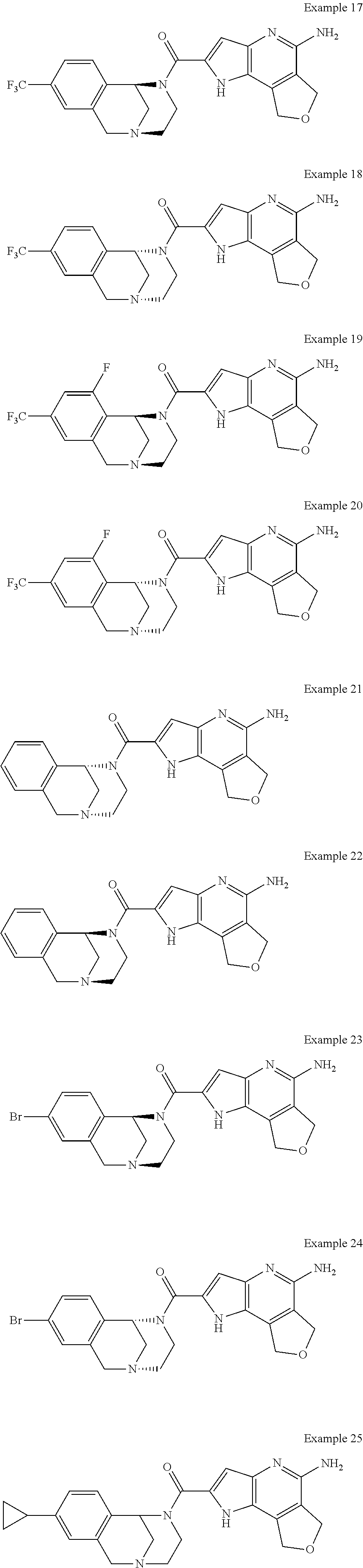

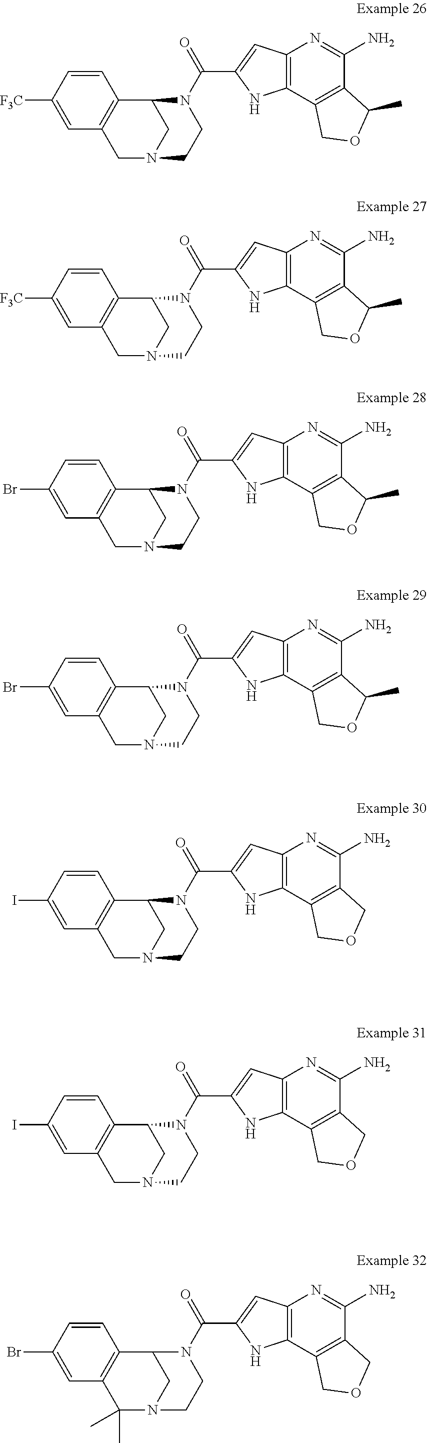

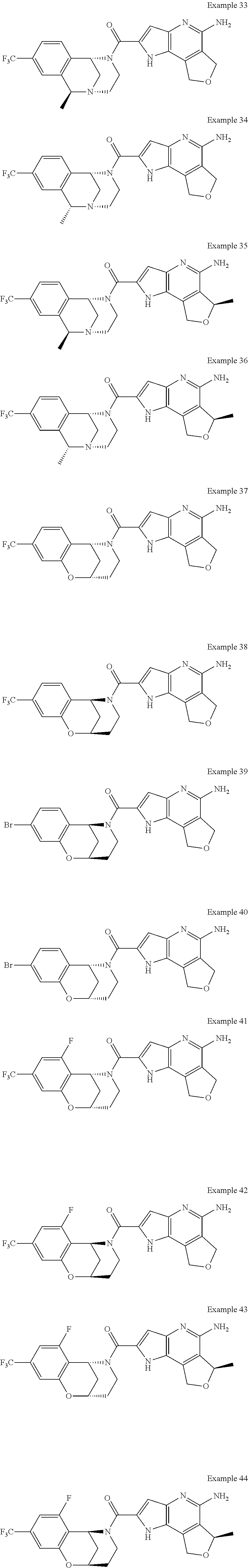

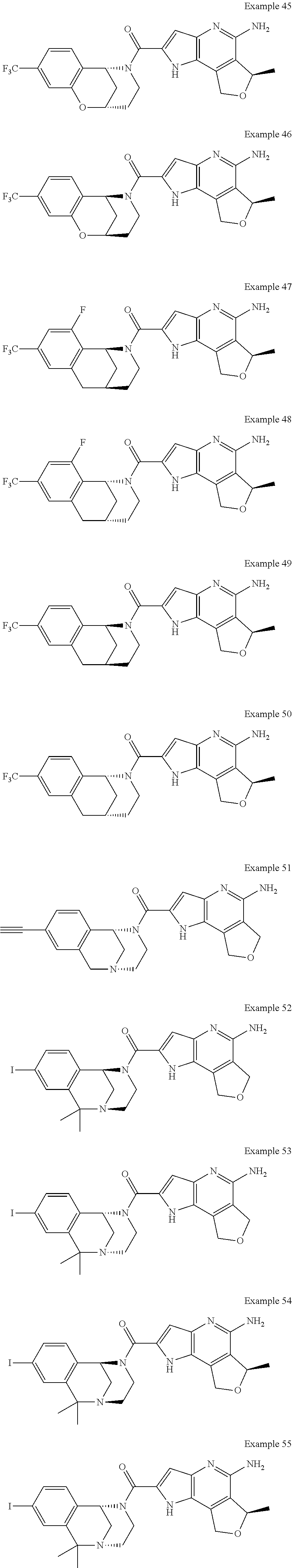

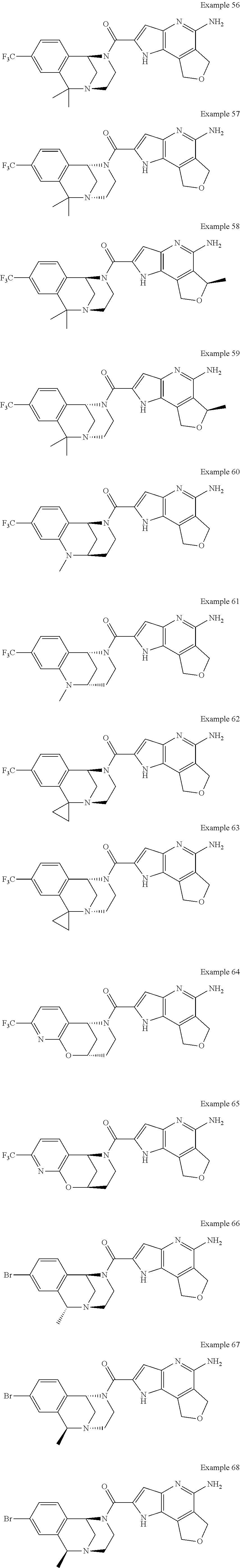

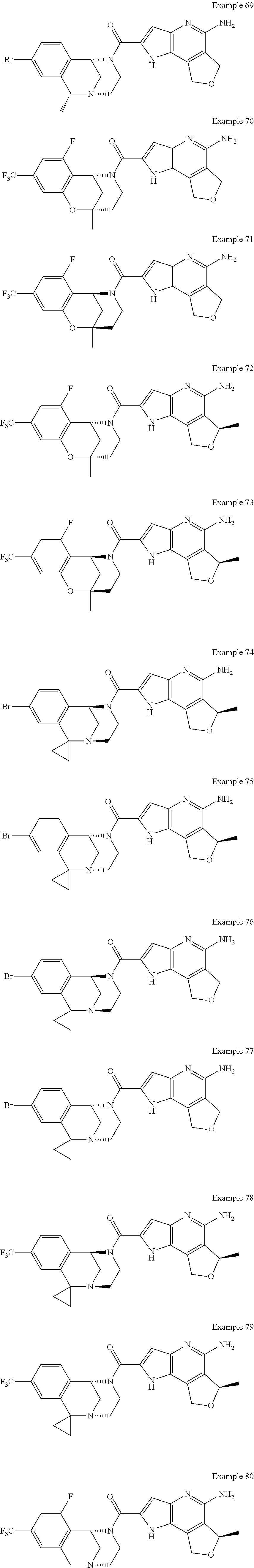

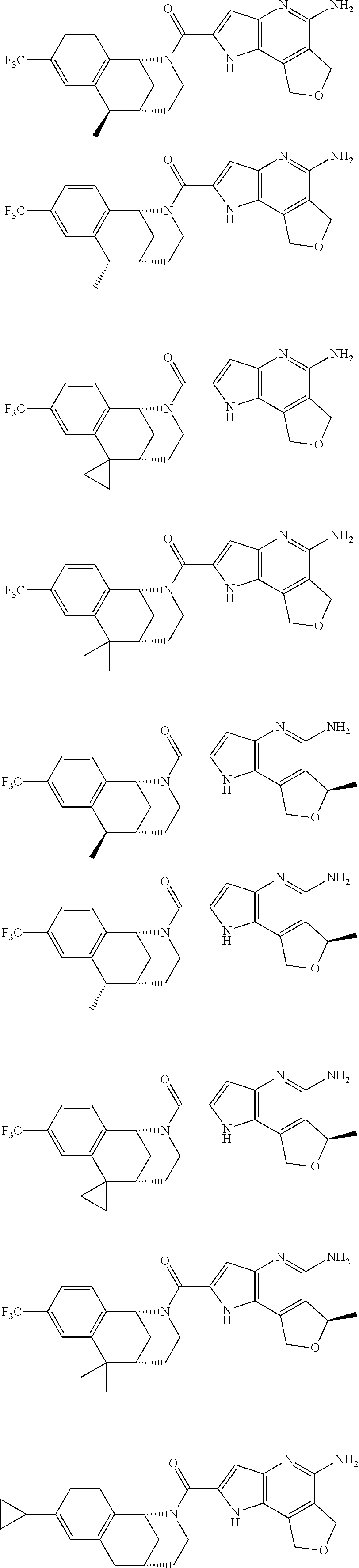

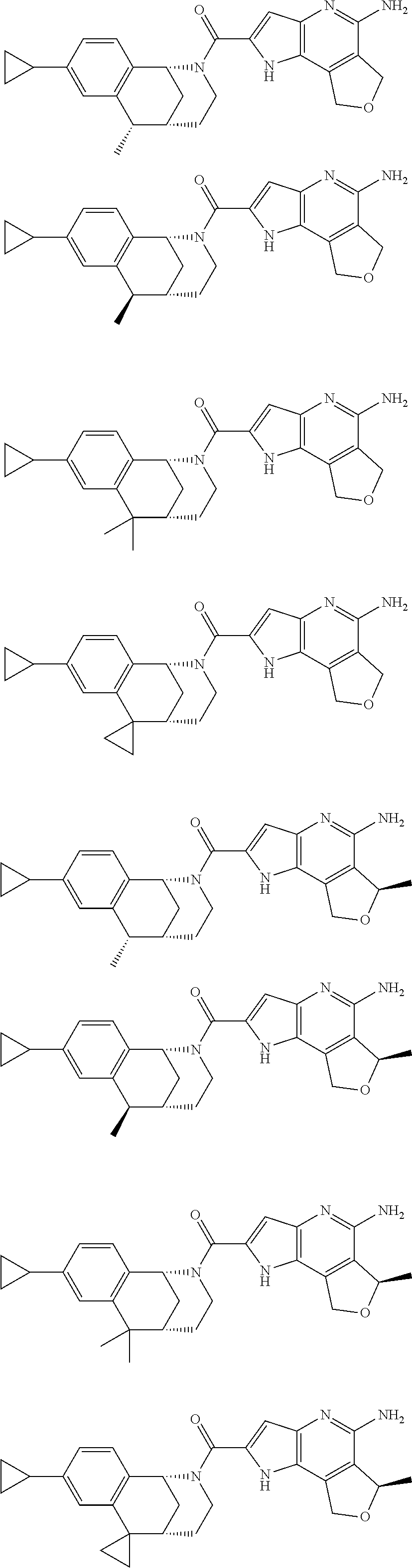

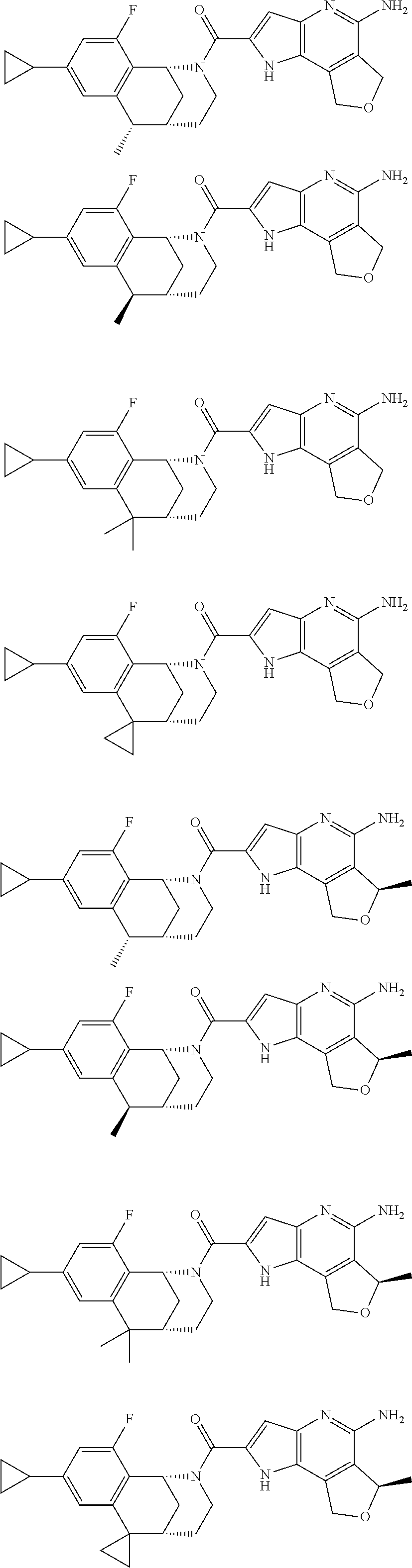

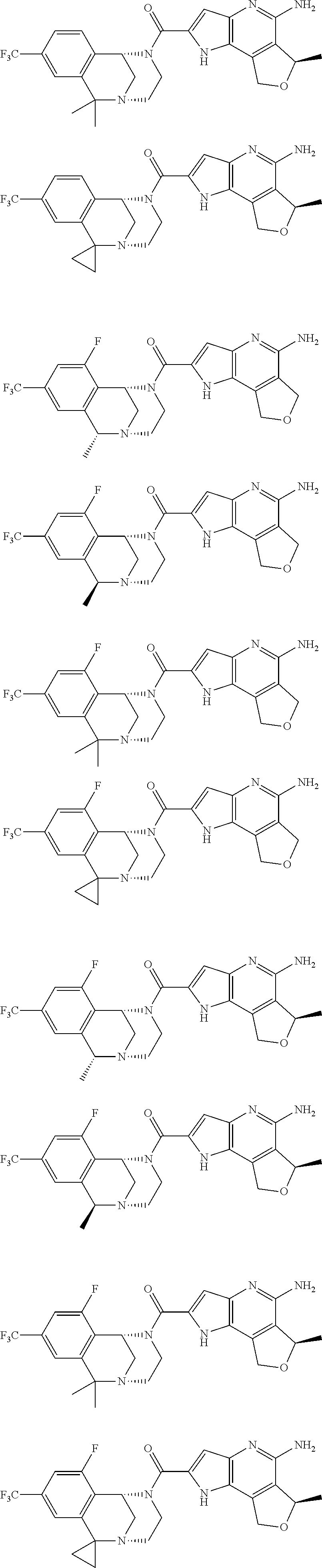

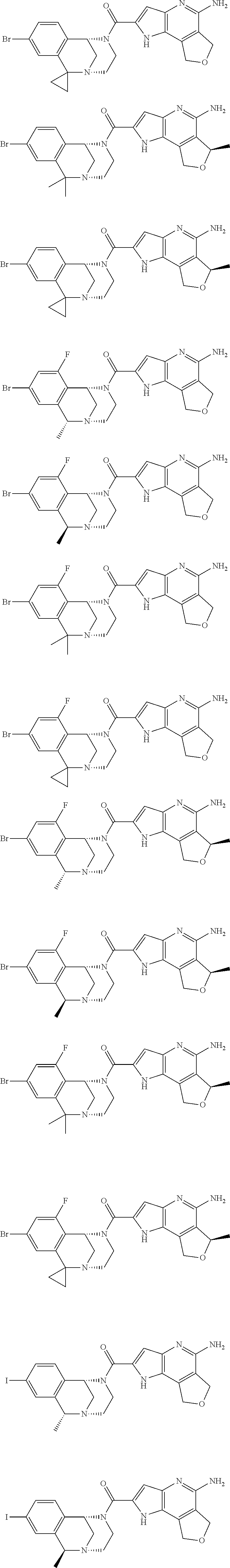

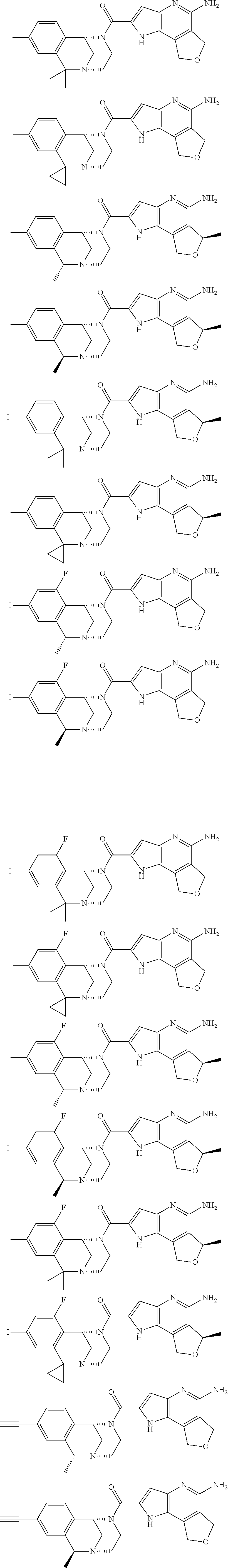

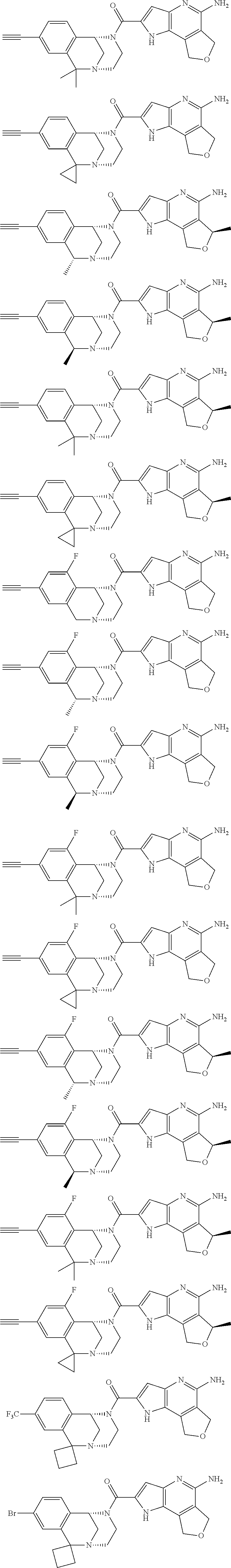

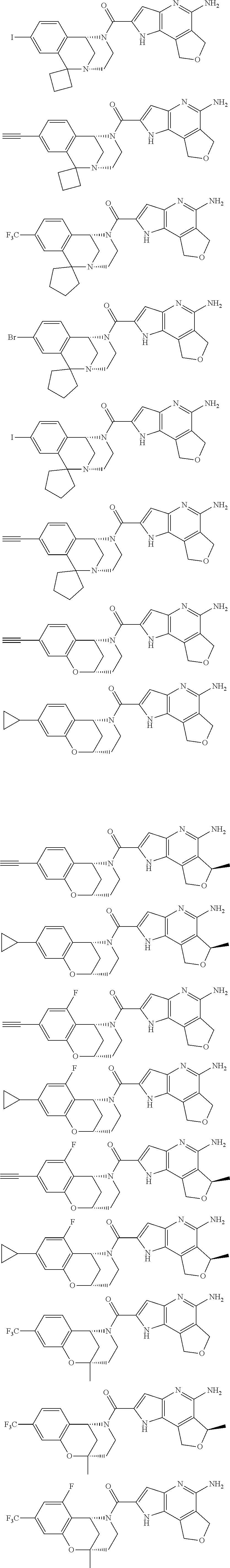

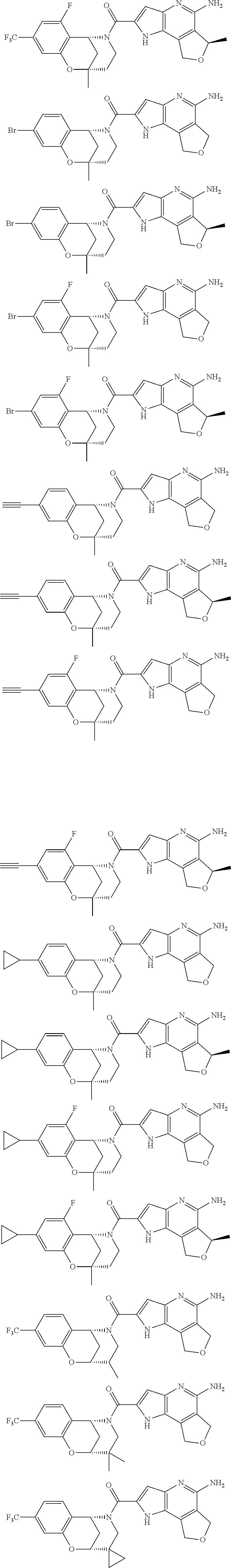

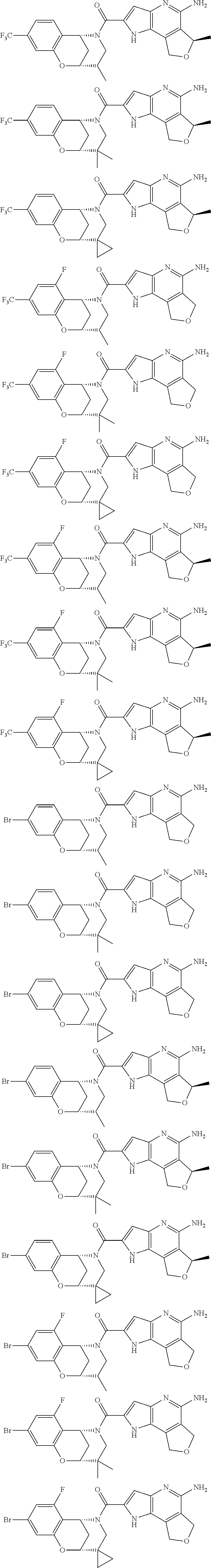

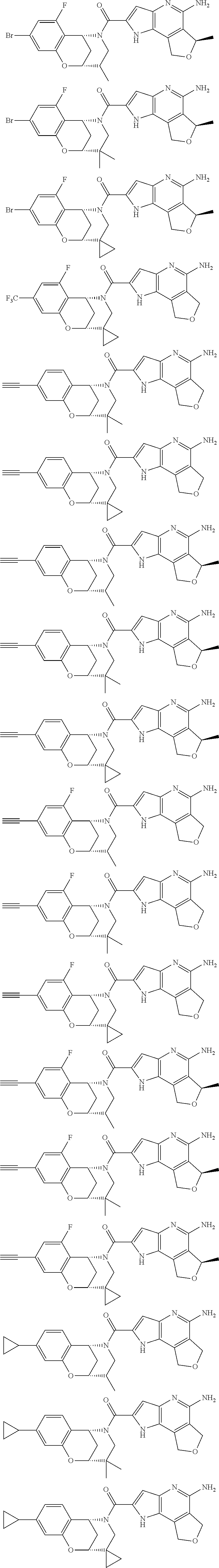

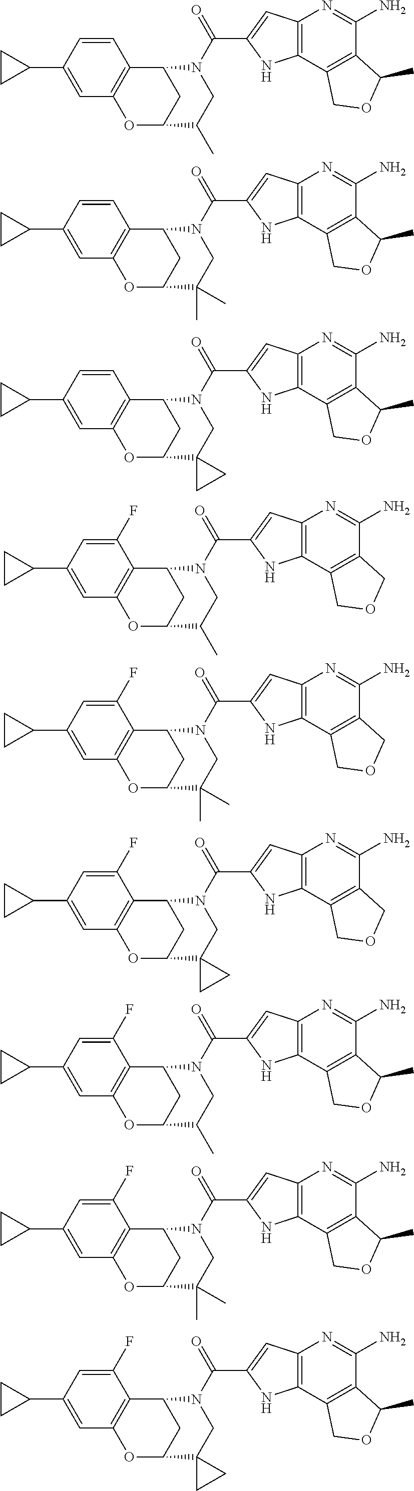

Aspect 30. A pharmaceutical composition comprising a compound of any one of Aspects 1-29, or a hydrate thereof, or a solvate thereof, or an N-oxide thereof, or a pharmaceutically acceptable salt thereof, or a stereoisomer thereof, or a tautomer thereof, or a deuterated analog thereof, and a pharmaceutically acceptable excipient.
Aspect 31. A method of inhibiting PRMT5 activity, comprising administering to a subject in need thereof an effective amount of a compound according to any one of Aspects 1-29, or a hydrate thereof, or a solvate thereof, or an N-oxide thereof, or a pharmaceutically acceptable salt, stereoisomer, tautomer, or a deuterated analog thereof.
Aspect 32. A method of treating a disease modulated by PRMT5, comprising administering to a subject in need thereof an effective amount of a compound according to any one of Aspects 1-29, or a hydrate thereof, or a solvate thereof, or an N-oxide thereof, or a pharmaceutically acceptable salt, stereoisomer, tautomer, or a deuterated analog thereof.
Aspect 33. The method of Aspect 32, wherein the disease is cancer.
Aspect 34. The method of Aspect 33, wherein the cancer comprises a methylthioadenosine phosphorylase (MTAP)-null solid tumor.

The compound of Formula (I) may contain asymmetric or chiral centers, and therefore, exist in different stereoisomeric forms, such as enantiomers, diastereomers, or atropisomers. All stereoisomeric forms of the compounds of Formula (I) as well as mixtures thereof, including racemic mixtures, form part of the present invention.

It should also be noted that the compound of Formula (I) can include E and Z isomers, or a mixture thereof, and cis and trans isomers or a mixture thereof.

It is also possible that the compound of Formula (I) may exist in different tautomeric forms, and all such forms are within the scope of the present invention.

In the compound of Formula (I), the atoms may exhibit their natural abundances, or one or more of the atoms may be artificially enriched in a particular isotope having the same atomic number, but an atomic mass or mass number different from the atomic mass or mass number predominantly found in nature. All suitable isotopic variations of the compound of Formula (I) are within the scope of the present invention. For example, different isotopic forms of hydrogen (H) include protium (¹H) and deuterium (²H).

### Methods for Making Compounds

### General Synthesis

Compounds disclosed herein, including salts thereof, can be prepared using known organic synthesis techniques and can be synthesized according to any of numerous possible synthetic routes.

The reaction for preparing compounds disclosed herein can be carried out in suitable solvents which can be readily selected by one of skill in the art of organic synthesis. Suitable solvents can be substantially non-reactive with the starting materials, the intermediates, or products at the temperatures at which the reactions are carried out, e.g., temperatures which can range from the solvent's boiling temperature. A given reaction can be carried out in one solvent or mixture of solvents.

The selection of appropriate protecting group, can be readily determined by one skilled in the art. In the synthesis schemes, some protection/deprotection steps are not shown and can be incorporated before, after or in between any steps. The protecting group shown in the synthesis schemes may or may not be used based on reaction conditions. The sequences of reactions may vary and provide similar results.

Reactions can be monitored according to any suitable method known in the art, such as NMR, UV, HPLC, LC-MS and TLC. Compounds can be purified by a variety of methods, including prep-HPLC and silica gel chromatography. Unless specified, prep-HPLC uses a buffered acetonitrile/water systems and silica gel chromatography (including column chromatography and prep-TLC) uses PE/EtOAc or DCM/MeOH systems as mobile phases. NMR spectra are recorded using a Bruker or Varian instrument with preset pulse sequences.

### Synthesis of the Acid Building Blocks (Intermediate P)

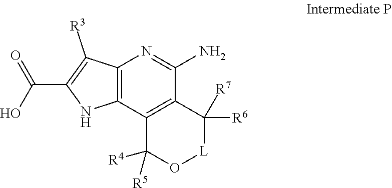

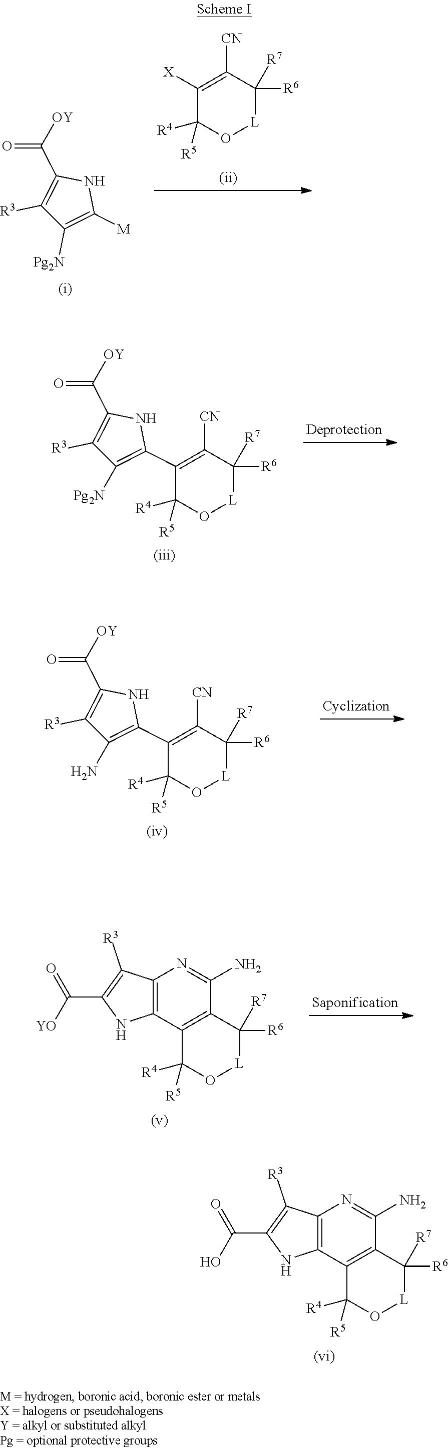

For example, Intermediate P can be formed as shown in Scheme I. Compound (i) and compound (ii) can be coupled together by transition metal catalyzed coupling to give compound (iii). Compound (iii) can be deprotected to give compound (iv). Compound (iv) can intramolecularly cyclize to give compound (v). Compound (v) can be saponified to give compound (vi) [i.e., Intermediate P].

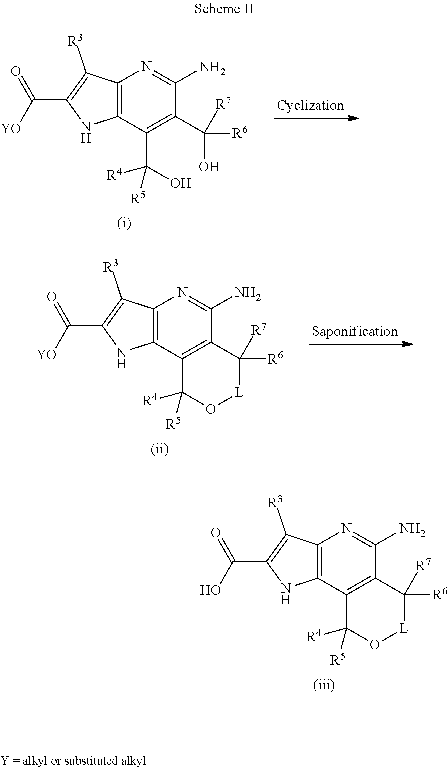

For example, Intermediate P can be formed as shown in Scheme II. Compound (i) can go through a dehydrative cyclization to give compound (ii). Compound (ii) can be saponified to give compound (iii) [i.e., Intermediate P].

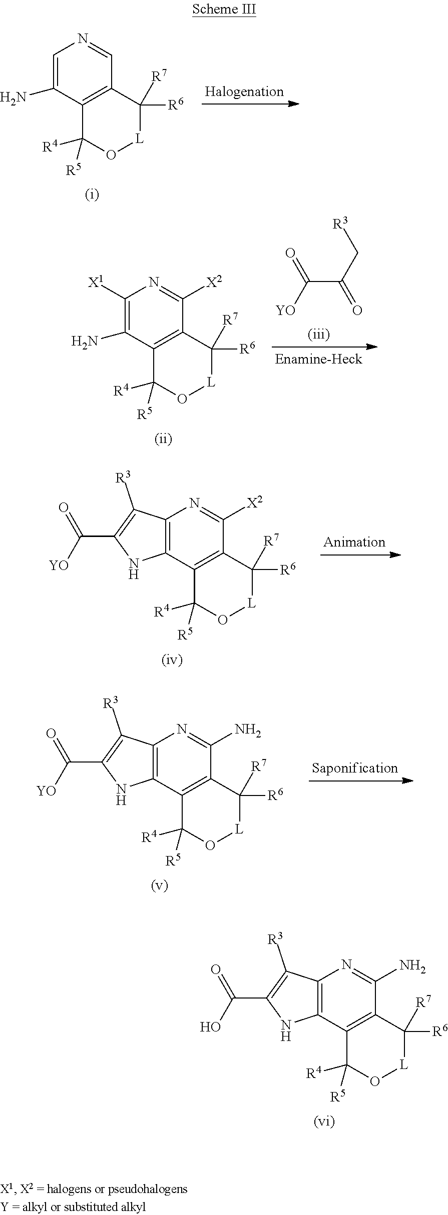

For example, Intermediate P can be formed as shown in Scheme III. Compound (i) can be halogenated to give compound (ii). Compound (ii) can react with compound (iii) through an enamine-Heck reaction to give compound (iv). Compound (iv) can be aminated to give compound (v). Compound (v) can be saponified to give compound (vi) [i.e., Intermediate P].

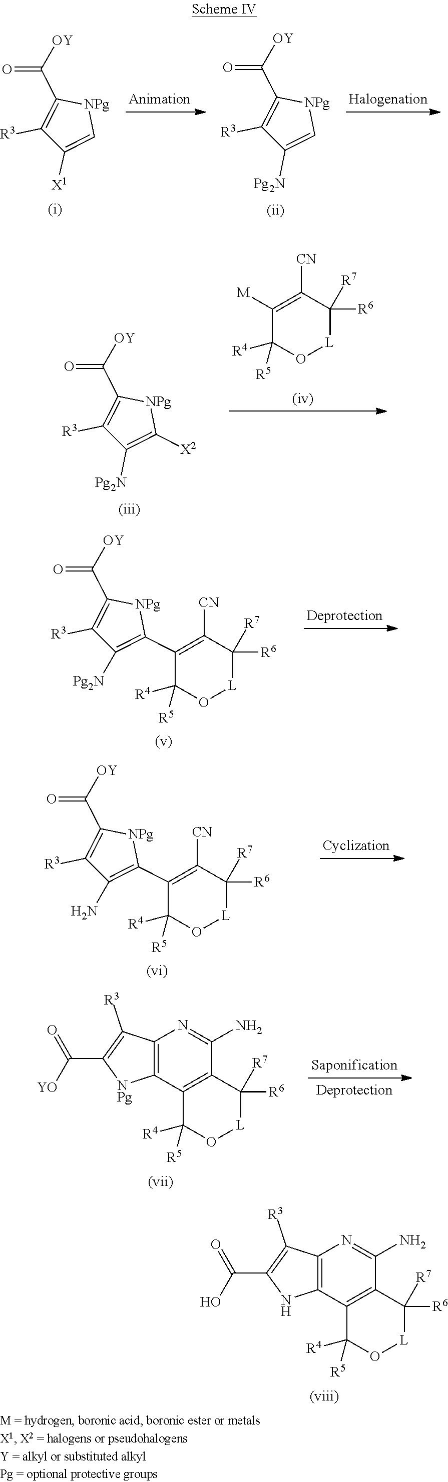

For example, Intermediate P can be formed as shown in Scheme IV. Compound (i) can be aminated, for example, via metal-catalyzed coupling reactions, to give compound (ii). Compound (ii) can be halogenated to give compound (iii). Compound (iii) and compound (iv) can be couple together by transition metal catalyzed coupling to give compound (v). Compound (v) can be deprotected to give compound (vi). Compound (vi) can intramolecularly cyclize to give compound (vii). Compound (vii) can be saponified followed by optional deprotection to give compound (viii) [i.e., Intermediate P].

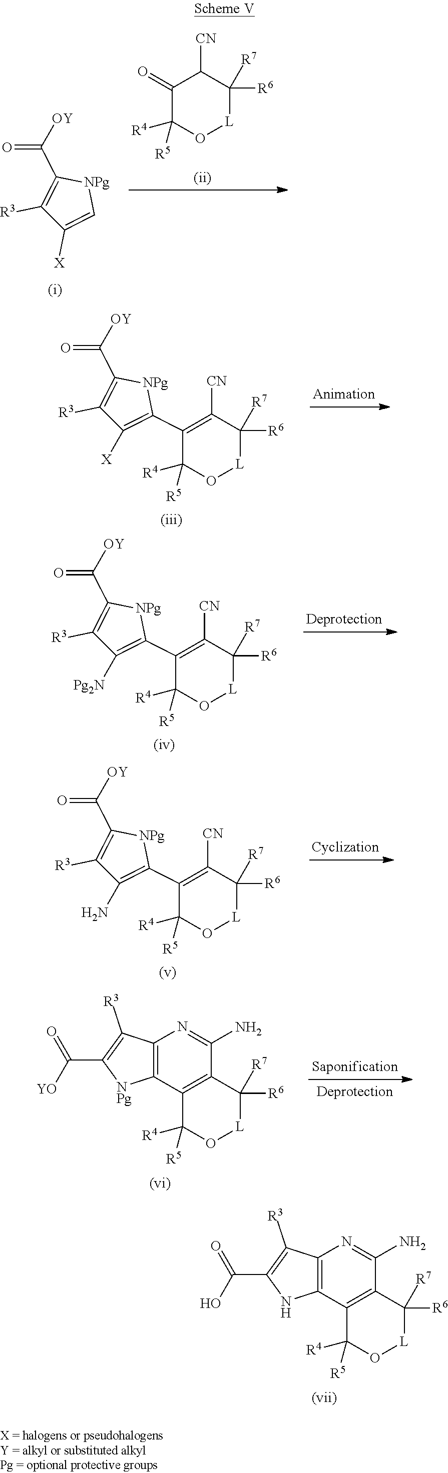

For example, Intermediate P can be formed as shown in Scheme V. Compound (i) can be condensed with compound (ii) to give compound (iii). Compound (iii) can be aminated, for example, via metal-catalyzed coupling reactions, to give compound (iv). Compound (iv) can be deprotected to give compound (v). Compound (v) can intramolecularly cyclize to give compound (vi). Compound (vi) can be saponified followed by optional deprotection to give compound (vii) [i.e., Intermediate P].

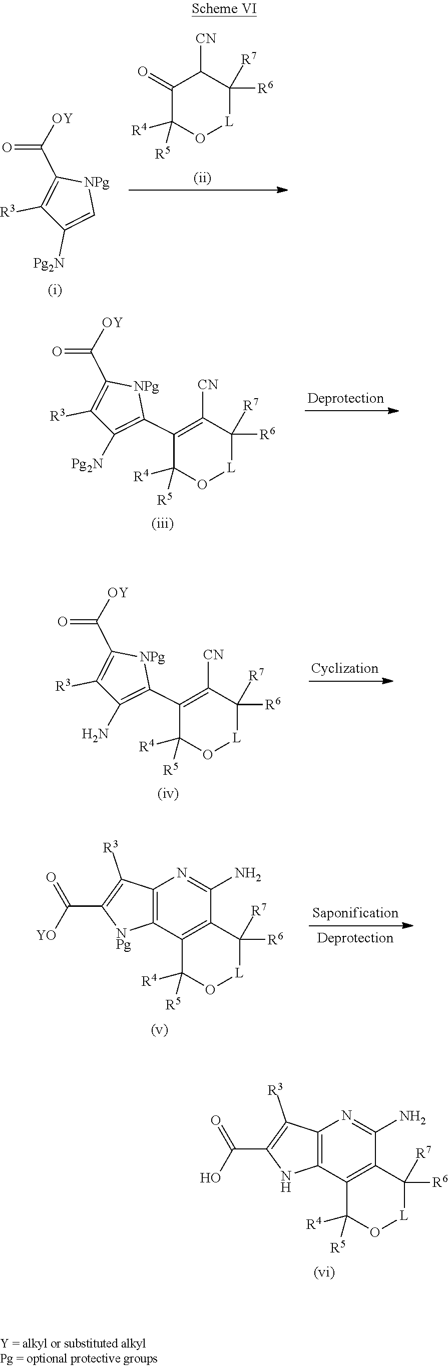

For example, Intermediate P can be formed as shown in Scheme VI. Compound (i) can be condensed with compound (ii) to give compound (iii). Compound (iii) can be deprotected to give compound (iv). Compound (iv) can intramolecularly cyclize to give compound (v). Compound (v) can be saponified followed by optional deprotection to give compound (vi) [i.e., Formula (I)].

### Synthesis of the Amine Building Blocks (Intermediate Q)

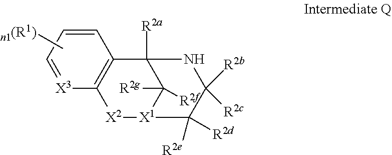

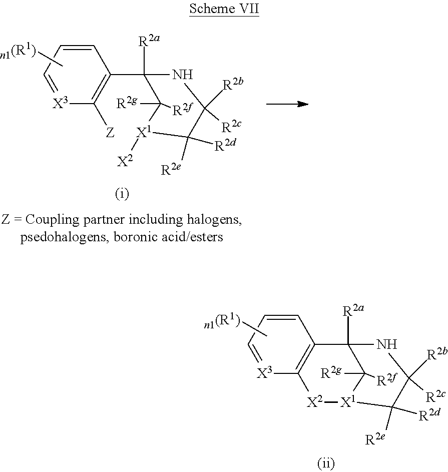

For example, Intermediate Q can be formed as shown in Scheme VII. Compound (i) can go through an intramolecular annulation reaction (including but not limited to SN_{Ar}, Chan-Lam and Buchwald reactions) to give compound (ii) [i.e., Intermediate Q].

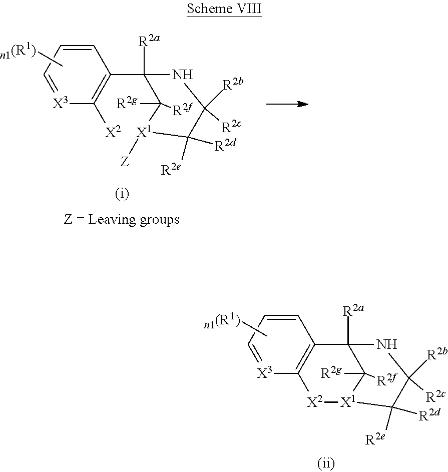

For example, Intermediate Q can be formed as shown in Scheme VIII. Compound (i) can go through an intramolecular substitution reaction to give compound (ii) [i.e., Intermediate Q].

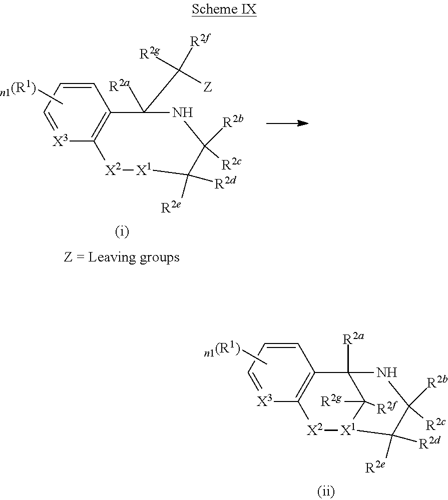

For example, Intermediate Q can be formed as shown in Scheme IX. Compound (i) can go through an intramolecular substitution reaction to give compound (ii) [i.e., Intermediate Q].

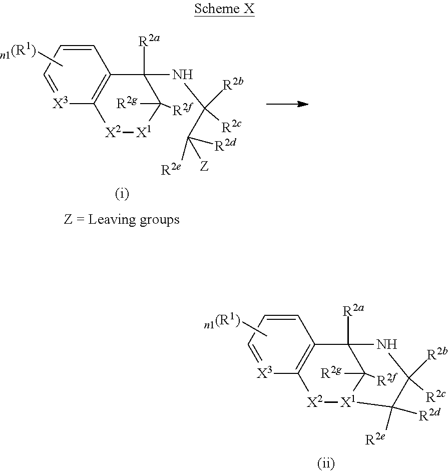

For example, Intermediate Q can be formed as shown in Scheme X. Compound (i) can go through an intramolecular substitution reaction to give compound (ii) [i.e., Intermediate Q].

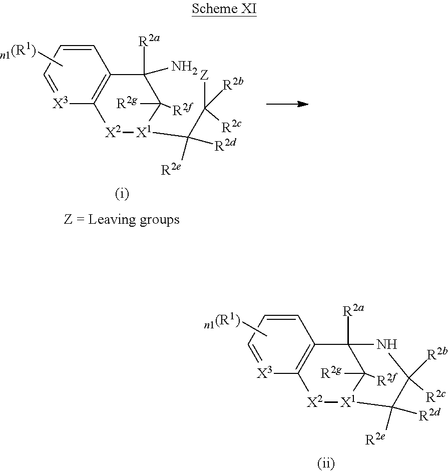

For example, Intermediate Q can be formed as shown in Scheme XI. Compound (i) can go through an intramolecular substitution reaction to give compound (ii) [i.e., Intermediate Q].

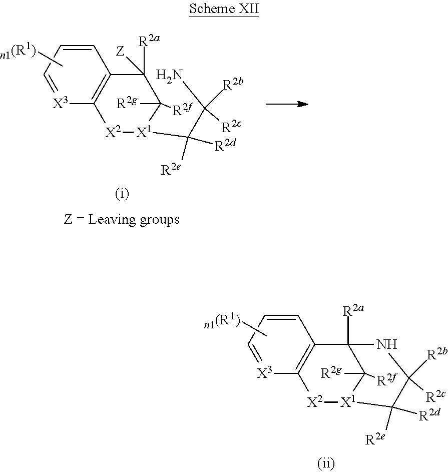

For example, Intermediate Q can be formed as shown in Scheme XII. Compound (i) can go through an intramolecular substitution reaction to give compound (ii) [i.e., Intermediate Q].

### Synthesis of Compounds of Formula (I)

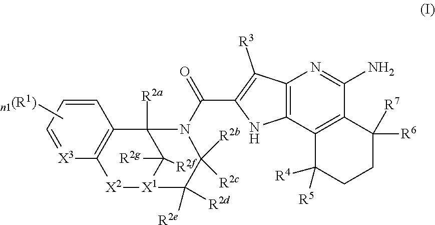

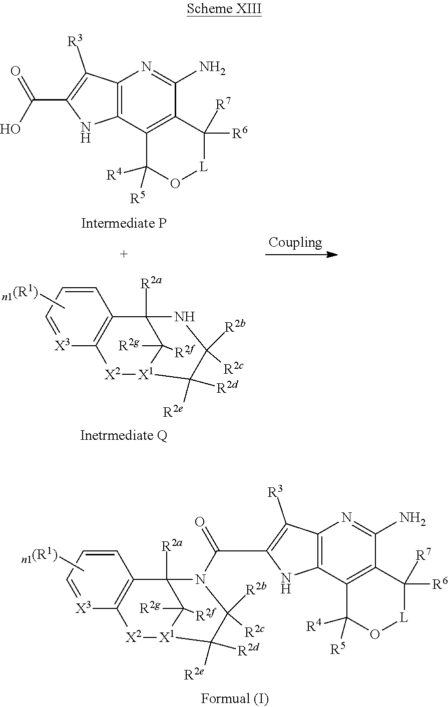

For example, compounds of Formula (I) can be formed as shown in Scheme XIII. Intermediate P can be coupled with Intermediate Q to give Formula (I).

### EXAMPLES

The examples below are intended to be purely exemplary and should not be considered to be limiting in any way. Unless otherwise specified, the experimental methods in the Examples described below are conventional methods. Unless otherwise specified, the reagents and materials are all commercially available. All solvents and chemicals employed are of analytical grade or chemical purity. Solvents are all redistilled before use. Anhydrous solvents are all prepared according to standard methods or reference methods.

### Abbreviations

In the following examples, the following abbreviations are used:
NMR nuclear magnetic resonance
UV ultraviolet
HPLC high performance liquid chromatography
prep preparation/preparative
LC-MS liquid chromatograph mass spectrometer
TLC thin layer chromatography
SFC supercritical fluid chromatography
tR retention time
PE petroleum ether
Et ethyl
Ac acetyl
dppf 1,1′-bis(diphenylphosphino)ferrocene
Me methyl
DCM dichloromethane
Boc tert-butyloxycarbonyl
NBS N-bromosuccinimide
Bu butyl
TIPS triisopropylsilyl
Tf trifluoromethanesulfonyl
SEM trimethylsilylethoxymethyl
BPO benzyl peroxide
BPD bis(pinacolato)diboron
DMSO dimethyl sulfoxide
XPhos 2-(dicyclohexylphosphino)-2′,4′,6′-triisopropylbiphenyl
THF tetrahydrofuran
TFA trifluoroacetic acid
MTBE methyl tert-butyl ether
Ph phenyl
DMF N,N-dimethylformamide
DIPEA diisopropylethylamine
AIBN 2,2′-azobis(isobutyronitrile)
DtBAD di-tert-butyl azodicarboxylate
Cbz benzyloxycarbonyl
HATU 1-[bis(dimethylamino)methylene]-1H-1,2,3-triazolo[4,5-b]pyridinium 3-oxid hexafluorophosphate
BOPCl bis(2-oxo-3-oxazolidinyl)phosphinic chloride
Bn benzyl
T3P propylphosphonic anhydride
Ms methanesulfonyl
TBS tert-butyldimethylsilyl
dba dibenzylideneacetone
Ns nitrobenzenesulfonyl

### EXAMPLES

### Example 1 & Example 2: (5-amino-6,8-dihydro-1H-furo[3,4-d]pyrrolo[3,2-b]pyridin-2-yl)((1S,5R)-8-(trifluoromethyl)-3,4,5,6-tetrahydro-1,5-methanobenzo[c]azocin-2(1H)-yl)methanone & (5-amino-6,8-dihydro-1H-furo[3,4-d]pyrrolo[3,2-b]pyridin-2-yl)((1R,5S)-8-(trifluoromethyl)-3,4,5,6-tetrahydro-1,5-methanobenzo[c]azocin-2(1H)-yl)methanone

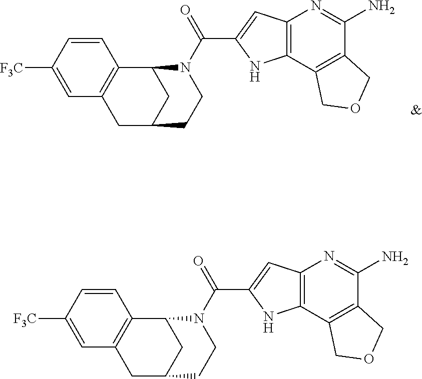 [SMILES: Nc1nc2cc(C(=O)N3CC[C@H]4Cc5cc(C(F)(F)F)ccc5[C@@H]3C4)[nH]c2c2c1COC2 ; Nc1nc2cc(C(=O)N3CC[C@@H]4Cc5cc(C(F)(F)F)ccc5[C@H]3C4)[nH]c2c2c1COC2]

### Step 1: ethyl 4-amino-1H-pyrrole-2-carboxylate

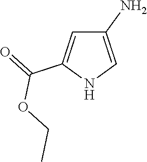

To a solution of ethyl 4-nitro-1H-pyrrole-2-carboxylate (9.0 g, 48.9 mmol) in MeOH (100 mL) was added Pd/C (10%, 5.2 g, 4.89 mmol). The mixture was stirred under hydrogen (15 psi) at 25° C. for 12 h. The mixture was filtered through a short pad of celite. The filtrate was concentrated under vacuum to give the title compound (7.0 g, 93%). LC-MS (M+H)⁺=155.2.

### Step 2: ethyl 4-((tert-butoxycarbonyl)amino)-1H-pyrrole-2-carboxylate

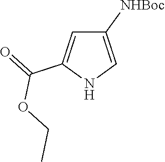 [SMILES: CCOC(=O)c1cc(NC(=O)OC(C)(C)C)c[nH]1]

To a solution of ethyl 4-amino-1H-pyrrole-2-carboxylate (7.0 g, 45.4 mmol) in DCM (80 mL) was added Boc₂O (14.9 g, 68.1 mmol) and Et₃N (13.8 g, 136 mmol). The mixture was stirred at 25° C. for 1 h. The mixture was diluted with water (100 mL) and then extracted with DCM (50 mL×3). The combined organic layer was washed with brine (50 mL×2), dried over Na₂SO₄, filtered and concentrated under reduced pressure. The residue was purified by silica gel chromatography (PE:EtOAc=1:0 to 1:1) to give the title compound (8.0 g, 69%). LC-MS (M+H-t-Bu)⁺=199.2.

### Step 3: ethyl 5-bromo-4-((tert-butoxycarbonyl)amino)-1H-pyrrole-2-carboxylate

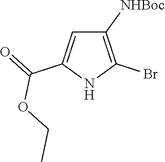 [SMILES: CCOC(=O)c1cc(NC(=O)OC(C)(C)C)c(Br)[nH]1]

To a solution of ethyl 4-((tert-butoxycarbonyl)amino)-1H-pyrrole-2-carboxylate (7.0 g, 27.5 mmol) in DCM (35 mL) was added NBS (4.90 g, 27.5 mmol). The mixture was stirred at 25° C. for 2 h. The mixture was diluted with water (50 mL) and then extracted with DCM (30 mL×3). The combined organic layer was washed with brine (30 mL×2), dried over Na₂SO₄, filtered and concentrated under reduced pressure. The residue was purified by silica gel chromatography (PE:EtOAc=5:1) to give the title compound (6.0 g, 65%). LC-MS (M+H-tBu)⁺=277.1.

### Step 4: ethyl 5-bromo-4-((tert-butoxycarbonyl)amino)-1-((2-(trimethylsilyl)ethoxy)methyl)-1H-pyrrole-2-carboxylate

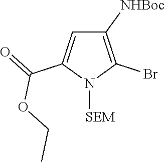 [SMILES: CCOC(=O)c1cc(NC(=O)OC(C)(C)C)c(Br)n1COCC[Si](C)(C)C]

To a solution of ethyl 5-bromo-4-((tert-butoxycarbonyl)amino)-1H-pyrrole-2-carboxylate (320 g, 960 mmol) in DMF (4000 mL) was added K₂CO₃ (332 g, 2.40 mol) and SEMCl (400 g, 2.40 mol). The mixture was stirred at 45° C. for 12 h and cooled to room temperature. Most of DMF was removed under reduced pressure. Water (1500 mL) was added, and the mixture was extracted with EtOAc (800 mL×3). The combined organic layer was washed with brine (500 mL×2), dried over Na₂SO₄, filtered and concentrated under reduced pressure. The residue was purified by silica gel chromatograph (PE:EtOAc=1:0 to 5:1) to give the title compound (310 g, 70%). LC-MS (M+Na)⁺=485.2.

### Step 5: ethyl 4-((tert-butoxycarbonyl)amino)-5-(4,4,5,5-tetramethyl-1,3,2-dioxaborolan-2-yl)-1-((2-(trimethylsilyl)ethoxy)methyl)-1H-pyrrole-2-carboxylate

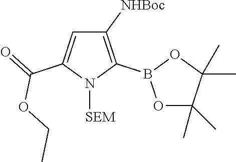 [SMILES: CCOC(=O)c1cc(NC(=O)OC(C)(C)C)c(B2OC(C)(C)C(C)(C)O2)n1COCC[Si](C)(C)C]

To a mixture of ethyl 5-bromo-4-((tert-butoxycarbonyl)amino)-1-((2-(trimethylsilyl)ethoxy)methyl)-1H-pyrrole-2-carboxylate (276 g, 596 mmol) and 4,4,5,5-tetramethyl-1,3,2-dioxaborolane (152 g, 1.19 mol) in dioxane (450 mL) was added allylpalladium chloride dimer (13.1 g, 35.7 mmol), triethylamine (301 g, 2.98 mol) and XPhos (56.8 g, 119 mmol). The mixture was stirred at 80° C. under nitrogen for 12 h. The mixture was cooled to room temperature, and the solid was filtered off. The filtrate was concentrated under reduced pressure to give the title compound (300 g, crude). The material was used in Example 1 & Example 2 Step 6 directly. LC-MS (M+Na)⁺=533.3.

### Step 6: ethyl 5-amino-1-((2-(trimethylsilyl)ethoxy)methyl)-6,8-dihydro-1H-furo[3,4-d]pyrrolo[3,2-b]pyridine-2-carboxylate

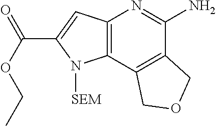 [SMILES: CCOC(=O)c1cc2nc(N)c3c(c2n1COCC[Si](C)(C)C)COC3]

To a solution of ethyl 4-((tert-butoxycarbonyl)amino)-5-(4,4,5,5-tetramethyl-1,3,2-dioxaborolan-2-yl)-1-((2-(trimethylsilyl)ethoxy)methyl)-1H-pyrrole-2-carboxylate (300 g crude) and 4-cyano-2,5-dihydrofuran-3-yl trifluoromethanesulfonate (214 g, 881 mmol) in dioxane (3900 mL) and water (780 mL) was added K₂CO₃ (244 g, 1.76 mol) and Pd(dppf)Cl₂·CH₂Cl₂ (24.0 g, 29.4 mmol). The mixture was stirred at 80° C. under nitrogen for 12 h. The mixture was cooled to room temperature, and most of dioxane was removed under reduced pressure. The remainder was diluted with water (1500 mL) and extracted with EtOAc (500 mL×3). The combined organic layer was washed with brine (500 mL×2), dried over Na₂SO₄, filtered and concentrated under reduced pressure. The residue was purified by silica gel chromatograph (PE:EtOAc=1:0 to 0:1) to give the title compound (100 g, 44% over 2 steps). ¹H NMR (400 MHz, DMSO-d6) δ 6.99 (s, 1H), 5.87 (s, 2H), 5.76 (s, 2H), 5.37-5.32 (m, 2H), 4.97-4.90 (m, 2H), 4.31 (q, J=7.0 Hz, 2H), 3.39 (t, J=7.8 Hz, 2H), 1.32 (t, J=7.0 Hz, 3H), 0.76 (t, J=7.8 Hz, 2H), −0.12 (s, 9H). LC-MS (M+H)⁺=378.2.

### Step 7: 5-amino-1-((2-(trimethylsilyl)ethoxy)methyl)-6,8-dihydro-1H-furo[3,4-d]pyrrolo[3,2-b]pyridine-2-carboxylic acid

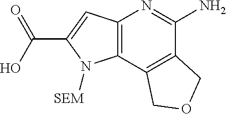 [SMILES: C[Si](C)(C)CCOCn1c(C(=O)O)cc2nc(N)c3c(c21)COC3]

To a solution of ethyl 5-amino-1-((2-(trimethylsilyl)ethoxy)methyl)-6,8-dihydro-1H-furo[3,4-d]pyrrolo[3,2-b]pyridine-2-carboxylate (65 g, 172 mmol) in MeOH (150 mL), THF (450 mL) and water (150 mL) was added LiOH·H₂O (14.5 g, 344 mmol). The mixture was stirred at 45° C. for 12 h. Most of organic solvents were removed under reduced pressure, and the remainder was diluted with water (200 mL). The mixture was washed with MTBE (100 mL×3). To the aqueous phase was added hydrochloric acid (1 M) until its pH reached 2-3. The solid was collected by filtration and then triturated with MTBE (500 mL) for 1 h. The solid was collected by filtration and dried under vacuum to give the title compound (49.4 g, 82%). ¹H NMR (400 MHz, DMSO-d6) δ 6.95 (s, 1H), 5.94 (br s, 2H), 5.80 (s, 2H), 5.36-5.32 (m, 2H), 4.96-4.91 (m, 2H), 3.40 (t, J=8.0 Hz, 2H), 0.76 (t, J=8.0 Hz, 2H), −0.12 (s, 9H); (—COOH not apparent). LC-MS (M+H)⁺=350.2.

### Step 8: 1-benzyl-4-(3-methoxybenzyl)-3,4-dihydropyridin-2(1H)-one

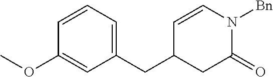 [SMILES: COc1cccc(CC2C=CN(Cc3ccccc3)C(=O)C2)c1]

To a solution of 1-benzylpyridin-2(1H)-one (4.6 g, 24.9 mmol) in DCM (100 mL) was added TIPSOTf (15.3 g, 50 mmol) at room temperature with stirring. After 30 min, 2,6-lutidine (5.35 g, 50 mmol) was added. The mixture was stirred for 30 min and then cooled to 0° C. (3-methoxybenzyl)magnesium bromide (0.5 M in diethyl ether, 100 mL, 50 mmol) was added at 0° C. dropwise. The mixture was warmed to room temperature and stirred for 1 h. Saturated NaHCO₃ (80 mL) was added to quench the reaction, and the organic layer was separated. The aqueous layer was extracted with DCM (100 mL×2). The organic layer was successively washed with hydrochloric acid (1 M, 60 mL) and brine (80 mL), dried over Na₂SO₄, filtered and concentrated under vacuum. The residue was purified by C18 reverse phase chromatograph (0.2% formic acid in H₂O:MeCN) to give the title compound (5.7 g, 75%). LC-MS (M+H)⁺=308.3.

### Step 9: 2-benzyl-8-methoxy-1,4,5,6-tetrahydro-1,5-methanobenzo[c]azocin-3(2H)-one

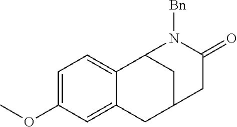 [SMILES: COc1ccc2c(c1)CC1CC(=O)N(Cc3ccccc3)C2C1]

To a solution of 1-benzyl-4-(3-methoxybenzyl)-3,4-dihydropyridin-2(1H)-one (5.7 g, 18.5 mmol) in MeCN (1 L) was added TIPSOTf (14.2 g, 46.3 mmol) with rapid stirring, and the mixture was stirred for 16 h at refluxing temperature. The mixture was cooled to room temperature and then quenched with saturated NaHCO₃ (500 mL). The mixture was extracted with EtOAc (1.5 L). The organic layer was washed with brine (800 mL), dried over Na₂SO₄, filtered and concentrated under vacuum. The residue was purified by silica gel chromatograph (PE:EtOAc=2:1) to give the title compound (3.5 g, 61%). LC-MS (M+H)⁺=308.3.

### Step 10: 2-benzyl-8-methoxy-1,2,3,4,5,6-hexahydro-1,5-methanobenzo[c]azocine

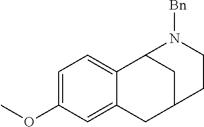 [SMILES: COc1ccc2c(c1)CC1CCN(Cc3ccccc3)C2C1]

To a solution of 2-benzyl-8-methoxy-1,4,5,6-tetrahydro-1,5-methanobenzo[c]azocin-3(2H)-one (2.0 g, 6.5 mmol) in THF (50 mL) was added BH₃ (1.0 M in THF, 9.7 mL, 9.7 mmol) at 0° C. The mixture was warmed to 60° C. and stirred for 2 h. The mixture was cooled to room temperature, followed by addition of HCl (1 M, 50 mL). The mixture was stirred for 2 h at 80° C. and then cooled to room temperature. The mixture was diluted with EtOAc (100 mL), successively washed with saturated NaHCO₃ (100 mL) and brine (50 mL), dried over Na₂SO₄, filtered and concentrated under vacuum. The residue was purified by silica gel chromatograph (PE:EtOAc=4:1) to give the title compound (1.6 g, 84%). LC-MS (M+H)⁺=294.3.

### Step 11: 2-benzyl-1,2,3,4,5,6-hexahydro-1,5-methanobenzo[c]azocin-8-ol

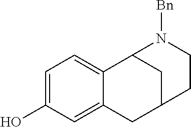 [SMILES: Oc1ccc2c(c1)CC1CCN(Cc3ccccc3)C2C1]

To a solution of 2-benzyl-8-methoxy-1,2,3,4,5,6-hexahydro-1,5-methanobenzo[c]azocine (1.6 g, 5.5 mmol) in DCM (30 mL) was added BBr₃ (1.0 M in DCM, 10.9 mL, 10.9 mmol) at 0° C. The mixture was warmed to room temperature and stirred for 2 h. The mixture was concentrated under reduced pressure and re-dissolved with EtOAc (80 mL). The mixture was successively washed with saturated NaHCO₃ (20 mL), water (20 mL) and brine (20 mL), dried over Na₂SO₄, filtered and concentrated under vacuum. The residue was purified by silica gel chromatograph (PE:EtOAc=3:1) to give the title compound (1.3 g, 86%). LC-MS (M+H)⁺=280.3.

### Step 12: 2-benzyl-1,2,3,4,5,6-hexahydro-1,5-methanobenzo[c]azocin-8-yl trifluoromethanesulfonate

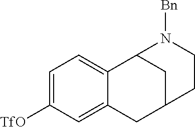 [SMILES: O=S(=O)(Oc1ccc2c(c1)CC1CCN(Cc3ccccc3)C2C1)C(F)(F)F]

To a solution of 2-benzyl-1,2,3,4,5,6-hexahydro-1,5-methanobenzo[c]azocin-8-ol (300 mg, 1.07 mmol) in DCM (20 mL) was added Et₃N (140 mg, 1.39 mmol). The mixture was cooled to −78° C. followed by addition of Tf₂O (362 mg, 1.28 mmol). After 1 h, the mixture was diluted with DCM (30 mL), washed with water (20 mL) and brine (20 mL), dried over Na₂SO₄, filtered and concentrated under vacuum. The residue was purified by silica gel chromatograph (PE:EtOAc=3:1) to give the title compound (330 mg, 75%). LC-MS (M+H)⁺=412.3.

### Step 13: 2-benzyl-8-(4,4,5,5-tetramethyl-1,3,2-dioxaborolan-2-yl)-1,2,3,4,5,6-hexahydro-1,5-methanobenzo[c]azocine

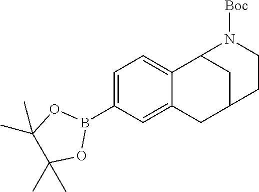 [SMILES: CC(C)(C)OC(=O)N1CCC2Cc3cc(B4OC(C)(C)C(C)(C)O4)ccc3C1C2]

To a solution of 2-benzyl-1,2,3,4,5,6-hexahydro-1,5-methanobenzo[c]azocin-8-yl trifluoromethanesulfonate (330 mg, 0.80 mmol) in dioxane (15 mL) was added BPD (244 mg, 0.96 mmol), Pd(dppf)Cl₂ (59 mg, 0.080 mmol), dppf (53 mg, 0.096 mmol) and KOAc (235 mg, 2.4 mmol). The mixture was stirred for 12 h at 80° C. under nitrogen and then cooled to room temperature. The mixture was diluted with EtOAc (40 mL), washed with water (20 mL) and brine (20 mL), dried over Na₂SO₄, filtered and concentrated under vacuum. The residue was purified by prep-TLC (PE:EtOAc=4:1) to give the title compound (220 mg, 71%). LC-MS (M+H)⁺=390.3.

### Step 14: 2-benzyl-8-(trifluoromethyl)-1,2,3,4,5,6-hexahydro-1,5-methanobenzo[c]azocine

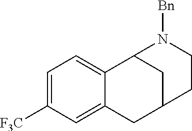 [SMILES: FC(F)(F)c1ccc2c(c1)CC1CCN(Cc3ccccc3)C2C1]

To a solution of 2-benzyl-8-(4,4,5,5-tetramethyl-1,3,2-dioxaborolan-2-yl)-1,2,3,4,5,6-hexahydro-1,5-methanobenzo[c]azocine (220 mg, 0.56 mmol) in DMF (8 mL) was added (1,10-phenanthroline)(trifluoromethyl)copper(I) (530 mg, 1.69 mmol). The mixture was stirred for 4 h at 60° C. then cooled to room temperature. The mixture was diluted with EtOAc (30 mL), washed with brine (10 mL×2), dried over Na₂SO₄, filtered and concentrated under vacuum. The residue was purified by prep-TLC (PE:EtOAc=4:1) to give the title compound (120 mg, 64%). LC-MS (M+H)⁺=332.3.

### Step 15: 8-(trifluoromethyl)-1,2,3,4,5,6-hexahydro-1,5-methanobenzo[c]azocine

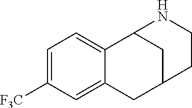 [SMILES: FC(F)(F)c1ccc2c(c1)CC1CCNC2C1]

To a solution of 2-benzyl-8-(trifluoromethyl)-1,2,3,4,5,6-hexahydro-1,5-methanobenzo[c]azocine (120 mg, 0.36 mmol) in EtOAc (10 mL) was added Pd/C (10%, 20 mg) under nitrogen. The flask was purged with hydrogen and the mixture was stirred for 16 h at room temperature. The solid was filtered off and the filtrate was concentrated under vacuum. The residue was purified by prep-TLC (MeOH:DCM=1:15) to give the title compound (60 mg, 69%). LC-MS (M+H)⁺=242.3.

### Step 16: (5-amino-1-((2-(trimethylsilyl)ethoxy)methyl)-6,8-dihydro-1H-furo[3,4-d]pyrrolo[3,2-b]pyridin-2-yl)(8-(trifluoromethyl)-3,4,5,6-tetrahydro-1,5-methanobenzo[c]azocin-2(1H)-yl)methanone

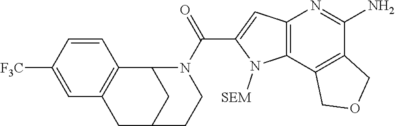 [SMILES: C[Si](C)(C)CCOCn1c(C(=O)N2CCC3Cc4cc(C(F)(F)F)ccc4C2C3)cc2nc(N)c3c(c21)COC3]

A mixture of 8-(trifluoromethyl)-1,2,3,4,5,6-hexahydro-1,5-methanobenzo[c]azocine (55 mg, 0.23 mol), 5-amino-1-((2-(trimethylsilyl)ethoxy)methyl)-6,8-dihydro-1H-furo[3,4-d]pyrrolo[3,2-b]pyridine-2-carboxylic acid (80 mg, 0.23 mmol), DIPEA (89 mg, 0.69 mmol) and BOPCl (88 mg, 0.35 mmol) in THF (5 mL) was stirred at 60° C. for 2 h and then cooled to room temperature. The mixture was diluted with EtOAc (20 mL), washed with water (10 mL), brine (10 mL), dried over Na₂SO₄, filtered and concentrated under vacuum. The residue was purified by prep-TLC with EtOAc to give the title compound (90 mg, 68%). LC-MS (M+H)⁺=573.3.

### Step 17: (5-amino-6,8-dihydro-1H-furo[3,4-d]pyrrolo[3,2-b]pyridin-2-yl)((1S,5R)-8-(trifluoromethyl)-3,4,5,6-tetrahydro-1,5-methanobenzo[c]azocin-2(1H)-yl)methanone & (5-amino-6,8-dihydro-1H-furo[3,4-d]pyrrolo[3,2-b]pyridin-2-yl)((1R,5S)-8-(trifluoromethyl)-3,4,5,6-tetrahydro-1,5-methanobenzo[c]azocin-2(1H)-yl)methanone

To a solution of (5-amino-1-((2-(trimethylsilyl)ethoxy)methyl)-6,8-dihydro-H-furo[3,4-d]pyrrolo[3,2-b]pyridin-2-yl)(8-(trifluoromethyl)-3,4,5,6-tetrahydro-1,5-methanobenzo[c]azocin-2(1H)-yl)methanone (90 mg, 0.16 mmol) in DCM (6 mL) was added TFA (2 mL). The mixture was stirred at room temperature for 1 h. The mixture was concentrated under reduced pressure. The residue was re-dissolved in methanolic ammonia (7 M, 8 mL). The mixture was stirred at room temperature for 1 h and then concentrated under reduced pressure. The residue was purified by prep-HPLC followed by chiral SFC to give Example 1 (20 mg, 29%) and Example 2 (20 mg, 29%).

Analytical chiral-SFC condition as below. Column: Lux Cellulose-3; Column size: 4.6×100 mm, 5 μm; Mobile phase: 4 mM methanolic NH₃:CO₂, 1:9 to 1:1 in 3 min, 1:1 for 2 min, 1:1 to 1:9 in 0.1 min, 1:9 for 1.9 min; Flow: 2.0 mL/min; Temperature: 35° C.; back pressure: 1500 psi.

Example 1: Analytical SFC tR=2.65 min. ¹H NMR (400 MHz, DMSO-d6, 80° C.) δ11.34 (s, 1H), 7.53 (s, 1H), 7.46 (d, J=8.2 Hz, 1H), 7.37-7.18 (m, 1H), 6.53 (s, 1H), 5.86-5.73 (m, 1H), 5.39 (s, 2H), 5.14 (s, 2H), 4.93 (s, 2H), 4.08 (dd, J=13.9, 5.0 Hz, 1H), 3.20 (dd, J=18.0, 6.5 Hz, 1H), 2.94 (d, J=18.5 Hz, 1H), 2.85-2.67 (m, 1H), 2.46-2.41 (m, 1H), 2.20-2.06 (m, 1H), 1.99-1.85 (m, 2H), 1.77-1.67 (m, 1H). LC-MS (M+H)⁺=443.4.

Example 2: Analytical SFC tR=2.86 min. ¹H NMR (400 MHz, DMSO-d6, 80° C.) δ11.34 (s, 1H), 7.53 (s, 1H), 7.46 (d, J=8.2 Hz, 1H), 7.37-7.18 (m, 1H), 6.53 (s, 1H), 5.86-5.73 (m, 1H), 5.39 (s, 2H), 5.14 (s, 2H), 4.93 (s, 2H), 4.08 (dd, J=13.9, 5.0 Hz, 1H), 3.20 (dd, J=18.0, 6.5 Hz, 1H), 2.94 (d, J=18.5 Hz, 1H), 2.85-2.67 (m, 1H), 2.46-2.41 (m, 1H), 2.20-2.06 (m, 1H), 1.99-1.85 (m, 2H), 1.77-1.67 (m, 1H). LC-MS (M+H)⁺=443.4.

### Example 3 & Example 4: (5-amino-6,8-dihydro-1H-furo[3,4-d]pyrrolo[3,2-b]pyridin-2-yl)((1R,5S)-3,4,5,6-tetrahydro-1,5-methanobenzo[c]azocin-2(1H)-yl)methanone & (5-amino-6,8-dihydro-1H-furo[3,4-d]pyrrolo[3,2-b]pyridin-2-yl)((1S,5R)-3,4,5,6-tetrahydro-1,5-methanobenzo[c]azocin-2(1H)-yl)methanone

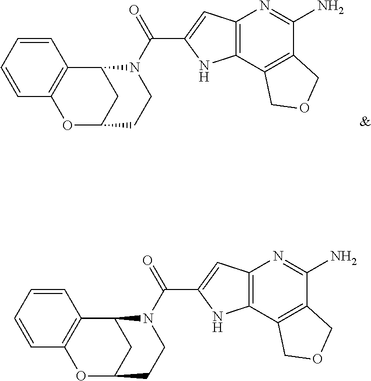 [SMILES: Nc1nc2cc(C(=O)N3CC[C@H]4C[C@@H]3c3ccccc3O4)[nH]c2c2c1COC2 ; Nc1nc2cc(C(=O)N3CC[C@@H]4C[C@H]3c3ccccc3O4)[nH]c2c2c1COC2]

### Step 1: 1,4-dibenzyl-3,4-dihydropyridin-2(1H)-one

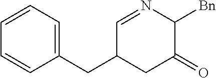 [SMILES: O=C1CC(Cc2ccccc2)C=NC1Cc1ccccc1]

The title compound (3.0 g, 33%) was prepared in a manner similar to that in Example 1 & Example 2 step 8 from 1-benzylpyridin-2(1H)-one and BnMgCl. LC-MS (M+H)⁺=278.3.

### Step 2: 2-benzyl-1,4,5,6-tetrahydro-1,5-methanobenzo[c]azocin-3(2H)-one

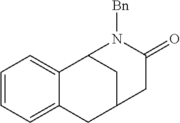 [SMILES: O=C1CC2Cc3ccccc3C(C2)N1Cc1ccccc1]

The title compound (260 mg, 73%) was prepared in a manner similar to that in Example 1 & Example 2 step 9 from 1,4-dibenzyl-3,4-dihydropyridin-2(1H)-one. LC-MS (M+H)⁺=278.3.

### Step 3: 2-benzyl-1,4,5,6-tetrahydro-1,5-methanobenzo[c]azocine-3(2H)-thione

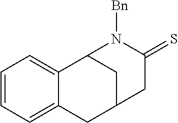 [SMILES: S=C1CC2Cc3ccccc3C(C2)N1Cc1ccccc1]

To a solution of 2-benzyl-1,4,5,6-tetrahydro-1,5-methanobenzo[c]azocin-3(2H)-one (260 mg, 0.93 mmol) in toluene (10 mL) was added Lawesson reagent (189 mg, 0.48 mmol). The mixture was stirred at 100° C. for 3 h and then cooled to room temperature. The mixture was diluted with EtOAc (40 mL), washed with saturated NaHCO₃ (20 mL), dried over Na₂SO₄, filtered and concentrated under vacuum. The residue was purified by prep-TLC (PE:EtOAc=4:1) to give the title compound (160 mg, 59%). LC-MS (M+H)⁺=294.3.

### Step 4: 1,2,3,4,5,6-hexahydro-1,5-methanobenzo[c]azocine

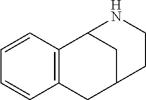 [SMILES: c1ccc2c(c1)CC1CCNC2C1]

To a solution of 2-benzyl-1,4,5,6-tetrahydro-1,5-methanobenzo[c]azocine-3(2H)-thione (160 mg, 0.54 mmol) in isopropanol (20 mL) was added Raney Ni (300 mg) under nitrogen. The flask was flushed with hydrogen and the mixture was stirred for 16 h at 70° C. The mixture was cooled to room temperature and solid was filtered off. The filtrate was collected and concentrated under vacuum. The residue was purified by purified by prep-TLC (MeOH:DCM=1:15) to give the title compound (80 mg, 85%). LC-MS (M+H)=174.3.

### Step 5: (5-amino-1-((2-(trimethylsilyl)ethoxy)methyl)-6,8-dihydro-1H-furo[3,4-d]pyrrolo[3,2-b]pyridin-2-yl)(3,4,5,6-tetrahydro-1,5-methanobenzo[c]azocin-2(1H)-yl)methanone

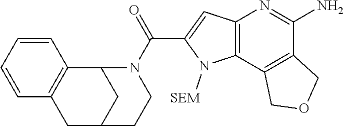 [SMILES: C[Si](C)(C)CCOCn1c(C(=O)N2CCC3Cc4ccccc4C2C3)cc2nc(N)c3c(c21)COC3]

The title compound (160 mg, 69%) was prepared in a manner similar to that in Example 1 & Example 2 step 16 from 1,2,3,4,5,6-hexahydro-1,5-methanobenzo[c]azocine and 5-amino-1-((2-(trimethylsilyl)ethoxy)methyl)-6,8-dihydro-1H-furo[3,4-d]pyrrolo[3,2-b]pyridine-2-carboxylic acid. LC-MS (M+H)⁺=505.5.

### Step 6: (5-amino-6,8-dihydro-1H-furo[3,4-d]pyrrolo[3,2-b]pyridin-2-yl)((1R,5S)-3,4,5,6-tetrahydro-1,5-methanobenzo[c]azocin-2(1H)-yl)methanone & (5-amino-6,8-dihydro-1H-furo[3,4-d]pyrrolo[3,2-b]pyridin-2-yl)((1S,5R)-3,4,5,6-tetrahydro-1,5-methanobenzo[c]azocin-2(1H)-yl)methanone

Example 3 (20 mg, 17%) and Example 4 (20 mg, 17%) were prepared in a manner similar to that in Example 1 & Example 2 step 17 from (5-amino-1-((2-(trimethylsilyl)ethoxy)methyl)-6,8-dihydro-1H-furo[3,4-d]pyrrolo[3,2-b]pyridin-2-yl)(3,4,5,6-tetrahydro-1,5-methanobenzo[c]azocin-2(1H)-yl)methanone, and the enantiomers were separated by chiral SFC.

Analytical chiral-SFC condition as below. Column: CHIRALPAK AS-H; Column size: 4.6×100 mm, 5 μm; Mobile phase: 4 mM methanolic NH₃:CO₂, 1:9 to 4:6 in 3 min, 4:6 for 2 min, 4:6 to 1:9 in 0.1 min, 1:9 for 1.9 min; Flow: 2.0 mL/min; Temperature: 35° C.; back pressure: 1500 psi.

Example 3: Analytical SFC tR=3.80 min. ¹H NMR (400 MHz, DMSO-d6, 80° C.) δ11.28 (s, 1H), 7.24-7.14 (m, 2H), 7.12-6.96 (m, 2H), 6.50 (s, 1H), 5.70 (s, 1H), 5.28 (s, 2H), 5.14 (s, 2H), 4.93 (s, 2H), 4.10-4.00 (m, 1H), 3.19-3.10 (m, 1H), 2.88-2.68 (m, 2H), 2.40 (s, 1H), 2.14-2.04 (m, 1H), 1.96-1.84 (m, 2H), 1.74-1.66 (m, 1H). LC-MS (M+H)⁺=375.3.

Example 4: Analytical SFC tR=4.23 min. ¹H NMR (400 MHz, DMSO-d6, 80° C.) δ11.28 (s, 1H), 7.24-7.14 (m, 2H), 7.12-6.96 (m, 2H), 6.50 (s, 1H), 5.70 (s, 1H), 5.28 (s, 2H), 5.14 (s, 2H), 4.93 (s, 2H), 4.10-4.00 (m, 1H), 3.19-3.10 (m, 1H), 2.88-2.68 (m, 2H), 2.40 (s, 1H), 2.14-2.04 (m, 1H), 1.96-1.84 (m, 2H), 1.74-1.66 (m, 1H). LC-MS (M+H)⁺=375.3.

### Example 5 & Example 6: (5-amino-6,8-dihydro-1H-furo[3,4-d]pyrrolo[3,2-b]pyridin-2-yl)((1R,5S)-8-methyl-3,4,5,6-tetrahydro-1,5-methanobenzo[c]azocin-2(1H)-yl)methanone & (5-amino-6,8-dihydro-1H-furo[3,4-d]pyrrolo[3,2-b]pyridin-2-yl)((1S,5R)-8-methyl-3,4,5,6-tetrahydro-1,5-methanobenzo[c]azocin-2(1H)-yl)methanone

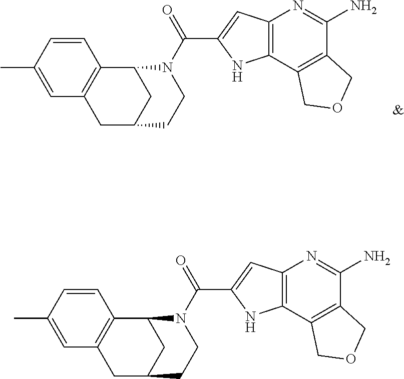 [SMILES: Cc1ccc2c(c1)C[C@H]1CCN(C(=O)c3cc4nc(N)c5c(c4[nH]3)COC5)[C@@H]2C1 ; Cc1ccc2c(c1)C[C@@H]1CCN(C(=O)c3cc4nc(N)c5c(c4[nH]3)COC5)[C@H]2C1]

### Step 1: 1-benzyl-4-(3-methylbenzyl)-3,4-dihydropyridin-2(1H)-one

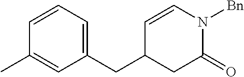 [SMILES: Cc1cccc(CC2C=CN(Cc3ccccc3)C(=O)C2)c1]

The title compound (3.5 g, 89%) was prepared in a manner similar to that in Example 1 & Example 2 step 8 from 1-benzylpyridin-2(1H)-one and (3-methylbenzyl)magnesium bromide. LC-MS (M+H)=292.3.

### Step 2: 2-benzyl-8-methyl-1,4,5,6-tetrahydro-1,5-methanobenzo[c]azocin-3(2H)-one

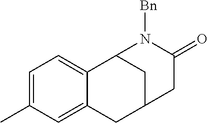 [SMILES: Cc1ccc2c(c1)CC1CC(=O)N(Cc3ccccc3)C2C1]

The title compound (2.5 g, 71%) was prepared in a manner similar to that in Example 1 & Example 2 step 9 from 1-benzyl-4-(3-methylbenzyl)-3,4-dihydropyridin-2(1H)-one. LC-MS (M+H)⁺=292.3.

### Step 3: 2-benzyl-8-methyl-1,4,5,6-tetrahydro-1,5-methanobenzo[c]azocine-3(2H)-thione

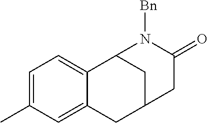 [SMILES: Cc1ccc2c(c1)CC1CC(=O)N(Cc3ccccc3)C2C1]

The title compound (300 mg, 63%) was prepared in a manner similar to that in Example 3 & Example 4 step 3 from 2-benzyl-8-methyl-1,4,5,6-tetrahydro-1,5-methanobenzo[c]azocin-3(2H)-one. LC-MS (M+H)⁺=308.3.

### Step 4: 8-methyl-1,2,3,4,5,6-hexahydro-1,5-methanobenzo[c]azocine

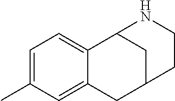

The title compound (150 mg, 82%) was prepared in a manner similar to that in Example 3 & Example 4 step 4 from 2-benzyl-8-methyl-1,4,5,6-tetrahydro-1,5-methanobenzo[c]azocine-3(2H)-thione. LC-MS (M+H)⁺=188.3.

### Step 5: (5-amino-1-((2-(trimethylsilyl)ethoxy)methyl)-6,8-dihydro-1H-furo[3,4-d]pyrrolo[3,2-b]pyridin-2-yl)(8-methyl-3,4,5,6-tetrahydro-1,5-methanobenzo[c]azocin-2(1H)-yl)methanone

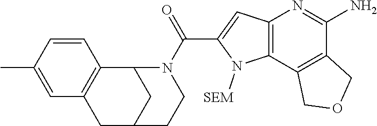 [SMILES: Cc1ccc2c(c1)CC1CCN(C(=O)c3cc4nc(N)c5c(c4n3COCC[Si](C)(C)C)COC5)C2C1]

The title compound (270 mg, 65%) was prepared in a manner similar to that in Example 1 & Example 2 step 16 from 8-methyl-1,2,3,4,5,6-hexahydro-1,5-methanobenzo[c]azocine and 5-amino-1-((2-(trimethylsilyl)ethoxy)methyl)-6,8-dihydro-1H-furo[3,4-d]pyrrolo[3,2-b]pyridine-2-carboxylic acid. LC-MS (M+H)⁺=519.5.

### Step 6: (5-amino-6,8-dihydro-1H-furo[3,4-d]pyrrolo[3,2-b]pyridin-2-yl)((1R,5S)-8-methyl-3,4,5,6-tetrahydro-1,5-methanobenzo[c]azocin-2(1H)-yl)methanone & (5-amino-6,8-dihydro-1H-furo[3,4-d]pyrrolo[3,2-b]pyridin-2-yl)((1S,5R)-8-methyl-3,4,5,6-tetrahydro-1,5-methanobenzo[c]azocin-2(1H)-yl)methanone

Example 5 (69 mg, 34%) and Example 6 (72 mg, 36%) were prepared in a manner similar to that in Example 1 & Example 2 step 17 from (5-amino-1-((2-(trimethylsilyl)ethoxy)methyl)-6,8-dihydro-1H-furo[3,4-d]pyrrolo[3,2-b]pyridin-2-yl)(8-methyl-3,4,5,6-tetrahydro-1,5-methanobenzo[c]azocin-2(1H)-yl)methanone, and the enantiomers were separated by chiral SFC.

Analytical chiral-SFC condition as below. Column: CHIRALPAK AS-H; Column size: 4.6×100 mm, 5 μm; Mobile phase: 4 mM methanolic NH₃:CO₂, 1:9 to 1:1 in 3 min, 1:1 for 2 min, 1:1 to 1:9 in 0.1 min, 1:9 for 1.9 min; Flow: 2.0 mL/min; Temperature: 35° C.; back pressure: 1500 psi.

Example 5: Analytical SFC tR=3.80 min. ¹H NMR (400 MHz, DMSO-d6, 80° C.) δ11.27 (s, 1H), 6.99-6.89 (m, 3H), 6.48 (s, 1H), 5.66 (s, 1H), 5.28 (s, 2H), 5.13 (s, 2H), 4.93 (s, 2H), 4.07-4.00 (m, 1H), 3.14-3.10 (m, 1H), 2.82-2.73 (m, 2H), 2.38 (s, 1H), 2.25 (s, 3H), 2.12-2.04 (m, 1H), 1.93-1.84 (m, 2H), 1.71-1.65 (m, 1H). LC-MS (M+H)⁺=389.3.

Example 6: Analytical SFC tR=4.22 min. ¹H NMR (400 MHz, DMSO-d6, 80° C.) δ11.27 (s, 1H), 6.99-6.89 (m, 3H), 6.48 (s, 1H), 5.66 (s, 1H), 5.28 (s, 2H), 5.13 (s, 2H), 4.93 (s, 2H), 4.07-4.00 (m, 1H), 3.14-3.10 (m, 1H), 2.82-2.73 (m, 2H), 2.38 (s, 1H), 2.25 (s, 3H), 2.12-2.04 (m, 1H), 1.93-1.84 (m, 2H), 1.71-1.65 (m, 1H). LC-MS (M+H)⁺=389.3.

### Example 7 & Example 8: (5-amino-6,8-dihydro-1H-furo[3,4-d]pyrrolo[3,2-b]pyridin-2-yl)((1R,5S)-8-cyclopropyl-10-fluoro-3,4,5,6-tetrahydro-1,5-methanobenzo[c]azocin-2(1H)-yl)methanone & (5-amino-6,8-dihydro-1H-furo[3,4-d]pyrrolo[3,2-b]pyridin-2-yl)((1S,5R)-8-cyclopropyl-10-fluoro-3,4,5,6-tetrahydro-1,5-methanobenzo[c]azocin-2(1H)-yl)methanone

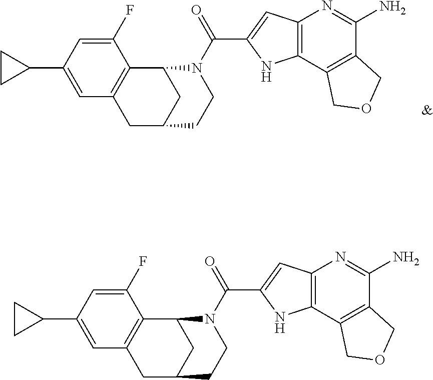 [SMILES: Nc1nc2cc(C(=O)N3CC[C@@H]4Cc5cc(C6CC6)cc(F)c5[C@H]3C4)[nH]c2c2c1COC2 ; Nc1nc2cc(C(=O)N3CC[C@H]4Cc5cc(C6CC6)cc(F)c5[C@@H]3C4)[nH]c2c2c1COC2]

### Step 1: 1-(bromomethyl)-3-cyclopropyl-5-fluorobenzene

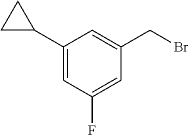 [SMILES: Fc1cc(CBr)cc(C2CC2)c1]

To a mixture of 1-cyclopropyl-3-fluoro-5-methylbenzene (29 g, 193 mmol) and BPO (4.6 g, 19 mmol) in CCl₄ (100 mL) was added NBS (70 g, 387 mmol) under nitrogen. The mixture was heated to reflux and stirred overnight. The mixture was cooled to room temperature and concentrated under reduced pressure. The residue was purified by silica gel chromatography with PE to give the title compound (6.5 g, 15%).

### Step 2: 1-benzyl-4-(3-cyclopropyl-5-fluorobenzyl)-3,4-dihydropyridin-2(1H)-one

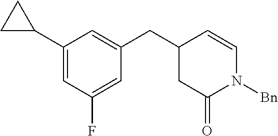 [SMILES: O=C1CC(Cc2cc(F)cc(C3CC3)c2)C=CN1Cc1ccccc1]

To a solution of 1-benzylpyridin-2(1H)-one (3.5 g, 18.9 mmol) in DCM (50 mL) was added TIPSOTf (11.6 g, 37.8 mmol) at room temperature with stirring. After 30 min, 2,6-lutidine (4.0 g, 37.8 mmol) was added. The mixture was stirred for 30 min and then cooled to 0° C. In parallel, to a solution of 1-(bromomethyl)-3-cyclopropyl-5-fluorobenzene (6.5 g, 28.4 mmol) in anhydrous ether (100 mL) was added magnesium turnings (1.4 g, 56.8 mmol) and iodine (720 mg, 2.8 mmol) under nitrogen. After the initiation of reaction, the mixture was heated at refluxing temperature for 2 h. The mixture was cooled to room temperature. The supernatant was transferred to an addition funnel and added dropwise to the aforementioned solution of 1-benzyl-2-((triisopropylsilyl)oxy)pyridin-1-ium trifluoromethanesulfonate and 2,6-lutidine in DCM at 0° C. The mixture was stirred at room temperature overnight. Water (100 mL) was added slowly and the mixture was extracted with EtOAc (100 mL). The organic layer was washed with 1 N hydrochloric acid (20 mL), dried over Na₂SO₄, filtered and concentrated under reduced pressure. The residue was purified by silica gel chromatograph (PE:EtOAc=1:0 to 5:1) to give the title compound (2.2 g, 23%). LC-MS (M+H)⁺=336.1.

### Step 3: 2-benzyl-8-cyclopropyl-10-fluoro-1,4,5,6-tetrahydro-1,5-methanobenzo[c]azocin-3(2H)-one

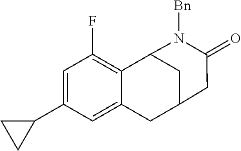 [SMILES: O=C1CC2Cc3cc(C4CC4)cc(F)c3C(C2)N1Cc1ccccc1]

The title compound (1.7 g, 77%) was prepared in a manner similar to that in Example 1 & Example 2 step 9 from 1-benzyl-4-(3-cyclopropyl-5-fluorobenzyl)-3,4-dihydropyridin-2(1H)-one. LC-MS (M+H)=336.1.

### Step 4: 2-benzyl-8-cyclopropyl-10-fluoro-1,2,3,4,5,6-hexahydro-1,5-methanobenzo[c]azocine

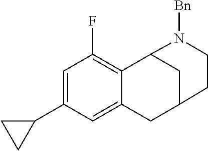 [SMILES: Fc1cc(C2CC2)cc2c1C1CC(CCN1Cc1ccccc1)C2]

To a solution of 2-benzyl-8-cyclopropyl-10-fluoro-1,4,5,6-tetrahydro-1,5-methanobenzo[c]azocin-3(2H)-one (770 mg, 2.3 mmol) in THF (10 mL) was added BH₃ (1.0 M in THF, 3.5 mL, 3.5 mmol) at 0° C. The mixture was warmed to 60° C. and stirred for 2 h. The mixture was cooled to room temperature, followed by the addition of concentrated hydrochloric acid (5 mL). The mixture was stirred for 0.5 h at 80° C. and then cooled to room temperature. The organic solvent was evaporated under reduced pressure, and the residue was partitioned between EtOAc (10 mL) and saturated NaHCO₃ (10 mL). The organic layer was separated and concentrated under reduced pressure to give the title compound (610 mg, 83%). LC-MS (M+H)⁺=322.1.

### Step 5: 8-cyclopropyl-10-fluoro-1,2,3,4,5,6-hexahydro-1,5-methanobenzo[c]azocine

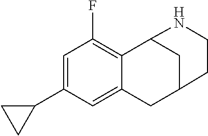 [SMILES: Fc1cc(C2CC2)cc2c1C1CC(CCN1)C2]

The title compound (90 mg, 20%) was prepared in a manner similar to that in Example 1 & Example 2 step 15 from 2-benzyl-8-cyclopropyl-10-fluoro-1,2,3,4,5,6-hexahydro-1,5-methanobenzo[c]azocine. LC-MS (M+H)=232.1.

### Step 6: (5-amino-1-((2-(trimethylsilyl)ethoxy)methyl)-6,8-dihydro-1H-furo[3,4-d]pyrrolo[3,2-b]pyridin-2-yl)(8-cyclopropyl-10-fluoro-3,4,5,6-tetrahydro-1,5-methanobenzo[c]azocin-2(1H)-yl)methanone

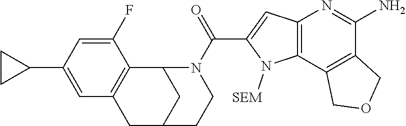 [SMILES: C[Si](C)(C)CCOCn1c(C(=O)N2CCC3Cc4cc(C5CC5)cc(F)c4C2C3)cc2nc(N)c3c(c21)COC3]

The title compound (150 mg, 68%) was prepared in a manner similar to that in Example 1 & Example 2 step 16 from 8-cyclopropyl-10-fluoro-1,2,3,4,5,6-hexahydro-1,5-methanobenzo[c]azocine and 5-amino-1-((2-(trimethylsilyl)ethoxy)methyl)-6,8-dihydro-1H-furo[3,4-d]pyrrolo[3,2-b]pyridine-2-carboxylic acid. LC-MS (M+H)=563.2.

### Step 7: (5-amino-6,8-dihydro-1H-furo[3,4-d]pyrrolo[3,2-b]pyridin-2-yl)((1R,5S)-8-cyclopropyl-10-fluoro-3,4,5,6-tetrahydro-1,5-methanobenzo[c]azocin-2(1H)-yl)methanone & (5-amino-6,8-dihydro-1H-furo[3,4-d]pyrrolo[3,2-b]pyridin-2-yl)((1S,5R)-8-cyclopropyl-10-fluoro-3,4,5,6-tetrahydro-1,5-methanobenzo[c]azocin-2(1H)-yl)methanone

Example 7 (37 mg, 32%) and Example 8 (33 mg, 29%) were prepared in a manner similar to that in Example 1 & Example 2 step 17 from (5-amino-1-((2-(trimethylsilyl)ethoxy)methyl)-6,8-dihydro-1H-furo[3,4-d]pyrrolo[3,2-b]pyridin-2-yl)(8-cyclopropyl-10-fluoro-3,4,5,6-tetrahydro-1,5-methanobenzo[c]azocin-2(1H)-yl)methanone, and the enantiomers were separated by chiral SFC.

Analytical chiral-SFC condition as below. Column: CHIRALPAK AS-H; Column size: 4.6×100 mm, 5 μm; Mobile phase: 4 mM methanolic NH₃:CO₂, 1:9 to 1:1 in 3 min, 1:1 for 2 min, 1:1 to 1:9 in 0.1 min, 1:9 for 1.9 min; Flow: 2.0 mL/min; Temperature: 35° C.; back pressure: 1500 psi.

Example 7: Analytical SFC tR=3.53 min. ¹H NMR (400 MHz, DMSO-d6) δ 11.59-11.37 (m, 1H), 6.88-6.74 (m, 1H), 6.70-6.29 (m, 2H), 6.05-6.75 (m, 1H), 5.68-5.49 (m, 2H), 5.21-5.02 (m, 2H), 5.00-4.83 (m, 2H), 4.21-3.90 (m, 1H), 3.18-2.91 (m, 2H), 2.90-2.71 (m, 1H), 2.42-1.56 (m, 6H), 1.02-0.84 (m, 2H), 0.75-0.59 (m, 2H). LC-MS (M+H)⁺=433.4.

Example 8: Analytical SFC tR=4.22 min. ¹H NMR (400 MHz, DMSO-d6) δ 11.59-11.37 (m, 1H), 6.88-6.74 (m, 1H), 6.70-6.29 (m, 2H), 6.05-6.75 (m, 1H), 5.68-5.49 (m, 2H), 5.21-5.02 (m, 2H), 5.00-4.83 (m, 2H), 4.21-3.90 (m, 1H), 3.18-2.91 (m, 2H), 2.90-2.71 (m, 1H), 2.42-1.56 (m, 6H), 1.02-0.84 (m, 2H), 0.75-0.59 (m, 2H). LC-MS (M+H)⁺=433.4.

### Example 9 & Example 10: (5-amino-6,8-dihydro-1H-furo[3,4-d]pyrrolo[3,2-b]pyridin-2-yl)((1S,5R)-8-fluoro-3,4,5,6-tetrahydro-1,5-methanobenzo[c]azocin-2(1H)-yl)methanone & (5-amino-6,8-dihydro-1H-furo[3,4-d]pyrrolo[3,2-b]pyridin-2-yl)((1R,5S)-8-fluoro-3,4,5,6-tetrahydro-1,5-methanobenzo[c]azocin-2(1H)-yl)methanone

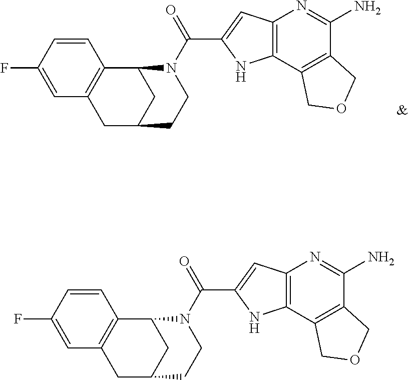 [SMILES: Nc1nc2cc(C(=O)N3CC[C@H]4Cc5cc(F)ccc5[C@@H]3C4)[nH]c2c2c1COC2 ; Nc1nc2cc(C(=O)N3CC[C@@H]4Cc5cc(F)ccc5[C@H]3C4)[nH]c2c2c1COC2]

### Step 1: 1-benzyl-4-(3-fluorobenzyl)-3,4-dihydropyridin-2(1H)-one

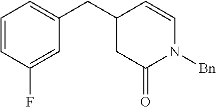 [SMILES: O=C1CC(Cc2cccc(F)c2)C=CN1Cc1ccccc1]

The title compound (5.3 g, 56%) was prepared in a manner similar to that in Example 1 & Example 2 step 8 from 1-benzylpyridin-2(1H)-one and (3-fluorobenzyl)magnesium bromide. LC-MS (M+H)⁺=296.1.

### Step 2: 2-benzyl-8-fluoro-1,4,5,6-tetrahydro-1,5-methanobenzo[c]azocin-3(2H)-one

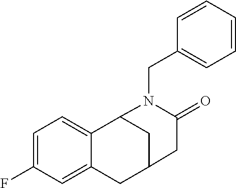 [SMILES: O=C1CC2Cc3cc(F)ccc3C(C2)N1Cc1ccccc1]

The title compound (1.06 g, 20%) was prepared in a manner similar to that in Example 1 & Example 2 step 9 from 1-benzyl-4-(3-fluorobenzyl)-3,4-dihydropyridin-2(1H)-one. LC-MS (M+H)⁺=296.1.

### Step 3: 8-fluoro-1,2,3,4,5,6-hexahydro-1,5-methanobenzo[c]azocine

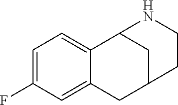 [SMILES: Fc1ccc2c(c1)CC1CCNC2C1]

The title compound (172 mg, 25%) was prepared in a manner similar to that in Example 1 & Example 2 step 10 & step 15 from 2-benzyl-8-fluoro-1,4,5,6-tetrahydro-1,5-methanobenzo[c]azocin-3(2H)-one. LC-MS (M+H)⁺=192.1.

### Step 4: (5-amino-1-((2-(trimethylsilyl)ethoxy)methyl)-6,8-dihydro-1H-furo[3,4-d]pyrrolo[3,2-b]pyridin-2-yl)(8-fluoro-3,4,5,6-tetrahydro-1,5-methanobenzo[c]azocin-2(1H)-yl)methanone

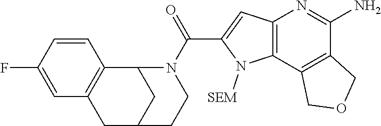 [SMILES: C[Si](C)(C)CCOCn1c(C(=O)N2CCC3Cc4cc(F)ccc4C2C3)cc2nc(N)c3c(c21)COC3]

The title compound (300 mg, 100%) was prepared in a manner similar to that in Example 1 & Example 2 step 16 from 8-fluoro-1,2,3,4,5,6-hexahydro-1,5-methanobenzo[c]azocine and 5-amino-1-((2-(trimethylsilyl)ethoxy)methyl)-6,8-dihydro-1H-furo[3,4-d]pyrrolo[3,2-b]pyridine-2-carboxylic acid. LC-MS (M+H)⁺=523.2.

### Step 5: (5-amino-6,8-dihydro-1H-furo[3,4-d]pyrrolo[3,2-b]pyridin-2-yl)((1S,5R)-8-fluoro-3,4,5,6-tetrahydro-1,5-methanobenzo[c]azocin-2(1H)-yl)methanone & (5-amino-6,8-dihydro-1H-furo[3,4-d]pyrrolo[3,2-b]pyridin-2-yl)((1R,5S)-8-fluoro-3,4,5,6-tetrahydro-1,5-methanobenzo[c]azocin-2(1H)-yl)methanone

Example 9 (46 mg, 20%) and Example 10 (46 mg, 20%) were prepared in a manner similar to that in Example 1 & Example 2 step 17 from (5-amino-1-((2-(trimethylsilyl)ethoxy)methyl)-6,8-dihydro-1H-furo[3,4-d]pyrrolo[3,2-b]pyridin-2-yl)(8-fluoro-3,4,5,6-tetrahydro-1,5-methanobenzo[c]azocin-2(1H)-yl)methanone, and the enantiomers were separated by chiral SFC.

Analytical chiral-SFC condition as below. Column: Lux Cellulose-3; Column size: 4.6×100 mm, m; Mobile phase: 4 mM methanolic NH₃:CO₂, 1:9 to 1:1 in 3 min, 1:1 for 2 min, 1:1 to 1:9 in 0.1 min, 1:9 for 1.9 min; Flow: 2.0 mL/min; Temperature: 35° C.; back pressure: 1500 psi.

Example 9: Analytical SFC tR=3.06 min. ¹H NMR (400 MHz, DMSO-d6, 80° C.) δ11.72 (s, 1H), 7.21 (s, 1H), 7.10-6.85 (m, 2H), 6.71 (s, 1H), 6.42 (s, 1H), 6.00 (s, 2H), 5.77 (s, 1H), 5.13 (s, 2H), 4.91 (s, 2H), 4.05 (s, 1H), 3.12-3.09 (m, 1H), 2.88 (d, J=28.6 Hz, 1H), 2.36 (s, 1H), 1.90 (t, J=17.8 Hz, 3H), 1.66 (s, 1H). LC-MS (M+H)⁺=393.1.

Example 10: Analytical SFC tR=4.43 min. ¹H NMR (400 MHz, DMSO-d6, 80° C.) δ11.72 (s, 1H), 7.21 (s, 1H), 7.10-6.85 (m, 2H), 6.71 (s, 1H), 6.42 (s, 1H), 6.00 (s, 2H), 5.77 (s, 1H), 5.13 (s, 2H), 4.91 (s, 2H), 4.05 (s, 1H), 3.12-3.09 (m, 1H), 2.88 (d, J=28.6 Hz, 1H), 2.36 (s, 1H), 1.90 (t, J=17.8 Hz, 3H), 1.66 (s, 1H). LC-MS (M+H)⁺=393.1.

### Example 11 & Example 12: (5-amino-6,8-dihydro-1H-furo[3,4-d]pyrrolo[3,2-b]pyridin-2-yl)((1R,5S)-8,10-difluoro-3,4,5,6-tetrahydro-1,5-methanobenzo[c]azocin-2(1H)-yl)methanone & (5-amino-6,8-dihydro-1H-furo[3,4-d]pyrrolo[3,2-b]pyridin-2-yl)((1S,5R)-8,10-difluoro-3,4,5,6-tetrahydro-1,5-methanobenzo[c]azocin-2(1H)-yl)methanone

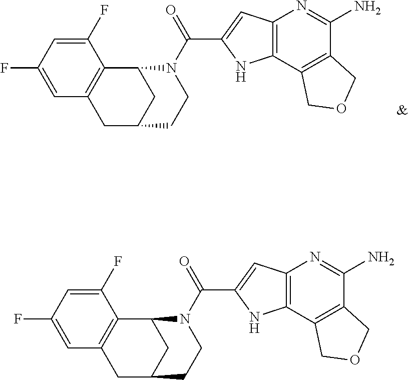 [SMILES: Nc1nc2cc(C(=O)N3CC[C@@H]4Cc5cc(F)cc(F)c5[C@H]3C4)[nH]c2c2c1COC2 ; Nc1nc2cc(C(=O)N3CC[C@H]4Cc5cc(F)cc(F)c5[C@@H]3C4)[nH]c2c2c1COC2]

### Step 1: 1-benzyl-4-(3,5-difluorobenzyl)-3,4-dihydropyridin-2(1H)-one

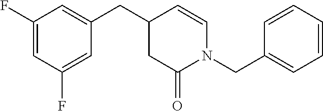 [SMILES: O=C1CC(Cc2cc(F)cc(F)c2)C=CN1Cc1ccccc1]

The title compound (4.2 g, 42%) was prepared in a manner similar to that in Example 1 & Example 2 step 8 from 1-benzylpyridin-2(1H)-one and (3,5-difluorobenzyl)magnesium bromide. LC-MS (M+H)⁺=314.1.

### Step 2: 2-benzyl-8,10-difluoro-1,4,5,6-tetrahydro-1,5-methanobenzo[c]azocin-3(2H)-one

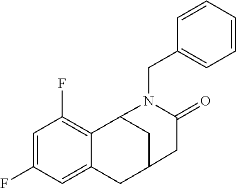 [SMILES: O=C1CC2Cc3cc(F)cc(F)c3C(C2)N1Cc1ccccc1]

The title compound (860 mg, 20%) was prepared in a manner similar to that in Example 1 & Example 2 step 9 from 1-benzyl-4-(3,5-difluorobenzyl)-3,4-dihydropyridin-2(1H)-one. LC-MS (M+H)⁺=314.1.

### Step 3: 8,10-difluoro-1,2,3,4,5,6-hexahydro-1,5-methanobenzo[c]azocine

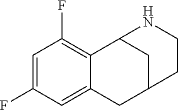 [SMILES: Fc1cc(F)c2c(c1)CC1CCNC2C1]

The title compound (170 mg, 30%) was prepared in a manner similar to that in Example 1 & Example 2 step 10 & step 15 from 2-benzyl-8,10-difluoro-1,4,5,6-tetrahydro-1,5-methanobenzo[c]azocin-3(2H)-one. LC-MS (M+H)⁺=210.1.

### Step 4: (5-amino-1-((2-(trimethylsilyl)ethoxy)methyl)-6,8-dihydro-1H-furo[3,4-d]pyrrolo[3,2-b]pyridin-2-yl)(8,10-difluoro-3,4,5,6-tetrahydro-1,5-methanobenzo[c]azocin-2(1H)-yl)methanone

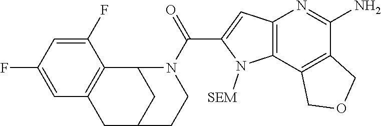 [SMILES: C[Si](C)(C)CCOCn1c(C(=O)N2CCC3Cc4cc(F)cc(F)c4C2C3)cc2nc(N)c3c(c21)COC3]

The title compound (200 mg, 56%) was prepared in a manner similar to that in Example 1 & Example 2 step 16 from 8,10-difluoro-1,2,3,4,5,6-hexahydro-1,5-methanobenzo[c]azocine and 5-amino-1-((2-(trimethylsilyl)ethoxy)methyl)-6,8-dihydro-1H-furo[3,4-d]pyrrolo[3,2-b]pyridine-2-carboxylic acid. LC-MS (M+H)⁺=541.2.

### Step 5: (5-amino-6,8-dihydro-1H-furo[3,4-d]pyrrolo[3,2-b]pyridin-2-yl)((1R,5S)-8,10-difluoro-3,4,5,6-tetrahydro-1,5-methanobenzo[c]azocin-2(1H)-yl)methanone & (5-amino-6,8-dihydro-1H-furo[3,4-d]pyrrolo[3,2-b]pyridin-2-yl)((1S,5R)-8,10-difluoro-3,4,5,6-tetrahydro-1,5-methanobenzo[c]azocin-2(1H)-yl)methanone

Example 11 (65 mg, 43%) and Example 12 (61 mg, 40%) were prepared in a manner similar to that in Example 1 & Example 2 step 17 from (5-amino-1-((2-(trimethylsilyl)ethoxy)methyl)-6,8-dihydro-1H-furo[3,4-d]pyrrolo[3,2-b]pyridin-2-yl)(8,10-difluoro-3,4,5,6-tetrahydro-1,5-methanobenzo[c]azocin-2(1H)-yl)methanone, and the enantiomers were separated by chiral SFC.

Analytical chiral-SFC condition as below. Column: CHIRALPAK AS-H; Column size: 4.6×100 mm, 5 μm; Mobile phase: 4 mM methanolic NH₃:CO₂, 1:9 to 1:1 in 3 min, 1:1 for 2 min, 1:1 to 1:9 in 0.1 min, 1:9 for 1.9 min; Flow: 2.0 mL/min; Temperature: 35° C.; back pressure: 1500 psi.

Example 11: Analytical SFC tR=2.99 min. ¹H NMR (400 MHz, DMSO-d6, 80° C.) δ11.49 (s, 1H), 6.7.02-6.84 (m, 2H), 6.65-6.39 (m, 1H), 5.98 (s, 1H), 5.55 (s, 2H), 5.11 (s, 2H), 4.90 (s, 2H), 4.09 (s, 1H), 3.30-3.02 (m, 1H), 2.99-2.75 (m, 1H), 2.40-2.25 (m, 2H), 2.10-1.64 (m, 4H). LC-MS (M+H)⁺=411.1.

Example 12: Analytical SFC tR=3.50 min. ¹H NMR (400 MHz, DMSO-d6, 80° C.) δ11.49 (s, 1H), 6.7.02-6.84 (m, 2H), 6.65-6.39 (m, 1H), 5.98 (s, 1H), 5.55 (s, 2H), 5.11 (s, 2H), 4.90 (s, 2H), 4.09 (s, 1H), 3.30-3.02 (m, 1H), 2.99-2.75 (m, 1H), 2.40-2.25 (m, 2H), 2.10-1.64 (m, 4H). LC-MS (M+H)⁺=411.1.

### Example 13 & 14: (5-amino-6,8-dihydro-1H-furo[3,4-d]pyrrolo[3,2-b]pyridin-2-yl)((1S,5R)-8-bromo-3,4,5,6-tetrahydro-1,5-methanobenzo[c]azocin-2(1H)-yl)methanone & (5-amino-6,8-dihydro-1H-furo[3,4-d]pyrrolo[3,2-b]pyridin-2-yl)((1R,5S)-8-bromo-3,4,5,6-tetrahydro-1,5-methanobenzo[c]azocin-2(1H)-yl)methanone

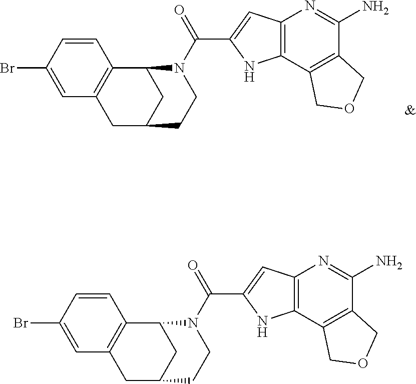 [SMILES: Nc1nc2cc(C(=O)N3CC[C@H]4Cc5cc(Br)ccc5[C@@H]3C4)[nH]c2c2c1COC2 ; Nc1nc2cc(C(=O)N3CC[C@@H]4Cc5cc(Br)ccc5[C@H]3C4)[nH]c2c2c1COC2]

### Step 1: 1-benzyl-4-(3-bromobenzyl)-3,4-dihydropyridin-2(1H)-one

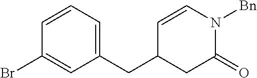 [SMILES: O=C1CC(Cc2cccc(Br)c2)C=CN1Cc1ccccc1]

The title compound (1.4 g, 42%) was prepared in a manner similar to that in Example 1 & Example 2 step 8 from 1-benzylpyridin-2(1H)-one and (3-bromobenzyl)magnesium bromide. LC-MS (M+H)⁺=356.2.

### Step 2: 2-benzyl-8-bromo-1,4,5,6-tetrahydro-1,5-methanobenzo[c]azocin-3(2H)-one

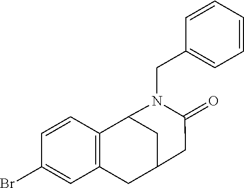 [SMILES: O=C1CC2Cc3cc(Br)ccc3C(C2)N1Cc1ccccc1]

The title compound (130 mg, 5%) was prepared in a manner similar to that in Example 1 & Example 2 step 9 from 1-benzyl-4-(3-bromobenzyl)-3,4-dihydropyridin-2(1H)-one. LC-MS (M+H)⁺=356.2.

### Step 3: 8-bromo-1,4,5,6-tetrahydro-15-methanobenzo[c]azocin-3(2H)-one

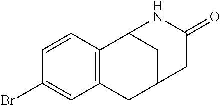 [SMILES: O=C1CC2Cc3cc(Br)ccc3C(C2)N1]

The title compound (60 mg, 62%) was prepared in a manner similar to that in Example 1 & Example 2 step 15 from 2-benzyl-8-bromo-1,4,5,6-tetrahydro-1,5-methanobenzo[c]azocin-3(2H)-one. LC-MS (M+H)⁺=266.2.

### Step 4: 8-bromo-1,2,3,4,5,6-hexahydro-1,5-methanobenzo[c]azocine

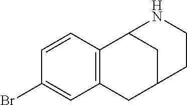 [SMILES: Brc1ccc2c(c1)CC1CCNC2C1]

The title compound (10 mg, 18%) was prepared in a manner similar to that in Example 1 & Example 2 step 10 from 8-bromo-1,4,5,6-tetrahydro-1,5-methanobenzo[c]azocin-3(2H)-one. LC-MS (M+H)⁺=252.2.

### Step 5: (5-amino-1-((2-(trimethylsilyl)ethoxy)methyl)-6,8-dihydro-1H-furo[3,4-d]pyrrolo[3,2-b]pyridin-2-yl)(8-bromo-3,4,5,6-tetrahydro-1,5-methanobenzo[c]azocin-2(1H)-yl)methanone

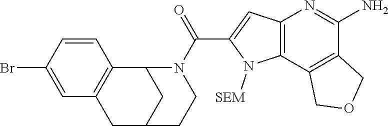 [SMILES: C[Si](C)(C)CCOCn1c(C(=O)N2CCC3Cc4cc(Br)ccc4C2C3)cc2nc(N)c3c(c21)COC3]

The title compound (15 mg, 65%) was prepared in a manner similar to that in Example 1 & ample 2 step 16 from 8-bromo-1,2,3,4,5,6-hexahydro-1,5-methanobenzo[c]azocine and 5-amino-1-((2-(trimethylsilyl)ethoxy)methyl)-6,8-dihydro-1H-furo[3,4-d]pyrrolo[3,2-b]pyridine-2-carboxylic acid. LC-MS (M+H)⁺=583.2.

### Step 6: (5-amino-6,8-dihydro-1H-furo[3,4-d]pyrrolo[3,2-b]pyridin-2-yl)((1S,5R)-8-bromo-3,4,5,6-tetrahydro-1,5-methanobenzo[c]azocin-2(1H)-yl)methanone & (5-amino-6,8-dihydro-1H-furo[3,4-d]pyrrolo[3,2-b]pyridin-2-yl)((1R,5S)-8-bromo-3,4,5,6-tetrahydro-1,5-methanobenzo[c]azocin-2(1H)-yl)methanone

Example 13 (2.5 mg, 13%) and Example 14 (2.1 mg, 9%) were prepared in a manner similar to that in Example 1 & Example 2 step 17 from (5-amino-1-((2-(trimethylsilyl)ethoxy)methyl)-6,8-dihydro-1H-furo[3,4-d]pyrrolo[3,2-b]pyridin-2-yl)(8-bromo-3,4,5,6-tetrahydro-1,5-methanobenzo[c]azocin-2(1H)-yl)methanone, and the enantiomers were separated by chiral SFC.

Analytical chiral-SFC condition as below. Column: Lux Cellulose-3; Column size: 4.6×100 mm, 5 μm; Mobile phase: 4 mM methanolic NH₃:CO₂, 1:9 to 1:1 in 3 min, 1:1 for 2 min, 1:1 to 1:9 in 0.1 min, 1:9 for 1.9 min; Flow: 2.0 mL/min; Temperature: 35° C.; back pressure: 1500 psi.

Example 13: Analytical SFC tR=3.73 min. ¹H NMR (400 MHz, DMSO-d6, 80° C.) δ11.28 (s, 1H), 7.39 (s, 1H), 7.33-7.25 (m, 1H), 7.01 (s, 1H), 6.50 (s, 1H), 5.69 (s, 1H), 5.29 (s, 2H), 5.16-4.10 (m, 2H), 4.97-4.87 (m, 2H), 4.11-4.01 (m, 1H), 3.22-3.15 (m, 1H), 2.87-2.73 (m, 2H), 2.41-2.36 (m, 1H), 2.16-2.02 (m, 1H), 1.95-1.84 (m, 2H), 1.73-1.65 (m, 1H). LC-MS (M+H)⁺=453.2.

Example 14: Analytical SFC tR=4.14 min. ¹H NMR (400 MHz, DMSO-d6, 80° C.) δ11.28 (s, 1H), 7.39 (s, 1H), 7.33-7.25 (m, 1H), 7.01 (s, 1H), 6.50 (s, 1H), 5.69 (s, 1H), 5.29 (s, 2H), 5.16-4.10 (m, 2H), 4.97-4.87 (m, 2H), 4.11-4.01 (m, 1H), 3.22-3.15 (m, 1H), 2.87-2.73 (m, 2H), 2.41-2.36 (m, 1H), 2.16-2.02 (m, 1H), 1.95-1.84 (m, 2H), 1.73-1.65 (m, 1H). LC-MS (M+H)⁺=453.2.

### Example 15 & Example 16: (5-amino-6,8-dihydro-1H-furo[3,4-d]pyrrolo[3,2-b]pyridin-2-yl)((1S,5R)-10-fluoro-8-(trifluoromethyl)-3,4,5,6-tetrahydro-1,5-methanobenzo[c]azocin-2(1H)-yl)methanone & (5-amino-6,8-dihydro-1H-furo[3,4-d]pyrrolo[3,2-b]pyridin-2-yl)((1R,5S)-10-fluoro-8-(trifluoromethyl)-3,4,5,6-tetrahydro-1,5-methanobenzo[c]azocin-2(1H)-yl)methanone

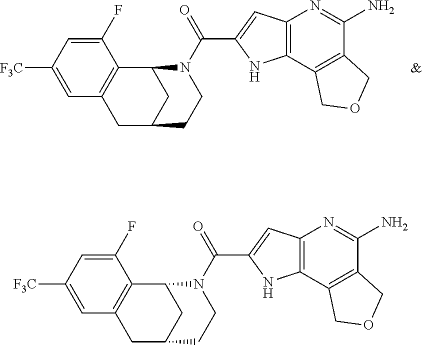 [SMILES: Nc1nc2cc(C(=O)N3CC[C@H]4Cc5cc(C(F)(F)F)cc(F)c5[C@@H]3C4)[nH]c2c2c1COC2 ; Nc1nc2cc(C(=O)N3CC[C@@H]4Cc5cc(C(F)(F)F)cc(F)c5[C@H]3C4)[nH]c2c2c1COC2]

### Step 1: 1-benzyl-4-(3-fluoro-5-methoxybenzyl)-3,4-dihydropyridin-2(1H)-one

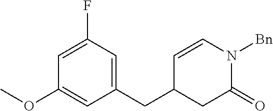 [SMILES: COc1cc(F)cc(CC2C=CN(Cc3ccccc3)C(=O)C2)c1]

To a solution of 1-benzylpyridin-2(1H)-one (23.0 g, 124 mmol) in anhydrous DCM (400 mL) was added TIPSOTf (69.7 g, 228 mmol) dropwise. The mixture was stirred at 70° C. for 1 h. The mixture was cooled to room temperature and 2,6-lutidine (24.4 g, 228 mmol) was added. After 1 h, the mixture was cooled to 0° C. and (3-fluoro-5-methoxybenzyl)magnesium bromide (456 mL, 0.5 M in diethyl ether, 228 mmol) was added dropwise. The mixture was warmed to room temperature and stirred overnight. The mixture was quenched with saturated NaHCO₃ (500 mL) and extracted with EtOAc (1 L). The organic layer was washed with brine (500 mL), dried over anhydrous Na₂SO₄, filtered, and concentrated under reduced pressure. The residue was purified by silica gel chromatograph (PE/EtOAc=1/1) to give the title compound (20.0 g, 50%). LC-MS (M+H)⁺=326.1.

### Step 2: 2-benzyl-10-fluoro-8-methoxy-1,4,5,6-tetrahydro-1,5-methanobenzo[c]azocin-3(2H)-one

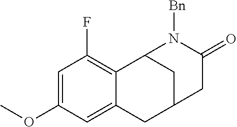 [SMILES: COc1cc(F)c2c(c1)CC1CC(=O)N(Cc3ccccc3)C2C1]

To a solution of 1-benzyl-4-(3-fluoro-5-methoxybenzyl)-3,4-dihydropyridin-2(1H)-one (3.6 g, 11 mmol) in MeCN (800 mL) was added TIPSOTf (8.47 g, 27.7 mmol). The mixture was stirred at refluxing temperature for 16 h. The mixture was cooled to room temperature and concentrated in vacuo. The residue was partitioned between EtOAc (100 mL) and saturated NaHCO₃ (50 mL). The organic layer was washed with brine (50 mL), dried over Na₂SO₄, filtered and concentrated under reduced pressure. The residue was purified by silica gel chromatograph (PE/EtOAc=4/1) to give the title compound (2.0 g, 56%). LC-MS (M+H)⁺=326.1.

### Step 3: 2-benzyl-10-fluoro-8-methoxy-1,2,3,4,5,6-hexahydro-1,5-methanobenzo[c]azocine

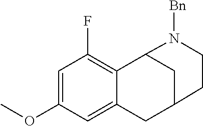 [SMILES: COc1cc(F)c2c(c1)CC1CCN(Cc3ccccc3)C2C1]

To a mixture of 2-benzyl-10-fluoro-8-methoxy-1,4,5,6-tetrahydro-1,5-methanobenzo[c]azocin-3(2H)-one (11.0 g, 33.8 mmol) in THF (200 mL) was added and BH₃ (1.0 M in THF, 44 mL, 44 mmol). The mixture was stirred at refluxing temperature for 16 h and cooled to room temperature. The mixture was cooled to room temperature, followed by addition of HCl (1 M, 150 mL). The mixture was stirred for 2 h at 80° C. and then cooled to room temperature. The mixture was diluted with EtOAc (300 mL), successively washed with saturated NaHCO₃ (300 mL) and brine (100 mL), dried over Na₂SO₄, filtered and concentrated under vacuum. The residue was purified by silica gel chromatograph (PE:EtOAc=4:1) to give the title compound (7.2 g, 68%). LC-MS (M+H)⁺=312.2.

### Step 4: 2-benzyl-10-fluoro-1,2,3,4,5,6-hexahydro-1,5-methanobenzo[c]azocin-8-ol

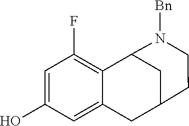 [SMILES: Oc1cc(F)c2c(c1)CC1CCN(Cc3ccccc3)C2C1]

To a solution of 2-benzyl-10-fluoro-8-methoxy-1,2,3,4,5,6-hexahydro-1,5-methanobenzo[c]azocine (7.2 g, 22.1 mmol) in anhydrous DCM (100 mL) was added BBr₃ (1.0 M in DCM, 66.4 mL, 66.4 mmol) dropwise at −78° C. under nitrogen. The mixture was warmed to room temperature and stirred for 6 h. The mixture was concentrated under reduced pressure and re-dissolved with EtOAc (200 mL). The mixture was washed with saturated NaHCO₃ (100 mL) and brine (100 mL), dried over Na₂SO₄, filtered and concentrated under reduced pressure. The residue was purified by silica gel chromatograph (PE/EtOAc=1/1) to give the title compound (4.0 g, 61%). LC-MS (M+H)=298.1.

### Step 5: 2-benzyl-10-fluoro-1,2,3,4,5,6-hexahydro-1,5-methanobenzo[c]azocin-8-yl trifluoromethanesulfonate

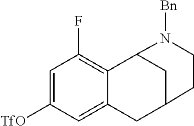 [SMILES: O=S(=O)(Oc1cc(F)c2c(c1)CC1CCN(Cc3ccccc3)C2C1)C(F)(F)F]

To a mixture of 2-benzyl-10-fluoro-1,2,3,4,5,6-hexahydro-1,5-methanobenzo[c]azocin-8-ol (4.0 g, 13.4 mmol) and Et₃N (1.9 g, 18.8 mmol) in anhydrous DCM (60 mL) was added Tf₂O (4.9 g, 17.5 mmol) dropwise at −78° C. under nitrogen. The mixture was stirred at −78° C. for 1 h. The mixture was diluted with DCM (100 mL) and successively washed with saturated NaHCO₃ (100 mL) and brine (100 mL). The organic layer was dried over Na₂SO₄, filtered and concentrated to give the title compound (5.7 g, 99%). LC-MS (M+H)=430.1.

### Step 6: 2-benzyl-10-fluoro-8-(4,4,5,5-tetramethyl-1,3,2-dioxaborolan-2-yl)-1,2,3,4,5,6-hexahydro-1,5-methanobenzo[c]azocine

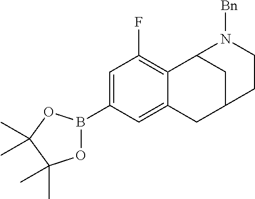 [SMILES: CC1(C)OB(c2cc(F)c3c(c2)CC2CCN(Cc4ccccc4)C3C2)OC1(C)C]

To a mixture of 2-benzyl-10-fluoro-1,2,3,4,5,6-hexahydro-1,5-methanobenzo[c]azocin-8-yl trifluoromethanesulfonate (5.7 g, 13.3 mmol) and BPD (3.4 g, 21.5 mmol) in dioxane (70 mL) was added Pd(dppf)Cl₂ (659 mg, 0.90 mmol) and AcOK (3.5 g, 36 mmol) under nitrogen. The mixture was stirred at refluxing temperature overnight and then cooled to room temperature. The mixture was diluted with EtOAc (50 mL) and washed with brine (30 mL). The aqueous layer was back extracted with EtOAc (100 mL). The combined organic layer was dried over Na₂SO₄, filtered and concentrated under reduced pressure. The residue was purified by silica gel chromatograph (PE/EtOAc=3/1) to give the title compound (4.0 g, 73%). LC-MS (M+H)⁺=408.1.

### Step 7: 2-benzyl-10-fluoro-8-(trifluoromethyl)-1,2,3,4,5,6-hexahydro-1,5-methanobenzo[c]azocine

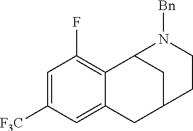 [SMILES: Fc1cc(C(F)(F)F)cc2c1C1CC(CCN1Cc1ccccc1)C2]

A mixture of 2-benzyl-10-fluoro-8-(4,4,5,5-tetramethyl-1,3,2-dioxaborolan-2-yl)-1,2,3,4,5,6-hexahydro-1,5-methanobenzo[c]azocine (2.0 g, 4.9 mmol) and (1,10-phenanthroline)(trifluoromethyl)copper(I) (1.83 g, 5.88 mmol) in anhydrous DMF (15 mL) was bubbled with air for 5 min in a sealed tube. The tube was sealed and the mixture was stirred at 50° C. for 3 h. The mixture was cooled to room temperature and diluted with EtOAc (100 mL). The mixture was successively washed with water (50 mL) and brine (50 mL). The organic layer was dried over Na₂SO₄, filtered and concentrated under reduced pressure. The residue was purified by silica gel chromatograph (PE/EtOAc=2/1) to give the title compound (0.80 g, 47%). LC-MS (M+H)⁺=350.1.

### Step 8: 10-fluoro-8-(trifluoromethyl)-1,2,3,4,5,6-hexahydro-1,5-methanobenzo[c]azocine

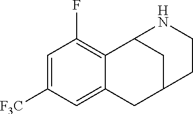 [SMILES: Fc1cc(C(F)(F)F)cc2c1C1CC(CCN1)C2]

To a mixture of 2-benzyl-10-fluoro-8-(trifluoromethyl)-1,2,3,4,5,6-hexahydro-1,5-methanobenzo[c]azocine (0.80 g, 2.28 mmol) in EtOAc (20 mL) was added Pd/C (10%, 100 mg) under nitrogen. The mixture was purged with hydrogen and stirred at 60° C. for 15 h. The solid was filtered off and the filtrated was concentrated under reduced pressure to give the title compound (0.52 g, 88%). LC-MS (M+H)⁺=260.2.

### Step 9: (5-amino-1-((2-(trimethylsilyl)ethoxy)methyl)-6,8-dihydro-1H-furo[3,4-d]pyrrolo[3,2-b]pyridin-2-yl)(10-fluoro-8-(trifluoromethyl)-3,4,5,6-tetrahydro-1,5-methanobenzo[c]azocin-2(1H)-yl)methanone

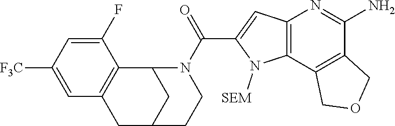 [SMILES: C[Si](C)(C)CCOCn1c(C(=O)N2CCC3Cc4cc(C(F)(F)F)cc(F)c4C2C3)cc2nc(N)c3c(c21)COC3]

A mixture of 10-fluoro-8-(trifluoromethyl)-1,2,3,4,5,6-hexahydro-1,5-methanobenzo[c]azocine (390 mg, 1.5 mmol), 5-amino-1-((2-(trimethylsilyl)ethoxy)methyl)-6,8-dihydro-1H-furo[3,4-d]pyrrolo[3,2-b]pyridine-2-carboxylic acid (527 mg, 1.5 mmol), BOPCl (576 mg, 2.25 mmol), and DIPEA (583 mg, 4.5 mmol) in THF (20 mL) was stirred at 60° C. for 16 h. The mixture was cooled to room temperature and concentrated in vacuo. The residue was partitioned between water (50 mL) and EtOAc (50 mL). The organic layer was dried over Na₂SO₄, filtered and concentrated under reduced pressure. The residue was purified by silica gel chromatograph (DCM:MeOH=20:1) to give the title compound (788 mg, 89%). LC-MS (M+H)⁺=591.2.

### Step 10: (5-amino-6,8-dihydro-1H-furo[3,4-d]pyrrolo[3,2-b]pyridin-2-yl)((1S,5R)-10-fluoro-8-(trifluoromethyl)-3,4,5,6-tetrahydro-1,5-methanobenzo[c]azocin-2(1H)-yl)methanone & (5-amino-6,8-dihydro-1H-furo[3,4-d]pyrrolo[3,2-b]pyridin-2-yl)((1R,5S)-10-fluoro-8-(trifluoromethyl)-3,4,5,6-tetrahydro-1,5-methanobenzo[c]azocin-2(1H)-yl)methanone

To a mixture of (5-amino-1-((2-(trimethylsilyl)ethoxy)methyl)-6,8-dihydro-1H-furo[3,4-d]pyrrolo[3,2-b]pyridin-2-yl)(10-fluoro-8-(trifluoromethyl)-3,4,5,6-tetrahydro-1,5-methanobenzo[c]azocin-2(1H)-yl)methanone (788 mg, 1.33 mmol) in DCM (5 mL) was added TFA (5 mL). The mixture was stirred at room temperature for 2 h. The mixture was concentrated under reduced pressure. The residue was re-dissolved in MeOH (10 mL) followed by addition of ammonium hydroxide (28%, 2 mL). The mixture was stirred at room temperature for 1 h and concentrated under reduced pressure. The residue was purified by prep-HPLC followed by chiral SFC to give Example 15 (150 mg, 25%) and Example 16 (157 mg, 26%).

Analytical chiral-SFC condition as below. Column: Lux Cellulose-3; Column size: 4.6×100 mm, 5 μm; Mobile phase: 4 mM methanolic NH₃:CO₂, 1:9 to 1:1 in 3 min, 1:1 for 2 min, 1:1 to 1:9 in 0.1 min, 1:9 for 1.9 min; Flow: 2.0 mL/min; Temperature: 35° C.; back pressure: 1500 psi.

Example 15: Analytical SFC tR=2.50 min. ¹H NMR (400 MHz, DMSO-d6, 80° C.) δ11.26 (s, 1H), 7.41 (s, 1H), 7.32 (d, J=9.4 Hz, 1H), 6.52 (s, 1H), 6.03 (s, 1H), 5.26 (s, 2H), 5.15-5.07 (m, 2H), 4.95-4.85 (m, 2H), 4.13-4.03 (m, 1H), 3.27-3.14 (m, 1H), 3.0-2.93 (m, 1H), 2.8-2.74 (m, 1H), 2.42-2.36 (m, 1H), 2.2-2.08 (m, 1H), 1.94-1.77 (m, 2H), 1.74-1.62 (m, 1H). LC-MS (M+H)⁺=461.3.

Example 16: Analytical SFC tR=2.77 min. ¹H NMR (400 MHz, DMSO-d6, 80° C.) δ11.26 (s, 1H), 7.41 (s, 1H), 7.32 (d, J=9.4 Hz, 1H), 6.52 (s, 1H), 6.03 (s, 1H), 5.26 (s, 2H), 5.15-5.07 (m, 2H), 4.95-4.85 (m, 2H), 4.13-4.03 (m, 1H), 3.27-3.14 (m, 1H), 3.0-2.93 (m, 1H), 2.8-2.74 (m, 1H), 2.42-2.36 (m, 1H), 2.2-2.08 (m, 1H), 1.94-1.77 (m, 2H), 1.74-1.62 (m, 1H). LC-MS (M+H)⁺=461.3.

### Example 17 & Example 18: (5-amino-6,8-dihydro-1H-furo[3,4-d]pyrrolo[3,2-b]pyridin-2-yl)((1R,5R)-8-(trifluoromethyl)-1,3,4,6-tetrahydro-2H-1,5-methanobenzo[f][1,4]diazocin-2-yl)methanone & (5-amino-6,8-dihydro-1H-furo[3,4-d]pyrrolo[3,2-b]pyridin-2-yl)((1S,5S)-8-(trifluoromethyl)-1,3,4,6-tetrahydro-2H-1,5-methanobenzo[f][1,4]diazocin-2-yl)methanone

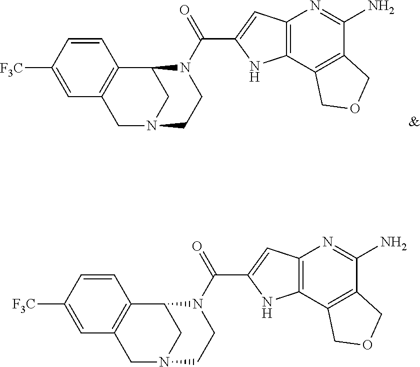 [SMILES: Nc1nc2cc(C(=O)N3CC[N@@]4Cc5cc(C(F)(F)F)ccc5[C@@H]3C4)[nH]c2c2c1COC2 ; Nc1nc2cc(C(=O)N3CC[N@]4Cc5cc(C(F)(F)F)ccc5[C@H]3C4)[nH]c2c2c1COC2]

### Step 1: ethyl 2-methyl-4-(trifluoromethyl)benzoate

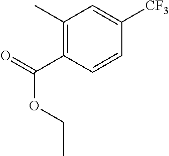 [SMILES: CCOC(=O)c1ccc(C(F)(F)F)cc1C]

To a mixture of 1-bromo-2-methyl-4-(trifluoromethyl)benzene (15.0 g, 62.8 mmol) and Et₃N (19.0 g, 188 mmol) in EtOH (80 mL) and DMF (10 mL) was added Pd(dppf)Cl₂ (2.3 g, 3.13 mmol) in a high pressure reactor under nitrogen. The reactor was purged with CO at 10 bar and heated to 90° C. with stirring. After 15 h, the mixture was cooled to room temperature and depressurized. The mixture was concentrated under reduced pressure. The residue was purified by silica gel chromatograph (PE/EtOAc=10/1) to give the title compound (11 g, 71%). LC-MS (M+H)⁺=233.2.

### Step 2: ethyl 2-(bromomethyl)-4-(trifluoromethyl)benzoate

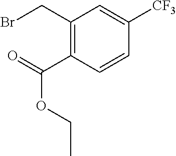 [SMILES: CCOC(=O)c1ccc(C(F)(F)F)cc1CBr]

To a solution of ethyl 2-methyl-4-(trifluoromethyl)benzoate (11 g, 47.4 mmol) in CCl₄ (150 mL) was added NBS (8.43 g, 47.4 mmol) and AIBN (200 mg, 1.2 mmol). The mixture was stirred at refluxing temperature for 16 h and cooled to room temperature. Solid was filtered off and the filtrate was diluted with DCM (150 mL). The organic layer was successively washed with saturated NaHCO₃ (150 mL) and brine (150 mL). The organic layer was dried over Na₂SO₄, filtered and concentrated under reduced pressure. The residue was purified by silica gel chromatograph (PE/EtOAc=10/1) to give the title compound (12 g, 82%). LC-MS (M+H)⁺=311.2.

### Step 3: ethyl 2-((benzyl(2-ethoxy-2-oxoethyl)amino)methyl)-4-(trifluoromethyl)benzoate

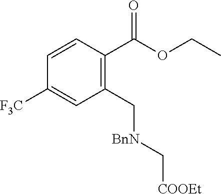 [SMILES: CCOC(=O)CN(Cc1ccccc1)Cc1cc(C(F)(F)F)ccc1C(=O)OCC]

A mixture of ethyl 2-(bromomethyl)-4-(trifluoromethyl)benzoate (12 g, 38.7 mmol), N-benzylglycine ethyl ester (11.2 g, 58 mmol) and Et₃N (5.9 mL, 58 mmol) in THF (200 mL) was stirred at refluxing temperature for 2 h. The mixture was cooled to room temperature and concentrated under reduced pressure. The residue was partitioned between ether (200 mL) and saturated NaHCO₃ (200 mL). The aqueous layer was extracted with EtOAc (200 mL×2). The combined organic layer was concentrated under reduced pressure. The crude was purified by silica gel chromatograph (PE/EtOAc=5/1) to give the title compound (10 g, 61%). LC-MS (M+H)⁺=424.2.

### Step 4: ethyl 2-benzyl-4-oxo-7-(trifluoromethyl)-1,2,3,4-tetrahydroisoquinoline-3-carboxylate

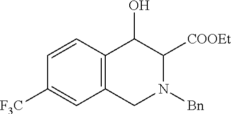 [SMILES: CCOC(=O)C1C(O)c2ccc(C(F)(F)F)cc2CN1Cc1ccccc1]

A mixture of ethyl 2-((benzyl(2-ethoxy-2-oxoethyl)amino)methyl)-4-(trifluoromethyl)benzoate (10 g, 23.5 mmol) and sodium ethoxide (20% in ethanol, 12 g, 35.2 mmol) in toluene (250 mL) was stirred at 110° C. for 5 h. The mixture was cooled to room temperature and diluted with EtOAc (250 mL) and water (250 mL). Hydrochloric acid (1 N) was added with stirring until the pH reached about 8. The organic layer was separated and dried over Na₂SO₄, filtered and concentrated under reduced pressure. The residue was purified by silica gel chromatograph (PE/EOAc=5/1) to give the title compound (8.5 g, 96%). LC-MS (M+H)⁺=378.2.

### Step 5: 2-benzyl-7-(trifluoromethyl)-2,3-dihydroisoquinolin-4(1H)-one

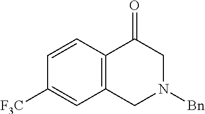 [SMILES: O=C1CN(Cc2ccccc2)Cc2cc(C(F)(F)F)ccc21]

To a mixture of ethyl 2-benzyl-4-oxo-7-(trifluoromethyl)-1,2,3,4-tetrahydroisoquinoline-3-carboxylate (8.5 g, 22.5 mmol) in EtOH (70 mL) was slowly added concentrated hydrochloric acid (100 mL). The mixture was stirred at refluxing temperature for 24 h. The mixture was cooled to room temperature, and ethanol was evaporated under reduced pressure. Saturated NaHCO₃ was added until the pH of the mixture reached about 8. The mixture was extracted with DCM (100 mL×2). The combined organic layer was washed with brine (100 mL), dried over Na₂SO₄, filtered and concentrated under reduced pressure. The residue was purified by silica gel chromatograph (PE/EtOAc=3/1) to give the title compound (3.5 g, 51%). LC-MS (M+H)⁺=306.2.

### Step 6: 4-hydroxy-7-(trifluoromethyl)-1,2,3,4-tetrahydroisoquinolin-2-ium acetate

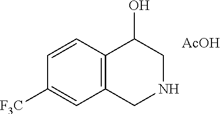 [SMILES: "AcOH" ; OC1CNCc2cc(C(F)(F)F)ccc21]

To a mixture of 2-benzyl-7-(trifluoromethyl)-2,3-dihydroisoquinolin-4(1H)-one (3.5 g, 11.5 mmol) in AcOH (10 mL) and EtOH (10 mL) was added Pd/C (10%, 300 mg) under nitrogen. The mixture was purged with hydrogen and stirred for 5 h. Solid was filtered off and the filtrate was concentrated under reduced pressure to give the title compound (2.2 g, 69%). LC-MS (M+H)⁺=218.2.

### Step 7: tert-butyl (2-(4-hydroxy-7-(trifluoromethyl)-3,4-dihydroisoquinolin-2(1H)-yl)ethyl)carbamate

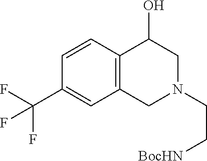 [SMILES: CC(C)(C)OC(=O)NCCN1Cc2cc(C(F)(F)F)ccc2C(O)C1]

A mixture of 4-hydroxy-7-(trifluoromethyl)-1,2,3,4-tetrahydroisoquinolin-2-ium acetate (651 mg, 2.35 mmol), tert-butyl (2-iodoethyl)carbamate (1.06 g, 3.9 mmol) and K₂CO₃ (1.04 g, 7.5 mmol) in MeCN (40 mL) was stirred at 40° C. for 36 h. The mixture was cooled to room temperature and concentrated under reduced pressure. The residue was partitioned between water (20 mL) and DCM (20 mL). The organic layer was dried over Na₂SO₄, filtered and concentrated under reduced pressure. The residue was purified by silica gel chromatograph (PE/EtOAc=4/1) to give the title compound (740 mg, 87%). LC-MS (M+H)⁺=361.2.

### Step 8: 2-(2-aminoethyl)-7-(trifluoromethyl)-1,2,3,4-tetrahydroisoquinolin-4-ol dihydrochloride

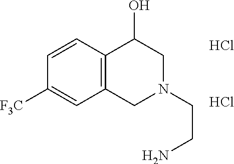 [SMILES: NCCN1Cc2cc(C(F)(F)F)ccc2C(O)C1 ; Cl ; Cl]

To a flask charged with tert-butyl (2-(4-hydroxy-7-(trifluoromethyl)-3,4-dihydroisoquinolin-2(1H)-yl)ethyl)carbamate (740 mg, 2.05 mmol) was added HCl in dioxane (4 N, 5 mL). After 30 min, the mixture was concentrated under reduced pressure to give the title compound (660 mg, 97%). LC-MS (M+H)⁺=261.1.

### Step 9: N-(2-(4-hydroxy-7-(trifluoromethyl)-3,4-dihydroisoquinolin-2(1H)-yl)ethyl)-2-nitrobenzenesulfonamide

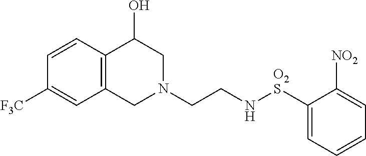 [SMILES: O=[N+]([O-])c1ccccc1S(=O)(=O)NCCN1Cc2cc(C(F)(F)F)ccc2C(O)C1]

To a solution of 2-(2-aminoethyl)-7-(trifluoromethyl)-1,2,3,4-tetrahydroisoquinolin-4-ol dihydrochloride (660 mg, 2.0 mmol) and Et₃N (606 mg, 6.0 mmol) in DCM (40 mL) was added 2-nitrobenzenesulfonyl chloride (442 mg, 2.0 mmol) in portions at 0° C. The mixture was stirred at room temperature for 1 h. The mixture was washed with water (20 mL). The aqueous layer was extracted with DCM (20 mL). The combined organic layer was dried over Na₂SO₄, filtered and concentrated under reduced pressure. The residue was purified by silica gel chromatograph (DCM/MeOH=30/1) to give the title compound (780 mg, 88%). LC-MS (M+H)=446.2.

### Step 10: 8-(trifluoromethyl)-1,3,4,6-tetrahydro-2H-1,5-methanobenzo[f][1,4]diazocine

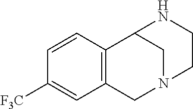 [SMILES: FC(F)(F)c1ccc2c(c1)CN1CCNC2C1]

To a mixture of N-(2-(4-hydroxy-7-(trifluoromethyl)-3,4-dihydroisoquinolin-2(1H)-yl)ethyl)-2-nitrobenzenesulfonamide (780 mg, 1.75 mmol) and PPh₃ (552 mg, 2.1 mmol) in THF (80 mL) was added DtBAD (483 mg, 2.1 mmol) in portions at 0° C. The mixture was stirred at room temperature for 6 h and then concentrated under reduced pressure. The residue was re-dissolved in THF (20 mL) and DMF (5 mL), followed by addition of 1-dodecanethiol (1155 mg, 5.25 mmol) and LiOH H₂O (220 mg, 5.25 mmol). The mixture was stirred at room temperature overnight and then concentrated under reduced pressure. The residue was partitioned between water (100 mL) and EtOAc (100 mL). The organic layer was separated, dried with Na₂SO₄, filtered and concentrated under reduced pressure. The residue was purified by silica gel chromatograph (DCM:MeOH=20:1) to give the title compound (280 mg, 66%). LC-MS (M+H)⁺=243.2.

### Step 11: (5-amino-1-((2-(trimethylsilyl)ethoxy)methyl)-6,8-dihydro-1H-furo[3,4-d]pyrrolo[3,2-b]pyridin-2-yl)(8-(trifluoromethyl)-1,3,4,6-tetrahydro-2H-1,5-methanobenzo[f][1,4]diazocin-2-yl)methanone

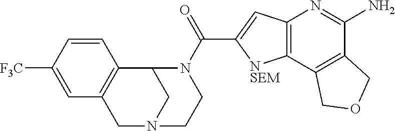 [SMILES: C[Si](C)(C)CCOCn1c(C(=O)N2CCN3Cc4cc(C(F)(F)F)ccc4C2C3)cc2nc(N)c3c(c21)COC3]

To a mixture of 8-(trifluoromethyl)-1,3,4,6-tetrahydro-2H-1,5-methanobenzo[f][1,4]diazocine (80 mg, 0.33 mmol), 5-amino-1-((2-(trimethylsilyl)ethoxy)methyl)-6,8-dihydro-H-furo[3,4-d]pyrrolo[3,2-b]pyridine-2-carboxylic acid (122 mg, 0.35 mmol) in THF (10 mL) was added BOPCl (126 mg, 0.49 mmol) and DIPEA (129 mg, 1.0 mmol) and the mixture was stirred at 60° C. for 3 h. The residue was concentrated under reduced pressure. The residue was partitioned between water (50 mL) and EtOAc (50 mL). The organic layer was dried with Na₂SO₄, filtered and concentrated under reduced pressure. The residue was purified by silica gel chromatograph (DCM:MeOH=20:1) to give the title compound (110 mg, 55%). LC-MS (M+H)⁺=574.4.

### Step 12: (5-amino-6,8-dihydro-1H-furo[3,4-d]pyrrolo[3,2-b]pyridin-2-yl)((1R,5R)-8-(trifluoromethyl)-1,3,4,6-tetrahydro-2H-1,5-methanobenzo[f][1,4]diazocin-2-yl)methanone & (5-amino-6,8-dihydro-1H-furo[3,4-d]pyrrolo[3,2-b]pyridin-2-yl)((1S,5S)-8-(trifluoromethyl)-1,3,4,6-tetrahydro-2H-1,5-methanobenzo[f][1,4]diazocin-2-yl)methanone

To a solution of (5-amino-1-((2-(trimethylsilyl)ethoxy)methyl)-6,8-dihydro-1H-furo[3,4-d]pyrrolo[3,2-b]pyridin-2-yl)(8-(trifluoromethyl)-1,3,4,6-tetrahydro-2H-1,5-methanobenzo[f][1,4]diazocin-2-yl)methanone (110 mg, 0.19 mmol) in DCM (5 mL) was added TFA (2 mL). The mixture was stirred at room temperature for 2 h and then concentrated under reduced pressure. The residue was re-dissolved in MeOH (5 mL) followed by addition of ammonium hydroxide (28%, 1 mL). The mixture was stirred at room temperature for 1 h and then concentrated under reduced pressure. The residue was purified by prep-HPLC followed by chiral SFC to give Example 17 (39 mg, 46%) and Example 18 (37 mg, 44%).

Analytical chiral-SFC condition as below. Column: CHIRALPAK Cellulose-3; Column size: 4.6×100 mm, 5 μm; Mobile phase: 4 mM methanolic NH₃:CO₂, 1:9 to 1:1 in 3 min, 1:1 for 2 min, 1:1 to 1:9 in 0.1 min, 1:9 for 1.9 min; Flow: 2.0 mL/min; Temperature: 35° C.; back pressure: 1500 psi.

Example 17: Analytical SFC tR=3.04 min. ¹H NMR (400 MHz, DMSO-d6) δ 11.52 (s, 1H), 7.72-6.87 (m, 3H), 6.84-6.25 (m, 1H), 5.78-5.35 (m, 3H), 5.12 (s, 2H), 4.90 (s, 2H), 4.30 (d, J=18.3 Hz, 1H), 4.07 (d, J=18.2 Hz, 1H), 4.00-3.63 (m, 1H), 3.60-3.23 (m, 1H), 3.23-2.84 (m, 4H). LC-MS (M+H)⁺=444.3.

Example 18: Analytical SFC tR=3.51 min. ¹H NMR (400 MHz, DMSO-d6) δ 11.52 (s, 1H), 7.72-6.87 (m, 3H), 6.84-6.25 (m, 1H), 5.78-5.35 (m, 3H), 5.12 (s, 2H), 4.90 (s, 2H), 4.30 (d, J=18.3 Hz, 1H), 4.07 (d, J=18.2 Hz, 1H), 4.00-3.63 (m, 1H), 3.60-3.23 (m, 1H), 3.23-2.84 (m, 4H). LC-MS (M+H)⁺=444.3.

### Example 19 & Example 20: (5-amino-6,8-dihydro-1H-furo[3,4-d]pyrrolo[3,2-b]pyridin-2-yl)((1R,5R)-10-fluoro-8-(trifluoromethyl)-1,3,4,6-tetrahydro-2H-1,5-methanobenzo[f][1,4]diazocin-2-yl)methanone & (5-amino-6,8-dihydro-1H-furo[3,4-d]pyrrolo[3,2-b]pyridin-2-yl)((1S,5S)-10-fluoro-8-(trifluoromethyl)-1,3,4,6-tetrahydro-2H-1,5-methanobenzo[f][1,4]diazocin-2-yl)methanone

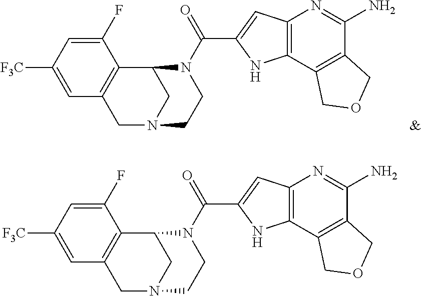

### Step 1: methyl (3-bromo-5-fluorobenzyl)glycinate

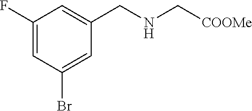 [SMILES: COC(=O)CNCc1cc(F)cc(Br)c1]

A mixture of methyl glycinate hydrochloride (12.5 g, 100 mmol), Et₃N (10.1 g, 100 mmol) and 3-bromo-5-fluorobenzaldehyde (10.1 g, 50 mmol) in DCM (400 mL) was stirred at room temperature for 30 min then cooled to 0° C., followed by addition of NaBH(OAc)₃ (26.4 g, 125 mmol) in portions. The mixture was warmed to room temperature and stirred for 12 h. Saturated NaHCO₃ (300 mL) was added slowly with vigorous stirring, and the organic layer was separated. The aqueous layer was back extracted with DCM (100 mL×3). The combined organic layer was concentrated under reduced pressure. The residue was purified by silica gel chromatograph (PE/EtOAc=3/1) to give the title compound (12.2 g, 89%). LC-MS (M+H)⁺=275.9.

### Step 2: methyl N-((benzyloxy)carbonyl)-N-(3-bromo-5-fluorobenzyl)glycinate

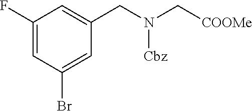 [SMILES: COC(=O)CN(Cc1cc(F)cc(Br)c1)C(=O)OCc1ccccc1]

To a mixture of methyl (3-bromo-5-fluorobenzyl)glycinate (11 g, 40 mmol) and Na₂CO₃ (12.7 g, 120 mmol) in DMF (100 mL) was added CbzCl (13.6 g, 80 mmol) dropwise at 0° C. The mixture was warmed to 40° C. and stirred overnight. The mixture was cooled to room temperature and diluted with water (400 mL). The mixture was extracted with EtOAc (100 mL×3). The combined organic layer was washed with brine (100 mL), dried over Na₂SO₄, filtered and concentrated under reduced pressure. The residue was purified by silica gel chromatograph (PE/EtOAc=5/1) to give the title compound (10.5 g, 64%). LC-MS (M+Na)⁺=432.1.

### Step 3: N-((benzyloxy)carbonyl)-N-(3-bromo-5-fluorobenzyl)glycine

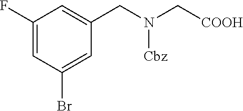 [SMILES: O=C(O)CN(Cc1cc(F)cc(Br)c1)C(=O)OCc1ccccc1]

To a mixture of methyl N-((benzyloxy)carbonyl)-N-(3-bromo-5-fluorobenzyl)glycinate (10.5 g, 25.7 mmol) in THF (100 mL) and water (50 mL) was added LiOH (1.85 g, 77.1 mmol). The mixture was stirred at room temperature for 2 h and then diluted with water (200 mL). The pH of the mixture was adjusted with hydrochloric acid (1 N) to about 3, and then the mixture was extracted with EtOAc (100 mL×3). The combined organic layer was dried with Na₂SO₄, filtered and concentrated under reduced pressure to give the title compound (10.0 g, 99%). LC-MS (M+Na)⁺=418.1.

### Step 4: 7-bromo-5-fluoro-2,3-dihydroisoquinolin-4(1H)-one & 5-bromo-7-fluoro-2,3-dihydroisoquinolin-4(1H)-one

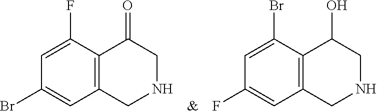

To a solution of N-((benzyloxy)carbonyl)-N-(3-bromo-5-fluorobenzyl)glycine (10.0 g, 25.3 mmol) in DCM (150 mL) was added SOCl₂ (5.94 g, 49.5 mmol) dropwise and the mixture was stirred at room temperature for 2 h. The mixture was concentrated under reduced pressure. The residue was re-dissolved in DCM (200 mL) and cooled to 0° C., followed by addition of AlCl₃ (17.5 g, 132 mmol) in portions. The mixture was stirred at room temperature overnight and then added into saturated NaHCO₃ (500 mL) slowly with stirring. The mixture was aged for 1 h and solid was filtered off. The solid was rinsed with DCM (500 mL×3). The organic layer of the filtrate was separated, dried over Na₂SO₄, filtered and concentrated under reduced pressure to give a mixture of 7-bromo-5-fluoro-2,3-dihydroisoquinolin-4(1H)-one and 5-bromo-7-fluoro-2,3-dihydroisoquinolin-4(1H)-one (HPLC about 1:4, 6.1 g, 99%). LC-MS (M+H)⁺=244.0.

### Step 5: 7-bromo-5-fluoro-1,2,3,4-tetrahydroisoquinolin-4-ol & 5-bromo-7-fluoro-1,2,3,4-tetrahydroisoquinolin-4-ol

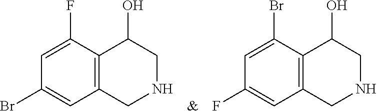

To a mixture of 7-bromo-5-fluoro-2,3-dihydroisoquinolin-4(1H)-one and 5-bromo-7-fluoro-2,3-dihydroisoquinolin-4(1H)-one (HPLC about 1:4, 6.1 g, 25.0 mmol in total) in MeOH (140 mL) was added NaBH₄ (1.14 g, 30 mmol) in portions. The mixture was stirred at room temperature for 1 h, followed by addition of acetone (5 mL). The mixture was concentrated under reduced pressure. The residue was purified by silica gel chromatograph (DCM/MeOH=15/1) to give 7-bromo-5-fluoro-1,2,3,4-tetrahydroisoquinolin-4-ol and 5-bromo-7-fluoro-1,2,3,4-tetrahydroisoquinolin-4-ol (HPLC about 1:4, 5.0 g, 81%). LC-MS (M+H)⁺=246.0.

### Step 6: tert-butyl 7-bromo-5-fluoro-4-hydroxy-3,4-dihydroisoquinoline-2(1H)-carboxylate & tert-butyl 5-bromo-7-fluoro-4-hydroxy-3,4-dihydroisoquinoline-2(1H)-carboxylate

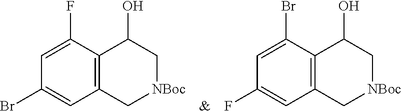

To a solution of 7-bromo-5-fluoro-1,2,3,4-tetrahydroisoquinolin-4-ol & 5-bromo-7-fluoro-1,2,3,4-tetrahydroisoquinolin-4-ol (HPLC about 1:4, 5.0 g, 20.4 mmol in total) and Et₃N (4.12 g, 40.8 mmol) in DCM (100 mL) was added Boc₂O (5.3 g, 24.5 mmol). The mixture was stirred at room temperature for 1 h, followed by addition of water (150 mL). The organic layer was separated and the aqueous layer was back extracted with DCM (100 mL×2). The combined organic layer was dried with Na₂SO₄, filtered and concentrated under reduced pressure. The residue was purified by silica gel chromatograph (PE/EtOAc=3/1) to give tert-butyl 7-bromo-5-fluoro-4-hydroxy-3,4-dihydroisoquinoline-2(1H)-carboxylate and tert-butyl 5-bromo-7-fluoro-4-hydroxy-3,4-dihydroisoquinoline-2(1H)-carboxylate (HPLC about 1:4, 6.8 g, 97%). LC-MS (M+H)⁺=346.0.

### Step 7: tert-butyl 7-bromo-5-fluoro-4-((2-hydroxyethyl)amino)-3,4-dihydroisoquinoline-2(1H)-carboxylate & tert-butyl 5-bromo-7-fluoro-4-((2-hydroxyethyl)amino)-3,4-dihydroisoquinoline-2(1H)-carboxylate

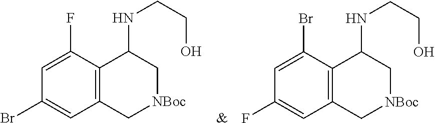

To a mixture of tert-butyl 7-bromo-5-fluoro-4-hydroxy-3,4-dihydroisoquinoline-2(1H)-carboxylate and tert-butyl 5-bromo-7-fluoro-4-hydroxy-3,4-dihydroisoquinoline-2(1H)-carboxylate (HPLC about 1:4, 6.8 g, 19.7 mmol in total) and methanesulfonic anhydride (5.22 g, 30 mmol) in THF (70 mL) was added Et₃N (4.04 g, 40 mmol) dropwise. The mixture was stirred at room temperature for 1 h. The mixture was diluted with EtOAc (300 mL) and successively washed with saturated NaHCO₃ (100 mL) and brine (100 mL). The organic layer was dried over Na₂SO₄, filtered and concentrated under reduced pressure. The residue was re-dissolved in DMF (40 mL) followed by addition of 2-aminoethan-1-ol (6.1 g, 100 mmol). The mixture was stirred at 50° C. for 6 h, cooled to room temperature and diluted with water (200 mL). The mixture was extracted with EtOAc (100 mL×3). The combined organic layer was washed with brine (100 mL), dried over Na₂SO₄, filtered and concentrated under reduced pressure. The residue was purified by silica gel chromatograph (DCM:MeOH=40:1) to give tert-butyl 7-bromo-5-fluoro-4-((2-hydroxyethyl)amino)-3,4-dihydroisoquinoline-2(1H)-carboxylate and tert-butyl 5-bromo-7-fluoro-4-((2-hydroxyethyl)amino)-3,4-dihydroisoquinoline-2(1H)-carboxylate (HPLC about 1:4, 5.1 g, 67%).

### Step 9: tert-butyl 8-bromo-10-fluoro-1,3,4,6-tetrahydro-2H-1,5-methanobenzo[f][1,4]diazocine-2-carboxylate

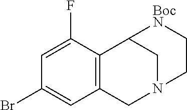 [SMILES: CC(C)(C)OC(=O)N1CCN2Cc3cc(Br)cc(F)c3C1C2]

To a mixture of tert-butyl 7-bromo-5-fluoro-4-((2-hydroxyethyl)amino)-3,4-dihydroisoquinoline-2(1H)-carboxylate and tert-butyl 5-bromo-7-fluoro-4-((2-hydroxyethyl)amino)-3,4-dihydroisoquinoline-2(1H)-carboxylate (HPLC about 1:4, 5.1 g, 13.1 mmol in total) in DCM (60 mL) was added SOCl₂ (3.12 g, 26.2 mmol) dropwise. The mixture was stirred at room temperature for 2 h. The mixture was concentrated under reduced pressure, and the residue was treated with HCl in dioxane (4.0 M, 50 mL). The mixture was stirred at room temperature for 1 h and then concentrated under reduced pressure. The residue was re-dissolved in DMF (150 mL) followed by addition of K₂CO₃ (4.49 g, 32.5 mmol). The mixture was stirred at 60° C. for 2 h and then cooled to room temperature. Boc₂O (5.3 g, 24.5 mmol) was added and the mixture was stirred at room temperature for 1 h. The mixture was diluted with water (500 mL) and extracted with EtOAc (100 mL×3). The combined organic layer was washed by brine (100 mL), dried over Na₂SO₄, filtered and concentrated under reduced pressure. The residue was purified by prep-HPLC to give the title compound (170 mg, 3%). LC-MS (M+H)⁺=371.2.

### Step 9: tert-butyl 10-fluoro-8-iodo-1,3,4,6-tetrahydro-2H-1,5-methanobenzo[f][1,4]diazocine-2-carboxylate

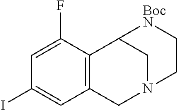 [SMILES: CC(C)(C)OC(=O)N1CCN2Cc3cc(I)cc(F)c3C1C2]

A mixture of tert-butyl 8-bromo-10-fluoro-1,3,4,6-tetrahydro-2H-1,5-methanobenzo[f][1,4]diazocine-2-carboxylate (170 mg, 0.46 mmol), CuI (87 mg, 0.46 mmol), NaI (300 mg, 2.0 mmol) and trans-N,N′-dimethylcyclohexane-1,2-diamine (71 mg, 0.50 mmol) in dioxane (5 mL) was stirred at refluxing temperature for 48 h under nitrogen. The mixture was cooled to room temperature, diluted with water (50 mL) and extracted with EtOAc (50 mL×3). The combined organic layer was washed with brine (50 mL), dried with Na₂SO₄, filtered and concentrated under reduced pressure. The residue was purified by prep-TLC (DCM:MeOH=20:1) to give the title compound (170 mg, 89%). LC-MS (M+H)⁺=419.3.

### Step 10: tert-butyl 10-fluoro-8-(trifluoromethyl)-1,3,4,6-tetrahydro-2H-1,5-methanobenzo[f][1,4]diazocine-2-carboxylate

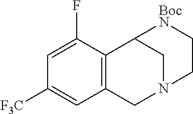 [SMILES: CC(C)(C)OC(=O)N1CCN2Cc3cc(C(F)(F)F)cc(F)c3C1C2]

A mixture of tert-butyl 10-fluoro-8-iodo-1,3,4,6-tetrahydro-2H-1,5-methanobenzo[f][1,4]diazocine-2-carboxylate (170 mg, 0.41 mmol) and (1,10-phenanthroline)(trifluoromethyl)copper(I) (385 mg, 1.23 mmol) in DMF (4 mL) was stirred at 80° C. for 12 h under nitrogen and then cooled to room temperature. The mixture was diluted with water (50 mL) and solid was filtered off. The filter cake was rinsed with EtOAc (100 mL). The organic layer of the filtrate was separated. The aqueous layer was extracted with EtOAc (50 mL×2). The combined organic layer was washed with brine (50 mL), dried over Na₂SO₄, filtered and concentrated under reduced pressure. The residue was purified by prep-TLC (DCM:MeOH=20:1) to give the title compound (126 mg, 85%). LC-MS (M+H)⁺=361.3.

### Step 11: 10-fluoro-8-(trifluoromethyl)-1,3,4,6-tetrahydro-2H-1,5-methanobenzo[f][1,4]diazocine dihydrochloride

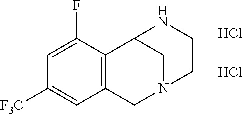 [SMILES: Fc1cc(C(F)(F)F)cc2c1C1CN(CCN1)C2 ; Cl ; Cl]

A mixture of tert-butyl 10-fluoro-8-(trifluoromethyl)-1,3,4,6-tetrahydro-2H-1,5-methanobenzo[f][1,4]diazocine-2-carboxylate (126 mg, 0.35 mmol) in HCl in dioxane (4.0 M, 4 mL) was stirred at room temperature for 1 h. The mixture was concentrated under reduced pressure to give the title compound (78 mg, 67%).

### Step 12: (5-amino-1-((2-(trimethylsilyl)ethoxy)methyl)-6,8-dihydro-1H-furo[3,4-d]pyrrolo[3,2-b]pyridin-2-yl)(10-fluoro-8-(trifluoromethyl)-1,3,4,6-tetrahydro-2H-1,5-methanobenzo[f][1,4]diazocin-2-yl)methanone

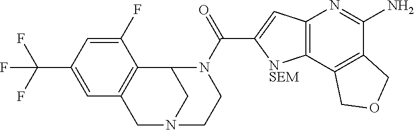 [SMILES: C[Si](C)(C)CCOCn1c(C(=O)N2CCN3Cc4cc(C(F)(F)F)cc(F)c4C2C3)cc2nc(N)c3c(c21)COC3]

To a mixture of 10-fluoro-8-(trifluoromethyl)-1,3,4,6-tetrahydro-2H-1,5-methanobenzo[f][1,4]diazocine dihydrochloride (78 mg, 0.23 mmol), 5-amino-1-((2-(trimethylsilyl)ethoxy)methyl)-6,8-dihydro-1H-furo[3,4-d]pyrrolo[3,2-b]pyridine-2-carboxylic acid (122 mg, 0.35 mmol) in THF (10 mL) was added BOPCl (126 mg, 0.49 mmol), DIPEA (155 mg, 1.2 mmol). The mixture was stirred at 60° C. for 3 h and then cooled to room temperature. The mixture was partitioned between water (50 mL) and EtOAc (50 mL). The organic layer was dried over Na₂SO₄, filtered and concentrated under reduced pressure. The residue was purified by silica gel chromatograph (DCM:MeOH=20:1) to give the title compound (82 mg, 59%). LC-MS (M+H)⁺=592.5.

### Step 13: (5-amino-6,8-dihydro-1H-furo[3,4-d]pyrrolo[3,2-b]pyridin-2-yl)((1R,5R)-10-fluoro-8-(trifluoromethyl)-1,3,4,6-tetrahydro-2H-1,5-methanobenzo[f][1,4]diazocin-2-yl)methanone & (5-amino-6,8-dihydro-1H-furo[3,4-d]pyrrolo[3,2-b]pyridin-2-yl)((1S,5S)-10-fluoro-8-(trifluoromethyl)-1,3,4,6-tetrahydro-2H-1,5-methanobenzo[f][1,4]diazocin-2-yl)methanone

To a solution of (5-amino-1-((2-(trimethylsilyl)ethoxy)methyl)-6,8-dihydro-1H-furo[3,4-d]pyrrolo[3,2-b]pyridin-2-yl)(10-fluoro-8-(trifluoromethyl)-1,3,4,6-tetrahydro-2H-1,5-methanobenzo[f][1,4]diazocin-2-yl)methanone (82 mg, 0.14 mmol) in DCM (5 mL) was added TFA (2 mL). The mixture was stirred at room temperature for 2 h and then concentrated under reduced pressure. The residue was re-dissolved in MeOH (5 mL) followed by addition of ammonium hydroxide (28%, 1 mL). The mixture was stirred at room temperature for 1 h and then concentrated under reduced pressure. The residue was purified by prep-HPLC followed by chiral SFC to give Example 19 (19 mg, 30%) and Example 20 (18 mg, 28%).

Analytical chiral-SFC condition as below. Column: CHIRALPAK AS-H; Column size: 4.6×100 mm, 5 μm; Mobile phase: 4 mM methanolic NH₃:CO₂, 1:9 to 1:1 in 3 min, 1:1 for 2 min, 1:1 to 1:9 in 0.1 min, 1:9 for 1.9 min; Flow: 2.0 mL/min; Temperature: 35° C.; back pressure: 1500 psi.

Example 19: Analytical SFC tR=2.64 min. ¹H NMR (400 MHz, DMSO-d6, 80° C.) δ11.26 (s, 1H), 7.45-7.28 (m, 2H), 6.53 (s, 1H), 5.83-5.68 (m, 1H), 5.27 (s, 2H), 5.18-5.04 (m, 2H), 4.91 (s, 2H), 4.32 (d, J=18.4 Hz, 1H), 4.10 (d, J=18.5 Hz, 1H), 3.86 (dd, J=13.4, 3.3 Hz, 1H), 3.37 (d, J=13.3 Hz, 1H), 3.20-2.86 (m, 4H). LC-MS (M+H)=462.3.

Example 20: Analytical SFC tR=3.00 min. ¹H NMR (400 MHz, DMSO-d6, 80° C.) δ11.26 (s, 1H), 7.45-7.28 (m, 2H), 6.53 (s, 1H), 5.83-5.68 (m, 1H), 5.27 (s, 2H), 5.18-5.04 (m, 2H), 4.91 (s, 2H), 4.32 (d, J=18.4 Hz, 1H), 4.10 (d, J=18.5 Hz, 1H), 3.86 (dd, J=13.4, 3.3 Hz, 1H), 3.37 (d, J=13.3 Hz, 1H), 3.20-2.86 (m, 4H). LC-MS (M+H)=462.3.

### Example 21 & Example 22: (5-amino-6,8-dihydro-1H-furo[3,4-d]pyrrolo[3,2-b]pyridin-2-yl)((1S,5S)-1,3,4,6-tetrahydro-2H-1,5-methanobenzo[f][1,4]diazocin-2-yl)methanone & (5-amino-6,8-dihydro-1H-furo[3,4-d]pyrrolo[3,2-b]pyridin-2-yl)((1R,5R)-1,3,4,6-tetrahydro-2H-1,5-methanobenzo[f][1,4]diazocin-2-yl)methanone

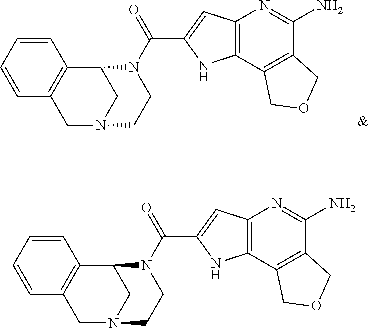 [SMILES: Nc1nc2cc(C(=O)N3CC[N@]4Cc5ccccc5[C@H]3C4)[nH]c2c2c1COC2 ; Nc1nc2cc(C(=O)N3CC[N@@]4Cc5ccccc5[C@@H]3C4)[nH]c2c2c1COC2]

### Step 1: ethyl 2-((benzyl(2-ethoxy-2-oxoethyl)amino)methyl)benzoate

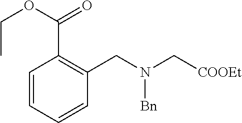 [SMILES: CCOC(=O)CN(Cc1ccccc1)Cc1ccccc1C(=O)OCC]

The title compound (8.9 g, 76%) was prepared in a manner similar to that in Example 17 & Example 18 step 3 from ethyl 2-(bromomethyl)benzoate and N-benzylglycine ethyl ester. LC-MS (M+H)⁺=356.2.

### Step 2: ethyl 2-benzyl-4-oxo-1,2,3,4-tetrahydroisoquinoline-3-carboxylate

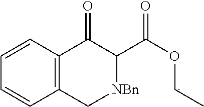 [SMILES: CCOC(=O)C1C(=O)c2ccccc2CN1Cc1ccccc1]

The title compound (6.1 g, 79%) was prepared in a manner similar to that in Example 17 & Example 18 step 4 from ethyl 2-((benzyl(2-ethoxy-2-oxoethyl)amino)methyl)benzoate. LC-MS (M+H)⁺=310.2.

### Step 3: 2-benzyl-2,3-dihydroisoquinolin-4(1H)-one

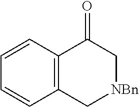 [SMILES: O=C1CN(Cc2ccccc2)Cc2ccccc21]

The title compound (4.1 g, 88%) was prepared in a manner similar to that in Example 17 & Example 18 step 5 from ethyl 2-benzyl-4-oxo-1,2,3,4-tetrahydroisoquinoline-3-carboxylate. LC-MS (M+H)⁺=238.0.

### Step 4: 4-hydroxy-1,2,3,4-tetrahydroisoquinolin-2-ium acetate

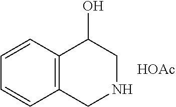 [SMILES: OC1CNCc2ccccc21 ; "HOAc"]

The title compound (1.1 g, 31%) was prepared in a manner similar to that in Example 17 & Example 18 step 6 from 2-benzyl-2,3-dihydroisoquinolin-4(1H)-one. LC-MS (M+H)=150.0.

### Step 5: tert-butyl (2-(4-hydroxy-3,4-dihydroisoquinolin-2(1H)-yl)ethyl)carbamate

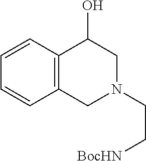 [SMILES: CC(C)(C)OC(=O)NCCN1Cc2ccccc2C(O)C1]

The title compound (405 mg, 97%) was prepared in a manner similar to that in Example 17 & Example 18 step 7 from 4-hydroxy-1,2,3,4-tetrahydroisoquinolin-2-ium acetate and tert-butyl (2-iodoethyl)carbamate. LC-MS (M+H)=293.2.

### Step 6: 2-(2-aminoethyl)-1,2,3,4-tetrahydroisoquinolin-4-ol dihydrochloride

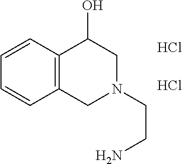 [SMILES: NCCN1Cc2ccccc2C(O)C1 ; Cl ; Cl]

The title compound (230 mg, 63%) was prepared in a manner similar to that in Example 17 & Example 18 step 8 from tert-butyl (2-(4-hydroxy-3,4-dihydroisoquinolin-2(1H)-yl)ethyl)carbamate. LC-MS (M+H)⁺=193.3.

### Step 7: N-(2-(4-hydroxy-3,4-dihydroisoquinolin-2(1H)-yl)ethyl)-2-nitrobenzenesulfonamide

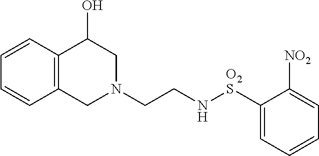 [SMILES: O=[N+]([O-])c1ccccc1S(=O)(=O)NCCN1Cc2ccccc2C(O)C1]

The title compound (300 mg, 92%) was prepared in a manner similar to that in Example 17 & Example 18 step 9 from 2-(2-aminoethyl)-1,2,3,4-tetrahydroisoquinolin-4-ol dihydrochloride and 2-nitrobenzenesulfonyl chloride. LC-MS (M+H)⁺=378.1.

### Step 8: 1,3,4,6-tetrahydro-2H-1,5-methanobenzo[f][1,4]diazocine

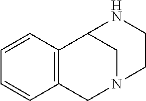 [SMILES: c1ccc2c(c1)CN1CCNC2C1]

The title compound (73 mg, 53%) was prepared in a manner similar to that in Example 17 & Example 18 step 10 from N-(2-(4-hydroxy-3,4-dihydroisoquinolin-2(1H)-yl)ethyl)-2-nitrobenzenesulfonamide. LC-MS (M+H)⁺=175.1.

### Step 9: (5-amino-1-((2-(trimethylsilyl)ethoxy)methyl)-6,8-dihydro-1H-furo[3,4-d]pyrrolo[3,2-b]pyridin-2-yl)(1,3,4,6-tetrahydro-2H-1,5-methanobenzo[f][1,4]diazocin-2-yl)methanone

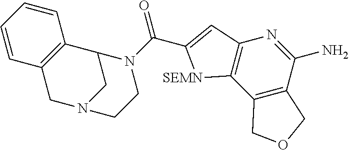 [SMILES: C[Si](C)(C)CCOCn1c(C(=O)N2CCN3Cc4ccccc4C2C3)cc2nc(N)c3c(c21)COC3]

The title compound (110 mg, 52%) was prepared in a manner similar to that in Example 17 & Example 18 step 11 from 1,3,4,6-tetrahydro-2H-1,5-methanobenzo[f][1,4]diazocine and 5-amino-1-((2-(trimethylsilyl)ethoxy)methyl)-6,8-dihydro-1H-furo[3,4-d]pyrrolo[3,2-b]pyridine-2-carboxylic acid. LC-MS (M+H)⁺=506.4.

### Step 10: (5-amino-6,8-dihydro-1H-furo[3,4-d]pyrrolo[3,2-b]pyridin-2-yl)((1S,5S)-1,3,4,6-tetrahydro-2H-1,5-methanobenzo[f][1,4]diazocin-2-yl)methanone & (5-amino-6,8-dihydro-1H-furo[3,4-d]pyrrolo[3,2-b]pyridin-2-yl)((1R,5R)-1,3,4,6-tetrahydro-2H-1,5-methanobenzo[f][1,4]diazocin-2-yl)methanone

Example 21 (29 mg, 36%) and Example 22 (29 mg, 36%) were prepared in a manner similar to that in Example 17 & Example 18 step 12 from (5-amino-1-((2-(trimethylsilyl)ethoxy)methyl)-6,8-dihydro-1H-furo[3,4-d]pyrrolo[3,2-b]pyridin-2-yl)(1,3,4,6-tetrahydro-2H-1,5-methanobenzo[f][1,4]diazocin-2-yl)methanone, and the enantiomers were separated by chiral SFC.

Analytical chiral-SFC condition as below. Column: CHIRALPAK AS-H; Column size: 4.6×100 mm, 5 μm; Mobile phase: 4 mM methanolic NH₃:CO₂, 1:9 to 1:1 in 3 min, 1:1 for 2 min, 1:1 to 1:9 in 0.1 min, 1:9 for 1.9 min; Flow: 2.0 mL/min; Temperature: 35° C.; back pressure: 1500 psi.

Example 21: Analytical SFC tR=3.17 min. ¹H NMR (400 MHz, DMSO-d6, 80° C.) δ11.29 (s, 1H), 7.34-6.96 (m, 4H), 6.53 (s, 1H), 5.45 (s, 1H), 5.29 (s, 2H), 5.14 (s, 2H), 4.93 (s, 2H), 4.27 (d, J=18.0, 1H), 4.02-3.77 (m, 2H), 3.42-2.88 (m, 5H). LC-MS (M+H)⁺=376.3.

Example 22: Analytical SFC tR=3.58 min. ¹H NMR (400 MHz, DMSO-d6, 80° C.) δ11.29 (s, 1H), 7.35-6.91 (m, 4H), 6.53 (s, 1H), 5.45 (s, 1H), 5.29 (s, 2H), 5.14 (s, 2H), 4.95 (s, 2H), 4.27 (d, J=18.0, 1H), 3.96-3.81 (m, 2H), 3.41-2.83 (m, 5H). LC-MS (M+H)⁺=376.3.

### Example 23 & Example 24: (5-amino-6,8-dihydro-1H-furo[3,4-d]pyrrolo[3,2-b]pyridin-2-yl)((1R,5R)-8-bromo-1,3,4,6-tetrahydro-2H-1,5-methanobenzo[f][1,4]diazocin-2-yl)methanone & (5-amino-6,8-dihydro-1H-furo[3,4-d]pyrrolo[3,2-b]pyridin-2-yl)((1S,5S)-8-bromo-1,3,4,6-tetrahydro-2H-1,5-methanobenzo[f][1,4]diazocin-2-yl)methanone

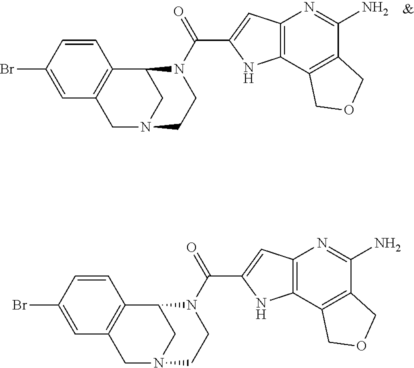 [SMILES: Nc1nc2cc(C(=O)N3CC[N@@]4Cc5cc(Br)ccc5[C@@H]3C4)[nH]c2c2c1COC2 ; Nc1nc2cc(C(=O)N3CC[N@]4Cc5cc(Br)ccc5[C@H]3C4)[nH]c2c2c1COC2]

### Step 1: methyl (3-bromobenzyl)glycinate

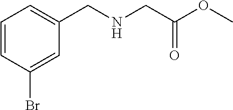

To a mixture of 3-bromobenzaldehyde (13.5 g, 73 mmol), methyl glycinate hydrochloride (13.7 g, 109 mmol) and Et₃N (16 ml, 109 mmol) in MeOH (200 mL) was added NaBH₃CN (6.9 g, 109 mmol), and the mixture was stirred at 70° C. overnight. The mixture was concentrated under reduced pressure, and the mixture was partitioned between water (100 mL) and EtOAc (500 mL). The organic layer was washed with brine (100 mL), dried over Na₂SO₄, filtered and concentrated under reduced pressure. The residue was purified by silica gel chromatograph (PE:EtOAc=2:1) to give the title compound (11.2 g, 59%) LC-MS (M+H)⁺=258.2.

### Step 2: methyl N-((benzyloxy)carbonyl)-N-(3-bromobenzyl)glycinate

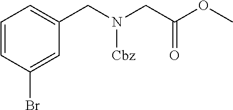 [SMILES: COC(=O)CN(Cc1cccc(Br)c1)C(=O)OCc1ccccc1]

To a mixture of methyl (3-bromobenzyl)glycinate (11.2 g, 43.4 mmol) and K₂CO₃ (18.0 g, 130 mmol) in DMF (100 mL) was added CbzCl (15.4 mL, 109 mmol) dropwise at room temperature. The mixture was stirred at 80° C. for 4 h and cooled to room temperature. The mixture was concentrated under reduced pressure. The residue was partitioned between EtOAc (200 mL) and water (100 mL). The organic layer was dried over Na₂SO₄, filtered and concentrated under reduced pressure. The residue was purified by silica gel chromatography (PE:EtOAc=4:1) to give the title compound (9.0 g, 53%). LC-MS (M+H)⁺=392.2.

### Step 3: N-((benzyloxy)carbonyl)-N-(3-bromobenzyl)glycine

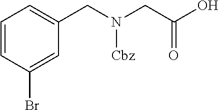 [SMILES: O=C(O)CN(Cc1cccc(Br)c1)C(=O)OCc1ccccc1]

To a mixture of methyl N-((benzyloxy)carbonyl)-N-(3-bromobenzyl)glycinate (9.0 g, 23 mmol) in MeOH (30 mL) was added a solution of NaOH (4.6 g, 115 mmol) in water (30 mL) dropwise. The mixture was stirred at room temperature for 2 h and concentrated under reduced pressure. The residue was dissolved in EtOAc (200 mL) and successively washed with 4 N hydrochloric acid (50 mL) and water (100 mL). The organic layer was dried over Na₂SO₄, filtered and concentrated under reduced pressure to give the title compound (8.5 g, 98%). LC-MS (M+H)⁺=378.2.

### Step 4: tert-butyl 7-bromo-4-oxo-3,4-dihydroisoquinoline-2(1H)-carboxylate

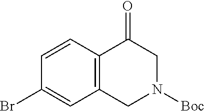 [SMILES: CC(C)(C)OC(=O)N1CC(=O)c2ccc(Br)cc2C1]

To a solution of N-((benzyloxy)carbonyl)-N-(3-bromobenzyl)glycine (10 g, 26 mmol) in DCM (100 mL) was added SOCl₂ (2.83 mL, 39 mmol) dropwise at room temperature, and the mixture was warmed to 45° C. for 3 h. The mixture was cooled to room temperature and concentrated under reduced pressure. The residue was re-dissolved in DCM (100 mL), followed by addition of AlCl₃ (13.8 g, 104 mmol) in portions. The mixture was stirred at room temperature for 12 h and then added into saturated NaHCO₃ (500 mL) slowly with stirring. The mixture was aged for 1 h and solid was filtered off. The solid was rinsed with DCM (100 mL). The organic layer of the filtrate was separated, washed with water (50 mL), dried over Na₂SO₄, filtered and concentrated under reduced pressure. The residue was re-dissolved in DCM (100 mL) followed by addition of Et₃N (12.7 mL, 88.5 mmol) and Boc₂O (9.6 g, 44.26 mmol). The mixture was stirred at room temperature for 2 h and diluted with DCM (100 mL). The mixture was washed with water (50 mL), dried over Na₂SO₄, filtered and concentrated under vacuum to give the title compound (7.0 g, 83%).

### Step 5: tert-butyl 7-bromo-4-hydroxy-3,4-dihydroisoquinoline-2(1H)-carboxylate

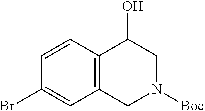 [SMILES: CC(C)(C)OC(=O)N1Cc2cc(Br)ccc2C(O)C1]

To a mixture of tert-butyl 7-bromo-4-oxo-3,4-dihydroisoquinoline-2(1H)-carboxylate (7.0 g, 22.1 mmol) in MeOH (100 mL) was added NaBH₄ (1.68 g, 44.2 mmol) in portions at room temperature. The mixture was stirred at room temperature for 1 h and concentrated under reduced pressure. The residue was partitioned between EtOAc (200 mL) and water (100 mL). The organic layer was dried over Na₂SO₄, filtered and concentrated under reduced pressure. The residue was purified by silica gel chromatography (PE:EtOAc=4:1) to give the title compound (3.0 g, 42%). LC-MS (M+H)⁺=328.2.

### Step 6: tert-butyl 7-bromo-4-((2-hydroxyethyl)amino)-3,4-dihydroisoquinoline-2(1H)-carboxylate

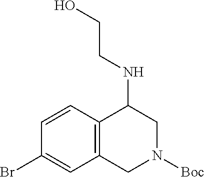 [SMILES: CC(C)(C)OC(=O)N1Cc2cc(Br)ccc2C(NCCO)C1]

To a mixture of tert-butyl 7-bromo-4-hydroxy-3,4-dihydroisoquinoline-2(1H)-carboxylate (2.0 g, 6.1 mmol) in DCM (100 mL) was added Et₃N (1.76 mL, 12.2 mmol) and MsCl (0.60 mL, 7.93 mmol) at room temperature. The mixture was stirred at room temperature for 1 h, diluted with DCM (100 mL) and washed with water (50 mL). The organic layer was dried over Na₂SO₄, filtered and concentrated under reduced pressure. To the residue was added 2-aminoethan-1-ol (6 mL) and DMF (6 mL) and the mixture was stirred at 80° C. for 3 h. The mixture was cooled to room temperature and partitioned between EtOAc (200 mL) and water (100 mL). The organic layer was dried over Na₂SO₄, filtered and concentrated under reduced pressure. The residue was purified by silica gel chromatograph (PE:EtOAc=1:1 to 0:1) to give the title compound (1.4 g, 63%). LC-MS (M+H)⁺=371.1.

### Step 7: 8-bromo-1,3,4,6-tetrahydro-2H-1,5-methanobenzo[f][1,4]diazocine

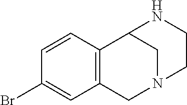 [SMILES: Brc1ccc2c(c1)CN1CCNC2C1]

To a mixture of tert-butyl 7-bromo-4-((2-hydroxyethyl)amino)-3,4-dihydroisoquinoline-2(1H)-carboxylate (2.2 g, 5.9 mmol) in THF (20 mL) was added SOCl₂ (2.1 mL, 29.5 mmol) at room temperature and the mixture was stirred at 60° C. for 2 h. The mixture was cooled to room temperature and concentrated under reduced pressure. The residue was re-dissolved in HCl in dioxane (4.0 M, 10 mL) and the mixture was stirred at room temperature for 2 h. The mixture was concentrated under reduced pressure. The residue was re-dissolved in DMF (10 mL) followed by addition of K₂CO₃ (4.0 g, 29.5 mmol). The mixture was stirred at 60° C. for 2 h and then cooled to room temperature. The mixture was partitioned between DCM (100 mL) and water (50 mL). The organic layer was dried over Na₂SO₄, filtered and concentrated under reduced pressure. The residue was purified by silica gel chromatograph (DCM:MeOH=20:1) to give the title compound (800 mg, 53%). LC-MS (M+H)⁺=253.2.

### Step 8: (5-amino-1-((2-(trimethylsilyl)ethoxy)methyl)-6,8-dihydro-1H-furo[3,4-d]pyrrolo[3,2-b]pyridin-2-yl)(8-bromo-1,3,4,6-tetrahydro-2H-1,5-methanobenzo[f][1,4]diazocin-2-yl)methanone

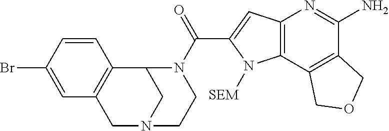 [SMILES: C[Si](C)(C)CCOCn1c(C(=O)N2CCN3Cc4cc(Br)ccc4C2C3)cc2nc(N)c3c(c21)COC3]

To a mixture of 5-amino-1-((2-(trimethylsilyl)ethoxy)methyl)-6,8-dihydro-1H-furo[3,4-d]pyrrolo[3,2-b]pyridine-2-carboxylic acid (275 mg, 0.79 mmol) and 8-bromo-1,3,4,6-tetrahydro-2H-1,5-methanobenzo[f][1,4]diazocine (200 mg, 0.79 mmol) in THF (5 mL) was added BOPCl (300 mg, 1.18 mmol) and DIPEA (0.73 mL, 3.95 mmol). The mixture was stirred at 60° C. for 2 h and then cooled to room temperature. The mixture was partitioned between water (10 mL) and EtOAc (50 mL). The organic layer was washed with brine (10 mL), dried over Na₂SO₄, filtered and concentrated under reduced pressure. The residue was purified by prep-TLC (DCM:MeOH=20:1) to give the title compound (100 mg, 21%). LC-MS (M+H)⁺=584.2.

### Step 9: (5-amino-6,8-dihydro-1H-furo[3,4-d]pyrrolo[3,2-b]pyridin-2-yl)((1R,5R)-8-bromo-1,3,4,6-tetrahydro-2H-1,5-methanobenzo[f][1,4]diazocin-2-yl)methanone & (5-amino-6,8-dihydro-1H-furo[3,4-d]pyrrolo[3,2-b]pyridin-2-yl)((1S,5S)-8-bromo-1,3,4,6-tetrahydro-2H-1,5-methanobenzo[f][1,4]diazocin-2-yl)methanone

A mixture of (5-amino-1-((2-(trimethylsilyl)ethoxy)methyl)-6,8-dihydro-1H-furo[3,4-d]pyrrolo[3,2-b]pyridin-2-yl)(8-bromo-1,3,4,6-tetrahydro-2H-1,5-methanobenzo[f][1,4]diazocin-2-yl)methanone (100 mg, 0.17 mmol) in TFA (2 mL) was stirred at room temperature for 0.5 h and concentrated under reduced pressure. The residue was dissolved in THF (4 mL) and ammonium hydroxide (28%, 1 mL). The mixture was stirred at room temperature for 2 h and concentrated under reduced pressure. The residue was purified by prep-HPLC followed by chiral SFC to give Example 23 (21 mg, 27%) and Example 24 (26 mg, 34%).

Analytical chiral-SFC condition as below. Column: YMC Cellulose-SB; Column size: 4.6×150 mm, 5 μm; Mobile phase: 4 mM methanolic NH₃:CO₂, 45:55 isocratic; Flow: 2.0 mL/min; Temperature: 35° C.; back pressure: 1500 psi.

Example 23: Analytical SFC tR=8.96 min. ¹H NMR (400 MHz, DMSO-d6, 80° C.) δ11.27 (s, 1H), 7.40-7.21 (m, 2H), 7.12-6.88 (m, 1H), 6.51 (s, 1H), 5.52-5.38 (m, 1H), 5.28 (s, 2H), 5.11 (s, 2H), 4.90 (s, 2H), 4.23 (d, J=18.2 Hz, 1H), 3.95 (d, J=18.2 Hz, 1H), 3.83 (dd, J=13.2, 3.5 Hz, 1H), 3.31 (d, J=13.2 Hz, 1H), 3.20-2.77 (m, 4H). LC-MS (M+H)⁺=454.3.

Example 24: Analytical SFC tR=21.94 min. ¹H NMR (400 MHz, DMSO-d6, 80° C.) δ11.27 (s, 1H), 7.40-7.21 (m, 2H), 7.12-6.88 (m, 1H), 6.51 (s, 1H), 5.52-5.38 (m, 1H), 5.28 (s, 2H), 5.11 (s, 2H), 4.90 (s, 2H), 4.23 (d, J=18.2 Hz, 1H), 3.95 (d, J=18.2 Hz, 1H), 3.83 (dd, J=13.2, 3.5 Hz, 1H), 3.31 (d, J=13.2 Hz, 1H), 3.20-2.77 (m, 4H). LC-MS (M+H)⁺=454.3.

### Example 25: (5-amino-6,8-dihydro-1H-furo[3,4-d]pyrrolo[3,2-b]pyridin-2-yl)(8-cyclopropyl-1,3,4,6-tetrahydro-2H-1,5-methanobenzo[f][1,4]diazocin-2-yl)methanone

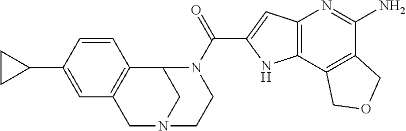 [SMILES: Nc1nc2cc(C(=O)N3CCN4Cc5cc(C6CC6)ccc5C3C4)[nH]c2c2c1COC2]

### Step 1: tert-butyl 8-bromo-1,3,4,6-tetrahydro-2H-1,5-methanobenzo[f][1,4]diazocine-2-carboxylate

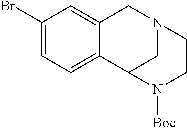 [SMILES: CC(C)(C)OC(=O)N1CCN2Cc3cc(Br)ccc3C1C2]

To a mixture of 8-bromo-1,3,4,6-tetrahydro-2H-1,5-methanobenzo[f][1,4]diazocine (300 mg, 1.18 mmol) in DCM (20 mL) was added Et₃N (0.5 mL, 3.54 mmol) and Boc₂O (385 mg, 1.77 mmol) at room temperature. After 2 h, the mixture was diluted with DCM (20 mL) and washed with water (20 mL). The organic layer was dried over Na₂SO₄, filtered, and concentrated under reduced pressure. The residue was purified by prep-TLC (EtOAc) to give the title compound (300 mg, 71%). LC-MS (M+H)=353.2.

### Step 2: tert-butyl 8-cyclopropyl-1,3,4,6-tetrahydro-2H-1,5-methanobenzo[f][1,4]diazocine-2-carboxylate

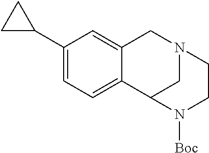 [SMILES: CC(C)(C)OC(=O)N1CCN2Cc3cc(C4CC4)ccc3C1C2]

To a mixture of tert-butyl 8-bromo-1,3,4,6-tetrahydro-2H-1,5-methanobenzo[f][1,4]diazocine-2-carboxylate (70 mg, 0.20 mmol), cyclopropylboronic acid (26 mg, 0.30 mmol) and K₂CO₃ (56 mg, 0.40 mmol) in toluene (12 mL) and water (3 mL), was added Pd₂dba₃ (9 mg, 0.010 mmol) and tricyclohexylphosphine (6 mg, 0.020 mmol) under nitrogen. The mixture was stirred at 100° C. for 12 h and cooled to room temperature. The mixture was poured into water (10 mL) and then extracted with EtOAc (50 mL). The organic layer was washed with brine (10 mL), dried over Na₂SO₄, filtered and concentrated under reduced pressure. The residue was purified by prep-TLC (EtOAc) to give the title compound (30 mg, 48%). LC-MS (M+H)=315.2.

### Step 3: 8-cyclopropyl-13,4,6-tetrahydro-2H-1,5-methanobenzo[f][1,4]diazocine dihydrochloride

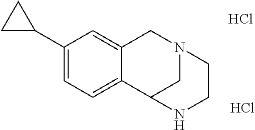 [SMILES: c1cc2c(cc1C1CC1)CN1CCNC2C1 ; Cl ; Cl]

The title compound (27 mg, 99%) was prepared in a manner similar to that in Example 19 & Example 20 step 11 from tert-butyl 8-cyclopropyl-1,3,4,6-tetrahydro-2H-1,5-methanobenzo[f][1,4]diazocine-2-carboxylate. LC-MS (M+H)=215.2.

### Step 4: (5-amino-1-((2-(trimethylsilyl)ethoxy)methyl)-6,8-dihydro-1H-furo[3,4-d]pyrrolo[3,2-b]pyridin-2-yl)(8-cyclopropyl-1,3,4,6-tetrahydro-2H-1,5-methanobenzo[f][1,4]diazocin-2-yl)methanone

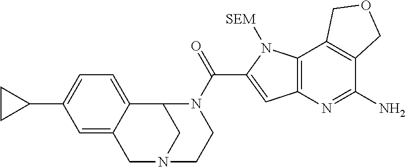 [SMILES: C[Si](C)(C)CCOCn1c(C(=O)N2CCN3Cc4cc(C5CC5)ccc4C2C3)cc2nc(N)c3c(c21)COC3]

The title compound (30 mg, 53%) was prepared in a manner similar to that in Example 19 & Example 20 step 12 from 8-cyclopropyl-1,3,4,6-tetrahydro-2H-1,5-methanobenzo[f][1,4]diazocine dihydrochloride and 5-amino-1-((2-(trimethylsilyl)ethoxy)methyl)-6,8-dihydro-1H-furo[3,4-d]pyrrolo[3,2-b]pyridine-2-carboxylic acid. LC-MS (M+H)⁺=546.3.

### Step 5: (5-amino-6,8-dihydro-1H-furo[3,4-d]pyrrolo[3,2-b]pyridin-2-yl)(8-cyclopropyl-1,3,4,6-tetrahydro-2H-1,5-methanobenzo[f][1,4]diazocin-2-yl)methanone

Example 25 (7 mg, 33%) was prepared in a manner similar to that in Example 17 & Example 18 step 12 from (5-amino-1-((2-(trimethylsilyl)ethoxy)methyl)-6,8-dihydro-1H-furo[3,4-d]pyrrolo[3,2-b]pyridin-2-yl)(8-cyclopropyl-1,3,4,6-tetrahydro-2H-1,5-methanobenzo[f][1,4]diazocin-2-yl)methanone. 1H NMR (400 MHz, DMSO-d6, 80° C.) δ11.25 (s, 1H), 8.11 (s, 1H), 6.89-6.83 (m, 3H), 6.49 (s, 1H), 5.37 (s, 1H), 5.26 (s, 2H), 5.11 (s, 2H), 4.90 (s, 2H), 4.22-3.83 (m, 3H), 3.04-2.98 (m, 3H), 2.96-2.94 (m, 2H), 1.86-1.82 (m, 1H), 0.88-0.87 (m, 2H), 0.61-0.60 (m, 2H). LC-MS (M+H)=416.4.

### Example 26 & Example 27: ((R)-5-amino-6-methyl-6,8-dihydro-1H-furo[3,4-d]pyrrolo[3,2-b]pyridin-2-yl)((1R,5R)-8-(trifluoromethyl)-1,3,4,6-tetrahydro-2H-1,5-methanobenzo[f][1,4]diazocin-2-yl)methanone & ((R)-5-amino-6-methyl-6,8-dihydro-1H-furo[3,4-d]pyrrolo[3,2-b]pyridin-2-yl)((1S,5S)-8-(trifluoromethyl)-1,3,4,6-tetrahydro-2H-1,5-methanobenzo[f][1,4]diazocin-2-yl)methanone

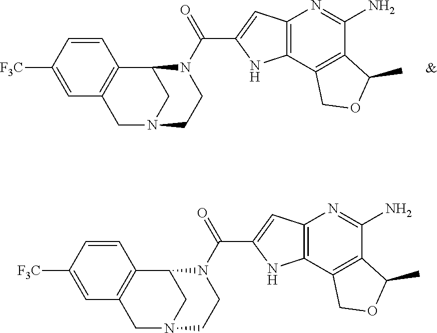 [SMILES: C[C@H]1OCc2c1c(N)nc1cc(C(=O)N3CC[N@@]4Cc5cc(C(F)(F)F)ccc5[C@@H]3C4)[nH]c21 ; C[C@H]1OCc2c1c(N)nc1cc(C(=O)N3CC[N@]4Cc5cc(C(F)(F)F)ccc5[C@H]3C4)[nH]c21]

### Step 1: 2-methyl-4-oxotetrahydrofuran-3-carbonitrile

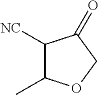

To a suspension of NaH (60%, 4.44 g, 0.11 mol) in THF (150 mL) was added methyl glycolate (20.0 g, 0.222 mol) at 0° C. The mixture was stirred at 60° C. for 30 min. Crotononitrile (17.8 g, 0.266 mmol) in THF (50 mL) was added dropwise at 60° C. The mixture was stirred at 70° C. for 2 h. The mixture was cooled to 0° C. and acidified with aqueous HCl (1 M) until its pH reached 4-5. The mixture was extracted with EtOAc (300 mL). The organic layer washed with water (100 mL) and brine (100 mL), dried over Na₂SO₄, filtered and concentrated under reduced pressure. The residue was purified by silica gel chromatography (PE/EtOAc=1/1) to give the title compound (10.0 g, 36%). ¹H NMR (500 MHz, CDCl₃) δ 4.42-4.27 (m, 2H), 4.26-4.18 (m, 2H), 3.23-2.98 (m, 1H), 1.63-1.27 (m, 3H).

### Step 2: 4-cyano-5-methyl-2,5-dihydrofuran-3-yl trifluoromethanesulfonate

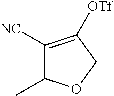 [SMILES: CC1OCC(OS(=O)(=O)C(F)(F)F)=C1C#N]

To a mixture of 2-methyl-4-oxotetrahydrofuran-3-carbonitrile (5.0 g, 40 mmol) and DIPEA (6.7 g, 52 mmol) in DCM (100 mL) was added Tf₂O (13.5 g, 48 mmol) dropwise at −78° C. and the mixture was stirred at −78° C. for 2 h. The mixture was diluted with DCM (100 mL), successively washed with saturated NaHCO₃ (50 mL) and brine (50 mL), dried over Na₂SO₄, filtered and concentrated under reduced pressure. The residue was purified by silica gel chromatography (PE/EtOAc=3/1) to give the title compound (8.5 g, 83%). ¹HNMR (500 MHz, CDCl₃) δ 5.13-5.06 (m, 1H), 4.94-4.76 (m, 2H), 1.49 (d, J=6.3 Hz, 3H).

### Step 3: ethyl 4-((tert-butoxycarbonyl)amino)-5-(4-cyano-5-methyl-2,5-dihydrofuran-3-yl)-1H-pyrrole-2-carboxylate

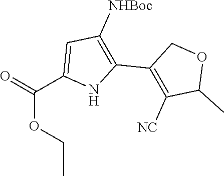 [SMILES: CCOC(=O)c1cc(NC(=O)OC(C)(C)C)c(C2=C(C#N)C(C)OC2)[nH]1]

To a solution of ethyl 5-bromo-4-((tert-butoxycarbonyl)amino)-1H-pyrrole-2-carboxylate (3.0 g, 9.0 mmol) and BPD (4.6 g, 18.0 mmol) in THF (50 mL) was added Pd₂(dba)₃ (0.41 g, 0.45 mmol), KOAc (2.65 g, 27.0 mmol) and XPhos (0.43 g, 0.90 mmol). The mixture was stirred at 75° C. for 3 h and cooled to room temperature. Solid was filtered off and the filtrate was concentrated under vacuum. The crude was re-dissolved in dioxane (100 mL) and water (20 mL), followed by addition of 4-cyano-5-methyl-2,5-dihydrofuran-3-yl trifluoromethanesulfonate (3.0 g, 11.7 mmol) Pd(dppf)Cl₂ (83 mg, 0.58 mmol) and NaHCO₃ (1.96 g, 23.4 mmol). The mixture was stirred at 80° C. for 5 h. The mixture was cooled to room temperature and diluted with EtOAc (100 mL). The mixture was washed with brine (50 mL), and the aqueous layer was back-extracted with EtOAc (50 mL). The combined organic layer was dried over Na₂SO₄, filtered and concentrated under reduced pressure. The residue was purified by silica gel chromatograph (PE/EtOAc=3/1) to give the title compound (2.0 g, 62%). LC-MS (M+H)⁺=362.1.

### Step 4: ethyl (S)-5-amino-6-methyl-6,8-dihydro-1H-furo[3,4-d]pyrrolo[3,2-b]pyridine-2-carboxylate & ethyl (R)-5-amino-6-methyl-6,8-dihydro-1H-furo[3,4-d]pyrrolo[3,2-b]pyridine-2-carboxylate

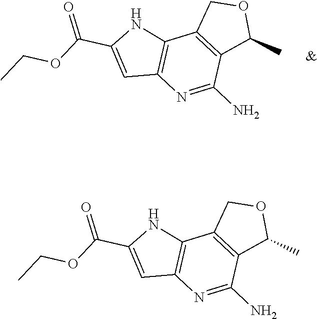

To the solution of ethyl 4-((tert-butoxycarbonyl)amino)-5-(4-cyano-5-methyl-2,5-dihydrofuran-3-yl)-1H-pyrrole-2-carboxylate (20 g, 55 mmol) in dioxane (100 mL) was added a solution of HCl in dioxane (100 mL, 4M) and the mixture was stirred at room temperature for 5 h. The mixture was concentrated under reduced pressure. The crude was re-dissolved in EtOH (200 mL) followed by addition of K₂CO₃ (27.6 g, 200 mmol). The mixture was stirred at 70° C. for 4 h then cooled to room temperature. Solid was filtered off and the filtrate was concentrated under reduced pressure. The residue was purified by silica gel chromatography (DCM:MeOH=10:1) to give a racemate (5.0 g). The material was separated by SFC to give ethyl (S)-5-amino-6-methyl-6,8-dihydro-1H-furo[3,4-d]pyrrolo[3,2-b]pyridine-2-carboxylate (2.25 g, 16%) and ethyl (R)-5-amino-6-methyl-6,8-dihydro-1H-furo[3,4-d]pyrrolo[3,2-b]pyridine-2-carboxylate (2.34 g, 16%).

Analytical chiral-SFC condition as below. Column: CHIRALPAK IC-3; Column size: 3.0×100 mm, 3 m; Mobile phase: 10 μM NH₃ in methanol:CO₂, 1:9 to 1:1 in 2 min, 1:1 for 1 min; Flow: 2.0 mL/min; Temperature: 35° C.; back pressure: 1500 psi.

ethyl (S)-5-amino-6-methyl-6,8-dihydro-1H-furo[3,4-d]pyrrolo[3,2-b]pyridine-2-carboxylate: Analytical SFC tR=2.01 min. LC-MS (M+H)⁺=262.1.

ethyl (R)-5-amino-6-methyl-6,8-dihydro-1H-furo[3,4-d]pyrrolo[3,2-b]pyridine-2-carboxylate: Analytical SFC tR=2.36 min. LC-MS (M+H)⁺=262.1.

### Step 5: (R)-5-amino-6-methyl-6,8-dihydro-1H-furo[3,4-d]pyrrolo[3,2-b]pyridine-2-carboxylic acid

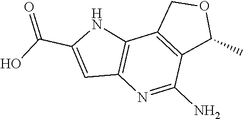 [SMILES: C[C@H]1OCc2c1c(N)nc1cc(C(=O)O)[nH]c21]

A mixture of ethyl (R)-5-amino-6-methyl-6,8-dihydro-1H-furo[3,4-d]pyrrolo[3,2-b]pyridine-2-carboxylate (1.5 g, 5.7 mmol) and LiOH·H₂O (0.32 g, 7.6 mmol) in MeOH (20 mL) and water (10 mL) was stirred at 50° C. for 3 h. The mixture was cooled to room temperature and concentrated under reduced pressure. The residue was purified by prep-HPLC to give the title compound (1.2 g, 90%). LC-MS (M+H)⁺=234.1.

### Step 6: ((R)-5-amino-6-methyl-6,8-dihydro-1H-furo[3,4-d]pyrrolo[3,2-b]pyridin-2-yl)((1R,5R)-8-(trifluoromethyl)-1,3,4,6-tetrahydro-2H-1,5-methanobenzo[f][1,4]diazocin-2-yl)methanone & ((R)-5-amino-6-methyl-6,8-dihydro-1H-furo[3,4-d]pyrrolo[3,2-b]pyridin-2-yl)((1S,5S)-8-(trifluoromethyl)-1,3,4,6-tetrahydro-2H-1,5-methanobenzo[f][1,4]diazocin-2-yl)methanone

To a mixture of 8-(trifluoromethyl)-1,3,4,6-tetrahydro-2H-1,5-methanobenzo[f][1,4]diazocine (96 mg, 0.40 mmol), (R)-5-amino-6-methyl-6,8-dihydro-1H-furo[3,4-d]pyrrolo[3,2-b]pyridine-2-carboxylic acid (93 mg, 0.40 mmol) in DMF (10 mL) was added T3P (50% in DMF, 1.27 g, 2.0 mmol) and DIPEA (361 mg, 2.8 mmol). The mixture was stirred at 60° C. for 3 h and then cooled to room temperature. Hydrochloric acid (4 N, 10 mL) was added slowly to the mixture, and the mixture was stirred at 60° C. for 6 h. The mixture was cooled to room temperature and poured to saturated NaHCO₃ (50 mL). The mixture was extracted with EtOAc (50 mL×3). The combined organic layer was washed with brine (50 mL), dried over Na₂SO₄, filtered and concentrated under reduced pressure. The residue was purified by prep-HPLC followed by chiral SFC to give Example 26 (12 mg, 7%) and Example 27 (11 mg, 6%).

Analytical chiral-SFC condition as below. Column: CHIRALPAK Cellulose-C; Column size: 4.6×100 mm, 5 μm; Mobile phase: 4 mM methanolic NH₃:CO₂, 45:55 isocratic; Flow: 2.0 mL/min; Temperature: 35° C.; back pressure: 1500 psi.

Example 26: Analytical SFC tR=2.16 min. ¹H NMR (400 MHz, DMSO-d6, 80° C.) δ11.50 (s, 1H), 7.65-6.82 (m, 3H), 6.80-6.23 (m, 1H), 5.65-4.85 (m, 6H), 4.41-3.62 (m, 3H), 3.26-2.81 (m, 5H), 1.31 (d, J=5.4 Hz, 3H). LC-MS (M+H)⁺=458.3.

Example 27: Analytical SFC tR=4.99 min. ¹H NMR (400 MHz, DMSO-d6, 80° C.) δ11.48 (s, 1H), 7.62-6.82 (m, 3H), 6.74-6.13 (m, 1H), 5.65-4.87 (m, 6H), 4.38-3.62 (m, 3H), 3.23-2.79 (m, 5H), 1.30 (d, J=5.6 Hz, 3H). LC-MS (M+H)⁺=458.3.

### Example 28 & Example 29: ((R)-5-amino-6-methyl-6,8-dihydro-1H-furo[3,4-d]pyrrolo[3,2-b]pyridin-2-yl)((1R,5R)-8-bromo-1,3,4,6-tetrahydro-2H-1,5-methanobenzo[f][1,4]diazocin-2-yl)methanone & ((R)-5-amino-6-methyl-6,8-dihydro-1H-furo[3,4-d]pyrrolo[3,2-b]pyridin-2-yl)((1S,5S)-8-bromo-1,3,4,6-tetrahydro-2H-1,5-methanobenzo[f][1,4]diazocin-2-yl)methanone

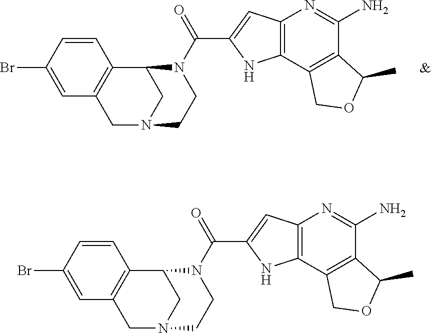 [SMILES: C[C@H]1OCc2c1c(N)nc1cc(C(=O)N3CC[N@@]4Cc5cc(Br)ccc5[C@@H]3C4)[nH]c21 ; C[C@H]1OCc2c1c(N)nc1cc(C(=O)N3CC[N@]4Cc5cc(Br)ccc5[C@H]3C4)[nH]c21]

Example 28 (40 mg, 11%) and Example 29 (45 mg, 12%) were prepared in a manner similar to that in Example 26 & Example 27 step 6 from (R)-5-amino-6-methyl-6,8-dihydro-1H-furo[3,4-d]pyrrolo[3,2-b]pyridine-2-carboxylic acid and 8-bromo-1,3,4,6-tetrahydro-2H-1,5-methanobenzo[f][1,4]diazocine, and the diastereomers were separated by chiral SFC.

Analytical chiral-SFC condition as below. Column: YMC Cellulose-SB; Column size: 4.6×100 mm, 5 μm; Mobile phase: 4 mM methanolic NH₃:CO₂, 45:55 isocratic; Flow: 2.0 mL/min; Temperature: 35° C.; back pressure: 1500 psi.

Example 28: Analytical SFC tR=7.24 min. ¹H NMR (400 MHz, DMSO-d6, 80° C.) δ11.27 (s, 1H), 7.34-7.31 (m, 2H), 7.00 (s, 1H), 6.50 (s, 2H), 5.42 (s, 1H), 5.18 (s, 1H), 5.16-5.11 (m, 3H), 5.04-5.01 (m, 1H), 3.83-3.67 (m, 3H), 3.29-2.88 (m, 5H), 1.36-1.35 (m, 3H). LC-MS (M+H)⁺=468.3.

Example 29: Analytical SFC tR=19.95 min. ¹H NMR (400 MHz, DMSO-d6, 80° C.) δ11.27 (s, 1H), 7.42-7.24 (m, 2H), 7.10-6.89 (m, 1H), 6.50 (s, 1H), 5.41 (s, 1H), 5.37-5.27 (m, 1H), 5.22-5.08 (m, 3H), 5.01 (d, J=12.9 Hz, 1H), 4.23 (d, J=18.1 Hz, 1H), 3.95 (d, J=18.1 Hz, 1H), 3.82 (dd, J=12.8, 3.3 Hz, 1H), 3.31 (d, J=13.1 Hz, 1H), 3.21-2.76 (m, 4H), 1.35 (d, J=6.1 Hz, 3H). LC-MS (M+H)⁺=468.3.

### Example 30 & Example 31: (5-amino-6,8-dihydro-1H-furo[3,4-d]pyrrolo[3,2-b]pyridin-2-yl)((1R,5R)-8-iodo-1,3,4,6-tetrahydro-2H-1,5-methanobenzo[f][1,4]diazocin-2-yl)methanone & (5-amino-6,8-dihydro-1H-furo[3,4-d]pyrrolo[3,2-b]pyridin-2-yl)((1S,5S)-8-iodo-1,3,4,6-tetrahydro-2H-1,5-methanobenzo[f][1,4]diazocin-2-yl)methanone

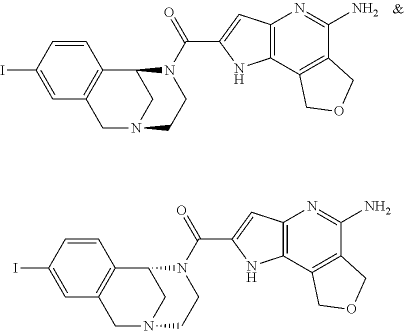 [SMILES: Nc1nc2cc(C(=O)N3CC[N@@]4Cc5cc(I)ccc5[C@@H]3C4)[nH]c2c2c1COC2 ; Nc1nc2cc(C(=O)N3CC[N@]4Cc5cc(I)ccc5[C@H]3C4)[nH]c2c2c1COC2]

### Step 1: tert-butyl 8-iodo-1,3,4,6-tetrahydro-2H-1,5-methanobenzo[f][1,4]diazocine-2-carboxylate

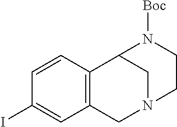 [SMILES: CC(C)(C)OC(=O)N1CCN2Cc3cc(I)ccc3C1C2]

To a mixture of tert-butyl 8-bromo-1,3,4,6-tetrahydro-2H-1,5-methanobenzo[f][1,4]diazocine-2-carboxylate (100 mg, 0.28 mmol) in dioxane (5 mL) was added NaI (170 mg, 1.14 mmol), CuI (27 mg, 0.14 mmol) and trans-N,N′-dimethyl-1,2-cyclohexanediamine (40 mg, 0.28 mmol) under nitrogen. The mixture was stirred for 16 h at 100° C. and cooled to room temperature. The mixture was poured into iced water (50 mL) and extracted with EtOAc (50 mL). The organic layer was washed with brine (50 mL), dried over Na₂SO₄, filtered and concentrated under reduced pressure. The residue was purified by silica gel chromatograph (DCM:MeOH=20:1) to give the title compound (100 mg, 88%). LC-MS (M+H)⁺=401.3.

### Step 2: 8-iodo-1,3,4,6-tetrahydro-2H-1,5-methanobenzo[f][1,4]diazocine

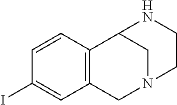 [SMILES: Ic1ccc2c(c1)CN1CCNC2C1]

To a solution of tert-butyl 8-iodo-1,3,4,6-tetrahydro-2H-1,5-methanobenzo[f][1,4]diazocine-2-carboxylate (100 mg, 0.25 mmol) in DCM (5 mL) was added HCl (4.0 M in dioxane, 10 mL). The mixture was stirred at room temperature for 3 h and then concentrated under reduced pressure. The residue was partitioned between saturated NaHCO₃ (50 mL) and DCM (100 mL). The organic layer was washed with brine (50 mL), dried over Na₂SO₄, filtered and concentrated under reduced pressure to give the title compound (75 mg, 100%). LC-MS (M+H)⁺=301.1.

### Step 3: (5-amino-1-((2-(trimethylsilyl)ethoxy)methyl)-6,8-dihydro-1H-furo[3,4-d]pyrrolo[3,2-b]pyridin-2-yl)(8-iodo-1,3,4,6-tetrahydro-2H-1,5-methanobenzo[f][1,4]diazocin-2-yl)methanone

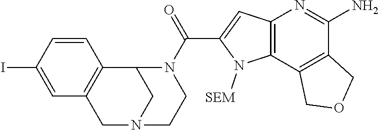 [SMILES: C[Si](C)(C)CCOCn1c(C(=O)N2CCN3Cc4cc(I)ccc4C2C3)cc2nc(N)c3c(c21)COC3]

A mixture of 8-iodo-1,3,4,6-tetrahydro-2H-1,5-methanobenzo[f][1,4]diazocine (75 mg, 0.25 mmol) and 5-amino-1-((2-(trimethylsilyl)ethoxy)methyl)-6,8-dihydro-1H-furo[3,4-d]pyrrolo[3,2-b]pyridine-2-carboxylic acid (104 mg, 0.30 mmol) in THF (5 mL) was added BOPCl (99 mg, 0.39 mmol) and DIPEA (77 mg, 0.60 mmol) and the mixture was stirred at 60° C. for 16 h. The mixture was cooled to room temperature and concentrated under reduced pressure. The residue was partitioned between water (50 mL) and EtOAc (50 mL). The organic layer was dried with Na₂SO₄, filtered and concentrated under reduced pressure. The residue was purified by silica gel chromatograph (DCM:MeOH=25:1) to give the title compound (110 mg, 70%). LC-MS (M+H)⁺=632.2.

### Step 4: (5-amino-6,8-dihydro-1H-furo[3,4-d]pyrrolo[3,2-b]pyridin-2-yl)((1R,5R)-8-iodo-1,3,4,6-tetrahydro-2H-1,5-methanobenzo[f][1,4]diazocin-2-yl)methanone & (5-amino-6,8-dihydro-1H-furo[3,4-d]pyrrolo[3,2-b]pyridin-2-yl)((1S,5S)-8-iodo-1,3,4,6-tetrahydro-2H-1,5-methanobenzo[f][1,4]diazocin-2-yl)methanone

A mixture of (5-amino-1-((2-(trimethylsilyl)ethoxy)methyl)-6,8-dihydro-1H-furo[3,4-d]pyrrolo[3,2-b]pyridin-2-yl)(8-iodo-1,3,4,6-tetrahydro-2H-1,5-methanobenzo[f][1,4]diazocin-2-yl)methanone (110 mg, 0.17 mmol) in DCM (6 mL) and TFA (6 mL) was stirred at room temperature for 3 h and concentrated under reduced pressure. The residue was treated with methanolic ammonia (7.0 M, 10 mL). The mixture was stirred at room temperature for 2 h and then concentrated under reduced pressure. The residue was partitioned between saturated NaHCO₃ (50 mL) and EtOAc (50 mL). The organic layer was concentrated under reduced pressure. The residue was purified by prep-TLC (DCM:MeOH=10:1) followed by chiral SFC to give Example 30 (7 mg, 8%) and Example 31 (8 mg, 9%).

Analytical chiral-SFC condition as below. Column: Lux Cellulose-3; Column size: 4.6×100 mm, m; Mobile phase: 4 mM methanolic NH₃:CO₂, 45:55 isocratic; Flow: 2.0 mL/min; Temperature: 35° C.; back pressure: 1500 psi.

Example 30: Analytical SFC tR=3.81 min. ¹H NMR (400 MHz, DMSO-d6, 80° C.) δ11.46 (s, 1H), 7.62-7.36 (m, 2H), 7.14-6.24 (m, 2H), 5.59-5.27 (m, 3H), 5.08 (s, 2H), 4.87 (s, 2H), 4.31-4.06 (m, 1H), 3.98-3.59 (m, 2H), 3.21-2.76 (m, 5H). LC-MS (M+H)⁺=502.2.

Example 31: Analytical SFC tR=6.00 min. ¹H NMR (400 MHz, DMSO-d6, 80° C.) δ11.46 (s, 1H), 7.62-7.36 (m, 2H), 7.16-6.15 (m, 2H), 5.70-5.25 (m, 3H), 5.08- (s, 2H), 4.87 (s, 2H), 4.41-4.09 (m, 1H), 4.08-3.56 (m, 2H), 3.25-2.75 (m, 5H). LC-MS (M+H)⁺=502.2.

### Example 32: (5-amino-6,8-dihydro-1H-furo[3,4-d]pyrrolo[3,2-b]pyridin-2-yl)(8-bromo-6,6-dimethyl-1,3,4,6-tetrahydro-2H-1,5-methanobenzo[f][1,4]diazocin-2-yl)methanone

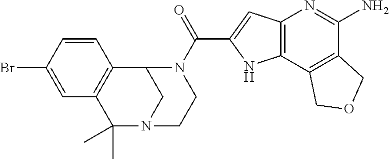 [SMILES: CC1(C)c2cc(Br)ccc2C2CN1CCN2C(=O)c1cc2nc(N)c3c(c2[nH]1)COC3]

### Step 1: tert-butyl (2-(3-bromophenyl)propan-2-yl)glycinate

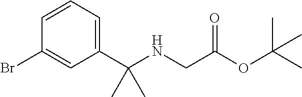 [SMILES: CC(C)(C)OC(=O)CNC(C)(C)c1cccc(Br)c1]

To a mixture of 2-(3-bromophenyl)propan-2-amine (5.0 g, 23.4 mmol) and K₂CO₃ (6.4 g, 46.7 mmol) in MeCN (100 mL) was added tert-butyl 2-bromoacetate (5.5 g, 28.0 mmol) and the mixture was stirred at 70° C. for 3 h. The mixture was cooled to room temperature and concentrated under reduced pressure. The residue was partitioned between water (50 mL) and EtOAc (200 mL). The organic layer was washed with brine (50 mL), dried over Na₂SO₄, filtered and concentrated under reduced pressure. The residue was purified by silica gel chromatograph (PE:EtOAc=5:1) to give the title compound (7.0 g, 91%). LC-MS (M+H)⁺=328.2.

### Step 2: (2-(3-bromophenyl)propan-2-yl)glycine hydrochloride

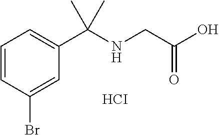

A mixture of tert-butyl (2-(3-bromophenyl)propan-2-yl)glycinate (7.0 g, 21 mmol) in TFA (10 mL) was stirred at 60° C. for 4 h and cooled to room temperature. The mixture was concentrated under vacuum. The residue was treated with HCl (4 M in dioxane, 10 mL) and the mixture was stirred for 10 min at room temperature. The mixture was concentrated under reduced pressure to give the title compound (6.6 g, 100%). LC-MS (M+H)⁺=272.2.

### Step 3: 7-bromo-1,1-dimethyl-2,3-dihydroisoquinolin-4(1H)-one

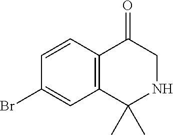

To a mixture of (2-(3-bromophenyl)propan-2-yl)glycine hydrochloride (29 g, 94 mmol) in DCM (600 mL) was added DMF (2 mL) and oxalyl chloride (11.2 mL, 128 mmol) dropwise at 0° C. The mixture was stirred at room temperature for 1 h and then concentrated under reduced pressure. The mixture was taken in DCM (600 mL) followed by addition of AlCl₃ (41.3 g, 311 mmol) at 0° C. in portions. The mixture was stirred at room temperature for 2 h and slowly added to saturated NaHCO₃ (1000 mL) with vigorous stirring. Solid was filtered off and the filter cake was rinsed with DCM (500 mL). The organic layer was washed with water, dried over Na₂SO₄, filtered and concentrated under reduced pressure to give the title compound (21 g, 88%). LC-MS (M+H)⁺=253.9.

### Step 4: 2-acetyl-7-bromo-1,1-dimethyl-2,3-dihydroisoquinolin-4(1H)-one

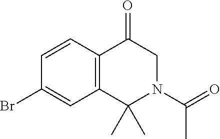 [SMILES: CC(=O)N1CC(=O)c2ccc(Br)cc2C1(C)C]

To a mixture of 7-bromo-1,1-dimethyl-2,3-dihydroisoquinolin-4(1H)-one (21 g, 82.7 mmol) in DCM (200 mL) was added Et₃N (25.1 g, 248 mmol) and AcCl (9.7 g, 124 mmol) at −30° C., and the mixture was stirred at −30° C. for 0.5 h. The mixture was washed with water (20 mL). The aqueous layer was back extracted with DCM (200 mL×3). The combined organic layer was dried over Na₂SO₄, filtered and concentrated under reduced pressure to give the title compound (23 g, 94%). LC-MS (M+H)⁺=295.9.

### Step 5: 1-(7-bromo-4-hydroxy-1,1-dimethyl-3,4-dihydroisoquinolin-2(1H)-yl)ethan-1-one

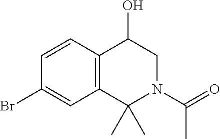 [SMILES: CC(=O)N1CC(O)c2ccc(Br)cc2C1(C)C]

To a mixture of 2-acetyl-7-bromo-1,1-dimethyl-2,3-dihydroisoquinolin-4(1H)-one (10 g, 33.7 mmol) in MeOH (100 mL), was added NaBH₄ (1.28 g, 33.7 mmol) in portions at 0° C., and the mixture was stirred at room temperature for 1 h. The mixture was concentrated to one third of the original volume, and then partitioned between EtOAc (200 mL) and water (100 mL). The organic layer was dried over Na₂SO₄, filtered and concentrated under reduced pressure. The residue was purified by silica gel chromatography (PE:EtOAc=3:1 to 0:1) to give the title compound (7.0 g, 70%). ¹H NMR (400 MHz, DMSO-d6) δ 7.55 (d, J=1.8 Hz, 1H), 7.41 (dd, J=8.4, 1.8 Hz, 1H), 7.34 (d, J=8.4 Hz, 1H), 5.62 (d, J=5.2 Hz, 1H), 4.60-4.53 (m, 1H), 3.61 (dd, J=13.2, 3.3 Hz, 1H), 3.24 (dd, J=13.2, 7.6 Hz, 1H), 2.11 (s, 3H), 1.74 (s, 3H), 1.69 (s, 3H). LC-MS (M+H)⁺=298.1.

### Step 6: 1-(7-bromo-4-((2-hydroxyethyl)amino)-1,1-dimethyl-3,4-dihydroisoquinolin-2(1H)-yl)ethan-1-one

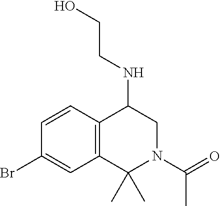 [SMILES: CC(=O)N1CC(NCCO)c2ccc(Br)cc2C1(C)C]

To a mixture of 1-(7-bromo-4-hydroxy-1,1-dimethyl-3,4-dihydroisoquinolin-2(1H)-yl)ethan-1-one (4.0 g, 13.4 mmol) in DCM (100 mL) was added Et₃N (3.9 mL, 26.8 mmol) and MsCl (1.55 mL, 20 mmol) at room temperature. The mixture was stirred at room temperature for 1 h and diluted with DCM (100 mL). The mixture was washed with water (50 mL), dried over Na₂SO₄, filtered and concentrated under reduced pressure. The residue was re-dissolved in DMF (32 mL) followed by addition of 2-aminoethan-1-ol (8 mL). The mixture was stirred at 80° C. for 3 h and cooled to room temperature. The mixture was partitioned between DCM (200 mL) and water (100 mL). The organic layer was dried over Na₂SO₄, filtered and concentrated under reduced pressure. The residue was purified by silica gel chromatograph (DCM:MeOH=20:1) to give the title compound (1.0 g, 22%). LC-MS (M+H)⁺=341.2.

### Step 7: 2-((7-bromo-1,1-dimethyl-1,2,3,4-tetrahydroisoquinolin-4-yl)amino)ethan-1-ol

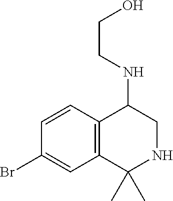 [SMILES: CC1(C)NCC(NCCO)c2ccc(Br)cc21]

To a mixture of 1-(7-bromo-4-((2-hydroxyethyl)amino)-1,1-dimethyl-3,4-dihydroisoquinolin-2(1H)-yl)ethan-1-one (1.2 g, 3.5 mmol) in water (3 mL) and dioxane (6 mL) was added concentrated hydrochloric acid (3 mL) and the mixture was stirred at 100° C. for 2 h. The mixture was cooled to room temperature and concentrated under reduced pressure. The residue was treated with DCM (10 mL) and methanolic ammonia (7 M, 4 mL). The mixture was concentrated under reduced pressure. The residue was purified by silica gel chromatograph (DCM:MeOH=20:1) to give the title compound (800 mg, 76%). LC-MS (M+H)⁺=299.1.

### Step 8: 8-bromo-6,6-dimethyl-1,3,4,6-tetrahydro-2H-1,5-methanobenzo[f][1,4]diazocine

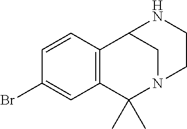 [SMILES: CC1(C)c2cc(Br)ccc2C2CN1CCN2]

To a mixture of 2-((7-bromo-1,1-dimethyl-1,2,3,4-tetrahydroisoquinolin-4-yl)amino)ethan-1-ol (10 mg, 0.033 mmol) in DCM (0.5 mL) was added Et₃N (0.014 ml, 0.099 mmol) and MsCl (5 mg, 0.043 mmol) at −40° C. and the mixture was stirred at −40° C. for 2 h. MeOH (0.5 mL) was added and the mixture was concentrated under reduced pressure. The residue was purified by prep-TLC (DCM:MeOH=20:1) to give the title compound (5 mg, 53%). LC-MS (M+H)=281.1.

### Step 9: (5-amino-1-((2-(trimethylsilyl)ethoxy)methyl)-6,8-dihydro-1H-furo[3,4-d]pyrrolo[3,2-b]pyridin-2-yl)(8-bromo-6,6-dimethyl-1,3,4,6-tetrahydro-2H-1,5-methanobenzo[f][1,4]diazocin-2-yl)methanone

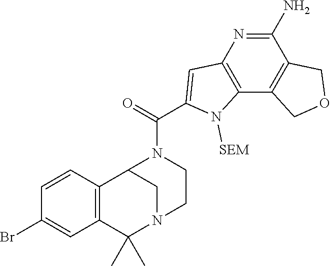 [SMILES: CC1(C)c2cc(Br)ccc2C2CN1CCN2C(=O)c1cc2nc(N)c3c(c2n1COCC[Si](C)(C)C)COC3]

To a mixture of 5-amino-1-((2-(trimethylsilyl)ethoxy)methyl)-6,8-dihydro-1H-furo[3,4-d]pyrrolo[3,2-b]pyridine-2-carboxylic acid (6 mg, 0.017 mmol) and 8-bromo-6,6-dimethyl-1,3,4,6-tetrahydro-2H-1,5-methanobenzo[f][1,4]diazocine (5 mg, 0.017 mmol) in THF (5 mL) was added BOPCl (6.4 mg, 0.025 mmol) and DIPEA (11 mg, 0.085 mmol). The mixture was stirred at 60° C. for 2 h and cooled to room temperature. The reaction mixture was partitioned between water (10 mL) and EtOAc (50 mL). The organic layer was washed with brine (10 mL), dried over Na₂SO₄, filtered and concentrated under reduced pressure. The residue was purified by prep-TLC (DCM:MeOH=10:1) to give the title compound (2 mg, 18%) LC-MS (M+H)⁺=612.2.

### Step 10: (5-amino-6,8-dihydro-1H-furo[3,4-d]pyrrolo[3,2-b]pyridin-2-yl)(8-bromo-6,6-dimethyl-1,3,4,6-tetrahydro-2H-1,5-methanobenzo[f][1,4]diazocin-2-yl)methanone

A mixture of (5-amino-1-((2-(trimethylsilyl)ethoxy)methyl)-6,8-dihydro-1H-furo[3,4-d]pyrrolo[3,2-b]pyridin-2-yl)(8-bromo-6,6-dimethyl-1,3,4,6-tetrahydro-2H-1,5-methanobenzo[f][1,4]diazocin-2-yl)methanone (2 mg, 0.003 mmol) in TFA (1 mL) was stirred at room temperature for 0.5 h and then concentrated under reduced pressure. The mixture was taken in THF (1 mL) and ammonium hydroxide (28%, 1 mL). The mixture was stirred at room temperature for 1 h and concentrated under reduced pressure. The residue was purified by prep-HPLC to give Example 32 (0.4 mg, 23%). ¹H NMR (400 MHz, DMSO-d6, 80° C.) δ11.27 (s, 1H), 7.53 (s, 1H), 7.29-7.28 (m, 1H), 6.5-6.49 (m, 1H), 5.38-5.30 (m, 3H), 5.11 (s, 2H), 4.90 (s, 2H), 1.56 (s, 3H), 1.31 (s, 3H). LC-MS (M+H)⁺=482.3.

### Example 33 & Example 34: (5-amino-6,8-dihydro-1H-furo[3,4-d]pyrrolo[3,2-b]pyridin-2-yl)((1S,5S,6S)-6-methyl-8-(trifluoromethyl)-1,3,4,6-tetrahydro-2H-1,5-methanobenzo[f][1,4]diazocin-2-yl)methanone & (5-amino-6,8-dihydro-1H-furo[3,4-d]pyrrolo[3,2-b]pyridin-2-yl)((1S,5S,6R)-6-methyl-8-(trifluoromethyl)-1,3,4,6-tetrahydro-2H-1,5-methanobenzo[f][1,4]diazocin-2-yl)methanone

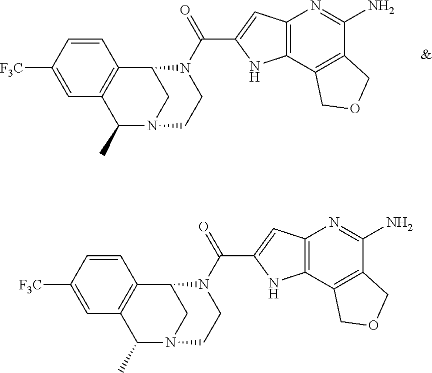 [SMILES: C[C@H]1c2cc(C(F)(F)F)ccc2[C@H]2C[N@]1CCN2C(=O)c1cc2nc(N)c3c(c2[nH]1)COC3 ; C[C@@H]1c2cc(C(F)(F)F)ccc2[C@H]2C[N@]1CCN2C(=O)c1cc2nc(N)c3c(c2[nH]1)COC3]

### Step 1: methyl (1-(3-bromophenyl)ethyl)glycinate

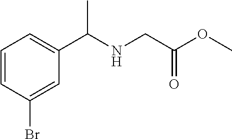

To a mixture of 1-(3-bromophenyl)ethan-1-one (15 g, 75.7 mmol) and methyl glycinate hydrochloride (28.3 g, 226 mmol) in MeOH (200 mL) was added Et₃N (33 mL, 227 mmol) and NaBH₃CN (7.1 g, 114 mmol). The mixture was stirred at 70° C. overnight and cooled to room temperature. The mixture was concentrated under reduced pressure. The residue was partitioned between water (100 mL) and EtOAc (500 mL). The organic layer was washed with brine (100 mL), dried over Na₂SO₄, filtered and concentrated under reduced pressure. The residue was purified by silica gel chromatograph (PE:EtOAc=3:1) to give the title compound (20 g, 97%). ¹H NMR (400 MHz, DMSO-d6) δ 7.51 (s, 1H), 7.41 (dd, J=9.2, 1.6 Hz, 1H), 7.33-7.24 (m, 2H), 3.74 (q, J=6.5 Hz, 1H), 3.58 (s, 3H), 3.19, 3.07 (ABq, J=17.2 Hz, 2H), 1.23 (d, J=6.4 Hz, 3H). LC-MS (M+H)⁺=272.1.

### Step 2: methyl N-((benzyloxy)carbonyl)-N-(1-(3-bromophenyl)ethyl)glycinate

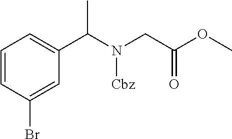 [SMILES: COC(=O)CN(C(=O)OCc1ccccc1)C(C)c1cccc(Br)c1]

To a mixture of methyl (1-(3-bromophenyl)ethyl)glycinate (21 g, 77 mmol) and K₂CO₃ (21 g, 154 mmol) in DMF (100 mL) was added CbzCl (22 mL, 154 mmol) dropwise at room temperature. The mixture was stirred at 80° C. for 4 h, cooled to room temperature and concentrated under reduced pressure. The residue was partitioned between EtOAc (200 mL) and water (100 mL). The organic layer was dried over Na₂SO₄, filtered and concentrated under reduced pressure. The residue was purified by silica gel chromatograph (PE/EtOAc=5/1) to give the title compound (19 g, 61%). LC-MS (M+H)⁺=406.1.

### Step 3: N-((benzyloxy)carbonyl)-N-(1-(3-bromophenyl)ethyl)glycine

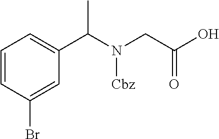 [SMILES: CC(c1cccc(Br)c1)N(CC(=O)O)C(=O)OCc1ccccc1]

To a mixture of methyl N-((benzyloxy)carbonyl)-N-(1-(3-bromophenyl)ethyl)glycinate (19.0 g, 46.9 mmol) in MeOH (60 mL) was added a solution of NaOH (9.3 g, 235 mmol) in water (60 mL). The mixture was stirred at room temperature for 2 h and MeOH was evaporated under reduced pressure. The remaining aqueous solution was partitioned between EtOAc (200 mL) and hydrochloric acid (4 N, 50 mL). The organic layer was with water (100 mL), dried over Na₂SO₄, filtered and concentrated under reduced pressure to give the title compound (18.3 g, 100%). LC-MS (M+H)=392.2.

### Step 4: tert-butyl 7-bromo-1-methyl-4-oxo-3,4-dihydroisoquinoline-2(1H)-carboxylate

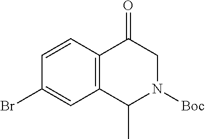 [SMILES: CC1c2cc(Br)ccc2C(=O)CN1C(=O)OC(C)(C)C]

To a solution of N-((benzyloxy)carbonyl)-N-(1-(3-bromophenyl)ethyl)glycine (5.0 g, 12.8 mmol) in DCM (100 mL) was added SOCl₂ (1.4 mL, 19.1 mmol) dropwise at room temperature. The mixture was stirred at After stirring at 45° C. for 3 h and cooled to room temperature. The mixture was concentrated in reduced pressure and re-dissolved in anhydrous DCM (100 mL). AlCl₃ (6.7 g, 51 mmol) was added in portions and the mixture was stirred at room temperature for 12 h. The mixture was carefully added to saturated NaHCO₃ (300 mL) with vigorous stirring. The solid was filtered off and the filter cake was rinsed with DCM (100 mL). The filtrate was washed with water (50 mL), dried over Na₂SO₄, filtered and concentrated under reduced pressure. The residue was immediately taken in DCM (100 mL) followed by addition of Et₃N (7.8 mL, 55 mmol) and Boc₂O (6.0 g, 27.5 mmol). The mixture was stirred at room temperature for 2 h and diluted with DCM (100 mL). The mixture was washed with water (50 mL), dried over Na₂SO₄, filtered and concentrated under reduced pressure. The residue was purified by silica gel chromatography (PE:EtOAc=10:1) to give the title compound (2.2 g, 51%).

### Step 5: tert-butyl (4R)-7-bromo-4-hydroxy-1-methyl-3,4-dihydroisoquinoline-2(1H)-carboxylate

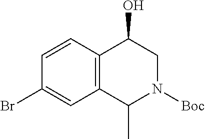 [SMILES: CC1c2cc(Br)ccc2[C@@H](O)CN1C(=O)OC(C)(C)C]

To a mixture of tert-butyl 7-bromo-1-methyl-4-oxo-3,4-dihydroisoquinoline-2(1H)-carboxylate (1.5 g, 4.4 mmol) in formic acid-Et₃N complex (5:2, 3 mL) was added [(S,S)-N-(2-amino-1,2-diphenylethyl)-p-toluenesulfonamide]chloro(p-cymene)ruthenium(II) (140 mg, 0.22 mmol) at room temperature under nitrogen. The mixture was stirred at room temperature for 12 h and then partitioned between EtOAc (100 mL) and water (20 mL). The organic layer was dried over Na₂SO₄, filtered and concentrated under reduced pressure. The residue was purified by silica gel chromatography (PE:EtOAc=4:1) to give the title compound (700 mg, 47%). LC-MS (M+H)⁺=342.2.

### Step 6: tert-butyl (4S)-7-bromo-4-((2-hydroxyethyl)amino)-1-methyl-3,4-dihydroisoquinoline-2(1H)-carboxylate

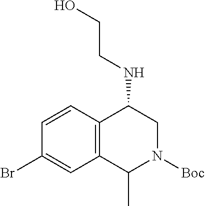 [SMILES: CC1c2cc(Br)ccc2[C@H](NCCO)CN1C(=O)OC(C)(C)C]

To a solution of tert-butyl (4R)-7-bromo-4-hydroxy-1-methyl-3,4-dihydroisoquinoline-2(1H)-carboxylate (600 mg, 1.8 mmol) in DCM (100 mL) was added Et₃N (0.70 mL, 5.25 mmol) and MsCl (0.176 mL, 2.3 mmol) at room temperature. The mixture was stirred at room temperature for 1 h and then diluted with DCM (100 mL). The mixture was washed with water (50 mL), dried over Na₂SO₄, filtered and concentrated under reduced pressure. The residue was dissolved in DMF (4 mL) followed by addition of 2-aminoethan-1-ol (2 mL). The mixture was stirred at 80° C. for 3 h and cooled to room temperature. The mixture was partitioned between EtOAc (200 mL) and water (100 mL). The organic layer was dried over Na₂SO₄, filtered and concentrated under reduced pressure. The residue was purified by silica gel chromatography (DCM:MeOH=40:1) to give the title compound (200 mg, 30%) LC-MS (M+H)⁺=385.1.

### Step 7: tert-butyl (1S,5S)-8-bromo-6-methyl-1,3,4,6-tetrahydro-2H-1,5-methanobenzo[f][1,4]diazocine-2-carboxylate

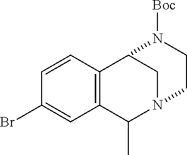 [SMILES: CC1c2cc(Br)ccc2[C@H]2C[N@]1CCN2C(=O)OC(C)(C)C]

To a mixture of tert-butyl (4S)-7-bromo-4-((2-hydroxyethyl)amino)-1-methyl-3,4-dihydroisoquinoline-2(1H)-carboxylate (200 mg, 0.52 mmol) in THF (20 mL) was added SOC12 (0.30 mL, 2.6 mmol) and the mixture was stirred at 60° C. for 2 h. The mixture was cooled to room temperature and concentrated under reduced pressure. The residue was taken in HCl (4 M in dioxane, 10 mL) and the mixture was stirred at room temperature for 2 h. The mixture was concentrated under reduced pressure and re-dissolved in DMF (10 mL). K₂CO₃ (4.0 g, 29.5 mmol) was added, and the mixture was stirred at 60° C. for 2 h. The mixture was cooled to room temperature and Boc₂O (170 mg, 0.78 mmol) was added. The mixture was stirred at room temperature for 1 h. The mixture was partitioned between DCM (100 mL) and water (50 mL). The organic layer was dried over Na₂SO₄, filtered and concentrated under reduced pressure. The residue was purified by silica gel chromatography (DCM:MeOH=20:1) to give the title compound (120 mg, 62%). LC-MS (M+H)⁺=367.2.

### Step 8: tert-butyl (1S,5S)-8-iodo-6-methyl-1,3,4,6-tetrahydro-2H-1,5-methanobenzo[f][1,4]diazocine-2-carboxylate

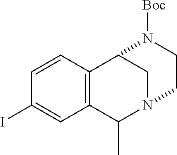 [SMILES: CC1c2cc(I)ccc2[C@H]2C[N@]1CCN2C(=O)OC(C)(C)C]

To a mixture of tert-butyl (1S,5S)-8-bromo-6-methyl-1,3,4,6-tetrahydro-2H-1,5-methanobenzo[f][1,4]diazocine-2-carboxylate (200 mg, 0.55 mmol), CuI (52 mg, 0.27 mmol) and NaI (330 mg, 2.2 mmol) in dioxane (10 mL) was added trans-N,N′-dimethyl-cyclohexane-1,2-diamine (78 mg, 0.55 mmol) under nitrogen. The mixture was stirred at 110° C. for 64 h and cooled to room temperature. The mixture was partitioned between EtOAc (100 mL) and water (20 mL). The organic layer was dried over Na₂SO₄, filtered and concentrated under reduced pressure. The residue was purified by silica gel chromatography (DCM:MeOH=20:1) to give the title compound (180 mg, 80%). LC-MS (M+H)⁺=415.2.

### Step 9: tert-butyl (1S,5S)-6-methyl-8-(trifluoromethyl)-1,3,4,6-tetrahydro-2H-1,5-methanobenzo[f][1,4]diazocine-2-carboxylate

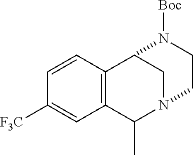 [SMILES: CC1c2cc(C(F)(F)F)ccc2[C@H]2C[N@]1CCN2C(=O)OC(C)(C)C]

To a mixture of tert-butyl (1S,5S)-8-iodo-6-methyl-1,3,4,6-tetrahydro-2H-1,5-methanobenzo[f][1,4]diazocine-2-carboxylate (180 mg, 0.435 mmol) in DMF (5 mL) was added (1,10-phenanthroline)(trifluoromethyl)copper(I) (408 mg, 1.3 mmol) under nitrogen. The mixture was stirred at 60° C. for 12 h and cooled to room temperature. The mixture was partitioned between EtOAc (50 mL) and water (20 mL). The organic layer was dried over Na₂SO₄, filtered and concentrated under reduced pressure. The residue was purified by prep-TLC (DCM:MeOH=20:1) to give the title compound (90 mg, 58%). LC-MS (M+H)⁺=357.2.

### Step 10: (1S,5S)-6-methyl-8-(trifluoromethyl)-1,3,4,6-tetrahydro-2H-1,5-methanobenzo[f][1,4]diazocine bis(trifluoroacetic acid)

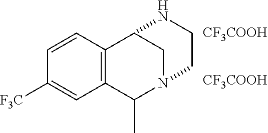 [SMILES: CC1c2cc(C(F)(F)F)ccc2[C@H]2C[N@]1CCN2 ; "CF3COOH" ; "CF3COOH"]

To a flask charged with tert-butyl (1S,5S)-6-methyl-8-(trifluoromethyl)-1,3,4,6-tetrahydro-2H-1,5-methanobenzo[f][1,4]diazocine-2-carboxylate (90 mg, 0.25 mmol) was added in TFA (2 mL). The mixture was stirred at room temperature for 2 h and concentrated under reduced pressure to give the title compound (122 mg, 100%).

### Step 11: (5-amino-1-((2-(trimethylsilyl)ethoxy)methyl)-6,8-dihydro-1H-furo[3,4-d]pyrrolo[3,2-b]pyridin-2-yl)((1S,5S′)-6-methyl-8-(trifluoromethyl)-1,3,4,6-tetrahydro-2H-1,5-methanobenzo[f][1,4]diazocin-2-yl)methanone

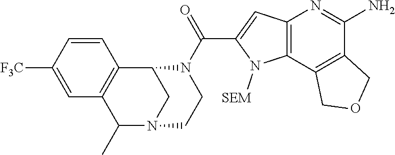 [SMILES: CC1c2cc(C(F)(F)F)ccc2[C@H]2C[N@]1CCN2C(=O)c1cc2nc(N)c3c(c2n1COCC[Si](C)(C)C)COC3]

To a mixture of 5-amino-1-((2-(trimethylsilyl)ethoxy)methyl)-6,8-dihydro-1H-furo[3,4-d]pyrrolo[3,2-b]pyridine-2-carboxylic acid (43 mg, 0.125 mmol) and (1S,5S)-6-methyl-8-(trifluoromethyl)-1,3,4,6-tetrahydro-2H-1,5-methanobenzo[f][1,4]diazocine bis(trifluoroacetic acid) (60 mg, 0.125 mmol) in THF (5 mL) was added BOPCl (48 mg, 0.187 mmol) and DIPEA (0.115 mL, 0.625 mmol). The mixture was stirred at 60° C. for 2 h and then cooled to room temperature. The mixture was partitioned between water (10 mL) and EtOAc (50 mL). The organic layer was washed with brine (10 mL), dried over Na₂SO₄, filtered and concentrated under reduced pressure. The residue was purified by prep-TLC (DCM:MeOH=10:1) to give the title compound (30 mg, 41%). LC-MS (M+H)⁺=588.3.

### Step 12: (5-amino-6,8-dihydro-1H-furo[3,4-d]pyrrolo[3,2-b]pyridin-2-yl)((1S,5S,6S)-6-methyl-8-(trifluoromethyl)-1,3,4,6-tetrahydro-2H-1,5-methanobenzo[f][1,4]diazocin-2-yl)methanone & (5-amino-6,8-dihydro-1H-furo[3,4-d]pyrrolo[3,2-b]pyridin-2-yl)((1S,5S,6R)-6-methyl-8-(trifluoromethyl)-1,3,4,6-tetrahydro-2H-1,5-methanobenzo[f][1,4]diazocin-2-yl)methanone

A mixture of (5-amino-1-((2-(trimethylsilyl)ethoxy)methyl)-6,8-dihydro-1H-furo[3,4-d]pyrrolo[3,2-b]pyridin-2-yl)((1S,5S)-6-methyl-8-(trifluoromethyl)-1,3,4,6-tetrahydro-2H-1,5-methanobenzo[f][1,4]diazocin-2-yl)methanone (30 mg, 0.051 mmol) in TFA (2 mL) was stirred at room temperature for 0.5 h and concentrated under reduced pressure. The mixture was taken in THF (4 mL) and ammonium hydroxide (28%, 1 mL). The mixture was stirred at room temperature for 1 h and concentrated under reduced pressure. The residue was purified by prep-HPLC followed by chiral SFC to give Example 33 (10 mg, 43%) and Example 34 (7 mg, 30%).

Example 33: ¹H NMR (400 MHz, DMSO-d6, 80° C.) δ11.27 (s, 1H), 7.55 (s, 1H), 7.48 (d, J=7.2 Hz, 1H), 7.33-7.16 (m, 1H), 6.51 (s, 1H), 5.52-5.38 (m, 1H), 5.27 (s, 2H), 5.11 (s, 2H), 4.90 (s, 2H), 4.15 (q, J=6.9 Hz, 1H), 3.79 (dd, J=12.9, 3.8 Hz, 1H), 3.31 (d, J=13.9 Hz, 1H), 3.26-3.13 (m, 2H), 3.02-2.94 (m, 1H), 2.92-2.77 (m, 1H), 1.37 (d, J=6.9 Hz, 3H). LC-MS (M+H)⁺=458.4.

Example 34: ¹H NMR (400 MHz, DMSO-d6, 80° C.) δ11.30 (s, 1H), 7.59 (s, 1H), 7.50 (d, J=8.1 Hz, 1H), 7.26 (s, 1H), 6.52 (s, 1H), 5.56-5.47 (m, 1H), 5.31 (s, 2H), 5.12 (s, 2H), 4.91 (s, 2H), 4.34-4.24 (m, 1H), 3.80 (dd, J=13.5, 2.7 Hz, 1H), 3.44 (d, J=13.5 Hz, 1H), 3.29-3.19 (m, 2H), 3.02-2.92 (m, 1H), 2.80-2.65 (m, 1H), 1.58 (d, J=7.1 Hz, 3H). LC-MS (M+H)⁺=458.4.

### Example 35 & Example 36: ((R)-5-amino-6-methyl-6,8-dihydro-1H-furo[3,4-d]pyrrolo[3,2-b]pyridin-2-yl)((1S,5S,6S)-6-methyl-8-(trifluoromethyl)-1,3,4,6-tetrahydro-2H-1,5-methanobenzo[f][1,4]diazocin-2-yl)methanone & ((R)-5-amino-6-methyl-6,8-dihydro-1H-furo[3,4-d]pyrrolo[3,2-b]pyridin-2-yl)((1S,5S,6R)-6-methyl-8-(trifluoromethyl)-1,3,4,6-tetrahydro-2H-1,5-methanobenzo[f][1,4]diazocin-2-yl)methanone

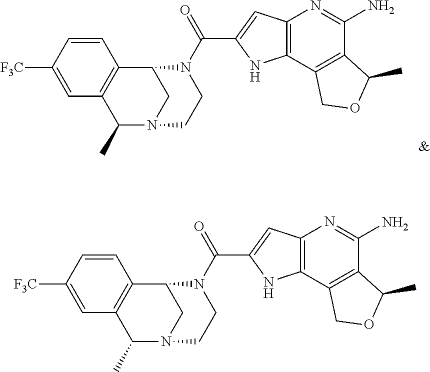 [SMILES: C[C@H]1OCc2c1c(N)nc1cc(C(=O)N3CC[N@@]4C[C@@H]3c3ccc(C(F)(F)F)cc3[C@@H]4C)[nH]c21 ; C[C@@H]1c2cc(C(F)(F)F)ccc2[C@H]2C[N@]1CCN2C(=O)c1cc2nc(N)c3c(c2[nH]1)CO[C@@H]3C]

To a mixture of (R)-5-amino-6-methyl-6,8-dihydro-1H-furo[3,4-d]pyrrolo[3,2-b]pyridine-2-carboxylic acid (30 mg, 0.125 mmol), (1S,5S)-6-methyl-8-(trifluoromethyl)-1,3,4,6-tetrahydro-2H-1,5-methanobenzo[f][1,4]diazocine bis(trifluoroacetic acid) (60 mg, 0.125 mmol) and DIPEA (0.23 ml, 1.23 mmol) in anhydrous DMF (10 mL) was added T3P (50% in DMF, 400 mg, 0.625 mmol) under nitrogen. The mixture was stirred at 60° C. for 2 h and cooled to room temperature. Hydrochloric acid (6 M, 9 mL) was added, and the mixture was stirred at 60° C. for 3 h. The mixture was cooled to room temperature and partitioned between saturated NaHCO₃ (100 mL) and EtOAc (100 mL). The organic layer was washed with brine (100 mL), dried over Na₂SO₄, filtered and concentrated under reduced pressure. The residue was purified by prep-HPLC followed by chiral SFC to give Example 35 (13 mg, 22%) and Example 36 (5 mg, 9%).

Example 35: ¹H NMR (400 MHz, DMSO-d6, 80° C.) δ11.27 (s, 1H), 7.55 (s, 1H), 7.48 (d, J=7.7 Hz, 1H), 7.37-7.14 (m, 1H), 6.51 (s, 1H), 5.54-5.40 (m, 1H), 5.37-5.26 (m, 1H), 5.24-5.09 (m, 3H), 5.02 (d, J=13.7 Hz, 1H), 4.14 (q, J=6.8 Hz, 1H), 3.79 (dd, J=13.8, 4.2 Hz, 1H), 3.30 (d, J=14.1 Hz, 1H), 3.26-3.14 (m, 2H), 3.02-2.78 (m, 2H), 1.40-1.32 (m, 6H). LC-MS (M+H)⁺=472.4.

Example 36: ¹H NMR (400 MHz, DMSO-d6, 80° C.) δ11.27 (s, 1H), 7.58 (s, 1H), 7.50 (d, J=7.7 Hz, 1H), 7.31-7.18 (m, 1H), 6.51 (s, 1H), 5.57-5.45 (m, 1H), 5.37-5.25 (m, 1H), 5.21-5.07 (m, 3H), 5.07-4.95 (m, 1H), 4.36-4.23 (m, 1H), 3.80 (d, J=10.2 Hz, 1H), 3.44 (d, J=13.2 Hz, 1H), 3.28-3.15 (m, 2H), 2.98-2.66 (m, 2H), 1.57 (d, J=7.1 Hz, 3H), 1.35 (d, J=6.1 Hz, 3H). LC-MS (M+H)⁺=472.4.

### Example 37 & Example 38: (5-amino-6,8-dihydro-1H-furo[3,4-d]pyrrolo[3,2-b]pyridin-2-yl)((2S,6R)-9-(trifluoromethyl)-3,4-dihydro-2H-2,6-methanobenzo[b][1,5]oxazocin-5(6H)-yl)methanone & (5-amino-6,8-dihydro-1H-furo[3,4-d]pyrrolo[3,2-b]pyridin-2-yl)((2R,6S)-9-(trifluoromethyl)-3,4-dihydro-2H-2,6-methanobenzo[b][1,5]oxazocin-5(6H)-yl)methanone

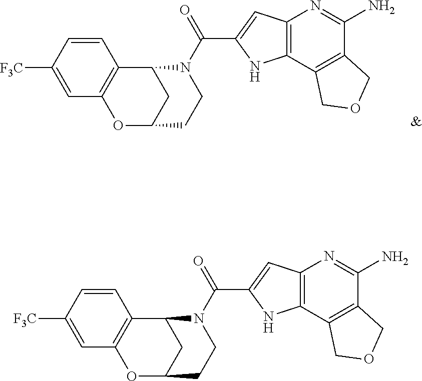 [SMILES: Nc1nc2cc(C(=O)N3CC[C@H]4C[C@@H]3c3ccc(C(F)(F)F)cc3O4)[nH]c2c2c1COC2 ; Nc1nc2cc(C(=O)N3CC[C@@H]4C[C@H]3c3ccc(C(F)(F)F)cc3O4)[nH]c2c2c1COC2]

### Step 1: 7-(trifluoromethyl)-4H-chromen-4-one

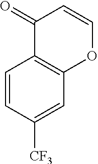 [SMILES: O=c1ccoc2cc(C(F)(F)F)ccc12]

To a mixture of 1-(2-hydroxy-4-(trifluoromethyl)phenyl)ethan-1-one (20 g, 98 mmol) in ethyl formate (200 mL) and THF (50 mL) was added NaH (60%, 11.8 g, 294 mmol) at 0° C. The mixture was stirred at 0° C. for 1 h and at room temperature for 2 h. MeOH (20 mL) followed by concentrated hydrochloric acid (150 mL) was slowly added to the mixture on a water bath at room temperature, and the mixture was stirred for 2 h at room temperature. The mixture was diluted with water (500 mL) and then extracted with EtOAc (500 mL). The organic layer was washed with brine (500 mL×2), dried over Na₂SO₄, filtered and concentrated under reduced pressure. The residue was purified by silica gel chromatograph (PE:EtOAc=3:1) to give the title compound (19 g, 90%). LC-MS (M+H)⁺=215.3.

### Step 2: 2-allyl-7-(trifluoromethyl)chroman-4-one

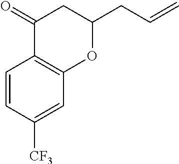 [SMILES: C=CCC1CC(=O)c2ccc(C(F)(F)F)cc2O1]

A mixture of CuBr·Me₂S (455 mg, 2.22 mmol) and (R)-1-[(Sₚ)-2-(dicyclohexylphosphino)ferrocenylethyl]diphenylphosphine (1.58 g, 2.66 mmol) in DCM (200 mL) was stirred at 25° C. for 10 min. The mixture was cooled to −78° C. followed by dropwise addition of allylmagnesium bromide (1.0 M in diethyl ether, 111 mL, 111 mmol). After 10 min, 7-(trifluoromethyl)-4H-chromen-4-one (19 g, 88.8 mmol) in DCM (50 mL) was added dropwise. The mixture was stirred at −78° C. for 2 h. The mixture was poured into saturated NH₄Cl (400 mL) and extracted with DCM (400 mL). The combine organic layer was washed with brine (400 mL), dried over Na₂SO₄, filtered and concentrated under reduced pressure. The residue was purified by silica gel chromatograph (PE:EtOAc=10:1) to give the title compound (13 g, 57%). LC-MS (M+H)⁺=257.3.

### Step 3: 2-(4-oxo-7-(trifluoromethyl)chroman-2-yl)acetaldehyde

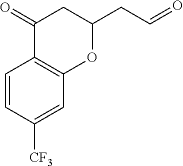 [SMILES: O=CCC1CC(=O)c2ccc(C(F)(F)F)cc2O1]

To a mixture of 2-allyl-7-(trifluoromethyl)chroman-4-one (13 g, 50.8 mmol) in dioxane (300 mL) and water (60 mL), was added potassium osmate (VI) dihydrate (1.87 g, 5.1 mmol), NaIO₄ (43.5 g, 203 mmol) and 2,6-lutidine (10.9 g, 101 mmol). The mixture was stirred at 25° C. for 3 h. Solid was filtered off and the filtrate was diluted with water (300 mL). The mixture was extracted with EtOAc (300 mL×2). The combined organic layer was successively washed with saturated Na₂S₂O₃ (300 mL), 1 N hydrochloric acid (300 mL), saturated NaHCO₃ (300 mL) and brine (300 mL×2), dried over Na₂SO₄, filtered and concentrated under reduced pressure to give the title compound (13 g, 99%). LC-MS (M+H)⁺=259.4.

### Step 4: 2-(2-hydroxyethyl)-7-(trifluoromethyl)chroman-4-ol

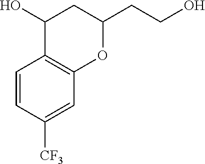 [SMILES: OCCC1CC(O)c2ccc(C(F)(F)F)cc2O1]

To a mixture of 2-(4-oxo-7-(trifluoromethyl)chroman-2-yl)acetaldehyde (13 g, 50.4 mmol) in MeOH (150 mL) was added NaBH₄ (3.83 g, 100.1 mmol) at 0° C. The mixture was stirred at room temperature for 2 h. The mixture was poured into water (400 mL) and extracted with EtOAc (400 mL×2). The combined organic layer was washed with brine (400 mL×2), dried over Na₂SO₄, filtered and concentrated under reduced pressure. The residue was purified by silica gel chromatography (PE:EtOAc=1:1) to give the title compound for (13 g, 98%). LC-MS (M+H)=263.1.

### Step 5: 2-(2-((tert-butyldimethylsilyl)oxy)ethyl)-7-(trifluoromethyl)chroman-4-ol

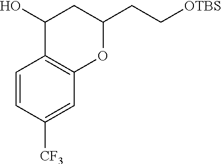 [SMILES: CC(C)(C)[Si](C)(C)OCCC1CC(O)c2ccc(C(F)(F)F)cc2O1]

To a mixture of 2-(2-hydroxyethyl)-7-(trifluoromethyl)chroman-4-ol (13 g, 49.5 mmol) in DCM (150 mL) was added imidazole (7.0 g, 103 mmol) and TBSCl (11.6 g, 77.3 mmol) at 0° C. The mixture was stirred at room temperature for 1 h. The mixture was washed water (400 mL), and the aqueous was extracted with DCM (400 mL). The combined organic layer was washed with brine (400 mL×2), dried over Na₂SO₄, filtered and concentrated under reduced pressure. The residue was purified by silica gel chromatograph (PE:EtOAc=3:1) to give the title compound (10.6 g, 57%). LC-MS (M+H)⁺=377.4.

### Step 6: tert-butyl (2-(2-((tert-butyldimethylsilyl)oxy)ethyl)-7-(trifluoromethyl)chroman-4-yl)((2-nitrophenyl)sulfonyl)carbamate

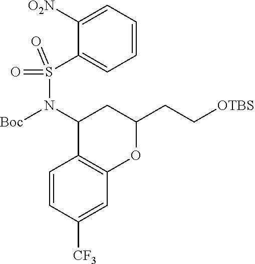 [SMILES: CC(C)(C)OC(=O)N(C1CC(CCO[Si](C)(C)C(C)(C)C)Oc2cc(C(F)(F)F)ccc21)S(=O)(=O)c1ccccc1[N+](=O)[O-]]

To a solution of 2-(2-((tert-butyldimethylsilyl)oxy)ethyl)-7-(trifluoromethyl)chroman-4-ol (10.6 g, 28.2 mmol), 2-NsNHBoc (10.2 g, 33.8 mmol) and PPh₃ (11.1 g, 42.3 mmol) in THF (30 mL) was added DtBAD (9.73 g, 42.3 mmol) in portions at 0° C. under nitrogen. The mixture was stirred at 65° C. for 16 h and cooled to room temperature. The mixture was concentrated under reduced pressure. The residue was purified by purified by silica gel chromatograph (PE:EtOAc=3:1) to give the title compound (12 g, 64%). LC-MS (M+H)⁺=661.3.

### Step 7: N-(2-(2-hydroxyethyl)-7-(trifluoromethyl)chroman-4-yl)-2-nitrobenzenesulfonamide

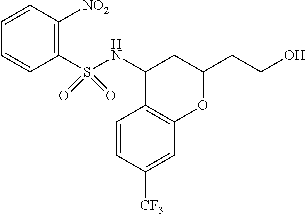 [SMILES: O=[N+]([O-])c1ccccc1S(=O)(=O)NC1CC(CCO)Oc2cc(C(F)(F)F)ccc21]

A mixture of tert-butyl (2-(2-((tert-butyldimethylsilyl)oxy)ethyl)-7-(trifluoromethyl)chroman-4-yl)((2-nitrophenyl)sulfonyl)carbamate (12 g, 18.2 mmol) in HCl (4.0 M in dioxane, 100 mL) was stirred at 45° C. for 16 h and then cooled to room temperature. The mixture was concentrated under reduced pressure. The residue was partitioned between saturated NaHCO₃ (100 mL) and EtOAc (200 mL). The organic layer was washed with brine (100 mL), dried over Na₂SO₄, filtered and concentrated under reduced pressure. The residue was purified by silica gel chromatograph (PE:EtOAc=1:1) to give the title compound (500 mg, 6%). LC-MS (M+H)⁺=447.2.

### Step 8: 5-((2-nitrophenyl)sulfonyl)-9-(trifluoromethyl)-3,4,5,6-tetrahydro-2H-2,6-methanobenzo[b][1,5]oxazocine

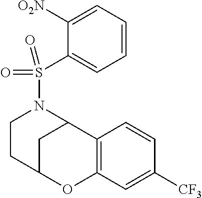 [SMILES: O=[N+]([O-])c1ccccc1S(=O)(=O)N1CCC2CC1c1ccc(C(F)(F)F)cc1O2]

To a mixture of N-(2-(2-hydroxyethyl)-7-(trifluoromethyl)chroman-4-yl)-2-nitrobenzenesulfonamide (500 mg, 1.12 mmol) and PPh₃ (881 mg, 3.36 mmol) in THF (200 mL), was added DtBAD (773 mg, 3.36 mmol) in portions at 0° C. under nitrogen. The mixture was stirred at room temperature for 16 h and then concentrated under reduced pressure. The residue was purified by silica gel chromatograph (PE:EtOAc=3:1) to give the title compound (424 mg, 88%). LC-MS (M+H)⁺=429.1.

### Step 9: 9-(trifluoromethyl)-3,4,5,6-tetrahydro-2H-2,6-methanobenzo[b][1,5]oxazocine

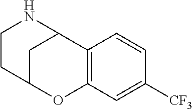 [SMILES: FC(F)(F)c1ccc2c(c1)OC1CCNC2C1]

A mixture of 5-((2-nitrophenyl)sulfonyl)-9-(trifluoromethyl)-3,4,5,6-tetrahydro-2H-2,6-methanobenzo[b][1,5]oxazocine (424 mg, 0.99 mmol), 1-dodecanethiol (807 mg, 3.99 mmol), LiOH H₂O (168 mg, 3.99 mmol) in DMF (1 mL) and THF (10 mL) was stirred at room temperature for 16 h. The mixture was partitioned between water (50 mL) and EtOAc (50 mL). The organic layer was washed with brine (50 mL×2), dried over Na₂SO₄, filtered and concentrated under reduced pressure. The residue was purified by silica gel chromatograph (DCM:MeOH=10:1) to give the title compound (150 mg, 62%). LC-MS (M+H)⁺=244.2.

### Step 10: (5-amino-1-((2-(trimethylsilyl)ethoxy)methyl)-6,8-dihydro-1H-furo[3,4-d]pyrrolo[3,2-b]pyridin-2-yl)(9-(trifluoromethyl)-3,4-dihydro-2H-2,6-methanobenzo[b][1,5]oxazocin-5(6H)-yl)methanone

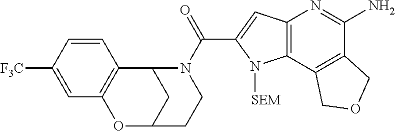 [SMILES: C[Si](C)(C)CCOCn1c(C(=O)N2CCC3CC2c2ccc(C(F)(F)F)cc2O3)cc2nc(N)c3c(c21)COC3]

To a mixture of 9-(trifluoromethyl)-3,4,5,6-tetrahydro-2H-2,6-methanobenzo[b][1,5]oxazocine (60 mg, 0.24 mmol), 5-amino-1-((2-(trimethylsilyl)ethoxy)methyl)-6,8-dihydro-H-furo[3,4-d]pyrrolo[3,2-b]pyridine-2-carboxylic acid (103 mg, 0.29 mmol) in THF (15 mL) was added BOPCl (87 mg, 0.34 mmol) and DIPEA (95 mg, 0.73 mmol). The mixture was stirred at 60° C. for 16 h and concentrated under reduced pressure. The residue was partitioned between water (50 mL) and EtOAc (50 mL). The organic layer was dried over Na₂SO₄, filtered and concentrated under reduced pressure. The residue was purified by silica gel chromatograph (DCM:MeOH=25:1) to give the title compound (110 mg, 77%). LC-MS (M+H)⁺=575.2.

### Step 11: (5-amino-6,8-dihydro-1H-furo[3,4-d]pyrrolo[3,2-b]pyridin-2-yl)((2S,6R)-9-(trifluoromethyl)-3,4-dihydro-2H-2,6-methanobenzo[b][1,5]oxazocin-5(6H)-yl)methanone & (5-amino-6,8-dihydro-1H-furo[3,4-d]pyrrolo[3,2-b]pyridin-2-yl)((2R,6S)-9-(trifluoromethyl)-3,4-dihydro-2H-2,6-methanobenzo[b][1,5]oxazocin-5(6H)-yl)methanone

A mixture of (5-amino-1-((2-(trimethylsilyl)ethoxy)methyl)-6,8-dihydro-1H-furo[3,4-d]pyrrolo[3,2-b]pyridin-2-yl)(9-(trifluoromethyl)-3,4-dihydro-2H-2,6-methanobenzo[b][1,5]oxazocin-5(6H)-yl)methanone (110 mg, 0.19 mmol) in DCM (6 mL) and TFA (6 mL) was stirred at room temperature for 3 h and then concentrated under reduced pressure. The residue was re-dissolved in methanolic ammonia (7 M, 12 mL). The mixture was stirred at room temperature for 2 h and then concentrated under reduced pressure. The residue was partitioned between saturated NaHCO₃ (50 mL) and EtOAc (50 mL). The organic layer was dried over Na₂SO₄, filtered and concentrated under reduced pressure. The residue was purified by prep-TLC followed by chiral SFC to give Example 37 (27 mg, 32%) and Example 38 (33 mg, 39%).

Analytical chiral-SFC condition as below. Column: CHIRALPAK AD-H; Column size: 4.6×100 mm, 5 μm; Mobile phase: 4 mM methanolic NH₃:CO₂, 45:55 isocratic; Flow: 2.0 mL/min; Temperature: 35° C.; back pressure: 1500 psi.

Example 37: Analytical SFC tR=3.17 min. ¹H NMR (400 MHz, DMSO-d6, 80° C.) δ11.31 (s, 1H), 7.34-7.21 (m, 1H), 7.20-7.08 (m, 2H), 6.58 (s, 1H), 5.78 (s, 1H), 5.29 (s, 2H), 5.12 (s, 2H), 5.01-4.79 (m, 3H), 4.24-4.11 (m, 1H), 2.92-2.80 (m, 1H), 2.28-2.22 (m, 1H), 2.12-1.93 (m, 3H). LC-MS (M+H)⁺=445.3.

Example 38: Analytical SFC tR=5.18 min. ¹H NMR (400 MHz, DMSO-d6, 80° C.) δ11.30 (s, 1H), 7.38-7.21 (m, 1H), 7.21-7.06 (m, 2H), 6.58 (s, 1H), 5.78 (s, 1H), 5.28 (s, 2H), 5.12 (s, 2H), 4.91 (s, 2H), 4.88-4.77 (m, 1H), 4.25-4.13 (m, 1H), 2.93-2.75 (m, 1H), 2.30-2.19 (m, 1H), 2.15-1.94 (m, 3H). LC-MS (M+H)⁺=445.3.

### Example 39 & Example 40: (5-amino-6,8-dihydro-1H-furo[3,4-d]pyrrolo[3,2-b]pyridin-2-yl)((2R,6S)-9-bromo-3,4-dihydro-2H-2,6-methanobenzo[b][1,5]oxazocin-5(6H)-yl)methanone & (5-amino-6,8-dihydro-1H-furo[3,4-d]pyrrolo[3,2-b]pyridin-2-yl)((2S,6R)-9-bromo-3,4-dihydro-2H-2,6-methanobenzo[b][1,5]oxazocin-5(6H)-yl)methanone

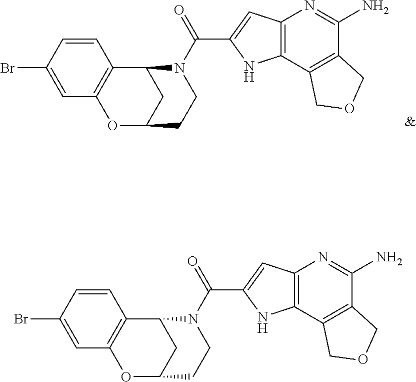 [SMILES: Nc1nc2cc(C(=O)N3CC[C@@H]4C[C@H]3c3ccc(Br)cc3O4)[nH]c2c2c1COC2 ; Nc1nc2cc(C(=O)N3CC[C@H]4C[C@@H]3c3ccc(Br)cc3O4)[nH]c2c2c1COC2]

### Step 1: 2-allyl-7-bromochroman-4-one

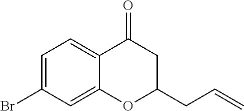

The title compound (3.1 g, 28%) was prepared in a manner similar to that in Example 37 & Example 38 step 2 from 7-bromo-4H-chromen-4-one and allylmagnesium bromide. LC-MS (M+H)⁺=266.9.

### Step 2: 2-(7-bromo-4-oxochroman-2-yl)acetaldehyde

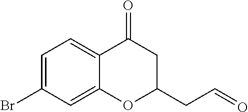 [SMILES: O=CCC1CC(=O)c2ccc(Br)cc2O1]

The title compound (2.4 g, 77%) was prepared in a manner similar to that in Example 37 & Example 38 step 3 from 2-allyl-7-bromochroman-4-one. LC-MS (M+H)⁺=269.0.

### Step 3: 7-bromo-2-(2-hydroxyethyl)chroman-4-ol

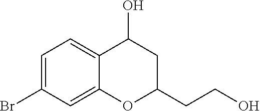 [SMILES: OCCC1CC(O)c2ccc(Br)cc2O1]

The title compound (2.0 g, 82%) was prepared in a manner similar to that in Example 37 & Example 38 step 4 from 2-(7-bromo-4-oxochroman-2-yl)acetaldehyde. LC-MS (M+Na)=295.2.

### Step 4: 7-bromo-2-(2-((tert-butyldimethylsilyl)oxy)ethyl)chroman-4-ol

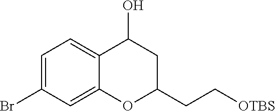 [SMILES: CC(C)(C)[Si](C)(C)OCCC1CC(O)c2ccc(Br)cc2O1]

The title compound (2.4 g, 85%) was prepared in a manner similar to that in Example 37 & Example 38 step 5 from 7-bromo-2-(2-hydroxyethyl)chroman-4-ol. LC-MS (M+Na)⁺=409.2.

### Step 5: tert-butyl (7-bromo-2-(2-((tert-butyldimethylsilyl)oxy)ethyl)chroman-4-yl)((2-nitrophenyl)sulfonyl)carbamate

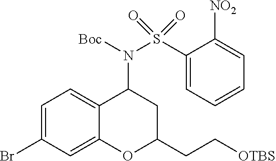 [SMILES: CC(C)(C)OC(=O)N(C1CC(CCO[Si](C)(C)C(C)(C)C)Oc2cc(Br)ccc21)S(=O)(=O)c1ccccc1[N+](=O)[O-]]

The title compound (2.0 g, 48%) was prepared in a manner similar to that in Example 37 & Example 38 step 6 from 7-bromo-2-(2-((tert-butyldimethylsilyl)oxy)ethyl)chroman-4-ol and 2-NsNHBoc. LC-MS (M+Na)⁺=693.3.

### Step 6: N-(7-bromo-2-(2-hydroxyethyl)chroman-4-yl)-2-nitrobenzenesulfonamide

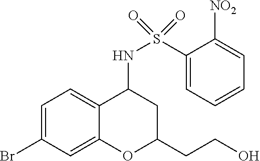 [SMILES: O=[N+]([O-])c1ccccc1S(=O)(=O)NC1CC(CCO)Oc2cc(Br)ccc21]

The title compound (840 mg, 62%) was prepared in a manner similar to that in Example 37 & Example 38 step 7 from tert-butyl (7-bromo-2-(2-((tert-butyldimethylsilyl)oxy)ethyl)chroman-4-yl)((2-nitrophenyl)sulfonyl)carbamate.

### Step 7: 9-bromo-5-((2-nitrophenyl)sulfonyl)-3,4,5,6-tetrahydro-2H-2,6-methanobenzo[b][1,5]oxazocine

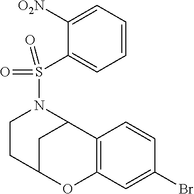 [SMILES: O=[N+]([O-])c1ccccc1S(=O)(=O)N1CCC2CC1c1ccc(Br)cc1O2]

The title compound (170 mg, 21%) was prepared in a manner similar to that in Example 37 & Example 38 step 8 from N-(7-bromo-2-(2-hydroxyethyl)chroman-4-yl)-2-nitrobenzenesulfonamide. LC-MS (M+H)⁺=439.0.

### Step 8: 9-bromo-3,4,5,6-tetrahydro-2H-2,6-methanobenzo[b][1,5]oxazocine

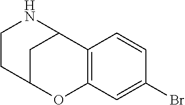 [SMILES: Brc1ccc2c(c1)OC1CCNC2C1]

The title compound (80 mg, 80%) was prepared in a manner similar to that in Example 37 & Example 38 step 9 from 9-bromo-5-((2-nitrophenyl)sulfonyl)-3,4,5,6-tetrahydro-2H-2,6-methanobenzo[b][1,5]oxazocine. LC-MS (M+H)=253.9.

### Step 9: (5-amino-1-((2-(trimethylsilyl)ethoxy)methyl)-6,8-dihydro-1H-furo[3,4-d]pyrrolo[3,2-b]pyridin-2-yl)(9-bromo-3,4-dihydro-2H-2,6-methanobenzo[b][1,5]oxazocin-5(6H)-yl)methanone

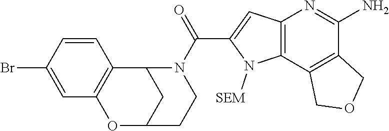 [SMILES: C[Si](C)(C)CCOCn1c(C(=O)N2CCC3CC2c2ccc(Br)cc2O3)cc2nc(N)c3c(c21)COC3]

The title compound (90 mg, 77%) was prepared in a manner similar to that in Example 37 & Example 38 step 10 from 9-bromo-3,4,5,6-tetrahydro-2H-2,6-methanobenzo[b][1,5]oxazocine. LC-MS (M+H)⁺=585.4.

### Step 10: (5-amino-6,8-dihydro-1H-furo[3,4-d]pyrrolo[3,2-b]pyridin-2-yl)((2R,6S)-9-bromo-3,4-dihydro-2H-2,6-methanobenzo[b][1,5]oxazocin-5(6H)-yl)methanone & (5-amino-6,8-dihydro-1H-furo[3,4-d]pyrrolo[3,2-b]pyridin-2-yl)((2S,6R)-9-bromo-3,4-dihydro-2H-2,6-methanobenzo[b][1,5]oxazocin-5(6H)-yl)methanone

Example 39 (16 mg, 23%) and Example 40 (19 mg, 27%) were prepared in a manner similar to that in Example 37 & Example 38 step 11 from (5-amino-1-((2-(trimethylsilyl)ethoxy)methyl)-6,8-dihydro-1H-furo[3,4-d]pyrrolo[3,2-b]pyridin-2-yl)(9-bromo-3,4-dihydro-2H-2,6-methanobenzo[b][1,5]oxazocin-5(6H)-yl)methanone, and the enantiomers were separated by chiral SFC.

Analytical chiral-SFC condition as below. Column: Lux Cellulose-3; Column size: 4.6×100 mm, m; Mobile phase: 4 mM methanolic NH₃:CO₂, 45:55 isocratic; Flow: 2.0 mL/min; Temperature: 35° C.; back pressure: 1500 psi.

Example 39: Analytical SFC tR=3.02 min. ¹H NMR (400 MHz, DMSO-d6, 80° C.) δ11.54 (s, 1H), 7.34-6.79 (m, 3H), 6.58 (s, 1H), 5.74 (s, 1H), 5.57 (s, 2H), 5.22-5.08 (m, 2H), 4.95-4.77 (m, 3H), 4.21 (s, 1H), 3.12-2.85 (m, 1H), 2.27-1.95 (m, 4H). LC-MS (M+H)⁺=455.2.

Example 40: Analytical SFC tR=4.01 min. ¹H NMR (400 MHz, DMSO-d6, 80° C.) δ11.50 (s, 1H), 7.23-6.75 (m, 3H), 6.54 (s, 1H), 5.68 (s, 1H), 5.53 (s, 2H), 5.08 (s, 2H), 4.93-4.72 (m, 3H), 4.16 (s, 1H), 2.99 (s, 1H), 2.22-1.78 (m, 4H). LC-MS (M+H)⁺=455.2.

### Example 41 & Example 42: (5-amino-6,8-dihydro-1H-furo[3,4-d]pyrrolo[3,2-b]pyridin-2-yl)((2S,6R)-7-fluoro-9-(trifluoromethyl)-3,4-dihydro-2H-2,6-methanobenzo[b][1,5]oxazocin-5(6H)-yl)methanone & (5-amino-6,8-dihydro-1H-furo[3,4-d]pyrrolo[3,2-b]pyridin-2-yl)((2R,6S)-7-fluoro-9-(trifluoromethyl)-3,4-dihydro-2H-2,6-methanobenzo[b][1,5]oxazocin-5(6H)-yl)methanone

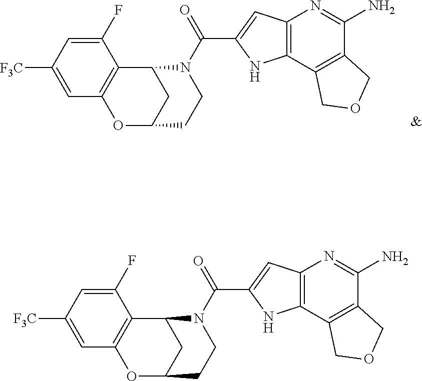 [SMILES: Nc1nc2cc(C(=O)N3CC[C@H]4C[C@@H]3c3c(F)cc(C(F)(F)F)cc3O4)[nH]c2c2c1COC2 ; Nc1nc2cc(C(=O)N3CC[C@@H]4C[C@H]3c3c(F)cc(C(F)(F)F)cc3O4)[nH]c2c2c1COC2]

### Step 1: 1-(4-bromo-2-fluoro-6-hydroxyphenyl)ethan-1-one

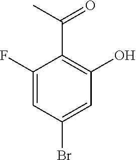

A mixture of 3-bromo-5-fluorophenol (50 g, 263 mmol) and AcCl (24.6 g, 316 mmol) was stirred for 30 min at 40° C., and then AlCl₃ (69.6 g, 523 mmol) was added. The mixture was stirred for 4 h at 140° C. The mixture was cooled to room temperature and poured into cold water (1000 mL). The mixture was extracted with EtOAc (1000 mL). The organic layer was washed with brine (500 mL), dried over Na₂SO₄, filtered and concentrated under reduced pressure. The residue was purified by silica gel chromatograph (PE:EtOAc=3:1) to give the title compound (40 g, 65%). LC-MS (M+H)⁺=233.1.

### Step 2: 7-bromo-5-fluoro-4H-chromen-4-one

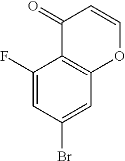 [SMILES: O=c1ccoc2cc(Br)cc(F)c12]

To a mixture of 1-(4-bromo-2-fluoro-6-hydroxyphenyl)ethan-1-one (40 g, 172 mmol) in ethyl formate (500 mL) and THF (100 mL) was added NaH (60%, 20.6 g, 517 mmol) at 0° C. After 1 h, the mixture was warmed to room temperature and stirred for 2 h. MeOH (50 mL) was slowly added followed by the addition of concentrated hydrochloric acid (150 mL). The mixture was stirred for 2 h at room temperature and diluted with water (1000 mL). The mixture was extracted with EtOAc (1000 mL). The organic layer was washed with brine (500 mL×2), dried over Na₂SO₄, filtered and concentrated under reduced pressure. The residue was purified by silica gel chromatograph (PE:EtOAc=3:1) to give the title compound (34.6 g, 83%). LC-MS (M+H)⁺=243.2.

### Step 3: 5-fluoro-7-(trifluoromethyl)-4H-chromen-4-one

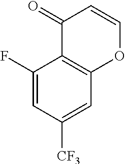 [SMILES: O=c1ccoc2cc(C(F)(F)F)cc(F)c12]

To a mixture of 7-bromo-5-fluoro-4H-chromen-4-one (20 g, 82.6 mmol) in DMF (500 mL), was added CuI (15.8 g, 82.6 mmol) and methyl 2,2-difluoro-2-(fluorosulfonyl)acetate (63.5 g, 330 mmol). The mixture was stirred for 16 h at 120° C. under nitrogen and cooled to room temperature. Solid was filtered off and the filtrate was diluted with saturated NaHCO₃ (500 mL). The mixture was extracted with EtOAc (500 mL×2). The organic layer was washed with brine (500 mL×2), dried over Na₂SO₄, filtered and concentrated under reduced pressure. The residue was purified by silica gel chromatography (PE:EtOAc=10:1) to give the title compound (10.5 g, 55%). LC-MS (M+H)⁺=233.4.

### Step 4: 2-allyl-5-fluoro-7-(trifluoromethyl)chroman-4-one

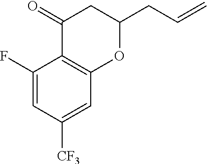 [SMILES: C=CCC1CC(=O)c2c(F)cc(C(F)(F)F)cc2O1]

A mixture of CuBr-Me₂S (232 mg, 1.1 mmol) and (R)-1-[(Sₚ)-2-(dicyclohexylphosphino)ferrocenylethyl]diphenylphosphine (806 mg, 1.3 mmol) in DCM (200 mL) was stirred for 10 min at room temperature. The mixture was cooled to −78° C. and allylmagnesium bromide (1.0 M in Et₂O, 56.6 mL, 56.6 mmol) was added dropwise. The mixture was stirred for 10 min at −78° C. followed by addition of 5-fluoro-7-(trifluoromethyl)-4H-chromen-4-one (10.5 g, 45.2 mmol) in DCM (50 mL). The mixture was stirred for 2 h at −78° C. The mixture was poured into saturated NH₄Cl (400 mL) and extracted with DCM (400 mL). The organic layer was washed with brine (400 mL), dried over Na₂SO₄, filtered and concentrated under reduced pressure. The residue was purified by silica gel chromatograph (PE:EtOAc=10:1) to give the title compound (5.0 g, 40%). LC-MS (M+H)⁺=275.1.

### Step 5: 2-(5-fluoro-4-oxo-7-(trifluoromethyl)chroman-2-yl)acetaldehyde

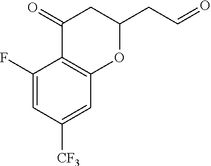 [SMILES: O=CCC1CC(=O)c2c(F)cc(C(F)(F)F)cc2O1]

To a solution of 2-allyl-5-fluoro-7-(trifluoromethyl)chroman-4-one (4.5 g, 16.4 mmol) in dioxane (200 mL) and water (40 mL) was added potassium osmate (VI) dihydrate (604 mg, 1.6 mmol), NaIO₄ (14 g, 65.7 mmol) and 2,6-lutidine (3.5 g, 32.8 mmol). The mixture was stirred at room temperature for 3 h. Solid was filtered off and the filtrate was diluted with water (300 mL) and then extracted with EtOAc (300 mL×2). The combined organic layer was successively washed with saturated Na₂S₂O₃ (300 mL), 1 N hydrochloric acid (300 mL), saturated NaHCO₃ (300 mL) and brine (300 mL), dried over Na₂SO₄, filtered and concentrated under reduced pressure to give the title compound (4.5 g, 100%). LC-MS (M+H)⁺=277.2.

### Step 6: 5-fluoro-2-(2-hydroxyethyl)-7-(trifluoromethyl)chroman-4-ol

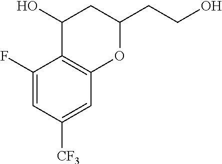 [SMILES: OCCC1CC(O)c2c(F)cc(C(F)(F)F)cc2O1]

To a solution of 2-(5-fluoro-4-oxo-7-(trifluoromethyl)chroman-2-yl)acetaldehyde (4.5 g, 16.3 mmol) in MeOH (60 mL) was added NaBH₄ (1.24 g, 32.6 mmol) at 0° C. The mixture was stirred at room temperature for 2 h. The mixture was poured into water (400 mL) and the mixture was extracted with EtOAc (400 mL×2). The combined organic layer was washed with brine (400 mL×2), dried over Na₂SO₄, filtered and concentrated under reduced pressure. The residue was purified by silica gel chromatography (PE:EtOAc=1:1) to give the title compound (4.0 g, 88%). LC-MS (M+H)=281.1.

### Step 7: 2-(2-((tert-butyldimethylsilyl)oxy)ethyl)-5-fluoro-7-(trifluoromethyl)chroman-4-ol

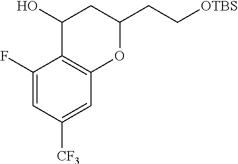 [SMILES: CC(C)(C)[Si](C)(C)OCCC1CC(O)c2c(F)cc(C(F)(F)F)cc2O1]

To a solution of 5-fluoro-2-(2-hydroxyethyl)-7-(trifluoromethyl)chroman-4-ol (4.0 g, 14.3 mmol) in DCM (50 mL) was added imidazole (1.9 g, 28.6 mmol) and TBSCl (3.2 g, 21.4 mmol) at 0° C. The mixture was stirred at room temperature for 1 h and partitioned between water (100 mL) and DCM (100 mL). The organic layer was washed with brine (100 mL), dried over Na₂SO₄, filtered and concentrated under reduced pressure. The residue was purified by silica gel chromatography (PE:EtOAc=3:1) to give the title compound (3.7 g, 66%). LC-MS (M+H)⁺=395.4.

### Step 8: tert-butyl (2-(2-((tert-butyldimethylsilyl)oxy)ethyl)-5-fluoro-7-(trifluoromethyl)chroman-4-yl)((2-nitrophenyl)sulfonyl)carbamate

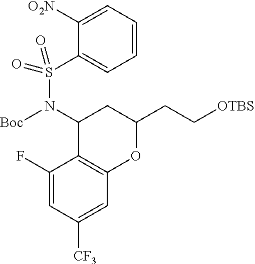 [SMILES: CC(C)(C)OC(=O)N(C1CC(CCO[Si](C)(C)C(C)(C)C)Oc2cc(C(F)(F)F)cc(F)c21)S(=O)(=O)c1ccccc1[N+](=O)[O-]]

To a mixture of 2-(2-((tert-butyldimethylsilyl)oxy)ethyl)-5-fluoro-7-(trifluoromethyl)chroman-4-ol (3.7 g, 9.4 mmol), 2-NsNHBoc (3.42 g, 11.3 mmol) and PPh₃ (3.72 g, 14.2 mmol) in THF (30 mL) was added DtBAD (3.26 g, 14.2 mmol) in portions at 0° C. under nitrogen. The mixture was stirred at 65° C. for 16 h and then cooled to room temperature. The mixture was concentrated under reduced pressure. The residue was purified by silica gel chromatography (PE:EtOAc=3:1) to give the title compound (3.0 g, 47%). LC-MS (M+H)⁺=679.2.

### Step 9: N-(5-fluoro-2-(2-hydroxyethyl)-7-(trifluoromethyl)chroman-4-yl)-2-nitrobenzenesulfonamide

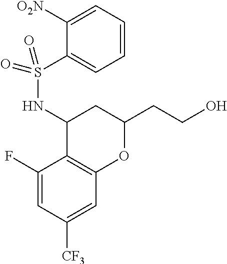 [SMILES: O=[N+]([O-])c1ccccc1S(=O)(=O)NC1CC(CCO)Oc2cc(C(F)(F)F)cc(F)c21]

A mixture of tert-butyl (2-(2-((tert-butyldimethylsilyl)oxy)ethyl)-5-fluoro-7-(trifluoromethyl)chroman-4-yl)((2-nitrophenyl)sulfonyl)carbamate (3.0 g, 4.4 mmol) and HCl (4 M in dioxane, 60 mL) was stirred at 45° C. for 16 h. The mixture was cooled to room temperature and concentrated under reduced pressure. The residue was partitioned between saturated NaHCO₃ (100 mL) and EtOAc (200 mL). The organic layer was washed with brine (100 mL), dried over Na₂SO₄, filtered and concentrated under reduced pressure. The residue was purified by silica gel chromatography (PE:EtOAc=1:1) to give the title compound (420 mg, 21%). LC-MS (M+H)⁺=465.3.

### Step 10: 7-fluoro-5-((2-nitrophenyl)sulfonyl)-9-(trifluoromethyl)-3,4,5,6-tetrahydro-2H-2,6-methanobenzo[b][1,5]oxazocine

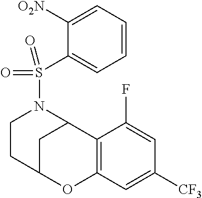 [SMILES: O=[N+]([O-])c1ccccc1S(=O)(=O)N1CCC2CC1c1c(F)cc(C(F)(F)F)cc1O2]

To a mixture of N-(5-fluoro-2-(2-hydroxyethyl)-7-(trifluoromethyl)chroman-4-yl)-2-nitrobenzenesulfonamide (420 mg, 0.91 mmol) and PPh₃ (714 mg, 2.71 mmol) in THF (200 mL) was added DtBAD (623 mg, 2.71 mmol) in portions at 0° C. under nitrogen. The mixture was stirred at room temperature for 16 h and concentrated under reduced pressure. The residue was purified by silica gel chromatography (PE:EtOAc=3:1) to give the title compound (403 mg, 99%). LC-MS (M+H)=447.1.

### Step 11: 7-fluoro-9-(trifluoromethyl)-3,4,5,6-tetrahydro-2H-2,6-methanobenzo[b][1,5]oxazocine

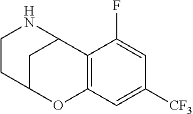 [SMILES: Fc1cc(C(F)(F)F)cc2c1C1CC(CCN1)O2]

A mixture of 7-fluoro-5-((2-nitrophenyl)sulfonyl)-9-(trifluoromethyl)-3,4,5,6-tetrahydro-2H-2,6-methanobenzo[b][1,5]oxazocine (403 mg, 0.90 mmol), 1-dodecanethiol (706 mg, 3.5 mmol), LiOH H₂O (147 mg, 3.5 mmol) in DMF (1 mL) and THF (10 mL) was stirred at room temperature for 16 h. The mixture was partitioned between water (50 mL) and EtOAc (50 mL). The organic layer was washed with brine (50 mL×2), dried over Na₂SO₄, filtered and concentrated under reduced pressure. The residue was purified by silica gel chromatography (DCM:MeOH=10:1) to give the title compound (236 mg, 99%). LC-MS (M+H)⁺=262.4.

### Step 12: (5-amino-1-((2-(trimethylsilyl)ethoxy)methyl)-6,8-dihydro-1H-furo[3,4-d]pyrrolo[3,2-b]pyridin-2-yl)(7-fluoro-9-(trifluoromethyl)-3,4-dihydro-2H-2,6-methanobenzo[b][1,5]oxazocin-5(6H)-yl)methanone

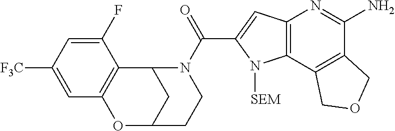 [SMILES: C[Si](C)(C)CCOCn1c(C(=O)N2CCC3CC2c2c(F)cc(C(F)(F)F)cc2O3)cc2nc(N)c3c(c21)COC3]

A mixture of 7-fluoro-9-(trifluoromethyl)-3,4,5,6-tetrahydro-2H-2,6-methanobenzo[b][1,5]oxazocine (43 mg, 0.16 mmol), 5-amino-1-((2-(trimethylsilyl)ethoxy)methyl)-6,8-dihydro-1H-furo[3,4-d]pyrrolo[3,2-b]pyridine-2-carboxylic acid (69 mg, 0.19 mmol), BOPCl (58 mg, 0.23 mmol) and DIPEA (64 mg, 0.49 mmol) in THF (10 mL) was stirred at 60° C. for 16 h. The mixture was cooled to room temperature and concentrated under reduced pressure. The residue was partitioned between water (50 mL) and EtOAc (50 mL). The organic layer was dried over Na₂SO₄, filtered and concentrated under reduced pressure. The residue was purified by silica gel chromatography (DCM:MeOH=25:1) to give the title compound (88 mg, 91%). LC-MS (M+H)⁺=593.2.

### Step 13: (5-amino-6,8-dihydro-1H-furo[3,4-d]pyrrolo[3,2-b]pyridin-2-yl)((2S,6R)-7-fluoro-9-(trifluoromethyl)-3,4-dihydro-2H-2,6-methanobenzo[b][1,5]oxazocin-5(6H)-yl)methanone & (5-amino-6,8-dihydro-1H-furo[3,4-d]pyrrolo[3,2-b]pyridin-2-yl)((2R,6S)-7-fluoro-9-(trifluoromethyl)-3,4-dihydro-2H-2,6-methanobenzo[b][1,5]oxazocin-5(6H)-yl)methanone

A mixture of (5-amino-1-((2-(trimethylsilyl)ethoxy)methyl)-6,8-dihydro-1H-furo[3,4-d]pyrrolo[3,2-b]pyridin-2-yl)(7-fluoro-9-(trifluoromethyl)-3,4-dihydro-2H-2,6-methanobenzo[b][1,5]oxazocin-5(6H)-yl)methanone (88 mg, 0.15 mmol) in DCM (6 mL) and TFA (6 mL) was stirred at room temperature for 3 h and then concentrated under reduced pressure. The residue was re-dissolved in methanolic ammonia (7 M, 12 mL). The mixture was stirred at room temperature for 2 h and then concentrated under reduced pressure. The residue was partitioned between saturated NaHCO₃ (50 mL) and EtOAc (50 mL). The organic layer was dried over Na₂SO₄, filtered and concentrated under reduced pressure. The residue was purified by prep-TLC followed by chiral SFC to give Example 41 (17 mg, 25%) and Example 42 (16 mg, 23%).

Analytical chiral-SFC condition as below. Column: Lux Cellulose-4; Column size: 4.6×100 mm, m; Mobile phase: 4 mM methanolic NH₃:CO₂, 45:55 isocratic; Flow: 2.0 mL/min; Temperature: 35° C.; back pressure: 1500 psi.

Example 41: Analytical SFC tR=2.96 min. ¹H NMR (400 MHz, DMSO-d6) δ 11.53 (s, 1H), 7.20-7.02 (m, 2H), 6.71-6.46 (m, 1H), 6.09-5.93 (m, 1H), 5.58 (s, 2H), 5.17-5.00 (m, 2H), 4.95-4.79 (m, 3H), 4.31-4.06 (m, 1H), 3.55-2.71 (m, 1H), 2.66-1.81 (m, 4H). LC-MS (M+H)⁺=463.3.

Example 42: Analytical SFC tR=3.71 min. ¹H NMR (400 MHz, DMSO-d6) δ 11.53 (s, 1H), 7.20-7.02 (m, 2H), 6.71-6.46 (m, 1H), 6.09-5.93 (m, 1H), 5.58 (s, 2H), 5.17-5.00 (m, 2H), 4.95-4.79 (m, 3H), 4.31-4.06 (m, 1H), 3.55-2.71 (m, 1H), 2.66-1.81 (m, 4H). LC-MS (M+H)⁺=463.2.

### Example 43 & Example 44: ((R)-5-amino-6-methyl-6,8-dihydro-1H-furo[3,4-d]pyrrolo[3,2-b]pyridin-2-yl)((2S,6R)-7-fluoro-9-(trifluoromethyl)-3,4-dihydro-2H-2,6-methanobenzo[b][1,5]oxazocin-5(6H)-yl)methanone & ((R)-5-amino-6-methyl-6,8-dihydro-1H-furo[3,4-d]pyrrolo[3,2-b]pyridin-2-yl)((2R,6S)-7-fluoro-9-(trifluoromethyl)-3,4-dihydro-2H-2,6-methanobenzo[b][1,5]oxazocin-5(6H)-yl)methanone

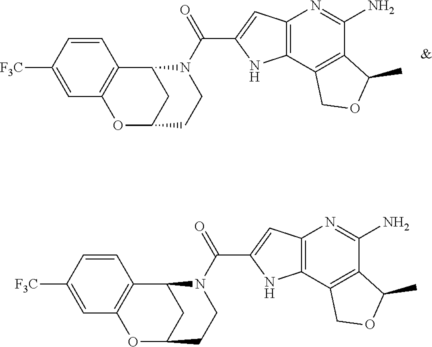 [SMILES: C[C@H]1OCc2c1c(N)nc1cc(C(=O)N3CC[C@H]4C[C@@H]3c3ccc(C(F)(F)F)cc3O4)[nH]c21 ; C[C@H]1OCc2c1c(N)nc1cc(C(=O)N3CC[C@@H]4C[C@H]3c3ccc(C(F)(F)F)cc3O4)[nH]c21]

To a mixture of 7-fluoro-9-(trifluoromethyl)-3,4,5,6-tetrahydro-2H-2,6-methanobenzo[b][1,5]oxazocine (56 mg, 0.21 mmol), (R)-5-amino-6-methyl-6,8-dihydro-1H-furo[3,4-d]pyrrolo[3,2-b]pyridine-2-carboxylic acid (50 mg, 0.21 mmol) and DIPEA (193 mg, 1.5 mmol) in anhydrous DMF (9 mL) was added T3P (50% in DMF, 682 mg, 1.07 mmol) and the mixture was stirred at 55° C. for 16 h. The mixture was cooled to room temperature and hydrochloric acid (6 M, 9 mL) was added. The mixture was stirred at 60° C. for 3 h and cooled to room temperature. The mixture was partitioned between saturated NaHCO₃ (100 mL) and EtOAc (100 mL). The organic layer was washed with brine (100 mL), dried over Na₂SO₄, filtered and concentrated under reduced pressure. The residue was purified by prep-TLC (DCM:MeOH=20:1) followed by chiral SFC to give Example 43 (25 mg, 25%) and Example 44 (24 mg, 24%).

Analytical chiral-SFC condition as below. Column: Lux Cellulose-4; Column size: 4.6×100 mm, 5 μm; Mobile phase: 4 mM methanolic NH₃:CO₂, 45:55 isocratic; Flow: 2.0 mL/min; Temperature: 35° C.; back pressure: 1500 psi.

Example 43: Analytical SFC tR=2.39 min. ¹H NMR (400 MHz, DMSO-d6, 80° C.) δ11.31 (s, 1H), 7.11-6.95 (m, 2H), 6.58 (s, 1H), 6.07-5.96 (m, 1H), 5.37-5.26 (m, 1H), 5.23-5.09 (m, 3H), 5.01 (d, J=13.8 Hz, 1H), 4.93-4.83 (m, 1H), 4.20 (dd, J=13.7, 5.9 Hz, 1H), 2.96-2.82 (m, 1H), 2.33-2.20 (m, 1H), 2.11-1.83 (m, 3H), 1.36 (d, J=6.0 Hz, 3H). LC-MS (M+H)⁺=477.3.

Example 44: Analytical SFC tR=3.36 min. ¹H NMR (400 MHz, DMSO-d6, 80° C.) δ11.31 (s, 1H), 7.11-6.97 (m, 2H), 6.58 (s, 1H), 6.07-5.94 (m, 1H), 5.37-5.28 (m, 1H), 5.19 (s, 2H), 5.14 (dd, J=13.8, 3.4 Hz, 1H), 5.04 (dd, J=13.8, 1.3 Hz, 1H), 4.93-4.85 (m, 1H), 4.20 (dd, J=14.2, 6.1 Hz, 1H), 2.97-2.84 (m, 1H), 2.29 (d, J=12.9 Hz, 1H), 2.09-1.86 (m, 3H), 1.36 (d, J=6.1 Hz, 3H). LC-MS (M+H)⁺=477.4.

### Example 45 & Example 46: ((R)-5-amino-6-methyl-6,8-dihydro-1H-furo[3,4-d]pyrrolo[3,2-b]pyridin-2-yl)((2S,6R)-9-(trifluoromethyl)-3,4-dihydro-2H-2,6-methanobenzo[b][1,5]oxazocin-5(6H)-yl)methanone & ((R)-5-amino-6-methyl-6,8-dihydro-1H-furo[3,4-d]pyrrolo[3,2-b]pyridin-2-yl)((2R,6S)-9-(trifluoromethyl)-3,4-dihydro-2H-2,6-methanobenzo[b][1,5]oxazocin-5(6H)-yl)methanone

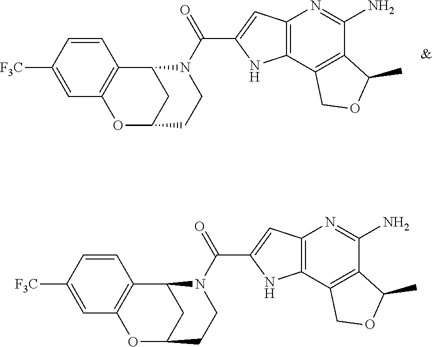 [SMILES: C[C@H]1OCc2c1c(N)nc1cc(C(=O)N3CC[C@H]4C[C@@H]3c3ccc(C(F)(F)F)cc3O4)[nH]c21 ; C[C@H]1OCc2c1c(N)nc1cc(C(=O)N3CC[C@@H]4C[C@H]3c3ccc(C(F)(F)F)cc3O4)[nH]c21]

To a mixture of (R)-5-amino-6-methyl-6,8-dihydro-1H-furo[3,4-d]pyrrolo[3,2-b]pyridine-2-carboxylic acid (58 mg, 0.25 mmol), 9-(trifluoromethyl)-3,4,5,6-tetrahydro-2H-2,6-methanobenzo[b][1,5]oxazocine (60 mg, 0.25 mmol) and DIPEA (223 mg, 1.73 mmol) in anhydrous DMF (9 mL) under nitrogen was added T3P (50% in DMF, 784 mg, 1.23 mmol) and the mixture was stirred at 55° C. for 16 h. The mixture was cooled to room temperature and hydrochloric acid (6 M, 9 mL) was added. The mixture was stirred at 60° C. for 3 h and cooled to room temperature. The mixture was partitioned between saturated NaHCO₃ (100 mL) and EtOAc (100 mL). The organic layer was washed with brine (100 mL), dried over Na₂SO₄, filtered and concentrated under reduced pressure. The residue was purified by prep-TLC (DCM:MeOH=20:1) followed by preparative chiral-HPLC to give Example 45 (27 mg, 24%) and Example 46 (26 mg, 23%).

Analytical chiral-HPLC as below. Column: CHIRALPAK IF; Column size: 4.6×150 mm, 5 m; Mobile phase: Hexane:(0.1% Et₂NH in EtOH)=4:6 isocratic; Flow: 1.0 mL/min; Temperature: 25° C.

Example 45: Analytical chiral-HPLC tR=5.52 min. ¹H NMR (400 MHz, DMSO-d6, 80° C.) 6 11.51 (s, 1H), 7.54-7.00 (m, 3H), 6.75-6.37 (m, 1H), 5.77 (s, 1H), 5.45 (s, 2H), 5.37-5.23 (m, 1H), 5.18-5.05 (m, 1H), 5.01-4.97 (m, 1H), 4.90-4.75 (m, 1H), 4.30-4.06 (m, 1H), 3.09-2.69 (m, 1H), 2.27-2.12 (m, 1H), 2.10-1.81 (m, 3H), 1.45-1.24 (m, 3H). LC-MS (M+H)⁺=459.4.

Example 46: Analytical chiral-HPLC tR=7.93 min. ¹H NMR (400 MHz, DMSO-d6, 80° C.) 6 11.51 (s, 1H), 7.60-6.95 (m, 3H), 6.83-6.28 (m, 1H), 5.92-5.66 (m, 1H), 5.45 (s, 2H), 5.36-5.22 (m, 1H), 5.18-4.95 (m, 2H), 4.90-4.78 (m, 1H), 4.31-4.04 (m, 1H), 3.17-2.79 (m, 1H), 2.31-2.12 (m, 1H), 2.09-1.79 (m, 3H), 1.45-1.27 (m, 3H). LC-MS (M+H)⁺=459.4.

### Example 47 & Example 48: ((R)-5-amino-6-methyl-6,8-dihydro-1H-furo[3,4-d]pyrrolo[3,2-b]pyridin-2-yl)((1S,5R)-10-fluoro-8-(trifluoromethyl)-3,4,5,6-tetrahydro-1,5-methanobenzo[c]azocin-2(1H)-yl)methanone & ((R)-5-amino-6-methyl-6,8-dihydro-1H-furo[3,4-d]pyrrolo[3,2-b]pyridin-2-yl)((1R,5S)-10-fluoro-8-(trifluoromethyl)-3,4,5,6-tetrahydro-1,5-methanobenzo[c]azocin-2(1H)-yl)methanone

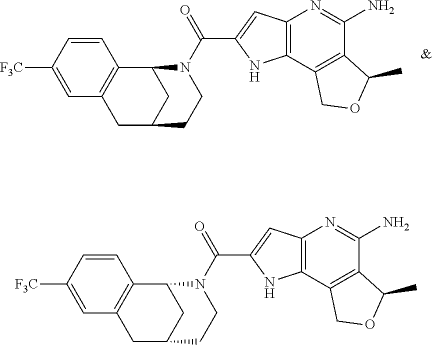 [SMILES: C[C@H]1OCc2c1c(N)nc1cc(C(=O)N3CC[C@H]4Cc5cc(C(F)(F)F)ccc5[C@@H]3C4)[nH]c21 ; C[C@H]1OCc2c1c(N)nc1cc(C(=O)N3CC[C@@H]4Cc5cc(C(F)(F)F)ccc5[C@H]3C4)[nH]c21]

To a mixture of 10-fluoro-8-(trifluoromethyl)-1,2,3,4,5,6-hexahydro-1,5-methanobenzo[c]azocine (60 mg, 0.23 mmol), (R)-5-amino-6-methyl-6,8-dihydro-1H-furo[3,4-d]pyrrolo[3,2-b]pyridine-2-carboxylic acid (54 mg, 0.23 mmol), DIPEA (92 mg, 0.71 mmol) in DMF (5 mL) was added HATU (203 mg, 0.53 mmol) and the mixture was stirred at room temperature for 16 h. The mixture was partitioned between water (20 mL) and EtOAc (20 mL). The organic layer was dried over Na₂SO₄, filtered and concentrated under reduced pressure. The residue was purified by prep-TLC (DCM:MeOH=10:1) followed by chiral SFC to give Example 47 (33 mg, 30%) and Example 48 (35 mg, 32%).

Analytical chiral-SFC condition as below. Column: Lux Cellulose-3; Column size: 4.6×100 mm, m; Mobile phase: 4 mM methanolic NH₃:CO₂, 1:9 to 1:1 in 3 min, 1:1 for 2 min, 1:1 to 1:9 in 0.1 min, 1:9 for 1.9 min; Flow: 2.0 mL/min; Temperature: 35° C.; back pressure: 1500 psi.

Example 47: Analytical SFC tR=2.09 min. ¹H NMR (400 MHz, DMSO-d6, 80° C.) δ11.26 (s, 1H), 7.41 (s, 1H), 7.32 (d, J=9.4 Hz, 1H), 6.51 (s, 1H), 6.03 (s, 1H), 5.36-5.26 (m, 1H), 5.16 (s, 2H), 5.15-5.11 (m, 1H), 5.15-4.98 (m, 1H), 4.12-4.02 (m, 1H), 3.25-3.15 (m, 1H), 3.01-2.93 (m, 1H), 2.82-2.71 (m, 1H), 2.43-2.39 (m, 1H), 2.19-2.09 (m, 1H), 1.92-1.79 (m, 2H), 1.72-1.63 (m, 1H), 1.36 (d, J=6.1 Hz, 3H). LC-MS (M+H)⁺=475.2.

Example 48: Analytical SFC tR=2.48 min. ¹H NMR (400 MHz, DMSO-d6, 80° C.) δ11.26 (s, 1H), 7.41 (s, 1H), 7.32 (d, J=9.4 Hz, 1H), 6.51 (s, 1H), 6.03 (s, 1H), 5.36-5.26 (m, 1H), 5.16 (s, 2H), 5.15-5.11 (m, 1H), 5.15-4.98 (m, 1H), 4.12-4.02 (m, 1H), 3.25-3.15 (m, 1H), 3.01-2.93 (m, 1H), 2.82-2.71 (m, 1H), 2.43-2.39 (m, 1H), 2.19-2.09 (m, 1H), 1.92-1.79 (m, 2H), 1.72-1.63 (m, 1H), 1.36 (d, J=6.1 Hz, 3H). LC-MS (M+H)⁺=475.2.

### Example 49 & Example 50: ((R)-5-amino-6-methyl-6,8-dihydro-1H-furo[3,4-d]pyrrolo[3,2-b]pyridin-2-yl)((1S,5R)-8-(trifluoromethyl)-3,4,5,6-tetrahydro-1,5-methanobenzo[c]azocin-2(1H)-yl)methanone & ((R)-5-amino-6-methyl-6,8-dihydro-1H-furo[3,4-d]pyrrolo[3,2-b]pyridin-2-yl)((1R,5S)-8-(trifluoromethyl)-3,4,5,6-tetrahydro-1,5-methanobenzo[c]azocin-2(1H)-yl)methanone

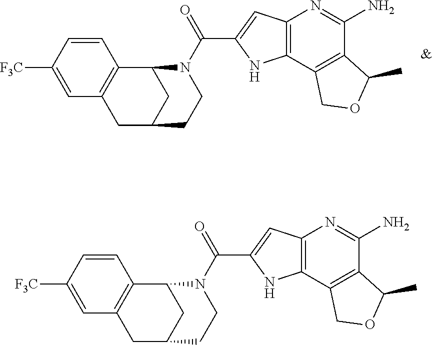 [SMILES: C[C@H]1OCc2c1c(N)nc1cc(C(=O)N3CC[C@H]4Cc5cc(C(F)(F)F)ccc5[C@@H]3C4)[nH]c21 ; C[C@H]1OCc2c1c(N)nc1cc(C(=O)N3CC[C@@H]4Cc5cc(C(F)(F)F)ccc5[C@H]3C4)[nH]c21]

A mixture of (R)-5-amino-6-methyl-6,8-dihydro-1H-furo[3,4-d]pyrrolo[3,2-b]pyridine-2-carboxylic acid (45 mg, 0.20 mmol), 8-(trifluoromethyl)-1,2,3,4,5,6-hexahydro-1,5-methanobenzo[c]azocine (47 mg, 0.20 mmol), T3P (50% in DMF, 620 mg, 0.98 mmol) and DIPEA (0.20 g, 1.56 mmol) in DMF (5 mL) was stirred at to 60° C. under nitrogen overnight. Concentrated hydrochloric acid (5 mL) was added and the mixture was stirred at to 60° C. for 2 h. The mixture was cooled to room temperature and partitioned between saturated NaHCO₃ (20 mL) and EtOAc (30 mL). The organic layer was dried over Na₂SO₄, filtered and concentrated under reduced pressure. The residue was purified by prep-TLC (DCM:MeOH=10:1) followed by chiral SFC to give Example 49 (10 mg, 11%) and Example 50 (10 mg, 11%).

Analytical chiral-SFC condition as below. Column: YMC Cellulose-C; Column size: 4.6×100 mm, 5 μm; Mobile phase: 4 mM methanolic NH₃:CO₂, 1:9 to 1:1 in 3 min, 1:1 for 2 min, 1:1 to 1:9 in 0.1 min, 1:9 for 1.9 min; Flow: 2.0 mL/min; Temperature: 35° C.; back pressure: 1500 psi.

Example 49: Analytical SFC tR=3.36 min. ¹H NMR (400 MHz, DMSO-d6, 80° C.) δ11.28 (s, 1H), 7.51 (s, 1H), 7.44 (d, J=8.0 Hz, 1H), 7.36-7.16 (m, 1H), 6.50 (s, 1H), 5.89-5.66 (m, 1H), 5.37-5.28 (m, 1H), 5.24-5.10 (m, 3H), 5.03 (d, J=13.8 Hz, 1H), 4.06 (dd, J=13.8, 5.4 Hz, 1H), 3.19 (dd, J=18.3, 6.7 Hz, 1H), 2.92 (d, J=18.2 Hz, 1H), 2.82-2.62 (m, 1H), 2.44-2.35 (m, 1H), 2.11 (d, J=12.7 Hz, 1H), 1.97-1.83 (m, 2H), 1.70 (d, J=13.5 Hz, 1H), 1.37 (d, J=6.1 Hz, 3H). LC-MS (M+H)⁺=457.1.

Example 50: Analytical SFC tR=3.69 min. ¹H NMR (400 MHz, DMSO-d6, 80° C.) δ11.27 (s, 1H), 7.51 (s, 1H), 7.44 (d, J=8.0 Hz, 1H), 7.34-7.19 (m, 1H), 6.50 (s, 1H), 5.84-5.73 (m, 1H), 5.38-5.28 (m, 1H), 5.25-5.10 (m, 3H), 5.03 (d, J=13.7 Hz, 1H), 4.06 (dd, J=13.9, 5.5 Hz, 1H), 3.19 (dd, J=18.3, 6.8 Hz, 1H), 2.92 (d, J=18.3 Hz, 1H), 2.81-2.65 (m, 1H), 2.45-2.39 (m, 1H), 2.11 (d, J=13.0 Hz, 1H), 1.97-1.84 (m, 2H), 1.69 (d, J=12.9 Hz, 1H), 1.37 (d, J=6.1 Hz, 3H). LC-MS (M+H)⁺=457.1.

### Example 51: (5-amino-6,8-dihydro-1H-furo[3,4-d]pyrrolo[3,2-b]pyridin-2-yl)((1S,5S)-8-ethynyl-1,3,4,6-tetrahydro-2H-1,5-methanobenzo[f][1,4]diazocin-2-yl)methanone

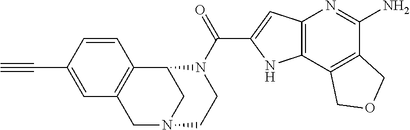 [SMILES: C#Cc1ccc2c(c1)C[N@@]1CCN(C(=O)c3cc4nc(N)c5c(c4[nH]3)COC5)[C@@H]2C1]

Step 1: (5-amino-6,8-dihydro-1H-furo[3,4-d]pyrrolo[3,2-b]pyridin-2-yl)((1S,5S)-8-((trimethylsilyl)ethynyl)-1,3,4,6-tetrahydro-2H-1,5-methanobenzo[f][1,4]diazocin-2-yl)methanone

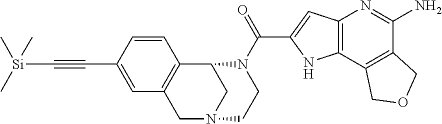 [SMILES: C[Si](C)(C)C#Cc1ccc2c(c1)C[N@@]1CCN(C(=O)c3cc4nc(N)c5c(c4[nH]3)COC5)[C@@H]2C1]

A mixture of (5-amino-6,8-dihydro-1H-furo[3,4-d]pyrrolo[3,2-b]pyridin-2-yl)((1S,5S)-8-iodo-1,3,4,6-tetrahydro-2H-1,5-methanobenzo[f][1,4]diazocin-2-yl)methanone (50 mg, 0.10 mmol), PdCl₂(PPh₃)₂ (70 mg, 0.10 mmol), CuI (19 mg, 0.10 mmol), Et₃N (0.06 mL, 0.40 mmol) and trimethylsilylacetylene (30 mg, 0.30 mmol) in DMF (2 mL) was stirred at 45° C. under nitrogen overnight. The mixture was cooled to room temperature and partitioned between water (10 mL) and EtOAc (10 mL). The organic layer was dried over Na₂SO₄, filtered and concentrated under reduced pressure. The residue was purified by prep-TLC (DCM/MeOH=15:1) to give the title compound (10 mg, 21%). LC-MS (M+H)⁺=472.2.

### Step 2: (5-amino-6,8-dihydro-1H-furo[3,4-d]pyrrolo[3,2-b]pyridin-2-yl)((1S,5S)-8-ethynyl-1,3,4,6-tetrahydro-2H-1,5-methanobenzo[f][1,4]diazocin-2-yl)methanone

A mixture of (5-amino-6,8-dihydro-1H-furo[3,4-d]pyrrolo[3,2-b]pyridin-2-yl)((1S,5S)-8-((trimethylsilyl)ethynyl)-1,3,4,6-tetrahydro-2H-1,5-methanobenzo[f][1,4]diazocin-2-yl)methanone (10 mg, 0.021 mmol) and K₂CO₃ (9 mg, 0.065 mmol) in methanol (5 mL) was stirred at room temperature for 2 h. The mixture was concentrated under reduced pressure and purified by prep-HPLC to give Example 51 (0.3 mg, 4%). ¹H NMR (400 MHz, DMSO-d6, 80° C.) δ11.47 (s, 1H), 7.24 (s, 2H), 6.77-6.55 (m, 1H), 6.48-6.29 (m, 1H), 5.52 (s, 2H), 5.08 (s, 2H), 4.87 (s, 2H), 4.29-4.10 (m, 2H), 3.92 (d, J=18.1 Hz, 1H), 3.08 (s, 2H), 1.96 (s, 1H). LC-MS (M+H)=400.1.

### Example 52 & Example 53: (5-amino-6,8-dihydro-1H-furo[3,4-d]pyrrolo[3,2-b]pyridin-2-yl)((1R,5R)-8-iodo-6,6-dimethyl-1,3,4,6-tetrahydro-2H-1,5-methanobenzo[f][1,4]diazocin-2-yl)methanone & (5-amino-6,8-dihydro-1H-furo[3,4-d]pyrrolo[3,2-b]pyridin-2-yl)((1S,5S)-8-iodo-6,6-dimethyl-1,3,4,6-tetrahydro-2H-1,5-methanobenzo[f][1,4]diazocin-2-yl)methanone

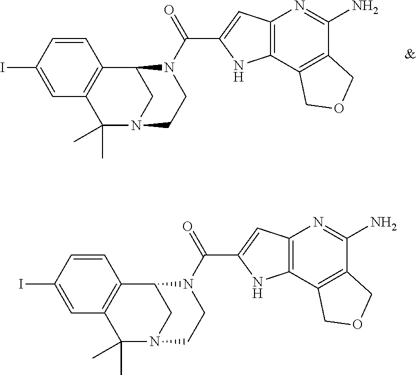 [SMILES: CC1(C)c2cc(I)ccc2[C@@H]2C[N@@]1CCN2C(=O)c1cc2nc(N)c3c(c2[nH]1)COC3 ; CC1(C)c2cc(I)ccc2[C@H]2C[N@]1CCN2C(=O)c1cc2nc(N)c3c(c2[nH]1)COC3]

### Step 1: tert-butyl 8-bromo-6,6-dimethyl-1,3,4,6-tetrahydro-2H-1,5-methanobenzo[f][1,4]diazocine-2-carboxylate

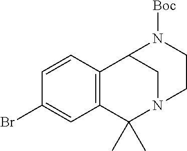 [SMILES: CC(C)(C)OC(=O)N1CCN2CC1c1ccc(Br)cc1C2(C)C]

To a mixture of 2-((7-bromo-1,1-dimethyl-1,2,3,4-tetrahydroisoquinolin-4-yl)amino)ethan-1-ol (541 mg, 1.8 mmol) in THF (20 mL) was added SOCl₂ (0.60 mL, 9.0 mmol) at room temperature. The mixture was stirred at 60° C. for 2 h, cooled to room temperature and concentrated under reduced pressure. The residue was taken in DMF (10 mL) followed by addition of K₂CO₃ (2.4 g, 18 mmol). The mixture was stirred at 80° C. for 2 h and cooled to room temperature. Boc₂O (784 mg, 3.6 mmol) was added and the mixture was stirred at room temperature for 2 h. The mixture was partitioned between DCM (100 mL) and water (50 mL). The organic layer was dried over Na₂SO₄, filtered and concentrated under reduced pressure. The residue was purified by silica gel chromatograph (PE:EtOAc=1:1) to give the title compound (620 mg, 89%). LC-MS (M+H)⁺=381.2.

### Step 2: tert-butyl 8-iodo-6,6-dimethyl-1,3,4,6-tetrahydro-2H-1,5-methanobenzo[f][1,4]diazocine-2-carboxylate

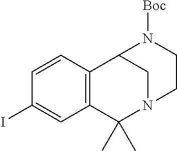 [SMILES: CC(C)(C)OC(=O)N1CCN2CC1c1ccc(I)cc1C2(C)C]

To a mixture of tert-butyl 8-bromo-6,6-dimethyl-1,3,4,6-tetrahydro-2H-1,5-methanobenzo[f][1,4]diazocine-2-carboxylate (620 mg, 1.63 mmol) and NaI (978 mg, 6.52 mmol) in dioxane (10 mL) was added CuI (154 mg, 0.81 mmol) and trans-N,N′-dimethylcyclohexane-1,2-diamine (232 mg, 1.63 mmol) under nitrogen. The mixture was stirred at 105° C. for 72 h and cooled to room temperature. The mixture was partitioned between EtOAc (200 mL) and water (100 mL). The organic layer was dried over Na₂SO₄, filtered and concentrated under reduced pressure. The residue was purified by silica gel chromatograph (PE:EtOAc=1:1) to give the title compound (580 mg, 83%). LC-MS (M+H)⁺=429.2.

### Step 3: 8-iodo-6,6-dimethyl-1,3,4,6-tetrahydro-2H-1,5-methanobenzo[f][1,4]diazocine dihydrochloride

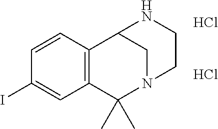 [SMILES: CC1(C)c2cc(I)ccc2C2CN1CCN2 ; Cl ; Cl]

A mixture of tert-butyl 8-iodo-6,6-dimethyl-1,3,4,6-tetrahydro-2H-1,5-methanobenzo[f][1,4]diazocine-2-carboxylate (200 mg, 0.47 mmol) in HCl (4 M in dioxane, 6 mL) was stirred at room temperature for 4 h. The mixture was concentrated under reduced pressure to give the title compound (186 mg, 100%).

### Step 4: (5-amino-1-((2-(trimethylsilyl)ethoxy)methyl)-6,8-dihydro-1H-furo[3,4-d]pyrrolo[3,2-b]pyridin-2-yl)(8-iodo-6,6-dimethyl-1,3,4,6-tetrahydro-2H-1,5-methanobenzo[f][1,4]diazocin-2-yl)methanone

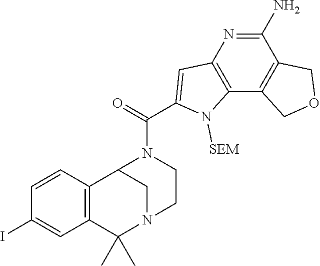 [SMILES: CC1(C)c2cc(I)ccc2C2CN1CCN2C(=O)c1cc2nc(N)c3c(c2n1COCC[Si](C)(C)C)COC3]

To a mixture of 5-amino-1-((2-(trimethylsilyl)ethoxy)methyl)-6,8-dihydro-1H-furo[3,4-d]pyrrolo[3,2-b]pyridine-2-carboxylic acid (106 mg, 0.30 mmol) and 8-iodo-6,6-dimethyl-1,3,4,6-tetrahydro-2H-1,5-methanobenzo[f][1,4]diazocine dihydrochloride (93 mg, 0.23 mmol) in THF (10 mL) was added BOPCl (114 mg, 0.45 mmol) and DIPEA (0.30 mL, 1.5 mmol). The mixture was stirred at 60° C. for 2 h and cooled to room temperature. The mixture was partitioned between water (10 mL) and EtOAc (50 mL). The organic layer was washed with brine (10 mL), dried over Na₂SO₄, filtered and concentrated under reduced pressure. The residue was purified by prep-TLC (DCM:MeOH=20:1) to give the title compound (100 mg, 66%). LC-MS (M+H)⁺=660.2.

### Step 5: (5-amino-6,8-dihydro-1H-furo[3,4-d]pyrrolo[3,2-b]pyridin-2-yl)((1R,5R)-8-iodo-6,6-dimethyl-1,3,4,6-tetrahydro-2H-1,5-methanobenzo[f][1,4]diazocin-2-yl)methanone & (5-amino-6,8-dihydro-1H-furo[3,4-d]pyrrolo[3,2-b]pyridin-2-yl)((1S,5S)-8-iodo-6,6-dimethyl-1,3,4,6-tetrahydro-2H-1,5-methanobenzo[f][1,4]diazocin-2-yl)methanone

A mixture of (5-amino-1-((2-(trimethylsilyl)ethoxy)methyl)-6,8-dihydro-1H-furo[3,4-d]pyrrolo[3,2-b]pyridin-2-yl)(8-iodo-6,6-dimethyl-1,3,4,6-tetrahydro-2H-1,5-methanobenzo[f][1,4]diazocin-2-yl)methanone (100 mg, 0.15 mmol) in TFA (4 mL) was stirred at room temperature for 0.5 h and concentrated under reduced pressure. The mixture was taken in THF (4 mL) and ammonium hydroxide (28%, 2 mL). The mixture was stirred at room temperature for 1 h and concentrated under reduced pressure. The residue was purified by prep-HPLC followed by chiral SFC to give Example 52 (10 mg, 13%) and Example 53 (10 mg, 13%).

Analytical chiral-SFC condition as below. Column: YMC Cellulose-C; Column size: 4.6×100 mm, 5 μm; Mobile phase: 4 mM methanolic NH₃:CO₂, 45:55 isocratic; Flow: 2.0 mL/min; Temperature: 35° C.; back pressure: 1500 psi.

Example 52: Analytical SFC tR=4.94 min. ¹H NMR (400 MHz, DMSO-d6, 80° C.) δ11.25 (s, 1H), 7.68 (s, 1H), 7.49-7.47 (m, 1H), 6.81 (s, 1H), 6.50 (s, 1H), 5.35 (s, 1H), 5.27 (s, 2H), 5.11 (s, 2H), 4.90 (s, 2H), 3.76-3.73 (m, 1H), 3.44-3.18 (m, 3H), 2.97-2.84 (m, 2H), 1.55 (s, 3H), 1.36 (s, 3H). LC-MS (M+H)⁺=530.2.

Example 53: Analytical SFC tR=8.97 min. ¹H NMR (400 MHz, DMSO-d6, 80° C.) δ11.25 (s, 1H), 7.68 (s, 1H), 7.49-7.47 (m, 1H), 6.81 (s, 1H), 6.50 (s, 1H), 5.35 (s, 1H), 5.27 (s, 2H), 5.11 (s, 2H), 4.90 (s, 2H), 3.76-3.73 (m, 1H), 3.44-3.18 (m, 3H), 2.97-2.84 (m, 2H), 1.55 (s, 3H), 1.36 (s, 3H). LC-MS (M+H)⁺=530.0.

### Example 54 & Example 55: ((R)-5-amino-6-methyl-6,8-dihydro-1H-furo[3,4-d]pyrrolo[3,2-b]pyridin-2-yl)((1R,5R)-8-iodo-6,6-dimethyl-1,3,4,6-tetrahydro-2H-1,5-methanobenzo[f][1,4]diazocin-2-yl)methanone & ((R)-5-amino-6-methyl-6,8-dihydro-1H-furo[3,4-d]pyrrolo[3,2-b]pyridin-2-yl)((1S,5S)-8-iodo-6,6-dimethyl-1,3,4,6-tetrahydro-2H-1,5-methanobenzo[f][1,4]diazocin-2-yl)methanone

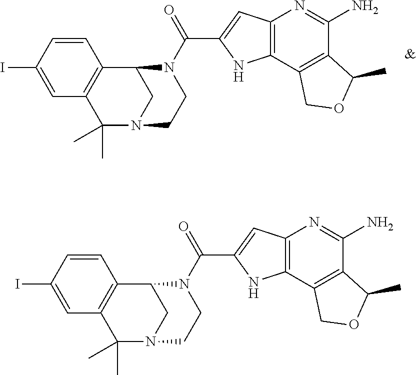 [SMILES: C[C@H]1OCc2c1c(N)nc1cc(C(=O)N3CC[N@]4C[C@H]3c3ccc(I)cc3C4(C)C)[nH]c21 ; C[C@H]1OCc2c1c(N)nc1cc(C(=O)N3CC[N@@]4C[C@@H]3c3ccc(I)cc3C4(C)C)[nH]c21]

To a mixture of (R)-5-amino-6-methyl-6,8-dihydro-1H-furo[3,4-d]pyrrolo[3,2-b]pyridine-2-carboxylic acid (71 mg, 0.30 mmol), 8-iodo-6,6-dimethyl-1,3,4,6-tetrahydro-2H-1,5-methanobenzo[f][1,4]diazocine dihydrochloride (93 mg, 0.23 mmol) and DIPEA (0.39 g, 3.0 mmol) in anhydrous DMF (10 mL) was added T3P (50% in DMF, 954 mg, 1.5 mmol) and the mixture was stirred at 60° C. for 16 h. The mixture was cooled to room temperature and hydrochloric acid (6 M, 9 mL) was added. The mixture was stirred 60° C. for 3 h and cooled to room temperature. The mixture was partitioned between saturated NaHCO₃ (100 mL) and EtOAc (100 mL). The organic layer was washed with brine (100 mL), dried over Na₂SO₄, filtered and concentrated under reduced pressure. The residue was purified by prep-TLC followed by chiral SFC to give Example 54 (10 mg, 8%) and Example 55 (10 mg, 8%).

Analytical chiral-SFC condition as below. Column: YMC Cellulose-C; Column size: 4.6×100 mm, 5 μm; Mobile phase: 4 mM methanolic NH₃:CO₂, 45:55 isocratic; Flow: 2.0 mL/min; Temperature: 35° C.; back pressure: 1500 psi.

Example 54: Analytical SFC tR=3.91 min. ¹H NMR (400 MHz, DMSO-d6, 80° C.) δ11.26 (s, 1H), 7.68 (s, 1H), 7.49-7.47 (m, 1H), 7.81 (s, 1H), 6.49 (s, 1H), 5.36-5.34 (m, 2H), 5.17-5.01 (m, 4H), 3.75-3.72 (m, 1H), 3.44-3.18 (m, 3H), 2.90-2.84 (m, 2H), 1.55 (s, 3H), 1.35 (s, 6H). LC-MS (M+H)⁺=544.0.

Example 55: Analytical SFC tR=8.76 min. ¹H NMR (400 MHz, DMSO-d6, 80° C.) δ11.26 (s, 1H), 7.68 (s, 1H), 7.49-7.47 (m, 1H), 7.81 (s, 1H), 6.49 (s, 1H), 5.36-5.34 (m, 2H), 5.17-5.01 (m, 4H), 3.75-3.72 (m, 1H), 3.44-3.18 (m, 3H), 2.90-2.84 (m, 2H), 1.55 (s, 3H), 1.35 (s, 6H). LC-MS (M+H)⁺=544.0.

### Example 56 & Example 57: (5-amino-6,8-dihydro-1H-furo[3,4-d]pyrrolo[3,2-b]pyridin-2-yl)((1R,5R)-6,6-dimethyl-8-(trifluoromethyl)-1,3,4,6-tetrahydro-2H-1,5-methanobenzo[f][1,4]diazocin-2-yl)methanone & (5-amino-6,8-dihydro-1H-furo[3,4-d]pyrrolo[3,2-b]pyridin-2-yl)((1S,5S)-6,6-dimethyl-8-(trifluoromethyl)-1,3,4,6-tetrahydro-2H-1,5-methanobenzo[f][1,4]diazocin-2-yl)methanone

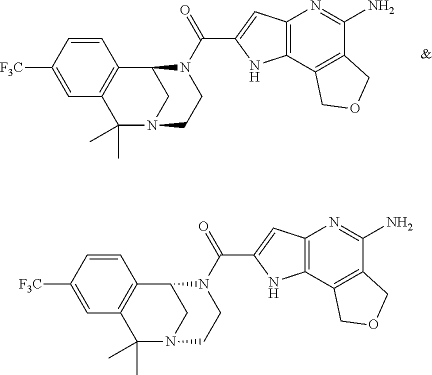 [SMILES: CC1(C)c2cc(C(F)(F)F)ccc2[C@@H]2C[N@@]1CCN2C(=O)c1cc2nc(N)c3c(c2[nH]1)COC3 ; CC1(C)c2cc(C(F)(F)F)ccc2[C@H]2C[N@]1CCN2C(=O)c1cc2nc(N)c3c(c2[nH]1)COC3]

### Step 1: tert-butyl 6,6-dimethyl-8-(4,4,5,5-tetramethyl-1,3,2-dioxaborolan-2-yl)-1,3,4,6-tetrahydro-2H-1,5-methanobenzo[f][1,4]diazocine-2-carboxylate

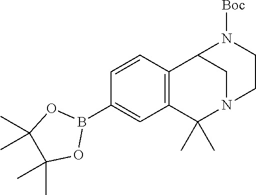 [SMILES: CC(C)(C)OC(=O)N1CCN2CC1c1ccc(B3OC(C)(C)C(C)(C)O3)cc1C2(C)C]

To a mixture of tert-butyl 8-iodo-6,6-dimethyl-1,3,4,6-tetrahydro-2H-1,5-methanobenzo[f][1,4]diazocine-2-carboxylate (440 mg, 1.03 mmol) and BPD (392 mg, 1.5 mmol) in dioxane (15 mL) was added Pd(dppf)Cl₂ (84 mg, 0.10 mmol) and KOAc (302 mg, 3.1 mmol) under nitrogen. The mixture was stirred at 100° C. for 12 h and then cooled to room temperature. The mixture was partitioned between EtOAc (200 mL) and water (100 mL). The organic layer was dried over Na₂SO₄, filtered and concentrated under reduced pressure. The residue was purified by silica gel chromatography (PE:EtOAc=1:1) to give the title compound (300 mg, 68%). LC-MS (M+H)⁺=429.2.

### Step 2: tert-butyl 6,6-dimethyl-8-(trifluoromethyl)-1,3,4,6-tetrahydro-2H-1,5-methanobenzo[f][1,4]diazocine-2-carboxylate

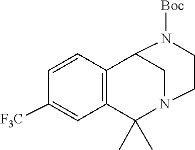 [SMILES: CC(C)(C)OC(=O)N1CCN2CC1c1ccc(C(F)(F)F)cc1C2(C)C]

To a mixture of tert-butyl 6,6-dimethyl-8-(4,4,5,5-tetramethyl-1,3,2-dioxaborolan-2-yl)-1,3,4,6-tetrahydro-2H-1,5-methanobenzo[f][1,4]diazocine-2-carboxylate (200 mg, 0.47 mmol) in DMF (5 mL) was added (1,10-phenanthroline)(trifluoromethyl)copper(I) (292 mg, 0.94 mmol) under nitrogen. The mixture was stirred at 60° C. for 2 h and cooled to room temperature. The residue was partitioned between EtOAc (200 mL) and water (100 mL). The organic layer was dried over Na₂SO₄, filtered and concentrated under reduced pressure. The residue was purified by silica gel chromatography (PE:EtOAc=1:1) to give the title compound (150 mg, 87%). LC-MS (M+H)⁺=371.2.

### Step 3: 6,6-dimethyl-8-(trifluoromethyl)-1,3,4,6-tetrahydro-2H-1,5-methanobenzo[f][1,4]diazocine dihydrochloride

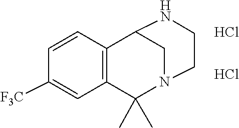 [SMILES: CC1(C)c2cc(C(F)(F)F)ccc2C2CN1CCN2 ; Cl ; Cl]

A mixture of tert-butyl 6,6-dimethyl-8-(trifluoromethyl)-1,3,4,6-tetrahydro-2H-1,5-methanobenzo[f][1,4]diazocine-2-carboxylate (170 mg, 0.46 mmol) in HCl (4 M in dioxane, 6 mL) was stirred at room temperature for 4 h. The mixture was concentrated under reduced pressure to give the title compound (157 mg, 100%). LC-MS (M+H)⁺=271.1.

### Step 4: (5-amino-1-((2-(trimethylsilyl)ethoxy)methyl)-6,8-dihydro-1H-furo[3,4-d]pyrrolo[3,2-b]pyridin-2-yl)(6,6-dimethyl-8-(trifluoromethyl)-1,3,4,6-tetrahydro-2H-1,5-methanobenzo[f][1,4]diazocin-2-yl)methanone

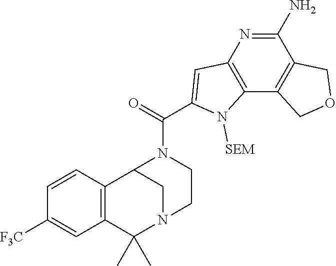 [SMILES: CC1(C)c2cc(C(F)(F)F)ccc2C2CN1CCN2C(=O)c1cc2nc(N)c3c(c2n1COCC[Si](C)(C)C)COC3]

To a mixture of 5-amino-1-((2-(trimethylsilyl)ethoxy)methyl)-6,8-dihydro-H-furo[3,4-d]pyrrolo[3,2-b]pyridine-2-carboxylic acid (116 mg, 0.33 mmol) and 6,6-dimethyl-8-(trifluoromethyl)-1,3,4,6-tetrahydro-2H-1,5-methanobenzo[f][1,4]diazocine (78 mg, 0.23 mmol) in THF (10 mL) was added BOPCl (125 mg, 0.495 mmol) and DIPEA (0.3 mL, 1.65 mmol). The mixture was stirred at 60° C. for 2 h and cooled to room temperature. The mixture was partitioned between water (10 mL) and EtOAc (50 mL). The organic layer was washed with brine (10 mL), dried over Na₂SO₄, filtered and concentrated under reduced pressure. The residue was purified by prep-TLC (DCM:MeOH=20:1) to give the title compound (100 mg, 72%). LC-MS (M+H)⁺=602.2.

### Step 5: (5-amino-6,8-dihydro-1H-furo[3,4-d]pyrrolo[3,2-b]pyridin-2-yl)((1R,5R)-6,6-dimethyl-8-(trifluoromethyl)-1,3,4,6-tetrahydro-2H-1,5-methanobenzo[f][1,4]diazocin-2-yl)methanone & (5-amino-6,8-dihydro-1H-furo[3,4-d]pyrrolo[3,2-b]pyridin-2-yl)((1S,5S)-6,6-dimethyl-8-(trifluoromethyl)-1,3,4,6-tetrahydro-2H-1,5-methanobenzo[f][1,4]diazocin-2-yl)methanone

A mixture of (5-amino-1-((2-(trimethylsilyl)ethoxy)methyl)-6,8-dihydro-1H-furo[3,4-d]pyrrolo[3,2-b]pyridin-2-yl)(6,6-dimethyl-8-(trifluoromethyl)-1,3,4,6-tetrahydro-2H-1,5-methanobenzo[f][1,4]diazocin-2-yl)methanone (100 mg, 0.17 mmol) in TFA (4 mL) was stirred at room temperature for 0.5 h and concentrated under reduced pressure. The mixture was taken in THF (4 mL) and ammonium hydroxide (28%, 2 mL). The mixture was stirred at room temperature for 2 h and concentrated under reduced pressure. The residue was purified by prep-HPLC followed by chiral SFC to give Example 56 (10 mg, 12%) and Example 57 (10 mg, 12%).

Analytical chiral-SFC condition as below. Column: YMC Cellulose-C; Column size: 4.6×100 mm, 5 μm; Mobile phase: 4 mM methanolic NH₃:CO₂, 45:55 isocratic; Flow: 2.0 mL/min; Temperature: 35° C.; back pressure: 1500 psi.

Example 56: Analytical SFC tR=2.27 min. ¹H NMR (400 MHz, DMSO-d6, 80° C.) δ11.28 (s, 1H), 7.65 (s, 1H), 7.45-7.47 (m, 1H), 7.25 (s, 1H), 6.52 (s, 1H), 5.47 (s, 1H), 5.28 (s, 2H), 5.11 (s, 2H), 4.90 (s, 2H), 3.79-3.76 (m, 1H), 3.48-3.50 (m, 1H), 3.21-3.22 (m, 1H), 3.10-3.05 (m, 1H), 2.83 (s, 1H), 1.61 (s, 3H), 1.40 (s, 3H). LC-MS (M+H)⁺=472.3.

Example 57: Analytical SFC tR=3.75 min. ¹H NMR (400 MHz, DMSO-d6, 80° C.) δ11.28 (s, 1H), 7.65 (s, 1H), 7.45-7.47 (m, 1H), 7.25 (s, 1H), 6.52 (s, 1H), 5.47 (s, 1H), 5.28 (s, 2H), 5.11 (s, 2H), 4.90 (s, 2H), 3.79-3.76 (m, 1H), 3.48-3.50 (m, 1H), 3.21-3.22 (m, 1H), 3.10-3.05 (m, 1H), 2.83 (s, 1H), 1.61 (s, 3H), 1.40 (s, 3H). LC-MS (M+H)⁺=472.3.

### Example 58 & Example 59: ((R)-5-amino-6-methyl-6,8-dihydro-1H-furo[3,4-d]pyrrolo[3,2-b]pyridin-2-yl)((1R,5R)-6,6-dimethyl-8-(trifluoromethyl)-1,3,4,6-tetrahydro-2H-1,5-methanobenzo[f][1,4]diazocin-2-yl)methanone & ((R)-5-amino-6-methyl-6,8-dihydro-1H-furo[3,4-d]pyrrolo[3,2-b]pyridin-2-yl)((1S,5S)-6,6-dimethyl-8-(trifluoromethyl)-1,3,4,6-tetrahydro-2H-1,5-methanobenzo[f][1,4]diazocin-2-yl)methanone

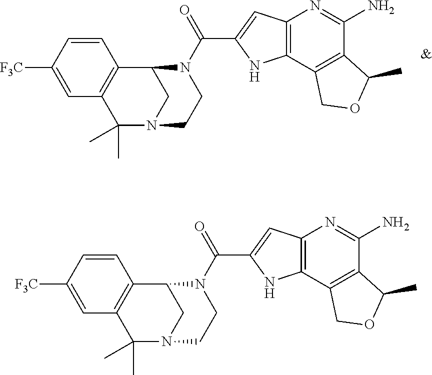 [SMILES: C[C@H]1OCc2c1c(N)nc1cc(C(=O)N3CC[N@]4C[C@H]3c3ccc(C(F)(F)F)cc3C4(C)C)[nH]c21 ; C[C@H]1OCc2c1c(N)nc1cc(C(=O)N3CC[N@@]4C[C@@H]3c3ccc(C(F)(F)F)cc3C4(C)C)[nH]c21]

To a mixture of (R)-5-amino-6-methyl-6,8-dihydro-1H-furo[3,4-d]pyrrolo[3,2-b]pyridine-2-carboxylic acid (77 mg, 0.33 mmol), 6,6-dimethyl-8-(trifluoromethyl)-1,3,4,6-tetrahydro-2H-1,5-methanobenzo[f][1,4]diazocine (78 mg, 0.23 mmol) and DIPEA (0.43 g, 3.0 mmol) in anhydrous DMF (10 mL) was added T3P (50% in DMF, 1.06 g, 1.7 mmol) and the mixture was stirred at 60° C. for 16 h. The mixture was cooled to room temperature and hydrochloric acid (6 M, 9 mL) was added. The mixture was stirred 60° C. for 3 h and cooled to room temperature. The mixture was partitioned between saturated NaHCO₃ (100 mL) and EtOAc (100 mL). The organic layer was washed with brine (100 mL), dried over Na₂SO₄, filtered and concentrated under reduced pressure. The residue was purified by prep-TLC followed by chiral SFC to give Example 58 (10 mg, 9%) and Example 59 (10 mg, 9%).

Analytical chiral-SFC condition as below. Column: YMC Cellulose-C; Column size: 4.6×100 mm, 5 μm; Mobile phase: 4 mM methanolic NH₃:CO₂, 45:55 isocratic; Flow: 2.0 mL/min; Temperature: 35° C.; back pressure: 1500 psi.

Example 58: Analytical SFC tR=1.84 min. ¹H NMR (400 MHz, DMSO-d6, 80° C.) δ11.28 (s, 1H), 7.65 (s, 1H), 7.45-7.47 (m, 1H), 7.25 (s, 1H), 6.51 (s, 1H), 5.47 (s, 1H), 5.32 (s, 1H), 5.18-5.01 (m, 4H), 3.79-3.76 (m, 1H), 3.48-3.50 (m, 1H), 3.21-3.22 (m, 1H), 3.10-3.05 (m, 1H), 2.83 (s, 1H), 1.61 (s, 3H), 1.40 (s, 3H), 1.22 (s, 3H). LC-MS (M+H)⁺=486.4.

Example 59: Analytical SFC tR=3.63 min. ¹H NMR (400 MHz, DMSO-d6, 80° C.) δ11.28 (s, 1H), 7.65 (s, 1H), 7.45-7.47 (m, 1H), 7.25 (s, 1H), 6.51 (s, 1H), 5.47 (s, 1H), 5.32 (s, 1H), 5.18-5.01 (m, 4H), 3.79-3.76 (m, 1H), 3.48-3.50 (m, 1H), 3.21-3.22 (m, 1H), 3.10-3.05 (m, 1H), 2.83 (s, 1H), 1.61 (s, 3H), 1.40 (s, 3H), 1.22 (s, 3H). LC-MS (M+H)⁺=486.3.

### Example 60 & Example 61: (5-amino-6,8-dihydro-1H-furo[3,4-d]pyrrolo[3,2-b]pyridin-2-yl)((2R,6S)-1-methyl-9-(trifluoromethyl)-1,3,4,6-tetrahydro-2,6-methanobenzo[b][1,5]diazocin-5(2H)-yl)methanone & (5-amino-6,8-dihydro-1H-furo[3,4-d]pyrrolo[3,2-b]pyridin-2-yl)((2S,6R)-1-methyl-9-(trifluoromethyl)-1,3,4,6-tetrahydro-2,6-methanobenzo[b][1,5]diazocin-5(2H)-yl)methanone

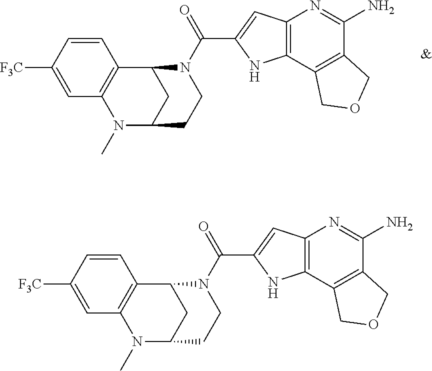 [SMILES: CN1c2cc(C(F)(F)F)ccc2[C@@H]2C[C@H]1CCN2C(=O)c1cc2nc(N)c3c(c2[nH]1)COC3 ; CN1c2cc(C(F)(F)F)ccc2[C@H]2C[C@@H]1CCN2C(=O)c1cc2nc(N)c3c(c2[nH]1)COC3]

### Step 1: 6-(2-nitro-4-(trifluoromethyl)phenyl)-5,6-dihydropyridin-2(1H)-one

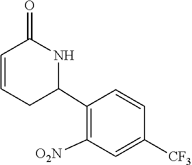 [SMILES: O=C1C=CCC(c2ccc(C(F)(F)F)cc2[N+](=O)[O-])N1]

To a mixture of 2-nitro-4-(trifluoromethyl)benzaldehyde (9.0 g, 41 mmol) and 3-butenamide (3.5 g, 41 mmol) was added Eaton's reagent (10 mL). The mixture was stirred at 45° C. overnight. The mixture was cooled to room temperature and slowly added to saturated NaHCO₃ (200 mL). The mixture was extracted with EtOAc (100 mL). The organic layer was dried over Na₂SO₄, filtered and concentrated under reduced pressure. The residue was purified by silica gel chromatography (PE:EtOAc=4:1) to give the title compound (1.4 g, 12%). LC-MS (M+H)⁺=287.0.

### Step 2: 9-(trifluoromethyl)-2,3,5,6-tetrahydro-2,6-methanobenzo[b][1,5]diazocin-4(1H)-one

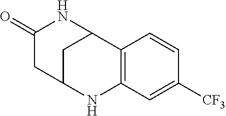 [SMILES: O=C1CC2CC(N1)c1ccc(C(F)(F)F)cc1N2]

To a mixture of 6-(2-nitro-4-(trifluoromethyl)phenyl)-5,6-dihydropyridin-2(1H)-one (1.4 g, 4.9 mmol) and NH₄Cl (1.4 g, 25 mmol) in EtOH (15 mL) and water (15 mL) was added iron powder (1.4 g, 25 mmol). The mixture was stirred at 70° C. for overnight. The mixture was cooled to room temperature and solid was filtered off. The filtrate was concentrated under reduced pressure. The residue was partitioned between EtOAc (50 mL) and water (30 mL). The organic layer was dried over Na₂SO₄, filtered and concentrated under reduced pressure. The residue was purified by silica gel chromatography (EtOAc) to give the title compound (550 mg, 44%). LC-MS (M+H)=257.0.

### Step 3: tert-butyl 9-(trifluoromethyl)-1,3,4,6-tetrahydro-2,6-methanobenzo[b][1,5]diazocine-5(2H)-carboxylate

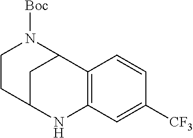 [SMILES: CC(C)(C)OC(=O)N1CCC2CC1c1ccc(C(F)(F)F)cc1N2]

To a mixture of 9-(trifluoromethyl)-2,3,5,6-tetrahydro-2,6-methanobenzo[b][1,5]diazocin-4(1H)-one (174 mg, 0.68 mmol) in THF (5 mL) was added BH₃ (1.0 M in THF, 4.0 mL, 4.0 mmol). The mixture was stirred at to 60° C. overnight and cooled to room temperature. Concentrated hydrochloric acid (2 mL) was added and the mixture was stirred at 60° C. for 2 h. The mixture was cooled to room temperature, and aqueous K₂CO₃ (3 N) solution was added slowly until the pH of mixture reaches 9. Boc₂O (440 mg, 2.0 mmol) was added and the mixture was stirred at room temperature for 4 h. The mixture was partitioned between water (10 mL) and EtOAc (20 mL). The organic layer was dried over Na₂SO₄, filtered and concentrated under reduced pressure. The residue was purified by prep-TLC (PE:EtOAc=1:1) to give the title compound (90 mg, 39%). LC-MS (M+H)⁺=343.1.

### Step 4: tert-butyl 1-methyl-9-(trifluoromethyl)-1,3,4,6-tetrahydro-2,6-methanobenzo[b][1,5]diazocine-5(2H)-carboxylate

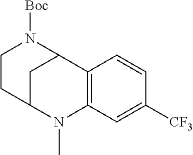 [SMILES: CN1c2cc(C(F)(F)F)ccc2C2CC1CCN2C(=O)OC(C)(C)C]

To a mixture of tert-butyl 9-(trifluoromethyl)-1,3,4,6-tetrahydro-2,6-methanobenzo[b][1,5]diazocine-5(2H)-carboxylate (90 mg, 0.26 mmol) and MeI (112 mg, 0.79 mmol) in THF (5 mL) was added NaH (60%, 32 mg, 0.79 mmol) under nitrogen. The mixture was stirred at 80° C. overnight and then cooled to room temperature. MeOH (5 mL) was added and the mixture was concentrated under reduced pressure. The residue was purified by prep-TLC (PE:EtOAc=3:1) to give the title compound (65 mg, 70%). LC-MS (M+H)⁺=357.1.

### Step 5: 1-methyl-9-(trifluoromethyl)-1,2,3,4,5,6-hexahydro-2,6-methanobenzo[b][1,5]diazocine hydrochloride

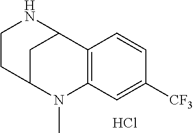 [SMILES: CN1c2cc(C(F)(F)F)ccc2C2CC1CCN2 ; Cl]

A mixture of tert-butyl 1-methyl-9-(trifluoromethyl)-1,3,4,6-tetrahydro-2,6-methanobenzo[b][1,5]diazocine-5(2H)-carboxylate (65 mg, 0.18 mmol) and HCl (4 M in dioxane, 5 mL) was stirred at room temperature for 2 h. The mixture was concentrated under reduced pressure to give the title compound (53 mg, 100%). LC-MS (M+H)=257.1.

### Step 6: (5-amino-1-((2-(trimethylsilyl)ethoxy)methyl)-6,8-dihydro-1H-furo[3,4-d]pyrrolo[3,2-b]pyridin-2-yl)(1-methyl-9-(trifluoromethyl)-1,3,4,6-tetrahydro-2,6-methanobenzo[b][1,5]diazocin-5(2H)-yl)methanone

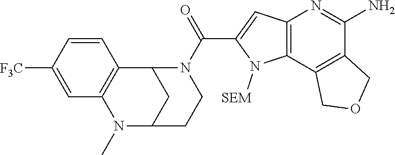 [SMILES: CN1c2cc(C(F)(F)F)ccc2C2CC1CCN2C(=O)c1cc2nc(N)c3c(c2n1COCC[Si](C)(C)C)COC3]

To a mixture of 5-amino-1-((2-(trimethylsilyl)ethoxy)methyl)-6,8-dihydro-1H-furo[3,4-d]pyrrolo[3,2-b]pyridine-2-carboxylic acid (83 mg, 0.24 mmol) and 1-methyl-9-(trifluoromethyl)-1,2,3,4,5,6-hexahydro-2,6-methanobenzo[b][1,5]diazocine hydrochloride (53 mg, 0.18 mmol) in THF (5 mL) was added BOPCl (70 mg, 0.27 mmol) and DIPEA (0.16 mL, 0.91 mmol) under nitrogen. The mixture was stirred at 80° C. for 8 h. The mixture was cooled to room temperature and partitioned between water (10 mL) and EtOAc (20 mL). The organic layer was dried over Na₂SO₄, filtered and concentrated under reduced pressure. The residue was purified by prep-TLC (DCM:MeOH=20:1) to give the title compound (105 mg, 100%). LC-MS (M+H)⁺=588.2.

### Step 7: (5-amino-6,8-dihydro-1H-furo[3,4-d]pyrrolo[3,2-b]pyridin-2-yl)((2R,6S)-1-methyl-9-(trifluoromethyl)-1,3,4,6-tetrahydro-2,6-methanobenzo[b][1,5]diazocin-5(2H)-yl)methanone & (5-amino-6,8-dihydro-1H-furo[3,4-d]pyrrolo[3,2-b]pyridin-2-yl)((2S,6R)-1-methyl-9-(trifluoromethyl)-1,3,4,6-tetrahydro-2,6-methanobenzo[b][1,5]diazocin-5(2H)-yl)methanone

Example 60 (16 mg, 19%) and Example 61 (16 mg, 19%) were prepared in a manner similar to that in Example 37 & Example 38 step 11 from (5-amino-1-((2-(trimethylsilyl)ethoxy)methyl)-6,8-dihydro-1H-furo[3,4-d]pyrrolo[3,2-b]pyridin-2-yl)(1-methyl-9-(trifluoromethyl)-1,3,4,6-tetrahydro-2,6-methanobenzo[b][1,5]diazocin-5(2H)-yl)methanone, and the enantiomers were separated by chiral SFC.

Analytical chiral-SFC condition as below. Column: Lux Cellulose-3; Column size: 4.6×100 mm, m; Mobile phase: 4 mM methanolic NH₃:CO₂, 1:9 to 1:1 in 3 min, 1:1 for 2 min, 1:1 to 1:9 in 0.1 min, 1:9 for 1.9 min; Flow: 2.0 mL/min; Temperature: 35° C.; back pressure: 1500 psi.

Example 60: Analytical SFC tR=2.77 min. ¹H NMR (400 MHz, DMSO-d6, 80° C.) δ11.67 (s, 1H), 7.28-6.43 (m, 4H), 6.00-5.52 (m, 3H), 5.11 (s, 2H), 4.89 (s, 2H), 4.12 (s, 1H), 3.71 (s, 1H), 2.99 (s, 3H), 2.82 (s, 1H), 2.31-1.78 (m 3H), 1.69 (s, 1H). LC-MS (M+H)⁺=458.1.

Example 61: Analytical SFC tR=3.02 min. ¹H NMR (400 MHz, DMSO-d6, 80° C.) δ11.67 (s, 1H), 7.28-6.43 (m, 4H), 6.00-5.52 (m, 3H), 5.11 (s, 2H), 4.89 (s, 2H), 4.12 (s, 1H), 3.71 (s, 1H), 2.99 (s, 3H), 2.82 (s, 1H), 2.31-1.78 (m 3H), 1.69 (s, 1H). LC-MS (M+H)⁺=458.1.

### Example 62 & Example 63: (5-amino-6,8-dihydro-1H-furo[3,4-d]pyrrolo[3,2-b]pyridin-2-yl)((1′R,5′R)-8′-(trifluoromethyl)-3′,4′-dihydrospiro[cyclopropane-1,6′-[1,5]methanobenzo[f][1,4]diazocin]-2′(1′H)-yl)methanone & (5-amino-6,8-dihydro-1H-furo[3,4-d]pyrrolo[3,2-b]pyridin-2-yl)((1'S,5'S)-8′-(trifluoromethyl)-3′,4′-dihydrospiro[cyclopropane-1,6′-[1,5]methanobenzo[f][1,4]diazocin]-2′(1′H)-yl)methanone

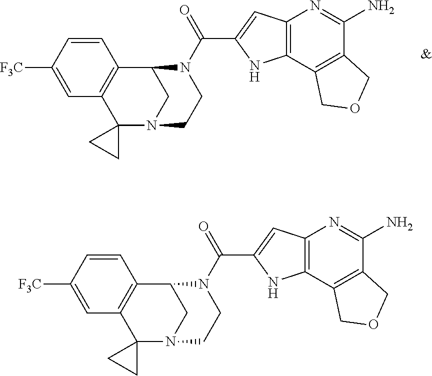 [SMILES: Nc1nc2cc(C(=O)N3CC[N@]4C[C@H]3c3ccc(C(F)(F)F)cc3C43CC3)[nH]c2c2c1COC2 ; Nc1nc2cc(C(=O)N3CC[N@@]4C[C@@H]3c3ccc(C(F)(F)F)cc3C43CC3)[nH]c2c2c1COC2]

### Step 1: ethyl (1-(3-bromophenyl)cyclopropyl)glycinate

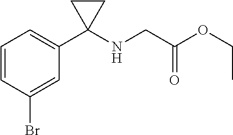 [SMILES: CCOC(=O)CNC1(c2cccc(Br)c2)CC1]

To a mixture of 1-(3-bromophenyl)cyclopropan-1-amine (3.0 g, 14.2 mmol) and K₂CO₃ (3.9 g, 28.4 mmol) in MeCN (100 mL) was added ethyl 2-bromoacetate (2.6 g, 15.6 mmol). The mixture was stirred at 60° C. for 12 h and cooled to room temperature. The mixture was concentrated under reduced pressure. The residue was partitioned between water (50 mL) and EtOAc (200 mL). The organic layer was washed with brine (50 mL), dried over Na₂SO₄, filtered and concentrated under reduced pressure. The residue was purified by silica gel chromatography (PE:EtOAc=5:1) to give the title compound (4.0 g, 95%). LC-MS (M+H)⁺=298.2.

### Step 2: ethyl N-((benzyloxy)carbonyl)-N-(1-(3-bromophenyl)cyclopropyl)glycinate

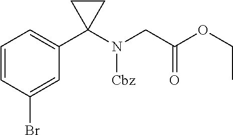 [SMILES: CCOC(=O)CN(C(=O)OCc1ccccc1)C1(c2cccc(Br)c2)CC1]

To a mixture of ethyl (1-(3-bromophenyl)cyclopropyl)glycinate (5.0 g, 16.7 mmol) and K₂CO₃ (4.6 g, 33.4 mmol) in MeCN (100 mL) was added CbzCl (3.0 ml, 21.7 mmol) dropwise. The mixture was stirred at 60° C. for 5 h and cooled to room temperature. The mixture was concentrated under reduced pressure. The residue was partitioned between EtOAc (200 mL) and water (100 mL). The organic layer was dried over Na₂SO₄, filtered and concentrated under reduced pressure. The residue was purified by silica gel chromatography (PE:EtOAc=15:1) to give the title compound (5.0 g, 69%). LC-MS (M+H)⁺=432.2.

### Step 3: N-((benzyloxy)carbonyl)-N-(1-(3-bromophenyl)cyclopropyl)glycine

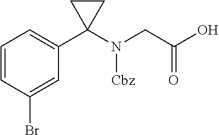 [SMILES: O=C(O)CN(C(=O)OCc1ccccc1)C1(c2cccc(Br)c2)CC1]

To a mixture of ethyl N-((benzyloxy)carbonyl)-N-(1-(3-bromophenyl)cyclopropyl)glycinate (5.0 g, 11.6 mmol) in MeOH (20 mL) was added a solution of NaOH (3.3 g, 84 mmol) in water (20 mL) dropwise. The mixture was stirred at room temperature for 2 h and then MeOH was removed under reduced pressure. The residue was partitioned between EtOAc (200 mL) and 4 N hydrochloric acid (50 mL). The organic layer was washed with water (100 mL), dried over Na₂SO₄, filtered and concentrated under reduced pressure to give the title compound (4.5 g, 96%). LC-MS (M+H)⁺=404.1.

### Step 4: 7′-bromo-2′,3′-dihydro-4′H-spiro[cyclopropane-1,1′-isoquinolin]-4′-one

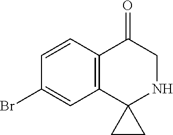 [SMILES: O=C1CNC2(CC2)c2cc(Br)ccc21]

To a mixture of N-((benzyloxy)carbonyl)-N-(1-(3-bromophenyl)cyclopropyl)glycine (5.6 g, 13.9 mmol) in DCM (100 mL), was added DMF (5 drops) and oxalyl chloride (1.3 mL, 15.2 mmol) dropwise at 0° C. The mixture was stirred at room temperature for 1 h and concentrated under reduced pressure. The residue was re-dissolved in DCM (100 mL) followed by addition of AlCl₃ (7.3 g, 55.4 mmol) in portions at 0° C. The mixture was stirred at room temperature for 12 h and then carefully added to saturated NaHCO₃ (300 mL) under vigorous stirring. Solid was filtered off and rinsed with DCM (300 mL). The organic layer was separated, washed with water (50 mL), dried over Na₂SO₄, filtered and concentrated under reduced pressure to give the title compound (2.9 g, 83%). LC-MS (M+H)⁺=252.0.

### Step 5: 1-(7′-bromo-4′-hydroxy-3′,4′-dihydro-2′H-spiro[cyclopropane-1,1′-isoquinolin]-2′-yl)ethan-1-one

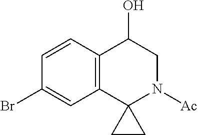 [SMILES: CC(=O)N1CC(O)c2ccc(Br)cc2C12CC2]

To a mixture of 7′-bromo-2′,3′-dihydro-4′H-spiro[cyclopropane-1,1′-isoquinolin]-4′-one (2.9 g, 11.5 mmol) in DCM (50 mL) was added Et₃N (5 mL, 34.5 mmol) and AcCl (1.2 mL, 17.2 mmol) at −30° C. The mixture was stirred at −30° C. for 0.5 h. The mixture was partitioned between water (20 mL) and DCM (100 mL). The organic layer was washed with brine (20 mL), dried over Na₂SO₄, filtered and concentrated under reduced pressure. The residue was re-dissolved in MeOH (100 mL) and NaBH₄ (437 mg, 11.5 mmol) was added in portions at 0° C. The mixture was stirred at room temperature and concentrated under reduced pressure. The residue was partitioned between EtOAc (200 mL) and water (100 mL). The organic layer was dried over Na₂SO₄, filtered and concentrated under reduced pressure. The residue was purified by silica gel chromatography (PE:EtOAc=3:1) to give the title compound (2.2 g, 64%). LC-MS (M+H)=296.1.

### Step 6: 1-(7′-bromo-4′-((2-hydroxyethyl)amino)-3′,4′-dihydro-2′H-spiro[cyclopropane-1,1′-isoquinolin]-2′-yl)ethan-1-one

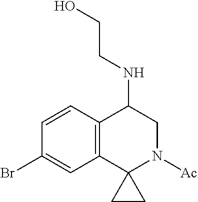 [SMILES: CC(=O)N1CC(NCCO)c2ccc(Br)cc2C12CC2]

To a mixture of 1-(7′-bromo-4′-hydroxy-3′,4′-dihydro-2′H-spiro[cyclopropane-1,1′-isoquinolin]-2′-yl)ethan-1-one (2.2 g, 7.4 mmol) in DCM (100 mL) was added Et₃N ((2.1 mL, 14.8 mmol) and MsCl (0.86 ml, 11.2 mmol) at room temperature and the mixture was stirred at room temperature for 1 h. The mixture was diluted with DCM (100 mL), washed with water (50 mL), dried over Na₂SO₄, filtered and concentrated under reduced pressure. The residue was re-dissolved in DMF (16 mL) followed by addition of 2-aminoethan-1-ol (4 mL). The mixture was stirred at 80° C. for 3 h and cooled to room temperature. The mixture was partitioned between DCM (200 mL) and water (100 mL). The organic layer was dried over Na₂SO₄, filtered and concentrated under reduced pressure. The residue was purified by silica gel chromatography (DCM:MeOH=40:1) to give the title compound (1.6 g, 64%). LC-MS (M+H)=339.2.

### Step 7: 2-((7′-bromo-3′,4′-dihydro-2′H-spiro[cyclopropane-1,1′-isoquinolin]-4′-yl)amino)ethan-1-ol

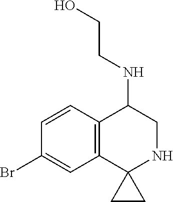 [SMILES: OCCNC1CNC2(CC2)c2cc(Br)ccc21]

To a mixture of 1-(7′-bromo-4′-((2-hydroxyethyl)amino)-3′,4′-dihydro-2′H-spiro[cyclopropane-1,1′-isoquinolin]-2′-yl)ethan-1-one (1.6 g, 5.38 mmol) in water (3 mL) and dioxane (6 mL) was added concentrated hydrochloric acid (3 mL) and the mixture was stirred at 100° C. for 2 h. The mixture was cooled to room temperature and concentrated under reduced pressure. The residue was treated with DCM (10 mL) and methanolic ammonia (7 M, 4 mL). The mixture was concentrated under reduced pressure. The residue was purified by silica gel chromatograph (DCM:MeOH=20:1) to give the title compound (1.2 g, 85%). LC-MS (M+H)⁺=297.1.

### Step 8: tert-butyl 8′-bromo-3′,4′-dihydrospiro[cyclopropane-1,6′-[1,5]methanobenzo[f][1,4]diazocine]-2′(1′H)-carboxylate

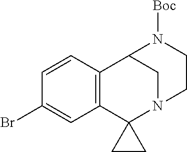 [SMILES: CC(C)(C)OC(=O)N1CCN2CC1c1ccc(Br)cc1C21CC1]

To a mixture of 2-((7′-bromo-3′,4′-dihydro-2′H-spiro[cyclopropane-1,1′-isoquinolin]-4′-yl)amino)ethan-1-ol (500 mg, 1.7 mmol) in THF (20 mL) was added SOCl₂ (1.2 mL, 17 mmol) at room temperature and the mixture was stirred at 60° C. for 2 h. The mixture was cooled to room temperature and concentrated under reduced pressure. The residue was re-dissolved in DMF (10 ml) followed by addition of K₂CO₃ (2.3 g, 17 mmol). The mixture was stirred at 80° C. for 2 h and cooled to room temperature. Boc₂O (741 mg, 3.4 mmol) was added and the mixture was stirred at room temperature for 1 h. The mixture was partitioned between DCM (100 mL) and water (50 mL). The organic layer was dried over Na₂SO₄, filtered and concentrated under reduced pressure. The residue was purified by silica gel chromatography (PE:EtOAc=1:1) to give the title compound (130 mg, 20%) LC-MS (M+H)⁺=379.2.

### Step 9: tert-butyl 8′-(4,4,5,5-tetramethyl-1,3,2-dioxaborolan-2-yl)-3′,4′-dihydrospiro[cyclopropane-1,6′-[1,5]methanobenzo[f][1,4]diazocine]-2′(1′H)-carboxylate

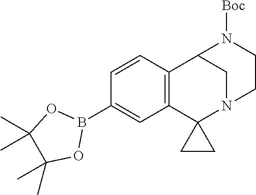 [SMILES: CC(C)(C)OC(=O)N1CCN2CC1c1ccc(B3OC(C)(C)C(C)(C)O3)cc1C21CC1]

To a mixture of tert-butyl 8′-bromo-3′,4′-dihydrospiro[cyclopropane-1,6′-[1,5]methanobenzo[f][1,4]diazocine]-2′(1′H)-carboxylate (80 mg, 0.21 mmol) and BPD (80 mg, 0.31 mmol) in dioxane (5 mL) was added Pd(dppf)Cl₂ (17 mg, 0.021 mmol) and KOAc (61 mg, 0.63 mmol) under nitrogen. The mixture was stirred at 100° C. for 12 h and cooled to room temperature. The mixture was partitioned between EtOAc (30 mL) and water (20 mL). The organic layer was dried over Na₂SO₄, filtered and concentrated under reduced pressure. The residue was purified by silica gel chromatography (PE:EtOAc=1:1) to give the title compound (40 mg, 44%). LC-MS (M+H)⁺=427.2.

### Step 10: tert-butyl 8′-(trifluoromethyl)-3′,4′-dihydrospiro[cyclopropane-1,6′-[1,5]methanobenzo[f][1,4]diazocine]-2′(1′H)-carboxylate

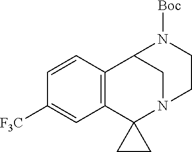 [SMILES: CC(C)(C)OC(=O)N1CCN2CC1c1ccc(C(F)(F)F)cc1C21CC1]

To a mixture of tert-butyl 8′-(4,4,5,5-tetramethyl-1,3,2-dioxaborolan-2-yl)-3′,4′-dihydrospiro[cyclopropane-1,6′-[1,5]methanobenzo[f][1,4]diazocine]-2′(1′H)-carboxylate (80 mg, 0.19 mmol) in DMF (5 mL), was added (1,10-phenanthroline)(trifluoromethyl)copper(I) (88 mg, 0.28 mmol). The mixture was stirred at 60° C. for 2 h and cooled to room temperature. The mixture was partitioned between EtOAc (100 mL) and water (30 mL). The organic layer was dried over Na₂SO₄, filtered and concentrated under reduced pressure. The residue product was purified by silica gel chromatography (PE:EtOAc=1:1) to give the title compound (30 mg, 43%). LC-MS (M+H)⁺=369.2.

### Step 11: 8′-(trifluoromethyl)-1′,2′,3′,4′-tetrahydrospiro[cyclopropane-1,6′-[1,5]methanobenzo[f][1,4]diazocine]dihydrochloride

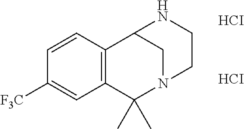

A mixture of tert-butyl 8′-(trifluoromethyl)-3′,4′-dihydrospiro[cyclopropane-1,6′-[1,5]methanobenzo[f][1,4]diazocine]-2′(1′H)-carboxylate (30 mg, 0.082 mmol) in HCl (4.0 M in dioxane, 4 mL) was stirred at room temperature for 4 h. The mixture was concentrated under reduced pressure to give the title compound (28 mg, 100%).

### Step 12: (5-amino-1-((2-(trimethylsilyl)ethoxy)methyl)-6,8-dihydro-1H-furo[3,4-d]pyrrolo[3,2-b]pyridin-2-yl)(8′-(trifluoromethyl)-3′,4′-dihydrospiro[cyclopropane-1,6′-[1,5]methanobenzo[f][1,4]diazocin]-2′(1′H)-yl)methanone

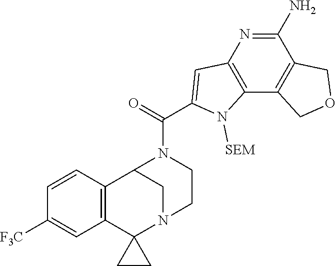 [SMILES: C[Si](C)(C)CCOCn1c(C(=O)N2CCN3CC2c2ccc(C(F)(F)F)cc2C32CC2)cc2nc(N)c3c(c21)COC3]

To a mixture of 5-amino-1-((2-(trimethylsilyl)ethoxy)methyl)-6,8-dihydro-1H-furo[3,4-d]pyrrolo[3,2-b]pyridine-2-carboxylic acid (28 mg, 0.080 mmol) and 8′-(trifluoromethyl)-1′,2′,3′,4′-tetrahydrospiro[cyclopropane-1,6′-[1,5]methanobenzo[f][1,4]diazocine]dihydrochloride (28 mg, 0.082 mmol) in THF (10 mL) was added BOPCl (30 mg, 0.12 mmol) and DIPEA (51 mg, 0.40 mmol). The mixture was stirred at 60° C. for 2 h and cooled to room temperature. The mixture was partitioned between water (10 mL) and EtOAc (50 mL). The organic layer was washed with brine (10 mL), dried over Na₂SO₄, filtered and concentrated under reduced pressure. The residue was purified by prep-TLC to give the title compound (20 mg, 41%). LC-MS (M+H)⁺=600.2.

### Step 13: (5-amino-6,8-dihydro-1H-furo[3,4-d]pyrrolo[3,2-b]pyridin-2-yl)((1′R,5′R)-8′-(trifluoromethyl)-3′,4′-dihydrospiro[cyclopropane-1,6′-[1,5]methanobenzo[f][1,4]diazocin]-2′(1′H)-yl)methanone & (5-amino-6,8-dihydro-1H-furo[3,4-d]pyrrolo[3,2-b]pyridin-2-yl)((1'S,5'S)-8′-(trifluoromethyl)-3′,4′-dihydrospiro[cyclopropane-1,6′-[1,5]methanobenzo[f][1,4]diazocin]-2′(1′H)-yl)methanone

A mixture of (5-amino-1-((2-(trimethylsilyl)ethoxy)methyl)-6,8-dihydro-1H-furo[3,4-d]pyrrolo[3,2-b]pyridin-2-yl)(8′-(trifluoromethyl)-3′,4′-dihydrospiro[cyclopropane-1,6′-[1,5]methanobenzo[f][1,4]diazocin]-2′(1′H)-yl)methanone (20 mg, 0.033 mmol) in TFA (4 mL) was stirred at room temperature for 0.5 h and concentrated under reduced pressure. The mixture was taken in THF (4 mL) and ammonium hydroxide (28%, 2 mL). The mixture was stirred at room temperature for 2 h and concentrated under reduced pressure. The residue was purified by prep-HPLC followed by chiral SFC to give Example 62 (5 mg, 32%) and Example 63 (5 mg, 32%).

Analytical chiral-SFC condition as below. Column: Lux Cellulose-3; Column size: 4.6×100 mm, m; Mobile phase: 4 mM methanolic NH₃:CO₂, 1:9 to 1:1 in 3 min, 1:1 for 2 min, 1:1 to 1:9 in 0.1 min, 1:9 for 1.9 min; Flow: 2.0 mL/min; Temperature: 35° C.; back pressure: 1500 psi.

Example 62: Analytical SFC tR=2.54 min. ¹H NMR (400 MHz, DMSO-d6, 80° C.) δ11.29 (s, 1H), 7.43 (d, J=7.8 Hz, 1H), 7.32-7.17 (m, 1H), 7.04-6.93 (m, 1H), 6.54 (s, 1H), 5.67-5.57 (m, 1H), 5.28 (s, 2H), 5.12 (s, 2H), 4.91 (s, 2H), 3.87 (d, J=12.7 Hz, 1H), 3.41 (d, J=13.0 Hz, 1H), 3.30 (d, J=13.2 Hz, 1H), 3.09-2.80 (m, 3H), 1.68-1.55 (m, 1H), 1.34-1.15 (m, 2H), 0.87-0.74 (m, 1H). LC-MS (M+H)⁺=470.2.

Example 63: Analytical SFC tR=2.80 min. ¹H NMR (400 MHz, DMSO-d6, 80° C.) δ11.29 (s, 1H), 7.43 (d, J=7.8 Hz, 1H), 7.32-7.17 (m, 1H), 7.04-6.93 (m, 1H), 6.54 (s, 1H), 5.67-5.57 (m, 1H), 5.28 (s, 2H), 5.12 (s, 2H), 4.91 (s, 2H), 3.87 (d, J=12.7 Hz, 1H), 3.41 (d, J=13.0 Hz, 1H), 3.30 (d, J=13.2 Hz, 1H), 3.09-2.80 (m, 3H), 1.68-1.55 (m, 1H), 1.34-1.15 (m, 2H), 0.87-0.74 (m, 1H). LC-MS (M+H)⁺=470.2.

### Example 64 & Example 65: (5-amino-6,8-dihydro-1H-furo[3,4-d]pyrrolo[3,2-b]pyridin-2-yl)((2S,6R)-9-(trifluoromethyl)-3,4-dihydro-2H-2,6-methanopyrido[2,3-b][1,5]oxazocin-5(6H)-yl)methanone & (5-amino-6,8-dihydro-1H-furo[3,4-d]pyrrolo[3,2-b]pyridin-2-yl)((2R,6S)-9-(trifluoromethyl)-3,4-dihydro-2H-2,6-methanopyrido[2,3-b][1,5]oxazocin-5(6H)-yl)methanone

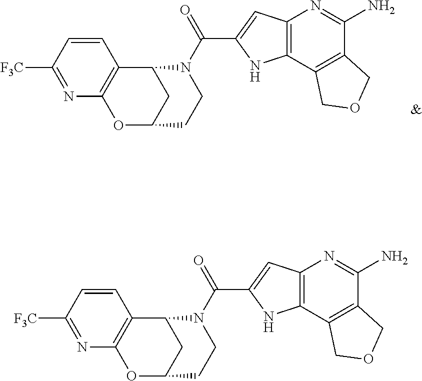 [SMILES: Nc1nc2cc(C(=O)N3CC[C@H]4C[C@@H]3c3ccc(C(F)(F)F)nc3O4)[nH]c2c2c1COC2 ; Nc1nc2cc(C(=O)N3CC[C@H]4C[C@@H]3c3ccc(C(F)(F)F)nc3O4)[nH]c2c2c1COC2]

### Step 1: 2-(2-chloro-6-(trifluoromethyl)pyridin-3-yl)piperidin-4-one

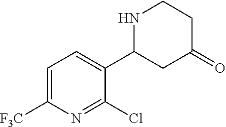 [SMILES: O=C1CCNC(c2ccc(C(F)(F)F)nc2Cl)C1]

A mixture of 2-chloro-6-(trifluoromethyl)nicotinaldehyde (5.0 g, 23.8 mmol), 4-aminobutan-2-one hydrochloride (2.95 g, 23.8 mmol), L-proline (0.60 g, 5.3 mmol), Et₃N (2.9 g, 28.6 mmol) and MgSO₄ (3.7 g, 31 mmol) in EtOH (100 mL) was stirred at room temperature for 24 h. Solid was filtered off and the filtrate was concentrated under reduced pressure. The residue was taken in EtOAc (100 mL), and the mixture was washed with saturated NaHCO₃ (50 mL) and brine (50 mL). The organic layer was dried over Na₂SO₄, filtered and concentrated under reduced pressure. The residue was purified by silica gel chromatography (DCM/MeOH=20/1) to give the title compound (1.2 g, 18%). LC-MS (M+H)⁺=279.1.

### Step 2: tert-butyl 2-(2-chloro-6-(trifluoromethyl)pyridin-3-yl)-4-oxopiperidine-1-carboxylate

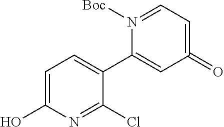

To a mixture of 2-(2-chloro-6-(trifluoromethyl)pyridin-3-yl)piperidin-4-one (1.2 g, 4.3 mmol) and Et₃N (1.3 g, 12.9 mmol) in DCM (20 mL) was added Boc₂O (1.9 g, 8.6 mmol). The mixture was stirred at room temperature for 16 h. The mixture was diluted with DCM (50 mL), washed with saturated NaHCO₃ (50 mL) and brine (50 mL). The organic layer was dried over Na₂SO₄, filtered and concentrated under reduced pressure. The residue was purified by silica gel chromatography (PE/EtOAc=2/1) to give the title compound (1.38 g, 85%). LC-MS (M+H)⁺=379.1.

### Step 3: tert-butyl 2-(2-chloro-6-(trifluoromethyl)pyridin-3-yl)-4-hydroxypiperidine-1-carboxylate

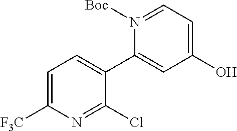

To a mixture of tert-butyl 2-(2-chloro-6-(trifluoromethyl)pyridin-3-yl)-4-oxopiperidine-1-carboxylate (1.38 g, 3.65 mmol) in MeOH (20 mL) was added NaBH₄ (277 mg, 7.3 mmol). The mixture was stirred at room temperature for 3 h and then concentrated under reduced pressure. The residue was partitioned between ether (50 mL) and saturated NaHCO₃ (50 mL). The aqueous layer was back extracted with EtOAc (50 mL×3). The combined organic layer was concentrated under reduced pressure and purified by silica gel chromatography (PE/EtOAc=1/1) to give the title compound (0.90 g, 65%). LC-MS (M+H)⁺=381.1.

### Step 4: tert-butyl 9-(trifluoromethyl)-3,4-dihydro-2H-2,6-methanopyrido[2,3-b][1,5]oxazocine-5(6H)-carboxylate

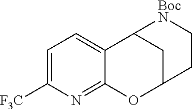 [SMILES: CC(C)(C)OC(=O)N1CCC2CC1c1ccc(C(F)(F)F)nc1O2]

A mixture of tert-butyl 2-(2-chloro-6-(trifluoromethyl)pyridin-3-yl)-4-hydroxypiperidine-1-carboxylate (0.80 g, 2.1 mmol) and NaH (60%, 160 mg, 4.0 mmol) in anhydrous THF (20 mL) was stirred at 80° C. under nitrogen for 3 h. The mixture was cooled to room temperature and diluted with water (50 mL). The mixture was extracted with EtOAc (50 mL×3). The combined organic layer was concentrated under reduced pressure. The residue was purified by silica gel chromatography (PE/EA=2/1) to give the title compound (0.45 g, 65%). LC-MS (M+H)⁺=345.1.

### Step 5: 9-(trifluoromethyl)-3,4,5,6-tetrahydro-2H-2,6-methanopyrido[2,3-b][1,5]oxazocine

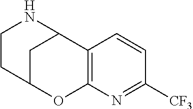 [SMILES: FC(F)(F)c1ccc2c(n1)OC1CCNC2C1]

A mixture of tert-butyl 9-(trifluoromethyl)-3,4-dihydro-2H-2,6-methanopyrido[2,3-b][1,5]oxazocine-5(6H)-carboxylate (0.45 g, 1.3 mmol) in HCl (4 M in dioxane, 10 mL) was stirred at room temperature for 2 h. The mixture was concentrated under reduced pressure. The residue was taken saturated in saturated NaHCO₃ (50 mL). The mixture was extracted with DCM (50 mL×2). The combined organic layer was washed with brine (50 mL), dried over Na₂SO₄, filtered and concentrated under reduced pressure to give the title compound (0.31 g, 96%). LC-MS (M+H)=245.1.

### Step 6: (5-amino-1-((2-(trimethylsilyl)ethoxy)methyl)-6,8-dihydro-1H-furo[3,4-d]pyrrolo[3,2-b]pyridin-2-yl)(9-(trifluoromethyl)-3,4-dihydro-2H-2,6-methanopyrido[2,3-b][1,5]oxazocin-5(6H)-yl)methanone

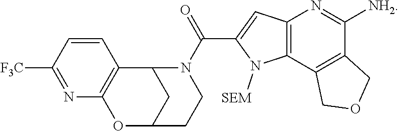 [SMILES: C[Si](C)(C)CCOCn1c(C(=O)N2CCC3CC2c2ccc(C(F)(F)F)nc2O3)cc2nc(N)c3c(c21)COC3]

A mixture of 9-(trifluoromethyl)-3,4,5,6-tetrahydro-2H-2,6-methanopyrido[2,3-b][1,5]oxazocine (60 mg, 0.24 mmol), 5-amino-1-((2-(trimethylsilyl)ethoxy)methyl)-6,8-dihydro-1H-furo[3,4-d]pyrrolo[3,2-b]pyridine-2-carboxylic acid (86 mg, 0.24 mmol), BOPCl (94 mg, 0.37 mmol) and DIPEA (258 mg, 0.49 mmol) in THF (10 mL) was stirred at 60° C. for 16 h. The mixture was cooled to room temperature and partitioned between water (50 mL) and EtOAc (50 mL). The organic layer was dried over Na₂SO₄, filtered and concentrated under reduced pressure. The residue was purified by silica gel chromatography (DCM:MeOH=20:1) to give the title compound (122 mg, 89%). LC-MS (M+H)⁺=576.2.

### Step 7: (5-amino-6,8-dihydro-1H-furo[3,4-d]pyrrolo[3,2-b]pyridin-2-yl)((2S,6R)-9-(trifluoromethyl)-3,4-dihydro-2H-2,6-methanopyrido[2,3-b][1,5]oxazocin-5(6H)-yl)methanone & (5-amino-6,8-dihydro-1H-furo[3,4-d]pyrrolo[3,2-b]pyridin-2-yl)((2R,6S)-9-(trifluoromethyl)-3,4-dihydro-2H-2,6-methanopyrido[2,3-b][1,5]oxazocin-5(6H)-yl)methanone

A mixture of (5-amino-1-((2-(trimethylsilyl)ethoxy)methyl)-6,8-dihydro-1H-furo[3,4-d]pyrrolo[3,2-b]pyridin-2-yl)(9-(trifluoromethyl)-3,4-dihydro-2H-2,6-methanopyrido[2,3-b][1,5]oxazocin-5(6H)-yl)methanone (122 mg, 0.21 mmol) in DCM (5 mL) and TFA (5 mL) was stirred at room temperature for 2 h. The mixture was concentrated under reduced pressure. The residue was taken in MeOH (10 mL) and ammonium hydroxide (28%, 2 mL). The mixture was stirred at room temperature for 1 h and concentrated under reduced pressure. The residue was purified by prep-HPLC followed by chiral SFC to give Example 64 (28 mg, 30%) and Example 65 (26 mg, 28%).

Analytical chiral-SFC condition as below. Column: Lux Cellulose-3; Column size: 4.6×100 mm, 5 μm; Mobile phase: 4 mM methanolic NH₃:CO₂, 1:9 to 1:1 in 3 min, 1:1 for 2 min, 1:1 to 1:9 in 0.1 min, 1:9 for 1.9 min; Flow: 2.0 mL/min; Temperature: 35° C.; back pressure: 1500 psi.

Example 64: Analytical SFC tR=3.41 min. ¹H NMR (400 MHz, DMSO-d6, 80° C.) δ11.31 (s, 1H), 7.77 (d, J=7.4 Hz, 1H), 7.38 (d, J=7.5 Hz, 1H), 6.59 (s, 1H), 5.84 (s, 1H), 5.30 (s, 2H), 5.12 (s, 2H), 5.01 (s, 1H), 4.91 (s, 2H), 4.28-4.11 (m, 1H), 2.93-2.79 (m, 1H), 2.31-2.21 (m, 1H), 2.16-1.92 (m, 3H). LC-MS (M+H)⁺=446.2.

Example 65: Analytical SFC tR=4.23 min. ¹H NMR (400 MHz, DMSO-d6, 80° C.) δ11.31 (s, 1H), 7.77 (d, J=7.4 Hz, 1H), 7.38 (d, J=7.5 Hz, 1H), 6.59 (s, 1H), 5.84 (s, 1H), 5.30 (s, 2H), 5.12 (s, 2H), 5.01 (s, 1H), 4.91 (s, 2H), 4.28-4.11 (m, 1H), 2.93-2.79 (m, 1H), 2.31-2.21 (m, 1H), 2.16-1.92 (m, 3H). LC-MS (M+H)⁺=446.2.

### Example 66 & Example 67: (5-amino-6,8-dihydro-1H-furo[3,4-d]pyrrolo[3,2-b]pyridin-2-yl)((1R,5R,6R)-8-bromo-6-methyl-1,3,4,6-tetrahydro-2H-1,5-methanobenzo[f][1,4]diazocin-2-yl)methanone & (5-amino-6,8-dihydro-1H-furo[3,4-d]pyrrolo[3,2-b]pyridin-2-yl)((1S,5S,6S)-8-bromo-6-methyl-1,3,4,6-tetrahydro-2H-1,5-methanobenzo[f][1,4]diazocin-2-yl)methanone

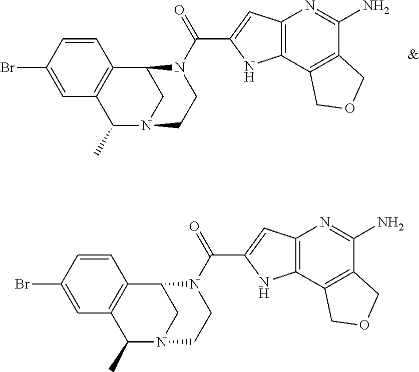 [SMILES: C[C@@H]1c2cc(Br)ccc2[C@@H]2C[N@@]1CCN2C(=O)c1cc2nc(N)c3c(c2[nH]1)COC3 ; C[C@H]1c2cc(Br)ccc2[C@H]2C[N@]1CCN2C(=O)c1cc2nc(N)c3c(c2[nH]1)COC3]

### Step 1: tert-butyl 7-bromo-4-hydroxy-1-methyl-3,4-dihydroisoquinoline-2(1H)-carboxylate

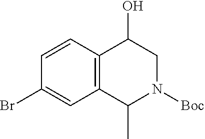 [SMILES: CC1c2cc(Br)ccc2C(O)CN1C(=O)OC(C)(C)C]

To a mixture of tert-butyl 7-bromo-1-methyl-4-oxo-3,4-dihydroisoquinoline-2(1H)-carboxylate (25 g, 73 mmol) in MeOH (150 mL) was added NaBH₄ (8.3 g, 220 mmol) in portions at 0° C. The mixture was stirred at 0° C. for 1 h. Most of MeOH was removed under reduced pressure. The residue was diluted with water (250 mL) and extracted with EtOAc (150 mL×3). The combined organic layer was washed with brine (50 mL×3), dried over Na₂SO₄, filtered and concentrated under reduced pressure. The residue was purified by silica gel chromatography (PE/EtOAc=10/1 to 4/1). The solid obtained was triturated in PE/MTBE (2:1 v/v, 30 mL). The solid (cis-isomers) was filtered off and rinsed with PE/MTBE (2:1 v/v, 10 mL). The mother liquor was collected and concentrated under reduced pressure. The residue was purified again by silica gel chromatography (PE/EtOAc=5/1) to give the title compound (1.5 g, 6%). LC-MS (M+H)⁺=342.2.

### Step 2: tert-butyl 7-bromo-4-((2-hydroxyethyl)amino)-1-methyl-3,4-dihydroisoquinoline-2(1H)-carboxylate

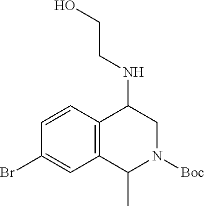 [SMILES: CC1c2cc(Br)ccc2C(NCCO)CN1C(=O)OC(C)(C)C]

To a mixture of tert-butyl 7-bromo-4-hydroxy-1-methyl-3,4-dihydroisoquinoline-2(1H)-carboxylate (1.5 g, 4.4 mmol) in DCM (100 mL) was added Et₃N (1.9 mL, 13 mmol) and MsCl (0.44 mL, 5.7 mmol) at room temperature and the mixture was stirred for 1 h. The mixture was partitioned between DCM (100 mL) and water (50 mL), dried over Na₂SO₄, filtered and concentrated under reduced pressure. The residue was re-dissolved in DMF (6 mL) followed by addition of 2-aminoethan-1-ol (3 mL). The mixture was stirred at 80° C. for 3 h and then cooled to room temperature. The residue was partitioned between EtOAc (200 mL) and water (100 mL). The organic layer was dried over Na₂SO₄, filtered and concentrated under reduced pressure. The residue was purified by silica gel chromatography (PE:EtOAc=1:1 to 0:1) to give the title compound (800 mg, 47%) LC-MS (M+H)⁺=385.2.

### Step 2: rac-tert-butyl (1R,5R,6R)-8-bromo-6-methyl-1,3,4,6-tetrahydro-2H-1,5-methanobenzo[f][1,4]diazocine-2-carboxylate & rac-tert-butyl (1R,5R,6S)-8-bromo-6-methyl-1,3,4,6-tetrahydro-2H-1,5-methanobenzo[f][1,4]diazocine-2-carboxylate

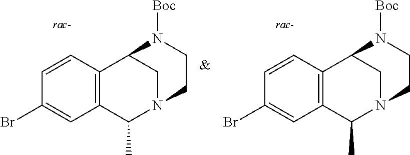 [SMILES: C[C@@H]1c2cc(Br)ccc2[C@@H]2C[N@@]1CCN2C(=O)OC(C)(C)C ; C[C@H]1c2cc(Br)ccc2[C@@H]2C[N@@]1CCN2C(=O)OC(C)(C)C]

To a mixture of tert-butyl 7-bromo-4-((2-hydroxyethyl)amino)-1-methyl-3,4-dihydroisoquinoline-2(1H)-carboxylate (800 mg, 2.1 mmol) in THF (20 mL) was added SOC12 (0.70 mL, 10 mmol) at room temperature. The mixture was stirred at 60° C. for 2 h and cooled to room temperature. The mixture was concentrated under reduced pressure. The residue was treated with HCl (4 N in dioxane, 10 mL). The mixture was stirred at room temperature for 2 h and concentrated under reduced pressure. The crude was re-dissolved in DMF (10 mL) followed by addition of K₂CO₃ (2.7 g, 20 mmol). The mixture was stirred at 80° C. for 2 h and cooled to room temperature. Boc₂O (872 mg, 4.0 mmol) was added and the mixture was stirred at room temperature for 1 h. The mixture was partitioned between DCM (100 mL) and water (50 mL), and the organic layer was dried over Na₂SO₄, filtered and concentrated under reduced pressure. The residue was purified by prep-TLC (PE:EtOAc=1:2) to give the title compounds.

rac-tert-butyl (1R,5R,6R)-8-bromo-6-methyl-1,3,4,6-tetrahydro-2H-1,5-methanobenzo[f][1,4]diazocine-2-carboxylate (250 mg, 32%). LC-MS (M+H)⁺=367.2.

rac-tert-butyl (1R,5R,6S)-8-bromo-6-methyl-1,3,4,6-tetrahydro-2H-1,5-methanobenzo[f][1,4]diazocine-2-carboxylate (220 mg, 28%). LC-MS (M+H)⁺=367.2

### Step 3: rac-(1R,5R,6R)-8-bromo-6-methyl-1,3,4,6-tetrahydro-2H-1,5-methanobenzo[f][1,4]diazocine dihydrochloride

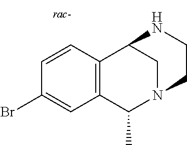

A mixture of rac-tert-butyl (1R,5R,6R)-8-bromo-6-methyl-1,3,4,6-tetrahydro-2H-1,5-methanobenzo[f][1,4]diazocine-2-carboxylate (250 mg, 0.68 mmol) in HCl (4 M in dioxane, 5 mL) was stirred at room temperature for 2 h. The mixture was concentrated under reduced pressure to give the title compound (233 mg, 100%).

### Step 4: rac-(5-amino-1-((2-(trimethylsilyl)ethoxy)methyl)-6,8-dihydro-1H-furo[3,4-d]pyrrolo[3,2-b]pyridin-2-yl)((1R,5R,6R)-8-bromo-6-methyl-1,3,4,6-tetrahydro-2H-1,5-methanobenzo[f][1,4]diazocin-2-yl)methanone

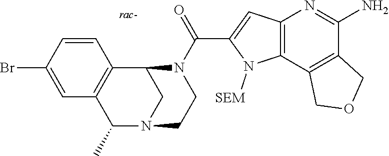 [SMILES: C[C@@H]1c2cc(Br)ccc2[C@@H]2C[N@@]1CCN2C(=O)c1cc2nc(N)c3c(c2n1COCC[Si](C)(C)C)COC3]

To a mixture of 5-amino-1-((2-(trimethylsilyl)ethoxy)methyl)-6,8-dihydro-1H-furo[3,4-d]pyrrolo[3,2-b]pyridine-2-carboxylic acid (237 mg, 0.68 mmol) and rac-(1R,5R,6R)-8-bromo-6-methyl-1,3,4,6-tetrahydro-2H-1,5-methanobenzo[f][1,4]diazocine dihydrochloride (233 mg, 0.68 mmol) in THF (20 mL) was added BOPCl (260 mg, 1.02 mmol) and DIPEA (0.60 mL, 3.4 mmol). The mixture was stirred at 60° C. for 2 h and then cooled to room temperature. The mixture was partitioned between water (10 mL) and EtOAc (50 mL). The organic layer was washed with brine (10 mL), dried over Na₂SO₄, filtered and concentrated under reduced pressure. The residue was purified by prep-TLC (DCM:MeOH=20:1) to give the title compound (300 mg, 74%). LC-MS (M+H)⁺=598.1.

### Step 5: (5-amino-6,8-dihydro-1H-furo[3,4-d]pyrrolo[3,2-b]pyridin-2-yl)((1R,5R,6R)-8-bromo-6-methyl-1,3,4,6-tetrahydro-2H-1,5-methanobenzo[f][1,4]diazocin-2-yl)methanone & (5-amino-6,8-dihydro-1H-furo[3,4-d]pyrrolo[3,2-b]pyridin-2-yl)((1S,5S,6S)-8-bromo-6-methyl-1,3,4,6-tetrahydro-2H-1,5-methanobenzo[f][1,4]diazocin-2-yl)methanone

A mixture of rac-(5-amino-1-((2-(trimethylsilyl)ethoxy)methyl)-6,8-dihydro-1H-furo[3,4-d]pyrrolo[3,2-b]pyridin-2-yl)((1R,5R,6R)-8-bromo-6-methyl-1,3,4,6-tetrahydro-2H-1,5-methanobenzo[f][1,4]diazocin-2-yl)methanone (300 mg, 0.50 mmol) in TFA (10 mL) was stirred at room temperature for 0.5 h and concentrated under reduced pressure. The mixture was taken in THF (10 mL) and ammonium hydroxide (28%, 3 mL). The mixture was stirred at room temperature for 2 h and concentrated under reduced pressure. The residue was purified by prep-HPLC followed by chiral SFC to give Example 66 (89 mg, 38%) and Example 67 (85 mg, 36%).

Analytical chiral-SFC condition as below. Column: YMC Cellulose-C; Column size: 4.6×100 mm, 5 μm; Mobile phase: 4 mM methanolic NH₃:CO₂, 45:55 isocratic; Flow: 2.0 mL/min; Temperature: 35° C.; back pressure: 1500 psi.

Example 66: Analytical SFC tR=4.16 min. ¹H NMR (400 MHz, DMSO-d6, 80° C.) δ11.27 (s, 1H), 7.42 (d, J=1.8 Hz, 1H), 7.32 (dd, J=8.1, 1.8 Hz, 1H), 7.14-6.88 (m, 1H), 6.50 (s, 1H), 5.42-5.33 (m, 1H), 5.33-5.22 (m, 2H), 5.18-5.07 (m, 2H), 4.96-4.85 (m, 2H), 4.05 (q, J=6.8 Hz, 1H), 3.84-3.73 (m, 1H), 3.26 (d, J=13.6 Hz, 1H), 3.23-3.13 (m, 2H), 3.02-2.95 (m, 1H), 2.94-2.80 (m, 1H), 1.34 (d, J=7.0 Hz, 3H). LC-MS (M+H)⁺=468.2.

Example 67: Analytical SFC tR=6.94 min. ¹H NMR (400 MHz, DMSO-d6, 80° C.) δ11.27 (s, 1H), 7.42 (d, J=1.8 Hz, 1H), 7.32 (dd, J=8.1, 1.8 Hz, 1H), 7.14-6.88 (m, 1H), 6.50 (s, 1H), 5.42-5.33 (m, 1H), 5.33-5.22 (m, 2H), 5.18-5.07 (m, 2H), 4.96-4.85 (m, 2H), 4.05 (q, J=6.8 Hz, 1H), 3.84-3.73 (m, 1H), 3.26 (d, J=13.6 Hz, 1H), 3.23-3.13 (m, 2H), 3.02-2.95 (m, 1H), 2.94-2.80 (m, 1H), 1.34 (d, J=7.0 Hz, 3H). LC-MS (M+H)⁺=468.3.

### Example 68 & Example 69: (5-amino-6,8-dihydro-1H-furo[3,4-d]pyrrolo[3,2-b]pyridin-2-yl)((1R,5R,6S)-8-bromo-6-methyl-1,3,4,6-tetrahydro-2H-1,5-methanobenzo[f][1,4]diazocin-2-yl)methanone & (5-amino-6,8-dihydro-1H-furo[3,4-d]pyrrolo[3,2-b]pyridin-2-yl)((1S,5S,6R)-8-bromo-6-methyl-1,3,4,6-tetrahydro-2H-1,5-methanobenzo[f][1,4]diazocin-2-yl)methanone

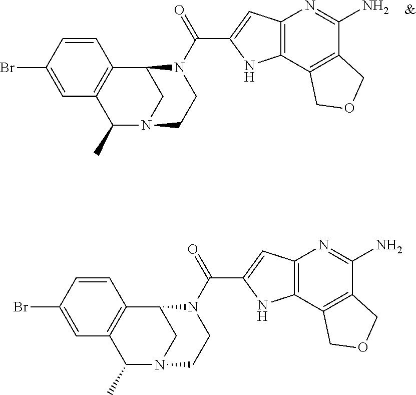 [SMILES: C[C@H]1c2cc(Br)ccc2[C@@H]2C[N@@]1CCN2C(=O)c1cc2nc(N)c3c(c2[nH]1)COC3 ; C[C@@H]1c2cc(Br)ccc2[C@H]2C[N@]1CCN2C(=O)c1cc2nc(N)c3c(c2[nH]1)COC3]

### Step 1: rac-(1R,5R,6S)-8-bromo-6-methyl-1,3,4,6-tetrahydro-2H-1,5-methanobenzo[f][1,4]diazocine dihydrochloride

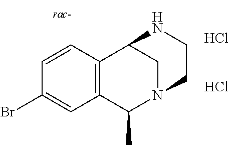

A mixture of rac-tert-butyl (1R,5R,6S)-8-bromo-6-methyl-1,3,4,6-tetrahydro-2H-1,5-methanobenzo[f][1,4]diazocine-2-carboxylate (220 mg, 0.60 mmol) in HCl (4 M in dioxane, 5 mL) was stirred at room temperature for 2 h. The mixture was concentrated under reduced pressure to give the title compound (204 mg, 100%).

### Step 2: rac-(5-amino-1-((2-(trimethylsilyl)ethoxy)methyl)-6,8-dihydro-1H-furo[3,4-d]pyrrolo[3,2-b]pyridin-2-yl)((1R,5R,6S)-8-bromo-6-methyl-1,3,4,6-tetrahydro-2H-1,5-methanobenzo[f][1,4]diazocin-2-yl)methanone

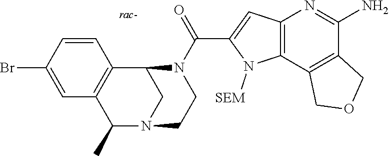 [SMILES: C[C@H]1c2cc(Br)ccc2[C@@H]2C[N@@]1CCN2C(=O)c1cc2nc(N)c3c(c2n1COCC[Si](C)(C)C)COC3]

To a mixture of 5-amino-1-((2-(trimethylsilyl)ethoxy)methyl)-6,8-dihydro-1H-furo[3,4-d]pyrrolo[3,2-b]pyridine-2-carboxylic acid (209 mg, 0.60 mmol) and rac-(1R,5R,6S)-8-bromo-6-methyl-1,3,4,6-tetrahydro-2H-1,5-methanobenzo[f][1,4]diazocine dihydrochloride (204 mg, 0.60 mmol) in THF (20 mL) was added BOPCl (228 mg, 0.90 mmol) and DIPEA (0.50 mL, 3.0 mmol). The mixture was stirred at 60° C. for 2 h and then cooled to room temperature. The mixture was partitioned between water (10 mL) and EtOAc (50 mL). The organic layer was washed with brine (10 mL), dried over Na₂SO₄, filtered and concentrated under reduced pressure. The residue was purified by prep-TLC (DCM:MeOH=20:1) to give the title compound (250 mg, 70%). LC-MS (M+H)⁺=598.1.

### Step 3: (5-amino-6,8-dihydro-1H-furo[3,4-d]pyrrolo[3,2-b]pyridin-2-yl)((1R,5R,6S)-8-bromo-6-methyl-1,3,4,6-tetrahydro-2H-1,5-methanobenzo[f][1,4]diazocin-2-yl)methanone & (5-amino-6,8-dihydro-1H-furo[3,4-d]pyrrolo[3,2-b]pyridin-2-yl)((1S,5S,6R)-8-bromo-6-methyl-1,3,4,6-tetrahydro-2H-1,5-methanobenzo[f][1,4]diazocin-2-yl)methanone

A mixture of rac-(5-amino-1-((2-(trimethylsilyl)ethoxy)methyl)-6,8-dihydro-1H-furo[3,4-d]pyrrolo[3,2-b]pyridin-2-yl)((1R,5R,6S)-8-bromo-6-methyl-1,3,4,6-tetrahydro-2H-1,5-methanobenzo[f][1,4]diazocin-2-yl)methanone (250 mg, 0.42 mmol) in TFA (10 mL) was stirred at room temperature for 0.5 h and concentrated under reduced pressure. The mixture was taken in THF (10 mL) and ammonium hydroxide (28%, 3 mL). The mixture was stirred at room temperature for 2 h and concentrated under reduced pressure. The residue was purified by prep-HPLC followed by chiral SFC to give Example 68 (58 mg, 30%) and Example 69 (59 mg, 30%).

Analytical chiral-SFC condition as below. Column: YMC Cellulose-C; Column size: 4.6×100 mm, 5 μm; Mobile phase: 4 mM methanolic NH₃:CO₂, 45:55 isocratic; Flow: 2.0 mL/min; Temperature: 35° C.; back pressure: 1500 psi.

Example 68: Analytical SFC tR=5.70 min. ¹H NMR (400 MHz, DMSO-d6, 80° C.) δ11.27 (s, 1H), 7.47 (s, 1H), 7.35 (d, J=8.3 Hz, 1H), 7.05-6.90 (m, 1H), 6.51 (s, 1H), 5.43 (s, 1H), 5.28 (s, 2H), 5.12 (s, 2H), 4.91 (d, J=2.5 Hz, 2H), 4.24 (q, J=7.0 Hz, 1H), 3.78 (d, J=9.6 Hz, 1H), 3.42 (d, J=12.7 Hz, 1H), 3.26-3.12 (m, 2H), 3.00-2.88 (m, 1H), 2.74 (s, 1H), 1.53 (d, J=7.1 Hz, 3H). LC-MS (M+H)+=468.2.

Example 69: Analytical SFC tR=10.22 min. ¹H NMR (400 MHz, DMSO-d6, 80° C.) δ11.27 (s, 1H), 7.47 (s, 1H), 7.35 (d, J=8.3 Hz, 1H), 7.05-6.90 (m, 1H), 6.51 (s, 1H), 5.43 (s, 1H), 5.28 (s, 2H), 5.12 (s, 2H), 4.91 (d, J=2.5 Hz, 2H), 4.24 (q, J=7.0 Hz, 1H), 3.78 (d, J=9.6 Hz, 1H), 3.42 (d, J=12.7 Hz, 1H), 3.26-3.12 (m, 2H), 3.00-2.88 (m, 1H), 2.74 (s, 1H), 1.53 (d, J=7.1 Hz, 3H). LC-MS (M+H)+=468.2.

### Example 70 & Example 71: (5-amino-6,8-dihydro-1H-furo[3,4-d]pyrrolo[3,2-b]pyridin-2-yl)((2S,6R)-7-fluoro-2-methyl-9-(trifluoromethyl)-3,4-dihydro-2H-2,6-methanobenzo[b][1,5]oxazocin-5(6H)-yl)methanone & (5-amino-6,8-dihydro-1H-furo[3,4-d]pyrrolo[3,2-b]pyridin-2-yl)((2R,6S)-7-fluoro-2-methyl-9-(trifluoromethyl)-3,4-dihydro-2H-2,6-methanobenzo[b][1,5]oxazocin-5(6H)-yl)methanone

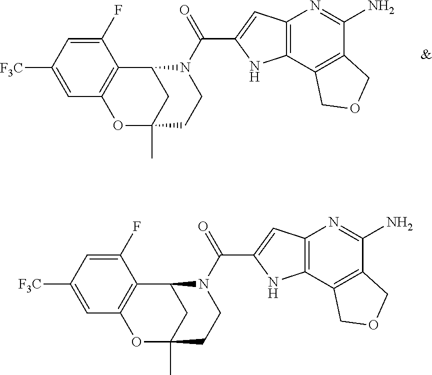 [SMILES: C[C@]12CCN(C(=O)c3cc4nc(N)c5c(c4[nH]3)COC5)[C@H](C1)c1c(F)cc(C(F)(F)F)cc1O2 ; C[C@@]12CCN(C(=O)c3cc4nc(N)c5c(c4[nH]3)COC5)[C@@H](C1)c1c(F)cc(C(F)(F)F)cc1O2]

### Step 1: 2-(2-bromo-6-fluoro-4-(trifluoromethyl)phenyl)piperidin-4-one

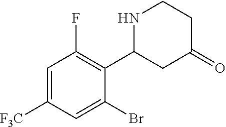 [SMILES: O=C1CCNC(c2c(F)cc(C(F)(F)F)cc2Br)C1]

To a mixture of 2-bromo-6-fluoro-4-(trifluoromethyl)benzaldehyde (5.0 g, 18.5 mmol) in EtOH (70 mL) was added 4-aminobutan-2-one hydrochloride (2.28 g, 18.5 mmol), MgSO₄ (2.89 g, 24.1 mmol), L-proline (468 mg, 4.1 mmol) and Et₃N (2.24 g, 22.2 mmol). The mixture was stirred at room temperature for 16 h then concentrated under reduced pressure. The residue was partitioned between water (300 mL) and EtOAc (300 mL). The aqueous layer was extracted with EtOAc (300 mL) and the combined organic layer was washed with brine (500 mL), dried over Na₂SO₄, filtered and concentrated under reduced pressure. The residue was purified by silica gel chromatograph (PE:EtOAc=1:1) to give the title compound (1.9 g, 30%). LC-MS (M+H)⁺=340.1.

### Step 2: tert-butyl 2-(2-bromo-6-fluoro-4-(trifluoromethyl)phenyl)-4-oxopiperidine-1-carboxylate

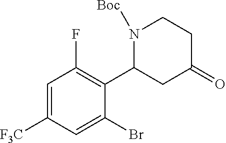 [SMILES: CC(C)(C)OC(=O)N1CCC(=O)CC1c1c(F)cc(C(F)(F)F)cc1Br]

To a mixture of 2-(2-bromo-6-fluoro-4-(trifluoromethyl)phenyl)piperidin-4-one (1.9 g, 5.6 mmol) in DCM (30 mL) was added Boc₂O (1.83 g, 8.4 mmol) and Et₃N (1.13 g, 11.2 mmol). The mixture was stirred at room temperature for 16 h and concentrated under reduced pressure. The residue was partitioned between water (100 mL) and DCM (100 mL). The organic layer was washed with brine (100 mL), dried over Na₂SO₄, filtered and concentrated under reduced pressure. The residue was purified by silica gel chromatograph (PE:EtOAc=10:1) to give the title compound (1.5 g, 61%). LC-MS (M+H)⁺=440.2.

### Step 3: tert-butyl 2-(2-bromo-6-fluoro-4-(trifluoromethyl)phenyl)-4-hydroxy-4-methylpiperidine-1-carboxylate

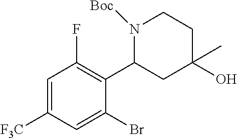 [SMILES: CC1(O)CCN(C(=O)OC(C)(C)C)C(c2c(F)cc(C(F)(F)F)cc2Br)C1]

To a mixture of tert-butyl 2-(2-bromo-6-fluoro-4-(trifluoromethyl)phenyl)-4-oxopiperidine-1-carboxylate (1.5 g, 3.4 mmol) in THF (40 mL) was added MeMgBr (5.1 mL, 1 M in CPME, 5.1 mmol) at 0° C. The mixture was stirred at room temperature for 2 h. The mixture was slowly added into water (100 mL) and then extracted with EtOAc (100 mL). The organic layer was washed with brine (2×100 mL), dried over Na₂SO₄, filtered and concentrated under reduced pressure. The residue was purified by silica gel chromatograph (PE:EtOAc=1:1) to give the title compound (830 mg, 54%). LC-MS (M+H)=456.1.

### Step 4: tert-butyl 7-fluoro-2-methyl-9-(trifluoromethyl)-3,4-dihydro-2H-2,6-methanobenzo[b][1,5]oxazocine-5(6H)-carboxylate

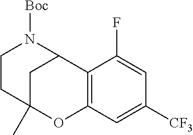 [SMILES: CC(C)(C)OC(=O)N1CCC2(C)CC1c1c(F)cc(C(F)(F)F)cc1O2]

To a mixture of tert-butyl 2-(2-bromo-6-fluoro-4-(trifluoromethyl)phenyl)-4-hydroxy-4-methylpiperidine-1-carboxylate (830 mg, 1.82 mmol) in dioxane (20 mL) was added Pd(OAc)₂ (41 mg, 0.18 mmol), Xphos (173 mg, 0.36 mmol) and Cs₂CO₃ (1.19 g, 3.65 mmol). The mixture was stirred at 100° C. for 16 h under nitrogen then cooled to room temperature. Solid was filtered off and the filtrate was concentrated under reduced pressure. The residue was partitioned between water (100 mL) and EtOAc (100 mL). The organic layer was washed with brine (2×100 mL), dried over Na₂SO₄, filtered and concentrated under reduced pressure. The residue was purified by silica gel chromatograph (PE:EtOAc=3:1) to give the title compound (600 mg, 88%). LC-MS (M+H)⁺=376.3.

### Step 5: 7-fluoro-2-methyl-9-(trifluoromethyl)-3,4,5,6-tetrahydro-2H-2,6-methanobenzo[b][1,5]oxazocine

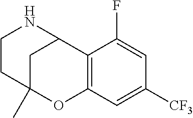 [SMILES: CC12CCNC(C1)c1c(F)cc(C(F)(F)F)cc1O2]

To tert-butyl 7-fluoro-2-methyl-9-(trifluoromethyl)-3,4-dihydro-2H-2,6-methanobenzo[b][1,5]oxazocine-5(6H)-carboxylate (600 mg, 1.6 mmol) was added HCl in dioxane (4 M, 15 mL) and the mixture was stirred at room temperature for 3 h. The mixture was concentrated under reduced pressure. The residue was partitioned between saturated NaHCO₃ (100 mL) and EtOAc (100 mL). The organic layer was washed with brine (100 mL), dried over Na₂SO₄, filtered and concentrated under reduced pressure. The residue was purified by silica gel chromatograph (DCM:MeOH=10:1) to give the title compound (250 mg, 57%). LC-MS (M+H)⁺=276.2.

### Step 6: (5-amino-1-((2-(trimethylsilyl)ethoxy)methyl)-6,8-dihydro-1H-furo[3,4-d]pyrrolo[3,2-b]pyridin-2-yl)(7-fluoro-2-methyl-9-(trifluoromethyl)-3,4-dihydro-2H-2,6-methanobenzo[b][1,5]oxazocin-5(6H)-yl)methanone

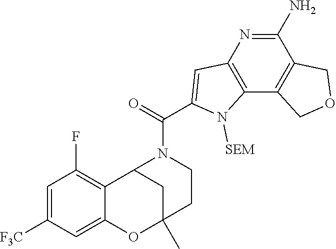 [SMILES: CC12CCN(C(=O)c3cc4nc(N)c5c(c4n3COCC[Si](C)(C)C)COC5)C(C1)c1c(F)cc(C(F)(F)F)cc1O2]

To a mixture of 7-fluoro-2-methyl-9-(trifluoromethyl)-3,4,5,6-tetrahydro-2H-2,6-methanobenzo[b][1,5]oxazocine (53 mg, 0.19 mmol) and 5-amino-1-((2-(trimethylsilyl)ethoxy)methyl)-6,8-dihydro-1H-furo[3,4-d]pyrrolo[3,2-b]pyridine-2-carboxylic acid (74 mg, 0.21 mmol) in THF (10 mL) was added BOPCl (64 mg, 0.25 mmol) and DIPEA (50 mg, 0.38 mmol) and the mixture was stirred at 60° C. for 16 h. The mixture was cooled to room temperature and concentrated under reduced pressure. The residue was partitioned between water (50 mL) and EtOAc (50 mL). The organic layer was dried with Na₂SO₄, filtered and concentrated under reduced pressure. The residue was purified by silica gel chromatograph (DCM:MeOH=25:1) to give the title compound (75 mg, 65%). LC-MS (M+H)⁺=607.2.

### Step 7: (5-amino-6,8-dihydro-1H-furo[3,4-d]pyrrolo[3,2-b]pyridin-2-yl)((2S,6R)-7-fluoro-2-methyl-9-(trifluoromethyl)-3,4-dihydro-2H-2,6-methanobenzo[b][1,5]oxazocin-5(6H)-yl)methanone & (5-amino-6,8-dihydro-1H-furo[3,4-d]pyrrolo[3,2-b]pyridin-2-yl)((2R,6S)-7-fluoro-2-methyl-9-(trifluoromethyl)-3,4-dihydro-2H-2,6-methanobenzo[b][1,5]oxazocin-5(6H)-yl)methanone

A mixture of (5-amino-1-((2-(trimethylsilyl)ethoxy)methyl)-6,8-dihydro-1H-furo[3,4-d]pyrrolo[3,2-b]pyridin-2-yl)(7-fluoro-2-methyl-9-(trifluoromethyl)-3,4-dihydro-2H-2,6-methanobenzo[b][1,5]oxazocin-5(6H)-yl)methanone (75 mg, 0.12 mmol) in DCM (6 mL) and TFA (6 mL) was stirred at room temperature for 3 h and concentrated under reduced pressure. The residue was taken in NH₃ in methanol (7 M, 3 mL) and stirred at room temperature for 2 h. The mixture was concentrated under reduced pressure. The residue was partitioned between saturated NaHCO₃ (50 mL) and EtOAc (50 mL). The organic layer was dried over Na₂SO₄, filtered and concentrated under reduced pressure. The residue was purified by prep-HPLC followed by chiral SFC to give Example 70 (14 mg, 24%) and Example 71 (10 mg, 17%).

Analytical chiral-SFC condition as below. Column: Lux Cellulose-4; Column size: 4.6×100 mm, m; Mobile phase: 4 mM methanolic NH₃:CO₂, 45:55 isocratic; Flow: 2.0 mL/min; Temperature: 35° C.; back pressure: 1500 psi.

Example 70: Analytical SFC tR=2.96 min. ¹H NMR (400 MHz, DMSO-d6) δ 11.50 (s, 1H), 7.20-6.98 (m, 2H), 6.76-6.41 (m, 1H), 6.08 (s, 1H), 5.53 (s, 2H), 5.22-5.03 (m, 2H), 4.88 (s, 2H), 4.25-4.05 (m, 1H), 3.09-2.64 (m, 1H), 2.32-2.11 (m, 1H), 2.01-1.77 (m, 3H), 1.40 (s, 3H). LC-MS (M+H)⁺=477.3.

Example 71: Analytical SFC tR=3.63 min. ¹H NMR (400 MHz, DMSO-d6) δ 11.50 (s, 1H), 7.15-6.98 (m, 2H), 6.72-6.44 (m, 1H), 6.08 (s, 1H), 5.53 (s, 2H), 5.24-4.99 (m, 2H), 4.88 (s, 2H), 4.30-4.07 (m, 1H), 3.11-2.79 (m, 1H), 2.25-2.03 (m, 1H), 2.02-1.80 (m, 3H), 1.40 (s, 3H). LC-MS (M+H)⁺=477.3.

### Example 72 & Example 73: ((R)-5-amino-6-methyl-6,8-dihydro-1H-furo[3,4-d]pyrrolo[3,2-b]pyridin-2-yl)((2S,6R)-7-fluoro-2-methyl-9-(trifluoromethyl)-3,4-dihydro-2H-2,6-methanobenzo[b][1,5]oxazocin-5(6H)-yl)methanone & ((R)-5-amino-6-methyl-6,8-dihydro-1H-furo[3,4-d]pyrrolo[3,2-b]pyridin-2-yl)((2R,6S)-7-fluoro-2-methyl-9-(trifluoromethyl)-3,4-dihydro-2H-2,6-methanobenzo[b][1,5]oxazocin-5(6H)-yl)methanone

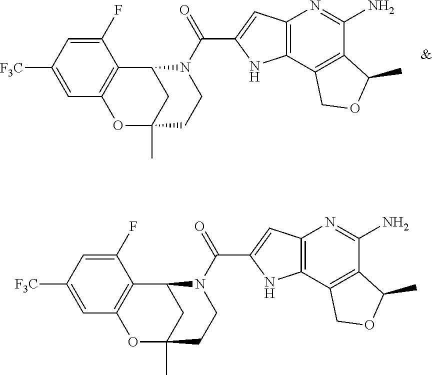 [SMILES: C[C@H]1OCc2c1c(N)nc1cc(C(=O)N3CC[C@@]4(C)C[C@@H]3c3c(F)cc(C(F)(F)F)cc3O4)[nH]c21 ; C[C@H]1OCc2c1c(N)nc1cc(C(=O)N3CC[C@]4(C)C[C@H]3c3c(F)cc(C(F)(F)F)cc3O4)[nH]c21]

To a mixture of (R)-5-amino-6-methyl-6,8-dihydro-1H-furo[3,4-d]pyrrolo[3,2-b]pyridine-2-carboxylic acid (50 mg, 0.21 mmol), 7-fluoro-2-methyl-9-(trifluoromethyl)-3,4,5,6-tetrahydro-2H-2,6-methanobenzo[b][1,5]oxazocine (59 mg, 0.21 mmol) and DIPEA (194 mg, 1.5 mmol) in anhydrous DMF (3 mL) under nitrogen was added T3P (341 mg, 1.07 mmol), and the mixture was stirred at 55° C. for 16 h. The mixture was cooled to room temperature followed by addition of hydrochloric acid (6 M, 9 mL). The mixture was stirred at 60° C. for 3 h then cooled to room temperature. The mixture was partitioned between saturated NaHCO₃ (100 mL) and EtOAc (100 mL). The organic layer was washed with brine (2×100 mL), dried over Na₂SO₄, filtered and concentrated under reduced pressure. The residue was purified by prep-HPLC followed by chiral SFC to give Example 72 (14 mg, 13%) and Example 73 (16 mg, 15%).

Analytical chiral-SFC condition as below. Column: Lux Cellulose-4; Column size: 4.6×100 mm, m; Mobile phase: 4 mM methanolic NH₃:CO₂, 45:55 isocratic; Flow: 2.0 mL/min; Temperature: 35° C.; back pressure: 1500 psi.

Example 72: Analytical SFC tR=2.34 min. ¹H NMR (400 MHz, DMSO-d6) δ 11.54 (s, 1H), 7.31-6.95 (m, 2H), 6.79-6.45 (m, 1H), 6.17-5.95 (m, 1H), 5.51 (s, 2H), 5.37-5.25 (m, 1H), 5.21-5.08 (m, 1H), 5.10-4.86 (m, 1H), 4.29-4.02 (m, 1H), 3.09-2.76 (m, 1H), 2.25-2.05 (m, 1H), 1.98-1.77 (m, 3H), 1.40 (s, 3H), 1.36-1.19 (m, 3H). LC-MS (M+H)⁺=491.3.

Example 73: Analytical SFC tR=3.24 min. ¹H NMR (400 MHz, DMSO-d6) δ 11.50 (s, 1H), 7.19-6.97 (m, 2H), 6.66-6.45 (m, 1H), 6.11-6.05 (m, 1H), 5.46 (s, 2H), 5.38-5.24 (m, 1H), 5.14-4.97 (m, 2H), 4.31-4.02 (m, 1H), 3.14-2.75 (m, 1H), 2.26-2.04 (m, 1H), 2.03-1.76 (m, 3H), 1.41 (s, 3H), 1.36-1.24 (m, 3H). LC-MS (M+H)⁺=491.3.

### Example 74 & Example 75: ((R)-5-amino-6-methyl-6,8-dihydro-1H-furo[3,4-d]pyrrolo[3,2-b]pyridin-2-yl)((1′R,5′R)-8′-bromo-3′,4′-dihydrospiro[cyclopropane-1,6′-[1,5]methanobenzo[f][1,4]diazocin]-2′(1′H)-yl)methanone & ((R)-5-amino-6-methyl-6,8-dihydro-1H-furo[3,4-d]pyrrolo[3,2-b]pyridin-2-yl)((1′S,5′S)-8′-bromo-3′,4′-dihydro[cyclopropane-1,6′-[1,5]methanobenzo[f][1,4]diazocin]-2′(1′H)-yl)methanone

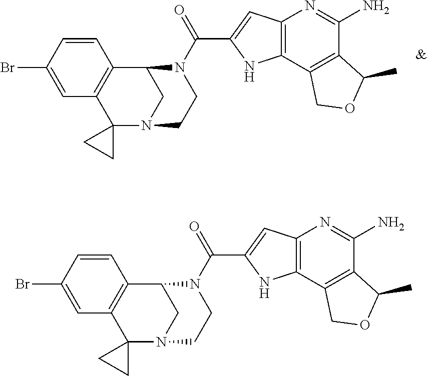 [SMILES: C[C@H]1OCc2c1c(N)nc1cc(C(=O)N3CC[N@]4C[C@H]3c3ccc(Br)cc3C43CC3)[nH]c21 ; C[C@H]1OCc2c1c(N)nc1cc(C(=O)N3CC[N@@]4C[C@@H]3c3ccc(Br)cc3C43CC3)[nH]c21]

### Step 1: 8′-bromo-1′,2′,3′,4′-tetrahydrospiro[cyclopropane-1,6′-[1,5]methanobenzo[f][1,4]diazocine]dihydrochloride

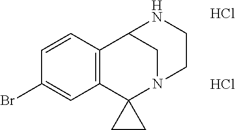 [SMILES: Cl ; Brc1ccc2c(c1)C1(CC1)N1CCNC2C1 ; Cl]

A mixture of tert-butyl 8′-bromo-3′,4′-dihydrospiro[cyclopropane-1,6′-[1,5]methanobenzo[f][1,4]diazocine]-2′(1′H)-carboxylate (500 mg, 1.3 mmol) in HCl in dioxane (4 M, 10 mL) was stirred at room temperature for 2 h. The mixture was concentrated under reduced pressure to give the title compound (460 mg, 100%). LC-MS (M+H)⁺=279.1.

### Step 2: ((R)-5-amino-6-methyl-6,8-dihydro-1H-furo[3,4-d]pyrrolo[3,2-b]pyridin-2-yl)((1′R,5′R)-8′-bromo-3′,4′-dihydrospiro[cyclopropane-1,6′-[1,5]methanobenzo[f][1,4]diazocin]-2′(1′H)-yl)methanone & ((R)-5-amino-6-methyl-6,8-dihydro-1H-furo[3,4-d]pyrrolo[3,2-b]pyridin-2-yl)((1'S,5'S)-8′-bromo-3′,4′-dihydrospiro[cyclopropane-1,6′-[1,5]methanobenzo[f][1,4]diazocin]-2′(1′H)-yl)methanone

To a mixture of (R)-5-amino-6-methyl-6,8-dihydro-1H-furo[3,4-d]pyrrolo[3,2-b]pyridine-2-carboxylic acid (41 mg, 0.18 mmol), 8′-bromo-1′,2′,3′,4′-tetrahydrospiro[cyclopropane-1,6′-[1,5]methanobenzo[f][1,4]diazocine]dihydrochloride (63 mg, 0.18 mmol) and DIPEA (0.23 mL, 1.26 mmol) in anhydrous DMF (10 mL) under nitrogen was added T3P (286 mg, 0.90 mmol), and the mixture was stirred at 60° C. for 16 h. The mixture was cooled to room temperature follow by addition of, hydrochloric acid (6 M, 9 mL). The mixture was stirred at 60° C. for 3 h and cooled to room temperature. The mixture was partitioned between saturated NaHCO₃ (100 mL) and EtOAc (100 mL). The organic layer was washed with brine (100 mL), dried over Na₂SO₄, filtered and concentrated under reduced pressure. The residue was purified by prep-TLC followed by SFC to give Example 74 (8 mg, 9%) and Example 75 (5 mg, 6%).

Analytical chiral-SFC condition as below. Column: YMC Cellulose-C; Column size: 4.6×100 mm, 5 μm; Mobile phase: 4 mM methanolic NH₃:CO₂, 45:55 isocratic; Flow: 2.0 mL/min; Temperature: 35° C.; back pressure: 1500 psi.

Example 74: Analytical SFC tR=3.77 min. ¹H NMR (400 MHz, DMSO-d6, 80° C.) δ11.28 (s, 1H), 7.28-7.26 (m, 2H), 6.90-6.89 (m, 2H), 6.50 (s, 1H), 5.51 (s, 1H), 5.17 (s, 1H), 5.13-5.02 (m, 4H), 3.35-3.33 (m, 1H), 3.36-3.23 (m, 2H), 3.02-2.96 (m, 2H), 1.62-1.60 (m, 1H), 1.22-1.16 (m, 6H), 1.14 (s, 2H), 0.76-0.74 (m, 1H). LC-MS (M+H)⁺=494.2.

Example 75: Analytical SFC tR=7.00 min. ¹H NMR (400 MHz, DMSO-d6, 80° C.) δ11.27 (s, 1H), 7.29-7.28 (m, 2H), 6.90-6.89 (m, 2H), 6.50 (s, 1H), 5.51 (s, 1H), 5.17 (s, 1H), 5.13-5.02 (m, 4H), 3.35-3.33 (m, 1H), 3.36-3.23 (m, 2H), 3.02-2.96 (m, 2H), 1.62-1.60 (m, 1H), 1.22-1.16 (m, 6H), 1.14 (s, 2H), 0.75-0.73 (m, 1H). LC-MS (M+H)⁺=494.2.

### Example 76 & Example 77: (5-amino-6,8-dihydro-1H-furo[3,4-d]pyrrolo[3,2-b]pyridin-2-yl)((1′R,5′R)-8′-bromo-3′,4′-dihydrospiro[cyclopropane-1,6′-[1,5]methanobenzo[f][1,4]diazocin]-2′(1′H)-yl)methanone & (5-amino-6,8-dihydro-1H-furo[3,4-d]pyrrolo[3,2-b]pyridin-2-yl)((1′S,5′S)-8′-bromo-3′,4′-dihydrospiro[cyclopropane-1,6′-[1,5]methanobenzo[f][1,4]diazocin]-2′(1′H)-yl)methanone

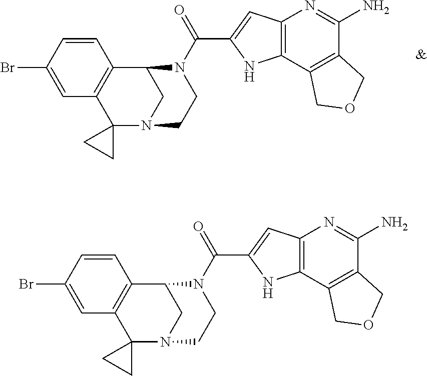 [SMILES: Nc1nc2cc(C(=O)N3CC[N@]4C[C@H]3c3ccc(Br)cc3C43CC3)[nH]c2c2c1COC2 ; Nc1nc2cc(C(=O)N3CC[N@@]4C[C@@H]3c3ccc(Br)cc3C43CC3)[nH]c2c2c1COC2]

### Step 1: (5-amino-1-((2-(trimethylsilyl)ethoxy)methyl)-6,8-dihydro-1H-furo[3,4-d]pyrrolo[3,2-b]pyridin-2-yl)(8′-bromo-3′,4′-dihydrospiro[cyclopropane-1,6′-[1,5]methanobenzo[f][1,4]diazocin]-2′(1′H)-yl)methanone

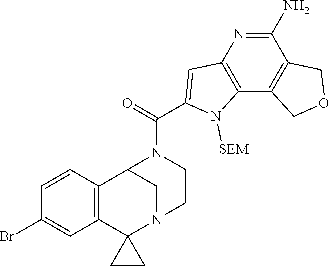 [SMILES: C[Si](C)(C)CCOCn1c(C(=O)N2CCN3CC2c2ccc(Br)cc2C32CC2)cc2nc(N)c3c(c21)COC3]

To a mixture of 5-amino-1-((2-(trimethylsilyl)ethoxy)methyl)-6,8-dihydro-1H-furo[3,4-d]pyrrolo[3,2-b]pyridine-2-carboxylic acid (63 mg, 0.18 mmol) and 8′-bromo-1′,2′,3′,4′-tetrahydrospiro[cyclopropane-1,6′-[1,5]methanobenzo[f][1,4]diazocine]dihydrochloride (63 mg, 0.18 mmol) in THF (5 mL) was added BOPCl (74 mg, 0.21 mmol) and DIPEA (0.17 mL, 0.90 mmol). The mixture was stirred at 60° C. for 2 h then cooled to room temperature. The mixture was partitioned between water (10 mL) and EtOAc (50 mL). The organic layer were washed with brine (10 mL), dried over Na₂SO₄, filtered and concentrated under reduced pressure. The residue was purified by prep-TLC to give the title compound (50 mg, 49%). LC-MS (M+H)=610.2.

### Step 2: (5-amino-6,8-dihydro-1H-furo[3,4-d]pyrrolo[3,2-b]pyridin-2-yl)((1′R,5′R)-8′-bromo-3′,4′-dihydrospiro[cyclopropane-1,6′-[1,5]methanobenzo[f][1,4]diazocin]-2′(1′H)-yl)methanone & (5-amino-6,8-dihydro-1H-furo[3,4-d]pyrrolo[3,2-b]pyridin-2-yl)((1′S,5′S)-8′-bromo-3′,4′-dihydrospiro[cyclopropane-1,6′-[1,5]methanobenzo[f][1,4]diazocin]-2′(1′H)-yl)methanone

A mixture of (5-amino-1-((2-(trimethylsilyl)ethoxy)methyl)-6,8-dihydro-1H-furo[3,4-d]pyrrolo[3,2-b]pyridin-2-yl)(8′-bromo-3′,4′-dihydrospiro[cyclopropane-1,6′-[1,5]methanobenzo[f][1,4]diazocin]-2′(1′H)-yl)methanone (50 mg, 0.082 mmol) in TFA (2 mL) was stirred at room temperature for 0.5 h then concentrated under reduced pressure. The residue was taken in THF (4 mL) and ammonium hydroxide (28%, 1 mL). The mixture was stirred at room temperature for 2 h and concentrated under reduced pressure. The residue was purified by prep-HPLC followed by SFC to give Example 76 (9 mg, 23%) and Example 77 (8 mg, 20%).

Analytical chiral-SFC condition as below. Column: Lux Cellulose-3; Column size: 4.6×100 mm, m; Mobile phase: 4 mM methanolic NH₃:CO₂, 1:9 to 1:1 in 3 min, 1:1 for 2 min, 1:1 to 1:9 in 0.1 min, 1:9 for 1.9 min; Flow: 2.0 mL/min; Temperature: 35° C.; back pressure: 1500 psi.

Example 76: Analytical SFC tR=3.48 min. ¹H NMR (400 MHz, DMSO-d6, 80° C.) δ11.30 (s, 1H), 7.28-7.27 (m, 1H), 6.97-6.90 (m, 2H), 6.63 (s, 1H), 5.52 (s, 1H), 5.29 (s, 2H), 5.12 (s, 2H), 4.90 (s, 2H), 3.86-3.84 (m, 1H), 3.36-3.23 (m, 3H), 3.06-2.94 (m, 3H), 1.56-1.53 (m, 1H), 1.14 (s, 1H), 0.77-0.75 (m, 1H). LC-MS (M+H)=480.2.

Example 77: Analytical SFC tR=3.85 min. ¹H NMR (400 MHz, DMSO-d6, 80° C.) δ1.29 (s, 1H), 7.28-7.26 (m, 1H), 6.98-6.90 (m, 2H), 6.53 (s, 1H), 5.51 (s, 1H), 5.29 (s, 2H), 5.12 (s, 2H), 4.90 (s, 2H), 3.86-3.84 (m, 1H), 3.36-3.23 (m, 3H), 3.06-2.94 (m, 3H), 1.56-1.53 (m, 1H), 1.14 (s, 1H), 0.83-0.75 (m, 1H). LC-MS (M+H)⁺=480.2.

### Example 78 & Example 79: ((R)-5-amino-6-methyl-6,8-dihydro-1H-furo[3,4-d]pyrrolo[3,2-b]pyridin-2-yl)((1′R,5′R)-8′-(trifluoromethyl)-3′,4′-dihydrospiro[cyclopropane-1,6′-[1,5]methanobenzo[f][1,4]diazocin]-2′(1′H)-yl)methanone & ((R)-5-amino-6-methyl-6,8-dihydro-1H-furo[3,4-d]pyrrolo[3,2-b]pyridin-2-yl)((1′S,5 S)-8′-(trifluoromethyl)-3′,4′-dihydrospiro[cyclopropane-1,6′-[1,5]methanobenzo[f][1,4]diazocin]-2′(1′H)-yl)methanone

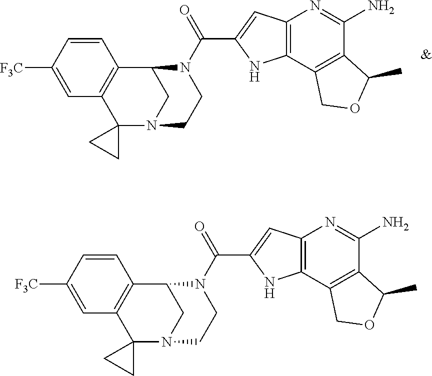 [SMILES: C[C@H]1OCc2c1c(N)nc1cc(C(=O)N3CC[N@]4C[C@H]3c3ccc(C(F)(F)F)cc3C43CC3)[nH]c21 ; C[C@H]1OCc2c1c(N)nc1cc(C(=O)N3CC[N@@]4C[C@@H]3c3ccc(C(F)(F)F)cc3C43CC3)[nH]c21]

To a mixture of (R)-5-amino-6-methyl-6,8-dihydro-1H-furo[3,4-d]pyrrolo[3,2-b]pyridine-2-carboxylic acid (41 mg, 0.18 mmol), 8′-(trifluoromethyl)-1′,2′,3′,4′-tetrahydrospiro[cyclopropane-1,6′-[1,5]methanobenzo[f][1,4]diazocine]dihydrochloride (63 mg, 0.18 mmol) and DIPEA (0.23 ml, 1.26 mmol) in anhydrous DMF (10 mL) under nitrogen was added T3P (286 mg, 0.90 mmol). The mixture was stirred at 60° C. for 16 h then cooled to room temperature. Hydrochloric acid (6 M, 9 mL) was added and the mixture was stirred at 60° C. for 3 h. The mixture was cooled to room temperature and partitioned between saturated NaHCO₃ (100 mL) and EtOAc (100 mL). The organic layer was washed with brine (100 mL), dried over Na₂SO₄, filtered and concentrated under reduced pressure. The residue was purified by prep-TLC followed by SFC to give Example 78 (10 mg, 11%) and Example 79 (10 mg, 11%).

Analytical chiral-SFC condition as below. Column: YMC Cellulose-C; Column size: 4.6×100 mm, 5 μm; Mobile phase: 4 mM methanolic NH₃:CO₂, 45:55 isocratic; Flow: 2.0 mL/min; Temperature: 35° C.; back pressure: 1500 psi.

Example 76: Analytical SFC tR=1.98 min. ¹H NMR (400 MHz, DMSO-d6, 80° C.) δ11.30 (s, 1H), 7.44-7.42 (m, 1H), 7.27 (s, 1H), 6.99 (s, 1H), 6.54 (s, 1H), 5.61 (s, 1H), 5.34-5.35 (m, 1H), 5.17-5.01 (m, 4H), 3.88-3.85 (m, 1H), 3.39-3.23 (m, 2H), 2.92-2.91 (m, 3H), 1.96-1.94 (m, 1H), 1.25-1.24 (m, 3H), 1.22-1.17 (m, 2H), 0.81-0.78 (m, 1H). LC-MS (M+H)⁺=484.3.

Example 77: Analytical SFC tR=3.43 min. ¹H NMR (400 MHz, DMSO-d6, 80° C.) δ11.29 (s, 1H), 7.46-7.45 (m, 1H), 7.27 (s, 1H), 6.99 (s, 1H), 6.51 (s, 1H), 5.61 (s, 1H), 5.34-5.35 (m, 1H), 5.17-5.01 (m, 4H), 3.88-3.85 (m, 1H), 3.39-3.23 (m, 2H), 2.92-2.91 (m, 3H), 1.96-1.94 (m, 1H), 1.25-1.24 (m, 3H), 1.22-1.17 (m, 2H), 0.81-0.78 (m, 1H). LC-MS (M+H)⁺=484.2.

### Example 80: ((R)-5-amino-6-methyl-6,8-dihydro-1H-furo[3,4-d]pyrrolo[3,2-b]pyridin-2-yl)((1S,5S)-10-fluoro-8-(trifluoromethyl)-1,3,4,6-tetrahydro-2H-1,5-methanobenzo[f][1,4]diazocin-2-yl)methanone

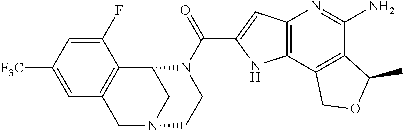 [SMILES: C[C@H]1OCc2c1c(N)nc1cc(C(=O)N3CC[N@]4Cc5cc(C(F)(F)F)cc(F)c5[C@H]3C4)[nH]c21]

Example 80 was obtained through the similar method of Example 20.

Analytical chiral-SFC condition as below. Column: YMC Cellulose-SB; Column size: 4.6×100 mm, 5 μm; Mobile phase: 4 mM methanolic NH₃:CO₂, 45:55 isocratic; Flow: 2.0 mL/min; Temperature: 35° C.; back pressure: 1500 psi.

Example 80: Analytical SFC tR=7.68 min. ¹H NMR (400 MHz, DMSO-d6, 80° C.) δ11.27 (s, 1H), 7.41-7.37 (m, 2H), 6.53 (s, 1H), 5.77 (s, 1H), 5.33 (s, 1H), 5.18-5.15 (m, 3H), 5.04-5.00 (m, 1H), 4.34-4.30 (m, 1H), 4.13-4.08 (m, 1H), 3.84-3.82 (m, 1H), 3.40-3.36 (m, 1H), 3.05-2.97 (m, 4H), 1.37-1.23 (m, 3H). LC-MS (M+H)⁺=476.3.

### Assays

### Cell Killing in HCT116 Isogenic Pair

HCT116 cell line (ATCC, CCL-247) was isolated from the colon of an adult male with colon cancer. It had a mutation in codon 13 of the ras proto-oncogene and was used as a positive control for PCR assays of mutation in this codon. HCT116-MTAP-KO was knocked-out-MTAP-gene based HCT116, and a single clone was passed for this assay. HCT116-mock-RNA-KO was knocked-out-mock-gene based HCT116 with MTAP wildtype genotype. The base medium for HCT116 isogenic pair was RPMI 1640, HEPES (Gibco, 22400105). Made the complete growth medium, added the following components to the base medium: fetal bovine serum to a final concentration of 10% (Gibco, 10099-141C). The cell line was grown in a humidified 5% CO₂ atmosphere at 37° C. and regularly tested for the presence of Mycoplasma with MycoAlert™ PLUS Mycoplasma Detection Kit (Lonza, LT07-710).

### Experimental

Seeded HCT116-mock-RNA-KO (4E2/well) or HCT116-MTAP-KO (4E2/well) cells into a 96-well plate (Greiner: 655090), 100 μL/well.
Incubated at 37° C., 5% CO₂, overnight.
Added 50 μL fresh growth-medium containing serial dilution of compounds for a final compound concentration of 0-10 μM to each well.
Incubated at 37° C., 5% CO₂, 6 days.
The cell viability was detected by Cell Titer-Glo (Promega, G7573). Added 70 μL of CellTiter-Glo® reagent in each well.
Incubated at room temperature for 10 minutes to stabilize luminescent signal.
Analyzed with Microplate Reader (TECAN, SPARK) and fit IC₅₀ using Prism® 9 or Prism® 10.

The compounds disclosed herein showed cell killing activity values as in Table 1

TABLE 1
Cell killing IC₅₀ (nM) for the compounds disclosed herein

| | | Cell killing | | | Cell killing |
|---|---|---|---|---|---|
| | Cell killing | IC₅₀ (nM) in | | Cell killing | IC₅₀ (nM) in |
| | IC₅₀ (nM) in | HCT116- | | IC₅₀ (nM) in | HCT116- |
| | HCT116- | mock-RNA- | | HCT116- | mock-RNA- |
| Example | MTAP-KO | KO | Example | MTAP-KO | KO |
| 1 | B | D | 2 | A | D |
| 3 | C | D | 4 | D | D |
| 5 | B | D | 6 | C | D |
| 7 | A | D | 8 | C | D |
| 9 | C | D | 10 | B | D |
| 11 | B | D | 12 | D | D |
| 13 | C | D | 14 | A | C |
| 15 | C | D | 16 | A | C |
| 17 | D | D | 18 | A | D |
| 19 | D | D | 20 | A | C |
| 21 | C | D | 22 | D | D |
| 23 | C | D | 24 | A | D |
| 25 | B | D | 26 | C | D |
| 27 | A | C | 28 | C | D |
| 29 | A | D | 30 | C | D |
| 31 | A | D | 32 | B | D |
| 33 | A | D | 34 | A | D |
| 35 | A | C | 36 | A | C |
| 37 | A | D | 38 | C | D |
| 39 | C | D | 40 | A | D |
| 41 | A | C | 42 | C | D |
| 43 | A | C | 44 | C | D |
| 45 | A | C | 46 | C | D |
| 47 | C | D | 48 | A | C |
| 49 | C | D | 50 | A | C |
| 51 | B | D | 52 | C | D |
| 53 | A | C | 54 | C | D |
| 55 | A | C | 56 | C | D |
| 57 | A | D | 58 | C | D |
| 59 | A | C | 60 | C | D |
| 61 | A | D | 62 | C | D |
| 63 | A | D | 64 | A | D |
| 65 | D | D | 66 | C | D |
| 67 | B | D | 68 | D | D |
| 69 | A | D | 70 | B | D |
| 71 | D | D | 72 | B | D |
| 73 | D | D | 74 | C | D |
| 75 | A | D | 76 | C | D |
| 77 | A | D | 78 | C | D |
| 79 | A | D | 80 | A | C |

The activity in Table 1 is categorised as “A”, “B”, and “C” based on the corresponding value according to the following rules.

| Category | Cell killing IC₅₀ (nM) |
|---|---|
| A | less than 20 |
| B | no less than 20, and less than 100 |
| C | no less than 100, and less than 1000 |
| D | no less than 1000, and less than 100000 |

Unless otherwise specified, to the extent that there is a discrepancy between a depicted chemical structure of a compound provided herein and a chemical name of a compound provided herein, the chemical structure shall control.

1-24. (canceled)

25. A compound, or a hydrate thereof, or a solvate thereof, or a pharmaceutically acceptable salt thereof, or a stereoisomer thereof, or a tautomer thereof, or a deuterated analog thereof, wherein the compound is selected from:

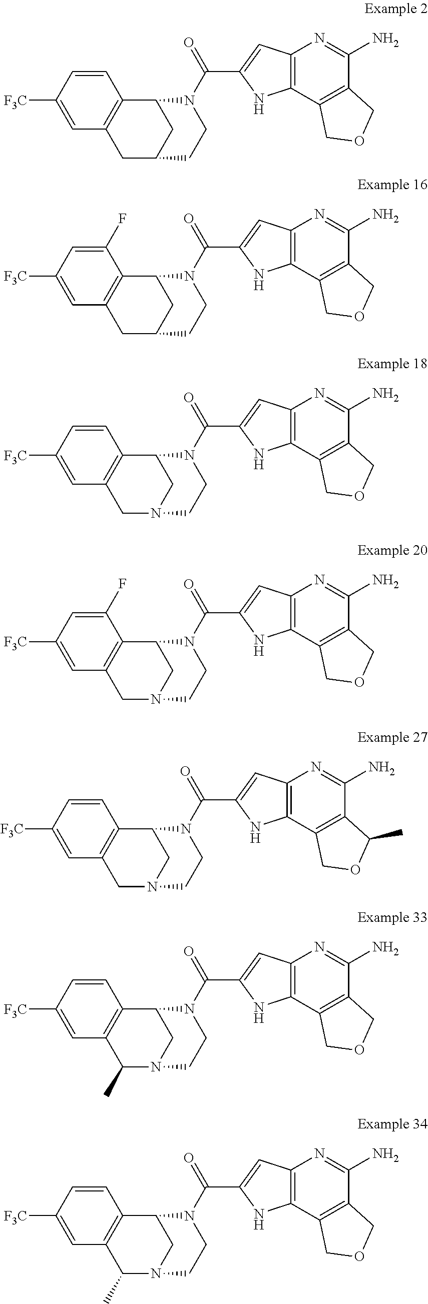

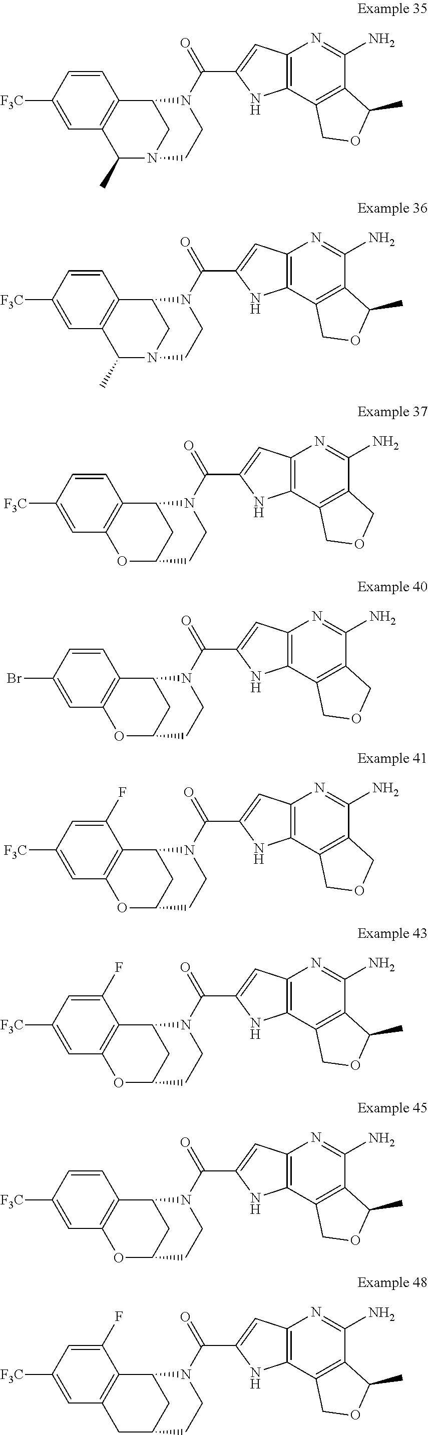

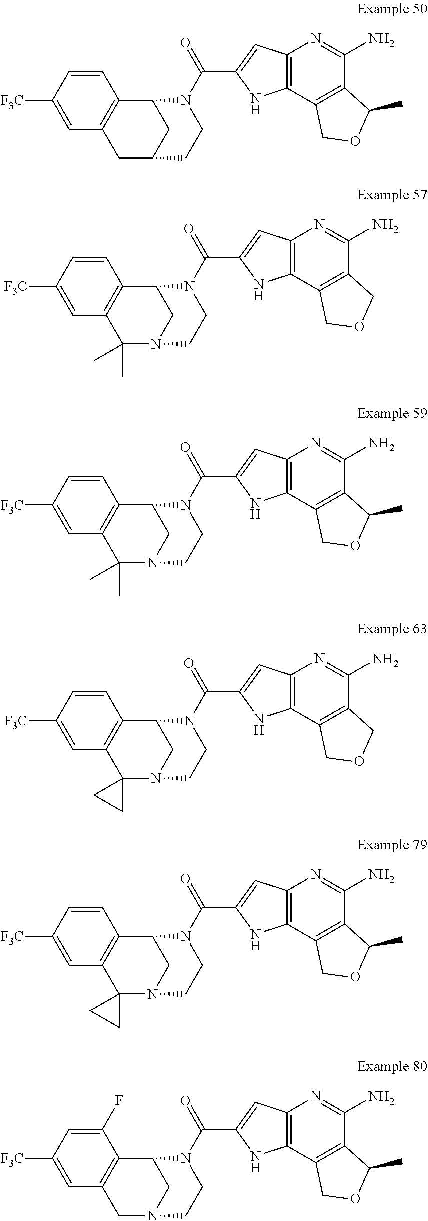 [SMILES: C[C@H]1OCc2c1c(N)nc1cc(C(=O)N3CC[C@@H]4Cc5cc(C(F)(F)F)ccc5[C@H]3C4)[nH]c21 ; CC1(C)c2cc(C(F)(F)F)ccc2[C@H]2C[N@]1CCN2C(=O)c1cc2nc(N)c3c(c2[nH]1)COC3 ; C[C@H]1OCc2c1c(N)nc1cc(C(=O)N3CC[N@@]4C[C@@H]3c3ccc(C(F)(F)F)cc3C4(C)C)[nH]c21 ; Nc1nc2cc(C(=O)N3CC[N@@]4C[C@@H]3c3ccc(C(F)(F)F)cc3C43CC3)[nH]c2c2c1COC2 ; C[C@H]1OCc2c1c(N)nc1cc(C(=O)N3CC[N@@]4C[C@@H]3c3ccc(C(F)(F)F)cc3C43CC3)[nH]c21 ; C[C@H]1OCc2c1c(N)nc1cc(C(=O)N3CC[N@]4Cc5cc(C(F)(F)F)cc(F)c5[C@H]3C4)[nH]c21]

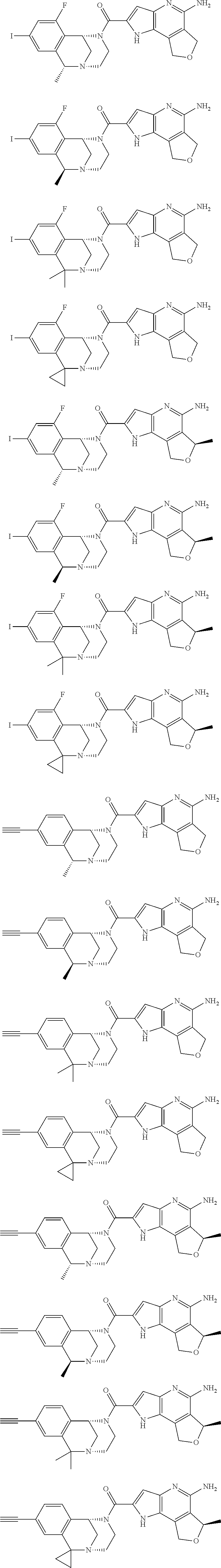

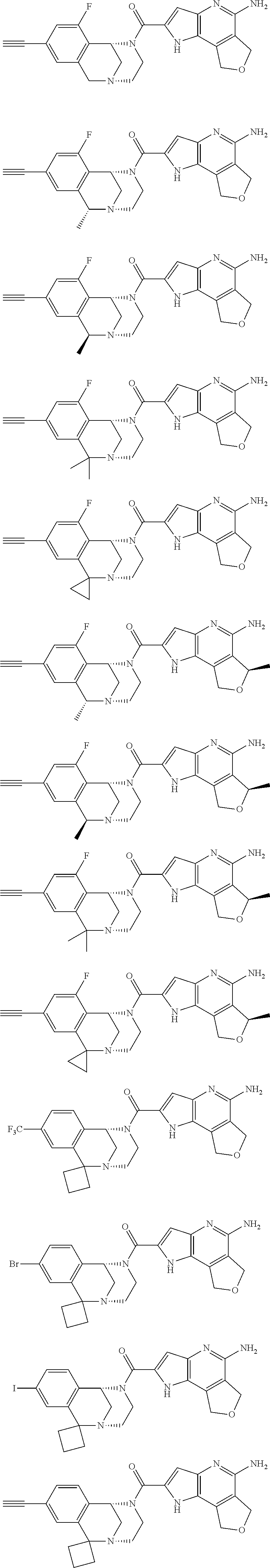

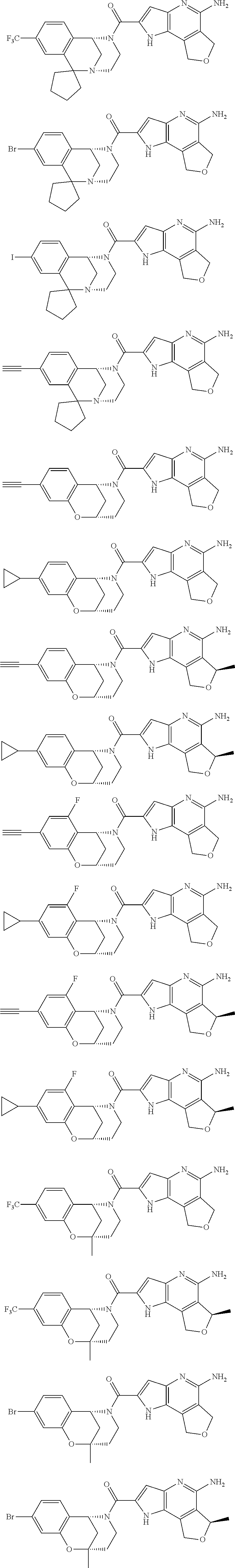

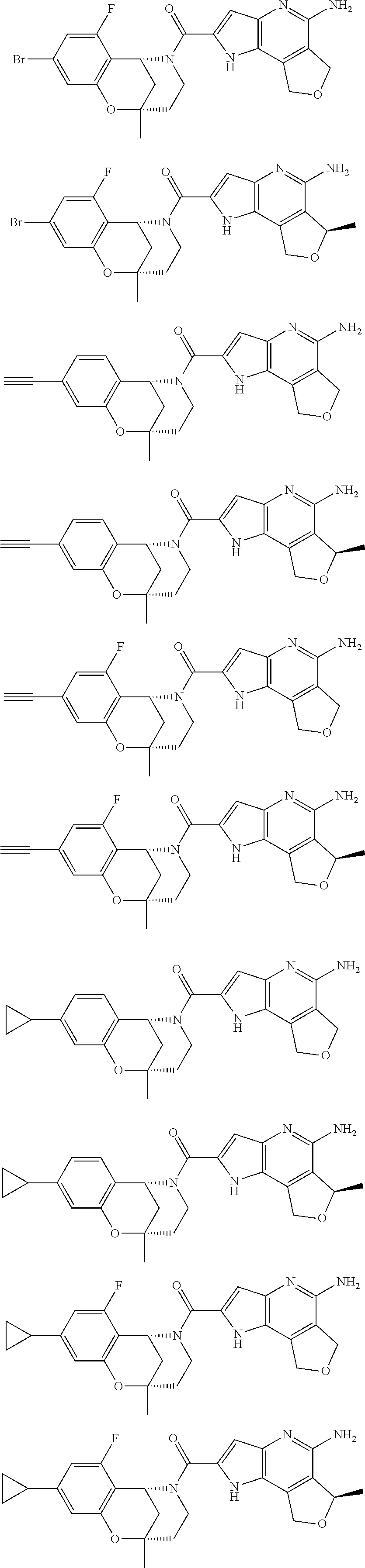

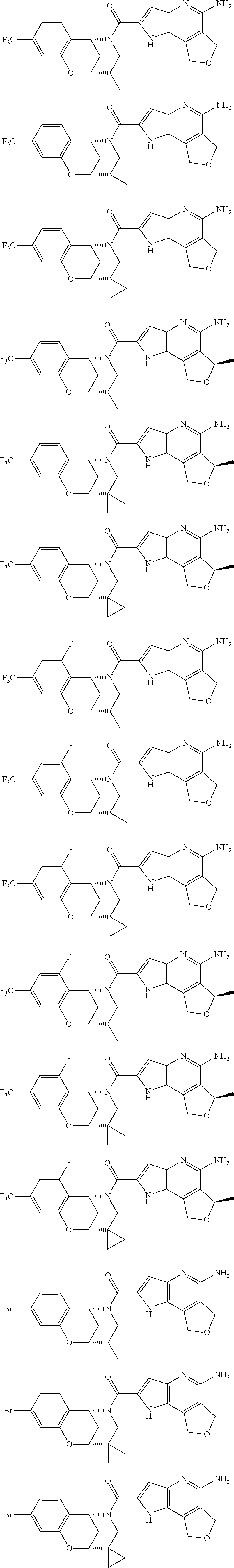

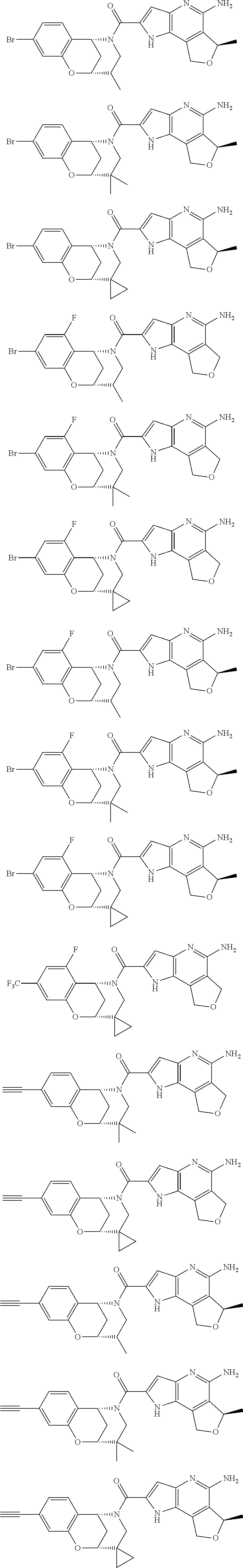

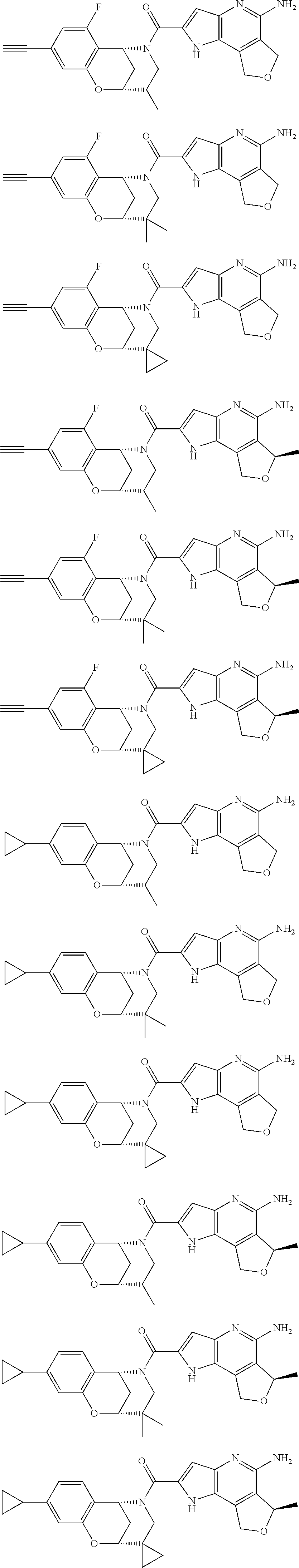

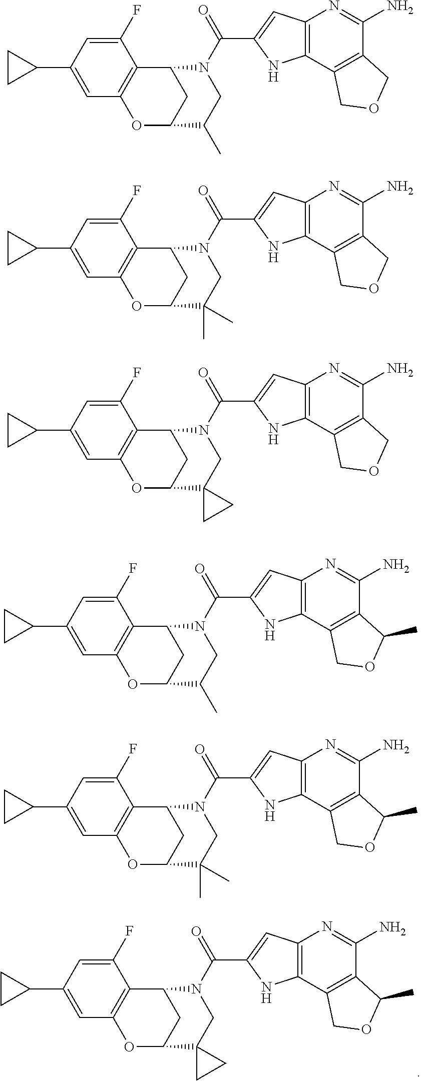

26. A pharmaceutical composition comprising a compound of claim 25, or a hydrate thereof, or a solvate thereof, or an N-oxide thereof, or a pharmaceutically acceptable salt thereof, or a stereoisomer thereof, or a tautomer thereof, or a deuterated analog thereof, and a pharmaceutically acceptable excipient.

27. A method of inhibiting PRMT5 activity, comprising administering to a subject in need thereof an effective amount of a compound according to claim 25, or a hydrate thereof, or a solvate thereof, or an N-oxide thereof, or a pharmaceutically acceptable salt, stereoisomer, tautomer, or a deuterated analog thereof.

28. A method of treating a disease modulated by PRMT5, comprising administering to a subject in need thereof an effective amount of a compound according to claim 25, or a hydrate thereof, or a solvate thereof, or an N-oxide thereof, or a pharmaceutically acceptable salt, stereoisomer, tautomer, or a deuterated analog thereof, optionally, the disease is cancer; optionally, the cancer comprises a methylthioadenosine phosphorylase (MTAP)-null solid tumor.

29. The compound of claim 25, or a hydrate thereof, or a solvate thereof, or a pharmaceutically acceptable salt thereof, wherein the compound is 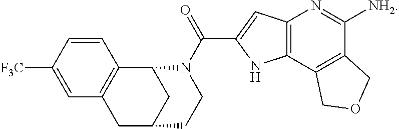 [SMILES: Nc1nc2cc(C(=O)N3CC[C@@H]4Cc5cc(C(F)(F)F)ccc5[C@H]3C4)[nH]c2c2c1COC2]
Example 2

30. The compound of claim 25, or a hydrate thereof, or a solvate thereof, or a pharmaceutically acceptable salt thereof, wherein the compound is 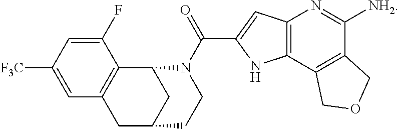 [SMILES: Nc1nc2cc(C(=O)N3CC[C@@H]4Cc5cc(C(F)(F)F)cc(F)c5[C@H]3C4)[nH]c2c2c1COC2]
Example 16

31. The compound of claim 25, or a hydrate thereof, or a solvate thereof, or a pharmaceutically acceptable salt thereof, wherein the compound is 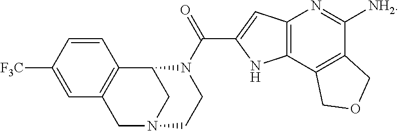 [SMILES: Nc1nc2cc(C(=O)N3CC[N@]4Cc5cc(C(F)(F)F)ccc5[C@H]3C4)[nH]c2c2c1COC2]
Example 18

32. The compound of claim 25, or a hydrate thereof, or a solvate thereof, or a pharmaceutically acceptable salt thereof, wherein the compound is 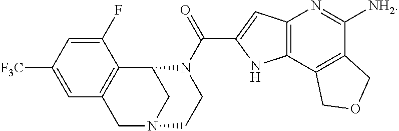 [SMILES: Nc1nc2cc(C(=O)N3CC[N@]4Cc5cc(C(F)(F)F)cc(F)c5[C@H]3C4)[nH]c2c2c1COC2]
Example 20

33. The compound of claim 25, or a hydrate thereof, or a solvate thereof, or a pharmaceutically acceptable salt thereof, wherein the compound is 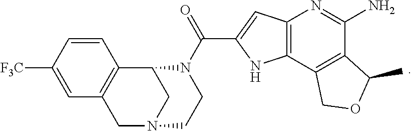 [SMILES: C[C@H]1OCc2c1c(N)nc1cc(C(=O)N3CC[N@]4Cc5cc(C(F)(F)F)ccc5[C@H]3C4)[nH]c21]
Example 27

34. The compound of claim 25, or a hydrate thereof, or a solvate thereof, or a pharmaceutically acceptable salt thereof, wherein the compound is 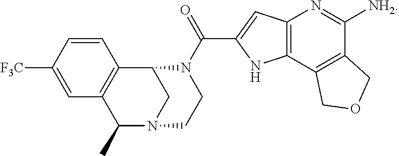 [SMILES: C[C@H]1c2cc(C(F)(F)F)ccc2[C@H]2C[N@]1CCN2C(=O)c1cc2nc(N)c3c(c2[nH]1)COC3]
Example 33

35. The compound of claim 25, or a hydrate thereof, or a solvate thereof, or a pharmaceutically acceptable salt thereof, wherein the compound is 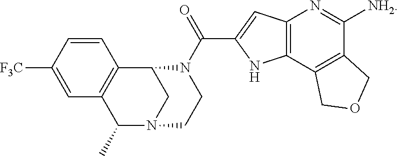 [SMILES: C[C@@H]1c2cc(C(F)(F)F)ccc2[C@H]2C[N@]1CCN2C(=O)c1cc2nc(N)c3c(c2[nH]1)COC3]
Example 34

36. The compound of claim 25, or a hydrate thereof, or a solvate thereof, or a pharmaceutically acceptable salt thereof, wherein the compound is 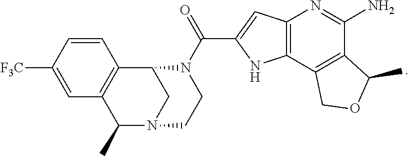 [SMILES: C[C@H]1OCc2c1c(N)nc1cc(C(=O)N3CC[N@@]4C[C@@H]3c3ccc(C(F)(F)F)cc3[C@@H]4C)[nH]c21]
Example 35

37. The compound of claim 25, or a hydrate thereof, or a solvate thereof, or a pharmaceutically acceptable salt thereof, wherein the compound is 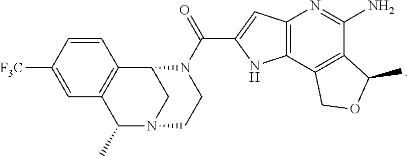 [SMILES: C[C@@H]1c2cc(C(F)(F)F)ccc2[C@H]2C[N@]1CCN2C(=O)c1cc2nc(N)c3c(c2[nH]1)CO[C@@H]3C]
Example 36

38. The compound of claim 25, or a hydrate thereof, or a solvate thereof, or a pharmaceutically acceptable salt thereof, wherein the compound is 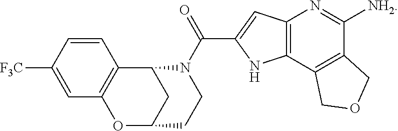 [SMILES: Nc1nc2cc(C(=O)N3CC[C@H]4C[C@@H]3c3ccc(C(F)(F)F)cc3O4)[nH]c2c2c1COC2]
Example 37

39. The compound of claim 25, or a hydrate thereof, or a solvate thereof, or a pharmaceutically acceptable salt thereof, wherein the compound is 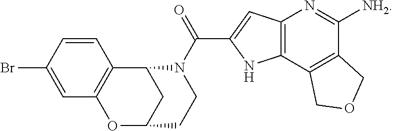 [SMILES: Nc1nc2cc(C(=O)N3CC[C@H]4C[C@@H]3c3ccc(Br)cc3O4)[nH]c2c2c1COC2]
Example 40

40. The compound of claim 25, or a hydrate thereof, or a solvate thereof, or a pharmaceutically acceptable salt thereof, wherein the compound is 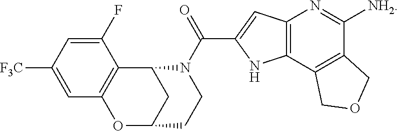 [SMILES: Nc1nc2cc(C(=O)N3CC[C@H]4C[C@@H]3c3c(F)cc(C(F)(F)F)cc3O4)[nH]c2c2c1COC2]
Example 41

41. The compound of claim 25, or a hydrate thereof, or a solvate thereof, or a pharmaceutically acceptable salt thereof, wherein the compound is 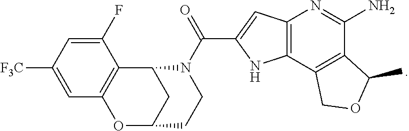 [SMILES: C[C@H]1OCc2c1c(N)nc1cc(C(=O)N3CC[C@H]4C[C@@H]3c3c(F)cc(C(F)(F)F)cc3O4)[nH]c21]
Example 43

42. The compound of claim 25, or a hydrate thereof, or a solvate thereof, or a pharmaceutically acceptable salt thereof, wherein the compound is 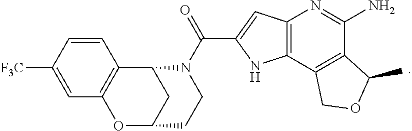 [SMILES: C[C@H]1OCc2c1c(N)nc1cc(C(=O)N3CC[C@H]4C[C@@H]3c3ccc(C(F)(F)F)cc3O4)[nH]c21]
Example 45

43. The compound of claim 25, or a hydrate thereof, or a solvate thereof, or a pharmaceutically acceptable salt thereof, wherein the compound is 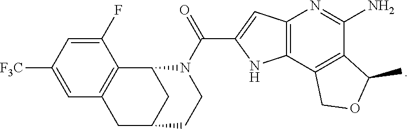 [SMILES: C[C@H]1OCc2c1c(N)nc1cc(C(=O)N3CC[C@@H]4Cc5cc(C(F)(F)F)cc(F)c5[C@H]3C4)[nH]c21]
Example 48

44. The compound of claim 25, or a hydrate thereof, or a solvate thereof, or a pharmaceutically acceptable salt thereof, wherein the compound is 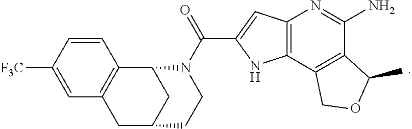 [SMILES: C[C@H]1OCc2c1c(N)nc1cc(C(=O)N3CC[C@@H]4Cc5cc(C(F)(F)F)ccc5[C@H]3C4)[nH]c21]
Example 50

45. The compound of claim 25, or a hydrate thereof, or a solvate thereof, or a pharmaceutically acceptable salt thereof, wherein the compound is 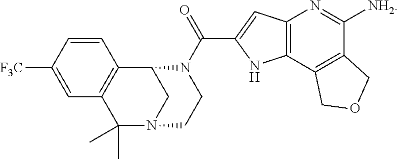 [SMILES: CC1(C)c2cc(C(F)(F)F)ccc2[C@H]2C[N@]1CCN2C(=O)c1cc2nc(N)c3c(c2[nH]1)COC3]
Example 57

46. The compound of claim 25, or a hydrate thereof, or a solvate thereof, or a pharmaceutically acceptable salt thereof, wherein the compound is 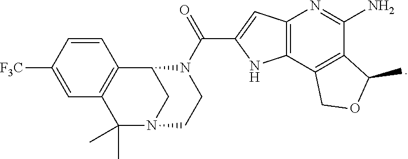 [SMILES: C[C@H]1OCc2c1c(N)nc1cc(C(=O)N3CC[N@@]4C[C@@H]3c3ccc(C(F)(F)F)cc3C4(C)C)[nH]c21]
Example 59

47. The compound of claim 25, or a hydrate thereof, or a solvate thereof, or a pharmaceutically acceptable salt thereof, wherein the compound is 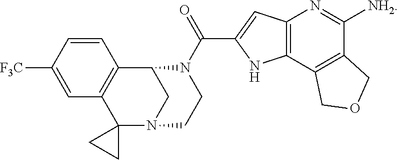 [SMILES: Nc1nc2cc(C(=O)N3CC[N@@]4C[C@@H]3c3ccc(C(F)(F)F)cc3C43CC3)[nH]c2c2c1COC2]
Example 63

48. The compound of claim 25, or a hydrate thereof, or a solvate thereof, or a pharmaceutically acceptable salt thereof, wherein the compound is 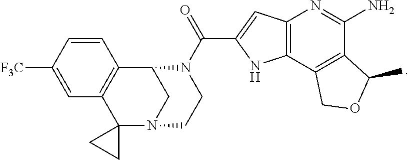 [SMILES: C[C@H]1OCc2c1c(N)nc1cc(C(=O)N3CC[N@@]4C[C@@H]3c3ccc(C(F)(F)F)cc3C43CC3)[nH]c21]
Example 79

49. The compound of claim 25, or a hydrate thereof, or a solvate thereof, or a pharmaceutically acceptable salt thereof, wherein the compound is 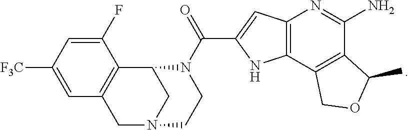 [SMILES: C[C@H]1OCc2c1c(N)nc1cc(C(=O)N3CC[N@]4Cc5cc(C(F)(F)F)cc(F)c5[C@H]3C4)[nH]c21]
Example 80
